SEQUENCE LISTING (258 sequences)

SEQ ID NO: 1 - DNA, 18 bp, Artificial Sequence
  Primer C375M
ctgaacatcccggccgac

SEQ ID NO: 2 - DNA, 19 bp, Artificial Sequence
  Primer A1-350M
agaaagcagcgatggagga

SEQ ID NO: 3 - DNA, 21 bp, Artificial Sequence
  Primer S1-34M
gcccaccagttcattaaggct

SEQ ID NO: 4 - DNA, 20 bp, Artificial Sequence
  Primer A2-320M
tcattaatggagcgtgggtg

SEQ ID NO: 5 - DNA, 20 bp, Artificial Sequence
  Primer S2-55M
cctggcatcactactgctat

SEQ ID NO: 6 - DNA, 18 bp, Artificial Sequence
  Primer C375
ctgaacatcacgcccaac

SEQ ID NO: 7 - DNA, 19 bp, Artificial Sequence
  Primer A1-350
aggaagcagcggtggacca

SEQ ID NO: 8 - DNA, 20 bp, Artificial Sequence
  Primer S1-34
gcccatcagtttattaaggc

SEQ ID NO: 9 - DNA, 20 bp, Artificial Sequence
  Primer A2-320
tcatttattgagcggggatg

SEQ ID NO: 10 - DNA, 20 bp, Artificial Sequence
  Primer S2-55
cctggcatcactactgctat

SEQ ID NO: 11 - DNA, 25 bp, Artificial Sequence
  Primer M1PR6
ccatgttccacaccgtattccagag

SEQ ID NO: 12 - DNA, 25 bp, Artificial Sequence
  Primer S4294M
gtgttctacggggatgcttatgacg

SEQ ID NO: 13 - DNA, 25 bp, Artificial Sequence
  Primer M1PF6
gactcagtattctctgctgccgtgg

SEQ ID NO: 14 - DNA, 25 bp, Artificial Sequence
  Primer A4556
ggctcaccagaatgcttcttccaga

SEQ ID NO: 15 - DNA, 342 bp, Hepatitis E Virus
  Clone USP-15
gcccatcagtttattaaggctcctggcattactactgccattgagcaggctgctctggctgcggccaattctgccttggcgaatgctgtggtggttcggccgtttttatctcgcgtgcaaaccgagattcttattaatttgatgcaaccccggcagttggttttccgccctgaggtactttggaatcaccctatccagcgggttatacataatgaattagaacagtactgccgggctcgggctggtcgttgcttggaggttggagctcacccaagatccattaatgacaaccccaacgttctgcatcggtgtttccttagaccggtcgggcgtgatgttcag

SEQ ID NO: 16 - DNA, 20 bp, Artificial Sequence
  Primer PA2-5560
taggttatactgccggcgca

SEQ ID NO: 17 - DNA, 20 bp, Artificial Sequence
  Primer S1-5287
ttctcagcccttcgcaatcc

SEQ ID NO: 18 - DNA, 20 bp, Artificial Sequence
  Primer S2-5310
atattcatccaaccaacccc

SEQ ID NO: 19 - DNA, 251 bp, Hepatitis E Virus
  Clone  b421
atattcatccaaccaaccccttcgccgccgatgtcgtttcacaacccggggctggaactcgccctcgacagccgccccgccccctcggttccgcttggcgtgaccagtcccagcgcccctccgttgccccccgtcgtcgatctaccccagctggggctgcgccgctaactgccatatcaccagcccctgatacagctcctgtacctgatgttgactcacgtggtgctattttgcgccggcagtataaccta

SEQ ID NO: 20 - DNA, 20 bp, Artificial Sequence
  Primer US4.2-69S/20
ttccgcttggcgtgaccagt

SEQ ID NO: 21 - DNA, 21 bp, Artificial Sequence
  Primer US4.4/144s
gctaactgccatatcaccagc

SEQ ID NO: 22 - DNA, 20 bp, Artificial Sequence
  Primer M6417a
cccttatcctgctgagcatt

SEQ ID NO: 23 - DNA, 24 bp, Artificial Sequence
  Primer M6371a
ttggctcgccattggctgagacaa

SEQ ID NO: 24 - DNA, 899 bp, Hepatitis E Virus
  Clone  df-orf2/3
gctaactgccatatcaccagcccctgatacagctcctgtacctgatgttgactcacgtggtgctattttgcgccggcagtacaatttgtctacgtccccgcttacatcatctgttgcttctggtactaatctggttctctatgctgccccgctgaaccctctcttgcctcttcaggatggcaccaacactcatattatggctactgaggcatctaattacgcccagtatcgggttgttcgggctacgattcgttatcgcccgttggtgccaaatgctgttggtggttatgctatctctatttctttctggcctcaaactacaactacccctacttctgttgacatgaattctatcacttctactgatgtcaggatcttggtccagcccggtatagcctccgagttagtcatccctagtgaacgccttcactaccgcaaccaaggctggcgctctgttgagaccacgggtgtggccgaagaggaggctacctccggtctggtaatgctttgtattcatggctcccctgttaactcctacactaatacaccttacaccggtgcattggggcttcttgattttgcattagaacttgaatttagaaatttgacacccgggaacactaacacccgtgtttcccggtatactagcacagcccgccaccggctgcgccgcggtgctgatgggaccgctgagctcaccaccacagcagccacacgcttcatgaaggatttgcattttactggtacgaacggcgttggtgaggtgggtcgtggtattgccctgactctgtttaatcttgctgatacgcttcttggtggtttaccgacagaattgatttcgtcggctgggggtcaactgttttactcccgccctgttgtctcagccaatggcgagccaa

SEQ ID NO: 25 - DNA, 20 bp, Artificial Sequence
  Primer USP 3s/20
tggcattactactgccattg

SEQ ID NO: 26 - DNA, 20 bp, Artificial Sequence
  Primer M902A
atcgatcggacatagacctc

SEQ ID NO: 27 - DNA, 846 bp, Hepatitis E Virus
  Clone  df-orf1
tggcattactactgccattgagcaggctgctctggctgcggccaattctgccttggcgaatgctgtggtggttcggccgtttttatctcgcgtgcaaaccgagattcttattaatttgatgcaaccccggcagttggttttccgccctgaggtactttggaatcaccctatccagcgggttatacataatgaattagaacagtactgccgggctcgggctggtcgttgcttggaggttggagctcacccaagatccattaatgacaaccccaacgttctgcatcggtgtttccttagaccggttggccgagatgttcagcgctggtactctgcccccacccgcggccctgcggctaattgccgccgctccgcgttgcgtggtctcccccccgctgaccgcacttactgctttgatggattctcccgttgtgcttttgctgcagagaccggtgtggctctttactctctgcatgacctttggccagctgatgttgcagaggctatggcccgccacgggatracacgcttgtatgccgcactgcaccttccccctgaggtgctgctaccacccggcacctaccacacaacctcgtatctcctgattcacgacggcgaccgcgctgttgtaacttacgagggcgatactagtgcgggctataatcatgatgtctccatacttcgtgcgtggatccgtactacaaaaatagttggtgatcatccgttggtcatagagcgtgtgcgggccattggatgtcattttgtgttgctgctcaccgcagcccctgagccgtcacccatgccttatgttccttaccctcgttcaacggaggtctatgtccgatcgat

SEQ ID NO: 28 - DNA, 23 bp, Artificial Sequence
  Primer 3750s
cttccatcagttggctgaggagc

SEQ ID NO: 29 - DNA, 23 bp, Artificial Sequence
  Primer 3900a
gccatgcggcagtgcacaatgtc

SEQ ID NO: 30 - DNA, 168 bp, Hepatitis E Virus
  Clone  HEV 167
cttccatcagttggctgaggagctgggccatcgcccggcccctgtcgccgccgtcttgcccccttgccctgagcttgagcagggcctgctctacatgccacaggagctcactgtgtccgatagtgtgttggtttttgagcttacggacattgtgcactgccgcatggc

SEQ ID NO: 31 - DNA, 25 bp, Artificial Sequence
  Primer 5000s
ctcgttcataacctgattggcatgc

SEQ ID NO: 32 - DNA, 23 bp, Artificial Sequence
  Primer uf-orf2/3 a3
ggactggtcacgccaagcggaac

SEQ ID NO: 33 - DNA, 424 bp, Hepatitis E Virus
  Clone  HEV 426
ctcgttcataacctgattggcatgctgcagaccatcgccgatggcaaggcccactttacagagactattaaacctgtacttgatctcacaaattccatcatacagcgggtggaatgaataacatgtcttttgcatcgcccatgggatcaccatgcgccctagggctgttctgttgttgttcctcatgtttctgcctatgctgcccgcgccaccggccggtcagccgtctggccgtcgccgtgggcggcgcagcggcggtgccggcggtggtttctggagtgacagggttgattctcagcccttcgccctcccctatattcatccaaccaaccccttcgccgccgatgtcgtttcacaacccggggctggaactcgccctcgacagccgccccgccccctcggttccgcttggcgtgaccagtcc

SEQ ID NO: 34 - DNA, 24 bp, Artificial Sequence
  Primer 167-sl
tctacatgccacaggagctcactg

SEQ ID NO: 35 - DNA, 27 bp, Artificial Sequence
  Primer 426-a3
gatggaatttgtgagatcaagtacagg

SEQ ID NO: 36 - DNA, 25 bp, Artificial Sequence
  Primer 167-s2
ctcactgtgtccgatagtgtgttgg

SEQ ID NO: 37 - DNA, 23 bp, Artificial Sequence
  Primer 426-a4
ccttgccatcggcgatggtctgc

SEQ ID NO: 38 - DNA, 1186 bp, Hepatitis E Virus
  Clone  HEV 1186
ctcactgtgtccgatagtgtgttggtttttgagcttacggatatagttcattgccgcatggccgctccaagccagcgaaaggctgttctctcaacacttgtggggaggtatggccgtaggacgaaactatatgaggcggcgcattcagatgttcgtgagtccctagctaggttcatccctactatcgggcctgttcaggctaccacatgtgagttgtatgagttggttgaggctatggtggagaaaggtcaggacggctctgcagtcttagagcttgatctttgtaatcgtgatgtctcgcgcatcacatttttccaaaaagwctgcaacaagtttacaactggtgagaccatcgcccacggcaaggttggccagggtatatcggcctggagtaagaccttctgcgctctgttcggcccgtggttccgcgccattgaaaaagaaatattggccctgctcccgcctaatatcttttatggcgacgcttatgaggagtcagtttttgccgccgctgtgtccggggcggggtcatgtatggtatttgaaaatgacttttcagagtttgacagtacccagaataatttctctcttggccttgagtgtgtggttatggaggagtgcggcatgcctcaatggctaattaggttgtaccatctggttcggtctgcctggattctgcaggcgccgaaggagtctcttaagggtttctggaagaagcattctggtgagcctggtacccttctttggaataccgtctggaatatggcgattatagcacattgctatgagttccgtgactttcgtgttgctgcctttaagggtgatgattcggtggtcctctgtagtgactaccgacagagccgcaatgcagctgccttaattgctggctgtgggctcaaattgaaggttgattaccgccctatcgggctgtatgctggggtggtggtggcccccggtttggggacactgcccgatgtggtgcgttttgctggtcggttgtctgaaaagaattggggccccggcccggaacgtgctgagcagctgcgtcttgctgtctgcgacttccttcgagggttgacgaatgttgcgcaggtctgtgttgatgttgtgtcccgtgtctatggagtcagccccgggctcgtacataaccttattggcatgctgcagaccatcgccgatggcaagg

SEQ ID NO: 39 - DNA, 25 bp, Artificial Sequence
  Primer orf1-s2
tcacccatgccttatgttccttacc

SEQ ID NO: 40 - DNA, 22 bp, Artificial Sequence
  Primer 1300a
ggcggcctgggatgtaatcacg

SEQ ID NO: 41 - DNA, 460 bp, Hepatitis E Virus
  Clone  HEV 459
tcacccatgccttatgttccttaccctcgttcaacggaggtgtatgtccggtccatatttggccctggcggctccccatccttgtttccgtcagcctgctctactaaatctactttccatgctgtcccggtgcatatctgggatcggctcatgctctttggtgccaccctggacgatcaggcgttttgctgttcacggctcatgacttacctccgtggtattagttacaaggtcactgtcggcgcgcttgtcgctaatgaggggtggaacgcctctgaagacgctcttactgcartgatcactgcagcttatttgactatttgccatcagcgttatctccgcacccaggcgatatccaagggcatgcgccggttgggggttgagcacgcccagaaatttatcacaagactctacagttggctatttgagaagtctggccgtgattacatcccaggccgcc

SEQ ID NO: 42 - DNA, 26 bp, Artificial Sequence
  Primer 459-s2
cagaaatttatcacaagactctacag

SEQ ID NO: 43 - DNA, 23 bp, Artificial Sequence
  Primer 1450a
aacactcctgaccgagccacttc

SEQ ID NO: 44 - DNA, 235 bp, Hepatitis E Virus
  Clone  HEV 216
cagaaatttatcacaagactctacagttggctatttgagaagtctggccgtgattatatccccggccgccagcttcagttctatgcacagtgccgacggtggctatctgcaggcttccacctagaccccagggtacttgtttttgatgagtcagtaccatgccgctgtaggacgtttttgaagaaagttgcgggtaaattctgctgttttatgaagtggctcggtcaggagtgtt

SEQ ID NO: 45 - DNA, 26 bp, Hepatitis E Virus
  us1 gap-sl
tatagatataacaggttcacccagcg

SEQ ID NO: 46 - DNA, 24 bp, Hepatitis E Virus
  usl gap-a0.5
gctgcaagaccctcacgcatgatg

SEQ ID NO: 47 - DNA, 23 bp, Hepatitis E Virus
  usl gap-s2
cggattatggttacaccctgagg

SEQ ID NO: 48 - DNA, 25 bp, Hepatitis E Virus
  us1 gap-a1
attcagttgggtaaaacgcttctgg

SEQ ID NO: 49 - DNA, 545 bp, Hepatitis E Virus
  us1-gap
cggattatggttacaccctgaggggttgctgggtattttcccccctttctcccctgggcatatctgggagtctgcgaaccccttttgcggggaggggactttgtatacccgaacttggtcaacatctggcttttctagtgatttctccccccctgaagcggccgctcctgctatggctgctaccccggggctgccccattctaccccacctgttagcgatatttgggtgctaccaccgccctcagaggagtttcaggttgatgcagcacctgtgccccctgcccctgaccctgctggattgcccggtcccgttgtgcttaccccccccccccctccccctgtgcataagccatcaatacccccgccttcccgtaaccgtcgtctcctctatacctatcctgacggcgctaaggtgtatgcagggtcactgtttgaatcagactgtgactggctggttaatgcctcaaacccgggccatcgtcccggaggtggcctctgccatgccttttaccaacgttttccagaagcgttttacccaactgaat

SEQ ID NO: 50 - DNA, 24 bp, Hepatitis E Virus
  us1-2600s
gtgctcaccataactgaggacacg

SEQ ID NO: 51 - DNA, 24 bp, Hepatitis E Virus
  us1-2600a
cgctgcatatgtaacagcaacagg

SEQ ID NO: 52 - DNA, 344 bp, Hepatitis E Virus
  us1-344
gtgctcaccataactgaggacacggcccgtacggccaacctggcattggagattgatgccgctacagaggtcggccgtgcttgtgccggttgcaccatcagccctggcattgtgcactatcagtttaccgccggggtcccgggctcgggcaagtcaaggtccatacaacagggagatgtcgatgtggtggttgtgcccacccgggagcttcgtaatagttggcgccgccggggttttgcggccttcacaccccacacagcggcccgtgttactatcggccgccgcgttgtgattgatgaggctccatctctcccgccacacctgttgctgttacatatgcagcg

SEQ ID NO: 53 - DNA, 23 bp, Hepatitis E Virus
  us1 3200s
gccgatgtgtgcgagctcatacg

SEQ ID NO: 54 - DNA, 25 bp, Hepatitis E Virus
  us1 3200a
atgattgtggtctctgtgaaggtgg

SEQ ID NO: 55 - DNA, 194 bp, Hepatitis E Virus
  us1-194
gccgatgtgtgcgagctcatacgcggagcctaccctaaaatccagaccacgagccgtgtgctacggtccctgttttggaatgaaccggccattggccagaagttggttytcacgcaggcggcaaaggctgctaaccctggtgcgattacggtccacgaagctcagggtgccaccttcacagagaccacaatcat

SEQ ID NO: 56 - DNA, 23 bp, Hepatitis E Virus
  HEV216-s1
cagtaccatgccgctgtaggacg

SEQ ID NO: 57 - DNA, 26 bp, Hepatitis E Virus
  us2-733a1
ccattagatgaaatctttacctgcag

SEQ ID NO: 58 - DNA, 26 bp, Hepatitis E Virus
  HEV216-s2
gtaggacgtttttgaagaaagttgcg

SEQ ID NO: 59 - DNA, 24 bp, Hepatitis E Virus
  us2-733a2
ggtgagctcataagtgaggctgtg

SEQ ID NO: 60 - DNA, 464 bp, Hepatitis E Virus
  us1-733wb
gtaggacgtttttgaagaaagttgcgggtaaattctgctgttttatgcggtggctcgggcaggagtgtacctgcttcttggagccggccgagggtttagtcggcgatcatggccatgacaacgaggcctatgagggttctgaggtcgacccggctgaacctgcacatcttgatgtttctgggacttacgccgtccacgggcaccagcttgaggccctctatagggcacttaatgtcccacaagatattgccgctcgagcttcccgactaacggcaactgttgagctcgttgcaagtccagaccgcttagagtgccgcaccgtgctcggtaataagaccttccggacgacggtggtcgacggcgcccatctagaggcgaatggccctgagcagtatgtcttatcatttgacgcctcccgtcagtctatgggggccgggtcgcacagcctcacttatgagctcacc

SEQ ID NO: 61 - DNA, 24 bp, Hepatitis E Virus
  us1 733s1
ttgagctcgttgcaagtccagacc

SEQ ID NO: 62 - DNA, 22 bp, Hepatitis E Virus
  us2851-r2
ccagaggttgaccaggttcggg

SEQ ID NO: 63 - DNA, 24 bp, Hepatitis E Virus
  us1 733s2
ccgtgctcggtaataagaccttcc

SEQ ID NO: 64 - DNA, 433 bp, Hepatitis E Virus
  us1-432
ccgtgctcggtaataagaccttccggacgacggtggtcgacggcgcccatctagaggcgaatggccctgagcagtatgtcttatcatttgacgcctcccgtcagtctatgggggccgggtcgcatagcctcacttatgagctcacccctgctggtttgcaggttaggatttcatctaatggtctggattgcactgctacattcccccccggtggagcccctagcgctgcgcccggggaggtggcagccttttgcagtgccctttatagatataacaggttcacccagcggcactcgctgactggcggattatggttacaccctgaggggttgctgggtattttcccccctttctcccctgggcatatctgggagtctgcgaaccccttttgcggggaggggactttgtatacccgaacctggtcaacctctgg

SEQ ID NO: 65 - DNA, 26 bp, Hepatitis E Virus
  us2851-f1
gactgtgattggttagtcaatgcctc

SEQ ID NO: 66 - DNA, 24 bp, Hepatitis E Virus
  us1 430-a1
cgtgtcctcagttatggtgagcac

SEQ ID NO: 67 - DNA, 26 bp, Hepatitis E Virus
  us1 430-a2
tattagcctcaaaccaatttgcagcg

SEQ ID NO: 68 - DNA, 382 bp, Hepatitis E Virus
  us1-382
gactgtgattggttagtcaatgcctcaaacccgggccatcgtcccggaggtggcctctgccatgccttttaccaacgttttccagaagcgttttacccaactgaattcatcatgcgtgagggtcttgcagcatacaccttgaccccgcgccctatcattcatgcagtcgctcccgattatagggttgagcagaacccgaagaggcttgaggcagcgtaccgtgaaacttgttcccgtcgtggcaccgctgcctacccgcttttgggttcgggtatataccaggtccctgttagcctcagttttgatgcctgggaacgtaatcaccgccccggcgatgagctttacttgaccgagcccgctgcaaattggtttgaggctaata

SEQ ID NO: 69 - DNA, 22 bp, Hepatitis E Virus
  us2-579-s1
cagaccacgagccgtgtgctac

SEQ ID NO: 70 - DNA, 25 bp, Hepatitis E Virus
  JE hev167-a1
ccaacacactatcggacacagtgag

SEQ ID NO: 71 - DNA, 22 bp, Hepatitis E Virus
  us2-579-s2
gctgctaaggctgccaaccctg

SEQ ID NO: 72 - DNA, 24 bp, Hepatitis E Virus
  JE hev167-a2
cagtgagctcctgtggcatgtaga

SEQ ID NO: 73 - DNA, 451 bp, Hepatitis E Virus
  us1-579wb
gctgctaaggctgccaaccctggtgcgattacggtccacgaagctcagggtgccaccttcacagagaccacaatcatagccacggccgacgccaggggccttatccagtcatcccgggctcatgctatagttgcacttactcgccacactgagaagtgtgttatcctggatgcccccggcctgcttcgtgaggtcggcatttcggatgtgattgtcaacaactttttccttgctggtggcgaggtcggccrccaccgcccttctgtgatacctcgcggtaaccctgatcaaaacctcgggactttacaggccttcccgccgtcctgtcaaattagtgcttaccatcagttggctgaggaactgggccatcgcccggcccctgtcgccgccgtcttgcccccttgccctgagcttgagcagggcctgctctacatgccacaggagctcactg

SEQ ID NO: 74 - DNA, 24 bp, Hepatitis E Virus
  us2-430s1
ggtatataccaggtccctgttagc

SEQ ID NO: 75 - DNA, 22 bp, Hepatitis E Virus
  us2-482-a1
ccgctgtgtgaggtgtgaaggc

SEQ ID NO: 76 - DNA, 24 bp, Hepatitis E Virus
  us2-430s2
gttagcctcagttttgatgcctgg

SEQ ID NO: 77 - DNA, 23 bp, Hepatitis E Virus
  us2-482-a2
gacgccagctgttacggagctcc

SEQ ID NO: 78 - DNA, 334 bp, Hepatitis E Virus
  us1-430wb
gttagcctcagttttgatgcctgggaacgtaatcaccgccccggcgatgagctttacttgaccgagcccgctgcaaattggtttgaggctaataagccggcgcagccggtgctcaccataactgaggacacggcccgtacggccaacctggcattggagattgatgccgctacagaggtcggccgtgcttgtgccggttgcaccatcagccctggcattgtgcactatcagtttaccgccggggtcccgggctcgggcaagtcaaggtccatacaacagggagatgtcgatgtggtggttgtgcccacccgggagctccgtaacagctggcgtc

SEQ ID NO: 79 - DNA, 23 bp, Hepatitis E Virus
  us2-482-s1
gatgtcgatgtggtggttgtgcc

SEQ ID NO: 80 - DNA, 23 bp, Hepatitis E Virus
  JE us2-579-a1
gtaatcgcaccagggttggcagc

SEQ ID NO: 81 - DNA, 23 bp, Hepatitis E Virus
  us2-482-s2
ggagctccgtaacagctggcgtc

SEQ ID NO: 82 - DNA, 22 bp, Hepatitis E Virus
  JE us2-579-a2
cagggttggcagccttagcagc

SEQ ID NO: 83 - DNA, 413 bp, Hepatitis E Virus
  us1-482wb
ggagctccgtaacagctggcgtcgccggggttttgcggccttcacaccccacacagcggcccgtgttactatcggccgccgcgttgtgattgatgaggctccatctctcccgccacacctgttgctgttacatatgcagcgggcctcctcggtccatctcctcggtgacccaaatcagatccctgctattgattttgagcacgccggcctggtccctgcgatccgtcccgagcttgcgccaacgagctggtggcrcgttacacaccgttgcccggccgatgtgtgcgagctcatacgcggagcctaccctaaaatccagaccacgagccgtgtgctacggtccctgttttggaatgaaccggccattggccagaagttggttytcacgcaggctgctaaggctgccaaccctg

SEQ ID NO: 84 - DNA, 37 bp, Artificial Sequence
  Oligo dT Adapter Primer
ggccacgcgtcgactagtacttttttttttttttttt

SEQ ID NO: 85 - DNA, 20 bp, Artificial Sequence
  AUAP Primer
ggccacgcgtcgactagtac

SEQ ID NO: 86 - DNA, 22 bp, Artificial Sequence
  Primer df-orf3-s1
gcgttggtgaggtgggtcgtgg

SEQ ID NO: 87 - DNA, 24 bp, Artificial Sequence
  Primer df-orf3-s2
cgcttcttggtggtttaccgacag

SEQ ID NO: 88 - DNA, 960 bp, Hepatitis E Virus
  Clone  HEV 3p RACE
cgcttcttggtggtttaccgacagaattgatttcgtcggctgggggtcaactgttttactcccgccctgttgtctcggccaatggcgagccaacagtaaagttatacacatctgttgagaatgcgcagcaagacaagggcatcaccattccacacgacatagatttaggtgactcccgtgtggttatccaggattatgataaccagcacgaacaagatcgacctaccccgtcacctgccccctcccgccctttctcagttcttcgtgccaatgatgttttgtggctctctctcactgccgctgagtacgrccagaccacgtatgggtcgtccaccaaccctatgtatgtctctgatacagtcacgcttgttaatgtagccactggtgctcaggctgttgcccgctctcttgactggtctaaagttactctggatggtcgccctcttactaccattcagcagtattctaagaaattttatgttctcccgcttcgsgggaagctgtccttttgggaggctggtacgaccaaggccggctacccgtataattataataccactgctagtgaccaaattttgattgagaacgcggccggtcaccgtgtcgccatttctacttataccactagtttgggtgccggccctacctcgatytctgcggtcggtgtactagctccacattcggcccttgctgttctcgaggatactgttgattatcctgctcgtgcccatacttttgatgatttctgcccggagtgtcgcacccttggtctgcagggttgtgcattccaatctactattgctgaacttcagcgtcttaaaatgaaggtaggtaaaacccgggagtcttaattaattccttttgtgcccccttcgcagttctctttggctttatttctcatttctgctttccgcgctnccctggaaaaaaaaaaaaaaaagtactagtcgacgcgtggcc

SEQ ID NO: 89 - DNA, 7202 bp, Hepatitis E Virus
  us1fu11
cctggcattactactgccattgagcaggctgctctggctgcggccaattctgccttggcgaatgctgtggtggttcggccgtttttatctcgcgtgcaaaccgagattcttattaatttgatgcaaccccggcagttggttttccgccctgaggtactttggaatcaccctatccagcgggttatacataatgaattagaacagtactgccgggctcgggctggtcgttgcttggaggttggagctcacccaagatccattaatgacaaccccaacgttctgcatcggtgtttccttagaccggttggccgagatgttcagcgctggtactctgcccccacccgcggccctgcggctaattgccgccgctccgcgttgcgtggtctcccccccgctgaccgcacttactgctttgatggattctcccgttgtgcttttgctgcagagaccggtgtggctctttactctctgcatgacctttggccagctgatgttgcagaggctatggcccgccacgggatracacgcttgtatgccgcactgcaccttccccctgaggtgctgctaccacccggcacctaccacacaacctcgtatctcctgattcacgacggcgaccgcgctgttgtaacttacgagggcgatactagtgcgggctataatcatgatgtctccatacttcgtgcgtggatccgtactacaaaaatagttggtgatcatccgttggtcatagagcgtgtgcgggccattggatgtcattttgtgttgctgctcaccgcagcccctgagccgtcacccatgccttatgttccttaccctcgttcaacggaggtgtatgtccggtccatatttggccctggcggctccccatccttgtttccgtcagcctgctctactaaatctactttccatgctgtcccggtgcatatctgggatcggctcatgctctttggtgccaccctggacgatcaggcgttttgctgttcacggctcatgacttacctccgtggtattagttacaaggtcactgtcggcgcgcttgtcgctaatgaggggtggaacgcctctgaagacgctcttactgcartgatcactgcagcttatttgactatttgccatcagcgttatctccgcacccaggcgatatccaagggcatgcgccggttgggggttgagcacgcccagaaatttatcacaagactctacagttggctatttgagaagtctggccgtgattatatccccggccgccagcttcagttctatgcacagtgccgacggtggctatctgcaggcttccacctagaccccagggtacttgtttttgatgagtcagtaccatgccgctgtaggacgtttttgaagaaagttgcgggtaaattctgctgttttatgcggtggctcgggcaggagtgtacctgcttcttggagccggccgagggtttagtcggcgatcatggccatgacaacgaggcctatgagggttctgaggtcgacccggctgaacctgcacatcttgatgtttctgggacttacgccgtccacgggcaccagcttgaggccctctatagggcacttaatgtcccacaagatattgccgctcgagcttcccgactaacggcaactgttgagctcgttgcaagtccagaccgcttagagtgccgcaccgtgctcggtaataagaccttccggacgacggtggtcgacggcgcccatctagaggcgaatggccctgagcagtatgtcttatcatttgacgcctcccgtcagtctatgggggccgggtcgcatagcctcacttatgagctcacccctgctggtttgcaggttaggatttcatctaatggtctggattgcactgctacattcccccccggtggagcccctagcgctgcgcccggggaggtggcagccttttgcagtgccctttatagatataacaggttcacccagcggcactcgctgactggcggattatggttacaccctgaggggttgctgggtattttcccccctttctcccctgggcatatctgggagtctgcgaaccccttttgcggggaggggactttgtatacccgaacttggtcaacatctggcttttctagtgatttctccccccctgaagcggccgctcctgctatggctgctaccccggggctgccccattctaccccacctgttagcgatatttgggtgctaccaccgccctcagaggagtttcaggttgatgcagcacctgtgccccctgcccctgaccctgctggattgcccggtcccgttgtgcttaccccccccccccctccccctgtgcataagccatcaatacccccgccttcccgtaaccgtcgtctcctctatacctatcctgacggcgctaaggtgtatgcagggtcactgtttgaatcagactgtgactggctggttaatgcctcaaacccgggccatcgtcccggaggtggcctctgccatgccttttaccaacgttttccagaagcgttttacccaactgaattcatcatgcgtgagggtcttgcagcatacaccttgaccccgcgccctatcattcatgcagtcgctcccgattatagggttgagcagaacccgaagaggcttgaggcagcgtaccgtgaaacttgttcccgtcgtggcaccgctgcctacccgcttttgggttcgggtatataccaggtccctgttagcctcagttttgatgcctgggaacgtaatcaccgccccggcgatgagctttacttgaccgagcccgctgcaaattggtttgaggctaataagccggcgcagccggtgctcaccataactgaggacacggcccgtacggccaacctggcattggagattgatgccgctacagaggtcggccgtgcttgtgccggttgcaccatcagccctggcattgtgcactatcagtttaccgccggggtcccgggctcgggcaagtcaaggtccatacaacagggagatgtcgatgtggtggttgtgcccacccgggagcttcgtaatagttggcgccgccggggttttgcggccttcacaccccacacagcggcccgtgttactatcggccgccgcgttgtgattgatgaggctccatctctcccgccacacctgttgctgttacatatgcagcgggcctcctcggtccatctcctcggtgacccaaatcagatccctgctattgattttgagcacgccggcctggtccctgcgatccgtcccgagcttgcgccaacgagctggtggcrcgttacacaccgttgcccggccgatgtgtgcgagctcatacgcggagcctaccctaaaatccagaccacgagccgtgtgctacggtccctgttttggaatgaaccggccattggccagaagttggttytcacgcaggcggcaaaggctgctaaccctggtgcgattacggtccacgaagctcagggtgccaccttcacagagaccacaatcatagccacggccgacgccaggggccttatccagtcatcccgggctcatgctatagttgcacttactcgccacactgagaagtgtgttatcctggatgcccccggcctgcttcgtgaggtcggcatttcggatgtgattgtcaacaactttttccttgctggtggcgaggtcggccrccaccgcccttctgtgatacctcgcggtaaccctgatcaaaacctcgggactttacaggccttcccgccgtcctgtcaaattagtgcttaccatcagttggctgaggaactgggccatcgcccggcccctgtcgccgccgtcttgcccccttgccctgagcttgagcagggcctgctctacatgccacaggagctcactgtgtccgatagtgtgttggtttttgagcttacggatatagttcattgccgcatggccgctccaagccagcgaaaggctgttctctcaacacttgtggggaggtatggccgtaggacgaaactatatgaggcggcgcattcagatgttcgtgagtccctagctaggttcatccctactatcgggcctgttcaggctaccacatgtgagttgtatgagttggttgaggctatggtggagaaaggtcaggacggctctgcagtcttagagcttgatctttgtaatcgtgatgtctcgcgcatcacatttttccaaaaagwctgcaacaagtttacaactggtgagaccatcgcccacggcaaggttggccagggtatatcggcctggagtaagaccttctgcgctctgttcggcccgtggttccgcgccattgaaaaagaaatattggccctgctcccgcctaatatcttttatggcgacgcttatgaggagtcagtttttgccgccgctgtgtccggggcggggtcatgtatggtatttgaaaatgacttttcagagtttgacagtacccagaataatttctctcttggccttgagtgtgtggttatggaggagtgcggcatgcctcaatggctaattaggttgtaccatctggttcggtctgcctggattctgcaggcgccgaaggagtctcttaagggtttctggaagaagcattctggtgagcctggtacccttctttggaataccgtctggaatatggcgattatagcacattgctatgagttccgtgactttcgtgttgctgcctttaagggtgatgattcggtggtcctctgtagtgactaccgacagagccgcaatgcagctgccttaattgctggctgtgggctcaaattgaaggttgattaccgccctatcgggctgtatgctggggtggtggtggcccccggtttggggacactgcccgatgtggtgcgttttgctggtcggttgtctgaaaagaattggggccccggcccggaacgtgctgagcagctgcgtcttgctgtctgcgacttccttcgagggttgacgaatgttgcgcaggtctgtgttgatgttgtgtcccgtgtctatggagtcagccccgggctcgtacataaccttattggcatgctgcagaccatcgccgatggcaaggcccactttacagagactattaaacctgtacttgatctcacaaattccatcatacagcgggtggaatgaataacatgtcttttgcatcgcccatgggatcaccatgcgccctagggctgttctgttgttgttcctcatgtttctgcctatgctgcccgcgccaccggccggtcagccgtctggccgtcgccgtgggcggcgcagcggcggtgccggcggtggtttctggagtgacagggttgattctcagcccttcgccctcccctatattcatccaaccaaccccttcgccgccgatgtcgtttcacaacccggggctggaactcgccctcgacagccgccccgccccctcggttccgcttggcgtgaccagtccaagcgcccctccgttgccccccgtcgtcgatctaccccagctggggctgcgccgctaactgccatatcaccagcccctgatacagctcctgtacctgatgttgactcacgtggtgctattttgcgccggcagtacaatttgtctacgtccccgcttacatcatctgttgcttctggtactaatctggttctctatgctgccccgctgaaccctctcttgcctcttcaggatggcaccaacactcatattatggctactgaggcatctaattacgcccagtatcgggttgttcgggctacgattcgttatcgcccgttggtgccaaatgctgttggtggttatgctatctctatttctttctggcctcaaactacaactacccctacttctgttgacatgaattctatcacttctactgatgtcaggatcttggtccagcccggtatagcctccgagttagtcatccctagtgaacgccttcactaccgcaaccaaggctggcgctctgttgagaccacgggtgtggccgaagaggaggctacctccggtctggtaatgctttgtattcatggctcccctgttaactcctacactaatacaccttacaccggtgcattggggcttcttgattttgcattagaacttgaatttagaaatttgacacccgggaacactaacacccgtgtttcccggtatactagcacagcccgccaccggctgcgccgcggtgctgatgggaccgctgagctcaccaccacagcagccacacgcttcatgaaggatttgcattttactggtacgaacggcgttggtgaggtgggtcgtggtattgccctgactctgtttaatcttgctgatacgcttcttggtggtttaccgacagaattgatttcgtcggctgggggtcaactgttttactcccgccctgttgtctcggccaatggcgagccaacagtaaagttatacacatctgttgagaatgcgcagcaagacaagggcatcaccattccacacgacatagatttaggtgactcccgtgtggttatccaggattatgataaccagcacgaacaagatcgacctaccccgtcacctgccccctcccgccctttctcagttcttcgtgccaatgatgttttgtggctctctctcactgccgctgagtacgrccagaccacgtatgggtcgtccaccaaccctatgtatgtctctgatacagtcacgcttgttaatgtagccactggtgctcaggctgttgcccgctctcttgactggtctaaagttactctggatggtcgccctcttactaccattcagcagtattctaagaaattttatgttctcccgcttcgsgggaagctgtccttttgggaggctggtacgaccaaggccggctacccgtataattataataccactgctagtgaccaaattttgattgagaacgcggccggtcaccgtgtcgccatttctacttataccactagtttgggtgccggccctacctcgatytctgcggtcggtgtactagctccacattcggcccttgctgttctcgaggatactgttgattatcctgctcgtgcccatacttttgatgatttctgcccggagtgtcgcacccttggtctgcagggttgtgcattccaatctactattgctgaacttcagcgtcttaaaatgaaggtaggtaaaacccgggagtcttaattaattccttttgtgcccccttcgcagttctctttggctttatttctcatttctgctttccgcgctccctggaaaaaaaaaaaaaaaa

SEQ ID NO: 90 - DNA, 7202 bp, Hepatitis E Virus; identical to SEQ ID NO: 89
  us1fu11

SEQ ID NO: 91 - protein, 1698 aa, Hepatitis E Virus
  Xaa = Unknown or Other at position 174
PGITTAIEQAALAAANSALANAVVVRPFLSRVQTEILINLMQPRQLVFRPEVLWNHPIQRVIHNELEQYCRARAGRCLEVGAHPRSINDNPNVLHRCFLRPVGRDVQRWYSAPTRGPAANCRRSALRGLPPADRTYCFDGFSRCAFAAETGVALYSLHDLWPADVAEAMARHGXTRLYAALHLPPEVLLPPGTYHTTSYLLIHDGDRAVVTYEGDTSAGYNHDVSILRAWIRTTKIVGDHPLVIERVRAIGCHFVLLLTAAPEPSPMPYVPYPRSTEVYVRSIFGPGGSPSLFPSACSTKSTFHAVPVHIWDRLMLFGATLDDQAFCCSRLMTYLRGISYKVTVGALVANEGWNASEDALTAXITAAYLTICHQRYLRTQAISKGMRRLGVEHAQKFITRLYSWLFEKSGRDYIPGRQLQFYAQCRRWLSAGFHLDPRVLVFDESVPCRCRTFLKKVAGKFCCFMRWLGQECTCFLEPAEGLVGDHGHDNEAYEGSEVDPAEPAHLDVSGTYAVHGHQLEALYRALNVPQDIAARASRLTATVELVASPDRLECRTVLGNKTFRTTVVDGAHLEANGPEQYVLSFDASRQSMGAGSHSLTYELTPAGLQVRISSNGLDCTATFPPGGAPSAAPGEVAAFCSALYRYNRFTQRHSLTGGLWLHPEGLLGIFPPFSPGHIWESANPFCGEGTLYTRTWSTSGFSSDFSPPEAAAPAMAATPGLPHSTPPVSDIWVLPPPSEEFQVDAAPVPPAPDPAGLPGPVVLTPPPPPPVHKPSIPPPSRNRRLLYTYPDGAKVYAGSLFESDCDWLVNASNPGHRPGGGLCHAFYQRFPEAFYPTEFIMREGLAAYTLTPRPIIHAVAPDYRVEQNPKRLEAAYRETCSRRGTAAYPLLGSGIYQVPVSLSFDAWERNHRPGDELYLTEPAANWFEANKPAQPVLTITEDTARTANLALEIDAATEVGRACAGCTISPGIVHYQFTAGVPGSGKSRSIQQGDVDVVVVPTRELRNSWRRRGFAAFTPHTAARVTIGRRVVIDEAPSLPPHLLLLHMQRASSVHLLGDPNQIPAIDFEHAGLVPAIRPELAPTSWWXVTHRCPADVCELIRGAYPKIQTTSRVLRSLFWNEPAIGQKLVXTQAAKAANPGAITVHEAQGATFTETTIIATADARGLIQSSRAHAIVALTRHTEKCVILDAPGLLREVGISDVIVNNFFLAGGEVGXHRPSVIPRGNPDQNLGTLQAFPPSCQISAYHQLAEELGHRPAPVAAVLPPCPELEQGLLYMPQELTVSDSVLVFELTDIVHCRMAAPSQRKAVLSTLVGRYGRRTKLYEAAHSDVRESLARFIPTIGPVQATTCELYELVEAMVEKGQDGSAVLELDLCNRDVSRITFFQKXCNKFTTGETIAHGKVGQGISAWSKTFCALFGPWFRAIEKEILALLPPNIFYGDAYEESVFAAAVSGAGSCMVFENDFSEFDSTQNNFSLGLECVVMEECGMPQWLIRLYHLVRSAWILQAPKESLKGFWKKHSGEPGTLLWNTVWNMAIIAHCYEFRDFRVAAFKGDDSVVLCSDYRQSRNAAALIAGCGLKLKVDYRPIGLYAGVVVAPGLGTLPDVVRFAGRLSEKNWGPGPERAEQLRLAVCDFLRGLTNVAQVCVDVVSRVYGVSPGLVHNLIGMLQTIADGKAHFTETIKPVLDLTNSIIQRVE

SEQ ID NO: 92 - protein, 660 aa, Hepatitis E Virus
  Xaa = Unknown or Other at position 481
MRPRAVLLLFLMFLPMLPAPPAGQPSGRRRGRRSGGAGGGFWSDRVDSQPFALPYIHPTNPFAADVVSQPGAGTRPRQPPRPLGSAWRDQSKRPSVAPRRRSTPAGAAPLTAISPAPDTAPVPDVDSRGAILRRQYNLSTSPLTSSVASGTNLVLYAAPLNPLLPLQDGTNTHIMATEASNYAQYRVVRATIRYRPLVPNAVGGYAISISFWPQTTTTPTSVDMNSITSTDVRILVQPGIASELVIPSERLHYRNQGWRSVETTGVAEEEATSGLVMLCIHGSPVNSYTNTPYTGALGLLDFALELEFRNLTPGNTNTRVSRYTSTARHRLRRGADGTAELTTTAATRFMKDLHFTGTNGVGEVGRGIALTLFNLADTLLGGLPTELISSAGGQLFYSRPVVSANGEPTVKLYTSVENAQQDKGITIPHDIDLGDSRVVIQDYDNQHEQDRPTPSPAPSRPFSVLRANDVLWLSLTAAEYXQTTYGSSTNPMYVSDTVTLVNVATGAQAVARSLDWSKVTLDGRPLTTIQQYSKKFYVLPLXGKLSFWEAGTTKAGYPYNYNTTASDQILIENAAGHRVAISTYTTSLGAGPTSXSAVGVLAPHSALAVLEDTVDYPARAHTFDDFCPECRTLGLQGCAFQSTIAELQRLKMKVGKTRES

SEQ ID NO: 93 - protein, 122 aa, Hepatitis E Virus
  ORF3 HEV US-1
MNNMSFASPMGSPCALGLFCCCSSCFCLCCPRHRPVSRLAVAVGGAAAVPAVVSGVTGLILSPSPSPIFIQPTPSPPMSFHNPGLELALDSRPAPSVPLGVTSPSAPPLPPVVDLPQLGLRR

SEQ ID NO: 94 - DNA, 20 bp, Artificial Sequence
  Primer US5P3S/20
tggcattactactgccattg

SEQ ID NO: 95 - DNA, 20 bp, Artificial Sequence
  Primer US5P45S/20
caattctgccttggcgaatg

SEQ ID NO: 96 - DNA, 20 bp, Artificial Sequence
  Primer US5P296A
aggaaacaccgatgcagaac

SEQ ID NO: 97 - DNA, 20 bp, Artificial Sequence
  Primer US5P243A/20
tccaacctccaagcaacgac

SEQ ID NO: 98 - DNA, 199 bp, Hepatitis E Virus
  Clone  199con
caattctgccttggcgaatgctgtggtggttcggccgtttctttctcgtgtgcaaactgagattcttattaatttgatgcaaccccggcagttggtcttccgccctgaggtgctttggaatcatcctatccagcgggttatacataatgaattagagcagtactgccgggcccgggctggtcgttgcttggaggttgga

SEQ ID NO: 99 - DNA, 25 bp, Hepatitis E Virus
  JE orf1-s
gttctgcatcggtgtttccttagac

SEQ ID NO: 100 - DNA, 26 bp, Hepatitis E Virus
  JE orf1-a
gaatcaggagatacgaggttgtgtgg

SEQ ID NO: 101 - DNA, 331 bp, Hepatitis E Virus
  us2-320
gttctgcatcggtgtttccttagaccggtcggccgagatgttcagcgctggtattctgcccctacccgtggtcctgcggccaattgccgccgctccgcgttgcgtggtctcccccctgtcgaccgcacctattgttttgatggattttcccgttgtgcttttgctgcagagaccggtgtggccctttactctttgcatgacctttggccagctgatgttgcagaggctatggcccgccatgggatgacacgcttatacgccgcactgcaccttccccccgaggtgctgctaccacccggcacctaccacacaacctcgtatctcctgattc

SEQ ID NO: 102 - DNA, 1186 bp, Hepatitis E Virus
  us2-1168
ctcactgtgtccgatagtgtgttggtttttgagcttacggatatagtccactgccgtatggccgccccaagccagcgaaaggctgttctctcaacgcttgtggggaggtacggccgtaggactaaattatatgaggcggcgcattcagatgtccgtgagtccctagcgaggtttatccccaccatcgggcctgttcgggctaccacatgtgagctgtacgagctggttgaagccatggtagagaagggtcaggacggatctgccgtcctagagctcgacctttgcaatcgtgacgtctcgcgcatcacatttttccaaaaggattgcaataagtttacaactggtgagactatcgcccatggcaaggttggccagggcatatcggcctggagcaagaccttctgtgctctgtttggcccgtggttccgcgccattgaaaaggaaatattggccctactcccgcctaatatcttttatggcgacgcctatgaggagtcagtgtttgctgccgctgtgtccggggcagggtcatgtatggtatttgaaaatgacttctcagagtttgacagtacccagaataatttctctctcggccttgagtgtgtggttatggaggagtgcggcatgccccaatggttaattaggttgtaccatctggtccggtcagcctggattttgcaggcgccgaaggagtctcttaaggggttttggaagaagcactctggtgagcctggtacccttctctggaacactgtctggaacatggcgattatagcacattgctaygagttccgtgactttcgtgttgccgccttcaagggtgatgattcagtggtcctctgtagtgactaccgacagrgccgtaacgcggctgccttaattgcaggctgtgggctcaaattgaaggttgattaccgccctatcgggctatatgctggagtggtggtggcccccggtttggggacactgcccgatgtggtgcgttttgccggtcggttatctgagaagaattggggccctggcccggagcgtgctgagcagctgcgtcttgctgtttgtgatttccttcgagggttgacgaatgttgcgcaggtctgtgttgatgttgtgtcccgtgtctatggagttagccccgggctggtacataaccttattggcatgctgcagaccatcgccgatggcaagg

SEQ ID NO: 103 - DNA, 23 bp, Hepatitis E Virus
  JE hevdf2/3 s1
gttccgcttggcgtgaccagtcc

SEQ ID NO: 104 - DNA, 23 bp, Hepatitis E virus
  JE hevdf2/3 a1
gagtcaacatcaggtacaggagc

SEQ ID NO: 105 - DNA, 130 bp, Hepatitis E Virus
  us2-135
gttccgcttggcgtgaccagtcccagcgcccctccgctgccccccgtcgtcgatctgccccagctggggctgcgccgctgactgccgtgtcaccggctcctgacacagctcctgtacctgatgttgactc

SEQ ID NO: 106 - DNA, 26 bp, Hepatitis E Virus
  JE hevdf1-s1
gatgtcattttgtgttgctgctcacc

SEQ ID NO: 107 - DNA, 23 bp, Hepatitis E Virus
  hev216 a1
cgtcctacagcggcatggtactg

SEQ ID NO: 108 - DNA, 564 bp, Hepatitis E Virus
  us2-563
tcacccatgccttatgttccttaccctcgttcaacggaggtgtatgtccggtctatatttggccctggcggctccccatccttgtttccatcagcctgctctactaaatctacctttcatgctgtcccggttcacatctgggatcrgctcatgctctttggtgccaccctgracgatcaggcgttctgctgttcacggcttatgacttacctccgtggtattagttataaggtcactgtcggtgcgcttgtcgctaatgaggggtggaacgcctctgaggatgctcttactgcagtgatcactgcggcctatctgaccatctgccatcagcgttaccttcgcacccaggcgatttccaagggcatgcgccggttggaggttgagcatgctcagaaatttatcacaagactctacagctggctatttgagaagtctggccgtgactacatccccggccgccagcttcaattttatgcacaatgccgacggtggctttctgcaggcttccacctaraccccaggrtgcttgtctttgatgaatcagtaccatgccgctgtaggacg

SEQ ID NO: 109 - DNA, 24 bp, Hepatitis E Virus
  USorf2.1′
gtggagctagtacaccgaccgcag

SEQ ID NO: 110 - DNA, 678 bp, Hepatitis E Virus
  us2-667
cgcttcttggtggtttaccgacagaattgatttcgtcggctgggggccaactgttttactcccgcccggttgtctcagccaatggcgagccaacagtaaagttatatacatctgttgagaatgcgcagcaagacaagggcatcaccattccacatgatatagacctgggtgactcccgtgtggttatccaggattatgataaccagcaygagcaagaccgacctactccgtcacctgccccctctcgccccttctcagttcttcgtgccaatgatgttttgtggctttccctcactgccgctgagtatgaccagactacgtatgggtcgtccaccaaccctatgtatgtctctgacacagttacgcttgttaatgtggctactggtgctcaggctgttgcccgctcccttgattggtctaaagttactctggacggccgcccccttactaccattcagcagtattctaagacattttatgttctcccgctccgcgggaagctgtccttttgggaggctggcacgactaaggccggctacccttacaattataatactaccgctagtgaccaaattttgattgagaatgcggccggccaccgtgtcgctatttccacctataccactagcttaggtgccggtcctacctcgatctctgcggtcggtgtactagctccac

SEQ ID NO: 111 - DNA, 23 bp, Hepatitis E Virus
  hev3301s
gtatgcgagctcatccgtggtgc

SEQ ID NO: 112 - DNA, 25 bp, Hepatitis E Virus
  JE hev167-a1
ccaacacactatcggacacagtgag

SEQ ID NO: 113 - DNA, 580 bp, Hepatitis E Virus
  us2-579
gtatgcgagctcatccgtggtgcctaccccaaaattcagaccacgagccgtgtgctacggtccctgttttggaacgaaccggccatcggccaaaagttggtttttacgcaggctgctaaggctgccaaccctggtgcgattacggttcacgaagctcagggtgctactttcacggagaccacaattatagccacggccgacgctaggggcctcattcagtcatcccgggcccatgctatagtcgcactcacccgccatactgagaagtgtgttattttggatgcccccggcttgttgcgcgaggtcggcatttcggatgttattgtcaataactttttccttgccggtggagaggtcggccatcaccgcccttctgtgatacctcgcggcaatcctgatcagaacctcgggactctacaggcctttccgccgtcatgtcagatcagtgcttaccatcagttggctgaggaactaggtcatcgcccggcccctgtcgccgccgtcttgcccccttgccctgagcttgagcagggcctgctctatatgccacaagaactcactgtgtccgatagtgtgttgg

SEQ ID NO: 114 - DNA, 26 bp, Hepatitis E Virus
  HEV459 s1
cagaaatttatcacaagactctacag

SEQ ID NO: 115 - DNA, 26 bp, Hepatitis E Virus
  HEV459 s3
ctctacagttggctatttgagaagtc

SEQ ID NO: 116 - DNA, 25 bp, Hepatitis E Virus
  JE1955a
ctataaagagctgagcagaaggcgg

SEQ ID NO: 117 - DNA, 734 bp, Hepatitis E Virus
  us2-733
ctctacagttggctatttgagaagtctggccgtgactacatccccggccgccagcttcaattttatgcacaatgccgacggtggctttctgcaggcttccacctaraccccaggrtgcttgtctttgatgaatcagtgccatgccgttgcaggacgtttttgaagaaggtcgcgggtaaattctgctgttttatgcggtggctggggcaggagtgtacctgcttcttggagccagccgagggtttagttggtgatcaaggtcatgacaacgaggcctatgaaggttctgaggtcgacccagctgagcctgcacatcttgatgtctcggggacttatgccgtccatgggcaccagcttgaggccctctatagggcacttaatgtcccacatgatattgccgctcgagcctcccgactaacggctactgttgagctcgttgctagtccggaccgcttagagtgccgcactgtacttggtaataagaccttccggacgacggtggttgatggcgcccatcttgaagcgaatggccctgaggagtatgttctgtcatttgacgcctctcgccagtctatgggggccgggtcgcacagcctcacttatgagctcacccctgccggtctgcaggtaaagatttcatctaatggtctggattgcactgccacattccccccyggtggcgcccctagcgccgcgccgggggaggtggccgccttctgctcagctctttatag

SEQ ID NO: 118 - DNA, 22 bp, Hepatitis E Virus
  JE 2950mex s
gtgtccccggctctggcaagtc

SEQ ID NO: 119 - DNA, 22 bp, Hepatitis E Virus
  JE us2-579-a2
cagggttggcagccttagcagc

SEQ ID NO: 120 - DNA, 483 bp, Hepatitis E Virus
  us2-482
gtgtccccggctctggcaagtcaaggtccatacaacagggagatgtcgatgtggtggttgtgcccacccgggagctccgtaacagctggcgtcgccggggttttgcggccttcacacctcacacagcggcccgtgttactatcggccgccgcgttgtgattgatgaggctccatctctcccaccgcacctgctgctgttacacatgcagcgggcctcctcggtccatctccttggtgatccaaaccagattcctgctattgattttgagcatgccggcctggtccccgcgatccgccccgagcttgcgccaacgagctggtggcacgttacacaccgttgcccggccgatgtgtgcgagctcatacgtggggcctaccccaaaattcagaccacgagccgtgtgctacggtccctgttttggaacgaaccggccatcggccaaaagttggtttttacgcaggctgctaaggctgccaaccctg

SEQ ID NO: 121 - DNA, 24 bp, Hepatitis E Virus
  JE 2600s
taacccaaagaggcttgaggctgc

SEQ ID NO: 122 - DNA, 22 bp, Hepatitis E Virus
  us2-482-a1
ccgctgtgtgaggtgtgaaggc

SEQ ID NO: 123 - DNA, 23 bp, Hepatitis E Virus
  us2-482-a2
gacgccagctgttacggagctcc

SEQ ID NO: 124 - DNA, 431 bp, Hepatitis E Virus
  us2-430
taacccaaagaggcttgaggctgcgtaccgggaaacttgctcccgtcgtggcaccgctgcctacccgcttttgggctcgggtatataccaggtccctgttagcctcagttttgatgcctgggaacgcaatcaccgccccggcgatgagctttacttgacagagcccgccgcagcctggtttgaggctaataagccggcgcagccggcgcttactataactgaggacacggcccgtacggccaacctggcattagagattgatgccgccacagaggttggccgtgcttgtgccggctgcaccatcagccccgggattgtgcactatcagtttaccgccggggtcccgggctcaggcaagtcaaggtccatacaacagggagatgtcgatgtggtggttgtgcccacccgggagctccgtaacagctggcgtc

SEQ ID NO: 125 - DNA, 22 bp, Hepatitis E Virus
  us2-orf2/3 s1
cgtcgtcgatctgccccagctg

SEQ ID NO: 126 - DNA, 25 bp, Hepatitis E Virus
  HEVConsORF2-a1
cttgttcrtgytggttrtcataatc

SEQ ID NO: 127 - DNA, 21 bp, Hepatitis E Virus
  us2-orf2/3 s2
cgctgactgccgtgtcaccgg

SEQ ID NO: 128 - DNA, 25 bp, Hepatitis E Virus
  HEVConsORF2-a2
gttcrtgytggttrtcataatcctg

SEQ ID NO: 129 - DNA, 1020 bp, Hepatitis E Virus
  us2-1019
cgctgactgccgtgtcaccggctcctgacacagcccctgtacctgatgttgactcacgtggtgctattctgcgccggcagtacaatttgtccacgtccccgctcacgtcatctgtcgcttcgggtactaatttggtcctctatgctgccccgctgaatcccctcttgcctctccaggatggtaccaacactcatattatggctactgaggcatccaattatgcccagtatcgggttgttcgagctacaatccgttatcgcccgctggtgccgaatgccgttggtggctatgccatttccatttctttctggccccaaactacaactacccctacttctgtcgatatgaattctattacttccacygatgttaggattttggttcagcccggtattgcctccgagctagtcatccccagtgagcgccttcattaccgtaatcaaggctggcgctctgttgagaccacgggtgtggctgaggaggaggctacttccggtctggtaatgctttgcattcatggctctcctgttaattcctacactaatacaccttacactggtgcgctggggcttcttgattttgcactagagcttgaatttaggaatttgacacccgggaacaccaacacccgtgtttcccggtataccagcacagcccgccaccggctgcgccgtggtgctgatgggactgctgagcttactaccacagcagccacacgtttcatgaaggacctgcacttcgctggcacgaatggcgttggtgaggtgggtcgtggtatcgccctgacactgttcaatctcgctgatacgcttctcggcggtttaccgacagaattgatttcgtcggctgggggccaactgttttactcccgcccggttgtctcagccaatggcgagccaacagtaaagttatatacatctgttgagaatgcgcagcaagacaagggcatcaccattccacatgatatagacctgggtgactcccgtgtggttatccaggattatgataaccagcaygaac

SEQ ID NO: 130 - DNA, 24 bp, Hepatitis E Virus
  us2 330s1
cagctgatgttgcagaggctatgg

SEQ ID NO: 131 - DNA, 24 bp, Hepatitis E Virus
  us2 563a1
gcaggctgatggaaacaaggatgg

SEQ ID NO: 132 - DNA, 407 bp, Hepatitis E Virus
  us2-406
cagctgatgttgcagaggctatggcccgccatgggatgacacgcttatacgccgcactgcaccttccccccgaggtgctgctaccacccggcacctaccacacaacctcgtacctcttgattcacgatggcaaccgcgctgttgtaacttacgagggcgatactagtgcgggctataatcatgatgtctccatacttcgtgcatggatccgtactactaaaatagttggtgaccatccattggtcatagagcgagtgcgggccattgggtgtcattttgtgctgctgctcaccgcagcccctgaaccgtcacctatgccttatgttccctaccctcgttcaacggaggtgtatgtccggtctatatttggccctggcggctccccatccttgtttccatcagcctgc

SEQ ID NO: 133 - DNA, 22 bp, Hepatitis E Virus
  us2-579 s1
cagaccacgagccgtgtgctac

SEQ ID NO: 134 - DNA, 23 bp, Hepatitis E Virus
  us2-1168 a1
ccacaagcgttgagagaacagcc

SEQ ID NO: 135 - DNA, 22 bp, Hepatitis E Virus
  us2-579 s2
gctgctaaggctgccaaccctg

SEQ ID NO: 136 - DNA, 547 bp, Hepatitis E Virus
  us2-579wb
gctgctaaggctgccaaccctggtgcgattacggttcacgaagctcagggtgctactttcacggagaccacaattatagccacggccgacgctaggggcctcattcagtcatcccgggcccatgctatagtcgcactcacccgccatactgagaagtgtgttattttggatgcccccggcttgttgcgcgaggtcggcatttcggatgttattgtcaataactttttccttgccggtggagaggtcggccatcaccgcccttctgtgatacctcgcggcaatcctgatcagaacctcgggactctacaggcctttccgccgtcatgtcagatcagtgcttaccatcagttggctgaggaactaggtcatcgcccggcccctgtcgccgccgtcttgcccccttgccctgagcttgagcagggcctgctctatatgccacaagaacttactgtgtccgatagcgtgctggtttttgagcttacggatatagtccactgccgtatggccgccccaagccagcgaaaggctgttctctcaacgcttgtgg

SEQ ID NO: 137 - DNA, 24 bp, Hepatitis E Virus
  us2-733s1
cacagcctcacttatgagctcacc

SEQ ID NO: 138 - DNA, 23 bp, Hepatitis E Virus
  us2-430a1
cggtgattgcgttcccaggcatc

SEQ ID NO: 139 - DNA, 26 bp, Hepatitis E Virus
  us2-733s2
ctgcaggtaaagatttcatctaatgg

SEQ ID NO: 140 - DNA, 24 bp, Hepatitis E Virus
  us2-430a2
ccaggcatcaaaactgaggctaac

SEQ ID NO: 141 - DNA, 903 bp, Hepatitis E Virus
  us2-851
ctgcaggtaaagatttcatctaatggtctggattgcactgccacattccccccyggtggcgcccctagcgccgcgccgggggaggtggcsgccttctgcagtgctctttatagatacaataggttcacccagcggcattcgctgacaggcggactatggctacatcctgaggggctgctgggtatcttccccccattctcccctgggcatatttgggagtctgctaaccccttttgcggtgaggggactttgtatacccgaacctggtcaacctctggtttttctagtgatttctccccccctgaggcggccgctcctgcttcggctgccgccccggggttgccctaccctactccacctgttagtgatatctgggtgttaccaccgccctcagaggaatctcatgttgatgcggcatctgtaccctctgttcctgagcctgctggattgaccagccctattgtgcttacccccccccccccccctcctcccgtgcgtaagccggcaacatccccgcctccccgcactcgccgtctcctttacacctaccccgacggcgccaaggtgtatgcggggtcattgtktgagtcagactgtgattggttagtcaatgcctcaaaccctggccatcgccccgggggtggcctctgccatgctttttatcaacgtttcccagaagcgttctactcgactgaattcatcatgcgcgagggccttgcagcatacactttaaccccgcgccctattatccatgcagtggctcccgactatagggttgagcaaaacccgaagaggcttgaggcagcgtaccgggaaacttgctcccgtcgtggcaccgctgcctacccgcttttgggctcgggtatataccaggtccctgttagcctcagttttgatgcctgg

SEQ ID NO: 142 - DNA, 24 bp, Hepatitis E Virus
  us2-1168s1
gcaggtctgtgttgatgttgtgtc

SEQ ID NO: 143 - DNA, 21 bp, Hepatitis E Virus
  us2-dforf2/3 a2
ccggtgacacggcagtcagcg

SEQ ID NO: 144 - DNA, 25 bp, Hepatitis E Virus
  us2-1168s2
gatgttgtgtcccgtgtctatggag

SEQ ID NO: 145 - DNA, 22 bp, Hepatitis E Virus
  us2 dforf2/3 a3
cagctggggcagatcgacgacg

SEQ ID NO: 146 - DNA, 503 bp, Hepatitis E Virus
  us2-502
gatgttgtgtcccgtgtctatggagttagccccgggctggtacataaccttattggcatgctgcagaccattgctgatggcaaggcccactttacagaraatattaaacctgtgcttgaccttacaaattccatcatacaacgggtggaatgaataacatgtcttttgcatcgcccatgggatcaccatgcgccctagggctgttctgttgttgctcttcgtgcttttgcctatgctgcccgcgccaccggccggccagccgtctggccgccgtcgtgggcggcgcagcggcggtgccggcggtggtttctggggtgacagggttgattctcagcccttcgccctcccctatattcatccaaccaaccccttcgccgccgatgtcgtttcacaacccggggctggaactcgccctcgacagccgccccgcccccttggytccgcttggcgtgaccagtcccagcgcccctccgctgccccccgtcgtcgatctgccccagctg

SEQ ID NO: 147 - DNA, 24 bp, Hepatitis E Virus
  HEVConsORF1-s1
ctggcatyactactgcyattgagc

SEQ ID NO: 148 - DNA, 23 bp, Hepatitis E Virus
  HEVConsORF1-a1
ccatcrarrcagtaagtgcggtc

SEQ ID NO: 149 - DNA, 418 bp, Hepatitis E Virus
  us2-orf1
ctggcattactactgctattgagcaggctgctctggctgcggctaattccgccttggcgaatgctgtggtggttcggccgtttctttctcgtgtgcaaactgagattcttattaatttgatgcaaccccggcagttggtcttccgccctgaggtgctttggaatcatcctatccagcgggttatacataatgaattagagcagtactgccgggcccgggctggtcgttgtttggaggttggagcccacccgaggtccattaatgacaaccctaatgtcttgcataggtgttttcttagaccggtcggccgagatgttcagcgctggtattctgcccctacccgtggtcctgcggccaattgccgccgctccgcgttgcgtggtctcccccctgtcgaccgcacttactgttttgatgg

SEQ ID NO: 150 - DNA, 24 bp, Hepatitis E Virus
  HEVConsORF2-s1
gacagaattratttcgtcggctgg

SEQ ID NO: 151 - DNA, 197 bp, Hepatitis E Virus
  us2-orf2
gacagaattgatttcgtcggctgggggccaactgttttactcccgcccggttgtctcagccaatggcgagccaacagtaaagttatatacatctgttgagaatgcgcagcaagacaagggcatcaccattccacatgatatagacctgggtgactcccgtgtggttatccaggattatgataaccagcaygagcaag

SEQ ID NO: 152 - DNA, 22 bp, Hepatitis E Virus
  HEVConsORF2-s2
gtygtctcrgccaatggcgagc

SEQ ID NO: 153 - DNA, 901 bp, Hepatitis E Virus
  us2-3p
gttgtctcagccaatggcgagccaacagtaaagttatatacatctgttgagaatgcgcagcaagacaagggcatcaccattccacatgatatagacctgggtgactcccgtgtggttatccaggattatgataaccagcaygagcaagaccgacctactccgtcacctgccccctctcgccccttctcagttcttcgtgccaatgatgttttgtggctttccctcactgccgctgagtatgaccagactacgtatgggtcgtccaccaaccctatgtatgtctctgacacagttacgcttgttaatgtggctactggtgctcaggctgttgcccgctcccttgattggtctaaagttactctggacggccgcccccttactaccattcagcagtattctaagacattttatgttctcccgctccgcgggaagctgtccttttgggaggctggcacgactaaggccggctacccttacaattataatactaccgctagtgaccaaattttgattgagaatgcggccggccaccgtgtcgctatttccacctataccactagcttaggtgccggtcctacctcgatctctgcggtcggcgtactggctccacactctgcccttgccgttcttgaggatactattgattaccccgcccgtgcccatacttttgatgatttttgcccggagtgccgtaccctaggtttgcagggttgtgcattccagtctactattgctgagctccagcgtttaaaaatgaaggtaggtaaaacccgggagtcttaattaattccttctgtgcccccttcgtagtttctttcgcttttatttcttatttctgctttccgcgctccctggaaaaaaaaaaaaaaaaaaaaaaaaaagtactagtcgacgcgtggcc

SEQ ID NO: 154 - DNA, 27 bp, Hepatitis E Virus
  us2-gap s1
tatagataacaataggttcacccagcg

SEQ ID NO: 155 - DNA, 25 bp, Hepatitis E Virus
  us2-gap a1
attcagtcgagtagaacgcttctgg

SEQ ID NO: 156 - DNA, 23 bp, Hepatitis E Virus
  us2-gap s2
cggactatggctacatcctgagg

SEQ ID NO: 157 - DNA, 26 bp, Hepatitis E Virus
  us2-gap a2
ttgactaaccaatcacagtctgactc

SEQ ID NO: 158 - DNA, 462 bp, Artificial Sequence
  13906-gap
cggactatggctacatcctgaggggctgctgggtatcttccccccattctcccctgggcatatttgggagtctgctaaccccttttgcggtgaggggactttgtatacccgaacctggtcaacctctggtttttctagtgatttctccccccctgaggcggccgctcctgcttcggctgccgccccggggttgccctaccctactccacctgttagtgatatctgggtgttaccaccgccctcagaggaatctcatgttgatgcggcatctgtaccctctgttcctgagcctgctggattgaccagccctattgtgcttacccccccccccccccctcctcccgtgcgtaagccggcaacatccccgcctccccgcactcgccgtctcctttacacctaccccgacggcgccaaggtgtatgcggggtcattgtttgagtcagactgtgattggttagtcaa

SEQ ID NO: 159 - DNA, 21 bp, Hepatitis E Virus
  us-575a
gccgggtggtagcagcacctc

SEQ ID NO: 160 - DNA, 24 bp, Hepatitis E Virus
  us-426s
cgttgtgcttttgctgcagagacc

SEQ ID NO: 161 - DNA, 22 bp, Hepatitis E Virus
  us-84a
gaaacggccgaaccaccacagc

SEQ ID NO: 162 - DNA, 24 bp, Hepatitis E Virus
  us-484s
cagctgatgttgcagaggctatgg

SEQ ID NO: 163 - DNA, 22 bp, Hepatitis E Virus
  us-78a
gccgaaccaccacagcattcgc

SEQ ID NO: 164 - DNA, 7277 bp, Hepatitis E Virus
  us2fu11
tcgacagggggcagaccacgtatgtggtcgatgccatggaggcccatcagttcattaaggctcctggcattactactgctattgagcaggctgctctggctgcggctaattccgccttggcgaatgctgtggtggttcggccgtttctttctcgtgtgcaaactgagattcttattaatttgatgcaaccccggcagttggtcttccgccctgaggtgctttggaatcatcctatccagcgggttatacataatgaattagagcagtactgccgggcccgggctggtcgttgtttggaggttggagcccacccgaggtccattaatgacaaccctaatgtcttgcataggtgttttcttagaccggtcggccgagatgttcagcgctggtattctgcccctacccgtggtcctgcggccaattgccgccgctccgcgttgcgtggtctcccccctgtcgaccgcacctattgttttgatggattttcccgttgtgcttttgctgcagagaccggtgtggccctttactctttgcatgacctttggccagctgatgttgcagaggctatggcccgccatgggatgacacgcttatacgccgcactgcaccttccccccgaggtgctgctaccacccggcacctaccacacaacctcgtacctcttgattcacgatggcaaccgcgctgttgtaacttacgagggcgatactagtgcgggctataatcatgatgtctccatacttcgtgcatggatccgtactactaaaatagttggtgaccatccattggtcatagagcgagtgcgggccattgggtgtcattttgtgctgctgctcaccgcagcccctgaaccgtcacctatgccttatgttccctaccctcgttcaacggaggtgtatgtccggtctatatttggccctggcggctccccatccttgtttccatcagcctgctctactaaatctacctttcatgctgtcccggttcacatctgggatcrgctcatgctctttggtgccaccctgracgatcaggcgttctgctgttcacggcttatgacttacctccgtggtattagttataaggtcactgtcggtgcgcttgtcgctaatgaggggtggaacgcctctgaggatgctcttactgcagtgatcactgcggcctatctgaccatctgccatcagcgttaccttcgcacccaggcgatttccaagggcatgcgccggttggaggttgagcatgctcagaaatttatcacaagactctacagctggctatttgagaagtctggccgtgactacatccccggccgccagcttcaattttatgcacaatgccgacggtggctttctgcaggcttccacctaraccccaggrtgcttgtctttgatgaatcagtgccatgccgttgcaggacgtttttgaagaaggtcgcgggtaaattctgctgttttatgcggtggctggggcaggagtgtacctgcttcttggagccagccgagggtttagttggtgatcaaggtcatgacaacgaggcctatgaaggttctgaggtcgacccagctgagcctgcacatcttgatgtctcggggacttatgccgtccatgggcaccagcttgaggccctctatagggcacttaatgtcccacatgatattgccgctcgagcctcccgactaacggctactgttgagctcgttgctagtccggaccgcttagagtgccgcactgtacttggtaataagaccttccggacgacggtggttgatggcgcccatcttgaagcgaatggccctgaggagtatgttctgtcatttgacgcctctcgccagtctatgggggccgggtcgcacagcctcacttatgagctcacccctgccggtctgcaggtaaagatttcatctaatggtctggattgcactgccacattccccccyggtggcgcccctagcgccgcgccgggggaggtggcsgccttctgcagtgctctttatagatacaataggttcacccagcggcattcgctgacaggcggactatggctacatcctgaggggctgctgggtatcttccccccattctcccctgggcatatttgggagtctgctaaccccttttgcggtgaggggactttgtatacccgaacctggtcaacctctggtttttctagtgatttctccccccctgaggcggccgctcctgcttcggctgccgccccggggttgccctaccctactccacctgttagtgatatctgggtgttaccaccgccctcagaggaatctcatgttgatgcggcatctgtaccctctgttcctgagcctgctggattgaccagccctattgtgcttacccccccccccccccctcctcccgtgcgtaagccggcaacatccccgcctccccgcactcgccgtctcctttacacctaccccgacggcgccaaggtgtatgcggggtcattgtktgagtcagactgtgattggttagtcaatgcctcaaaccctggccatcgccccgggggtggcctctgccatgctttttatcaacgtttcccagaagcgttctactcgactgaattcatcatgcgcgagggccttgcagcatacactttaaccccgcgccctattatccatgcagtggctcccgactatagggttgagcaaaacccgaagaggcttgaggcagcgtaccgggaaacttgctcccgtcgtggcaccgctgcctacccgcttttgggctcgggtatataccaggtccctgttagcctcagttttgatgcctgggaacgcaatcaccgccccggcgatgagctttacttgacagagcccgccgcagcctggtttgaggctaataagccggcgcagccggcgcttactataactgaggacacggcccgtacggccaacctggcattagagattgatgccgccacagaggttggccgtgcttgtgccggctgcaccatcagccccgggattgtgcactatcagtttaccgccggggtcccgggctcaggcaagtcaaggtccatacaacagggagatgtcgatgtggtggttgtgcccacccgggagctccgtaacagctggcgtcgccggggttttgcggccttcacacctcacacagcggcccgtgttactatcggccgccgcgttgtgattgatgaggctccatctctcccaccgcacctgctgctgttacacatgcagcgggcctcctcggtccatctccttggtgatccaaaccagattcctgctattgattttgagcatgccggcctggtccccgcgatccgccccgagcttgcgccaacgagctggtggcacgttacacaccgttgcccggccgatgtgtgcgagctcatacgtggggcctaccccaaaattcagaccacgagccgtgtgctacggtccctgttttggaacgaaccggccatcggccaaaagttggtttttacgcaggctgctaaggctgccaaccctggtgcgattacggttcacgaagctcagggtgctactttcacggagaccacaattatagccacggccgacgctaggggcctcattcagtcatcccgggcccatgctatagtcgcactcacccgccatactgagaagtgtgttattttggatgcccccggcttgttgcgcgaggtcggcatttcggatgttattgtcaataactttttccttgccggtggagaggtcggccatcaccgcccttctgtgatacctcgcggcaatcctgatcagaacctcgggactctacaggcctttccgccgtcatgtcagatcagtgcttaccatcagttggctgaggaactaggtcatcgcccggcccctgtcgccgccgtcttgcccccttgccctgagcttgagcagggcctgctctatatgccacaagaacttactgtgtccgatagcgtgctggtttttgagcttacggatatagtccactgccgtatggccgccccaagccagcgaaaggctgttctctcaacgcttgtggggaggtacggccgtaggactaaattatatgaggcggcgcattcagatgtccgtgagtccctagcgaggtttatccccaccatcgggcctgttcgggctaccacatgtgagctgtacgagctggttgaagccatggtagagaagggtcaggacggatctgccgtcctagagctcgacctttgcaatcgtgacgtctcgcgcatcacatttttccaaaaggattgcaataagtttacaactggtgagactatcgcccatggcaaggttggccagggcatatcggcctggagcaagaccttctgtgctctgtttggcccgtggttccgcgccattgaaaaggaaatattggccctactcccgcctaatatcttttatggcgacgcctatgaggagtcagtgtttgctgccgctgtgtccggggcagggtcatgtatggtatttgaaaatgacttctcagagtttgacagtacccagaataatttctctctcggccttgagtgtgtggttatggaggagtgcggcatgccccaatggttaattaggttgtaccatctggtccggtcagcctggattttgcaggcgccgaaggagtctcttaaggggttttggaagaagcactctggtgagcctggtacccttctctggaacactgtctggaacatggcgattatagcacattgctaygagttccgtgactttcgtgttgccgccttcaagggtgatgattcagtggtcctctgtagtgactaccgacagrgccgtaacgcggctgccttaattgcaggctgtgggctcaaattgaaggttgattaccgccctatcgggctatatgctggagtggtggtggcccccggtttggggacactgcccgatgtggtgcgttttgccggtcggttatctgagaagaattggggccctggcccggagcgtgctgagcagctgcgtcttgctgtttgtgatttccttcgagggttgacgaatgttgcgcaggtctgtgttgatgttgtgtcccgtgtctatggagttagccccgggctggtacataaccttattggcatgctgcagaccattgctgatggcaaggcccactttacagaraatattaaacctgtgcttgaccttacaaattccatcatacaacgggtggaatgaataacatgtcttttgcatcgcccatgggatcaccatgcgccctagggctgttctgttgttgctcttcgtgcttttgcctatgctgcccgcgccaccggccggccagccgtctggccgccgtcgtgggcggcgcagcggcggtgccggcggtggtttctggggtgacagggttgattctcagcccttcgccctcccctatattcatccaaccaaccccttcgccgccgatgtcgtttcacaacccggggctggaactcgccctcgacagccgccccgcccccttggytccgcttggcgtgaccagtcccagcgcccctccgctgccccccgtcgtcgatctgccccagctggggctgcgccgctgactgccgtgtcaccggctcctgacacagcccctgtacctgatgttgactcacgtggtgctattctgcgccggcagtacaatttgtccacgtccccgctcacgtcatctgtcgcttcgggtactaatttggtcctctatgctgccccgctgaatcccctcttgcctctccaggatggtaccaacactcatattatggctactgaggcatccaattatgcccagtatcgggttgttcgagctacaatccgttatcgcccgctggtgccgaatgccgttggtggctatgccatttccatttctttctggccccaaactacaactacccctacttctgtcgatatgaattctattacttccacygatgttaggattttggttcagcccggtattgcctccgagctagtcatccccagtgagcgccttcattaccgtaatcaaggctggcgctctgttgagaccacgggtgtggctgaggaggaggctacttccggtctggtaatgctttgcattcatggctctcctgttaattcctacactaatacaccttacactggtgcgctggggcttcttgattttgcactagagcttgaatttaggaatttgacacccgggaacaccaacacccgtgtttcccggtataccagcacagcccgccaccggctgcgccgtggtgctgatgggactgctgagcttactaccacagcagccacacgtttcatgaaggacctgcacttcgctggcacgaatggcgttggtgaggtgggtcgtggtatcgccctgacactgttcaatctcgctgatacgcttctcggcggtttaccgacagaattgatttcgtcggctgggggccaactgttttactcccgcccggttgtctcagccaatggcgagccaacagtaaagttatatacatctgttgagaatgcgcagcaagacaagggcatcaccattccacatgatatagacctgggtgactcccgtgtggttatccaggattatgataaccagcaygagcaagaccgacctactccgtcacctgccccctctcgccccttctcagttcttcgtgccaatgatgttttgtggctttccctcactgccgctgagtatgaccagactacgtatgggtcgtccaccaaccctatgtatgtctctgacacagttacgcttgttaatgtggctactggtgctcaggctgttgcccgctcccttgattggtctaaagttactctggacggccgcccccttactaccattcagcagtattctaagacattttatgttctcccgctccgcgggaagctgtccttttgggaggctggcacgactaaggccggctacccttacaattataatactaccgctagtgaccaaattttgattgagaatgcggccggccaccgtgtcgctatttccacctataccactagcttaggtgccggtcctacctcgatctctgcggtcggcgtactggctccacactctgcccttgccgttcttgaggatactattgattaccccgcccgtgcccatacttttgatgatttttgcccggagtgccgtaccctaggtttgcagggttgtgcattccagtctactattgctgagctccagcgtttaaaaatgaaggtaggtaaaacccgggagtcttaattaattccttctgtgcccccttcgtagtttctttcgcttttatttcttatttctgctttccgcgctccctggaaaaaaaaaaaaaaaaaaaaaaaaaa

SEQ ID NO: 165 - DNA, 7277 bp, Hepatitis E Virus; identical to SEQ ID NO: 164
  us2fu11

SEQ ID NO: 166 - protein, 1708 aa, Hepatitis E Virus
  Xaa = Unknown or Other at position 322
MEAHQFIKAPGITTAIEQAALAAANSALANAVVVRPFLSRVQTEILINLMQPRQLVFRPEVLWNHPIQRVIHNELEQYCRARAGRCLEVGAHPRSINDNPNVLHRCFLRPVGRDVQRWYSAPTRGPAANCRRSALRGLPPVDRTYCFDGFSRCAFAAETGVALYSLHDLWPADVAEAMARHGMTRLYAALHLPPEVLLPPGTYHTTSYLLIHDGNRAVVTYEGDTSAGYNHDVSILRAWIRTTKIVGDHPLVIERVRAIGCHFVLLLTAAPEPSPMPYVPYPRSTEVYVRSIFGPGGSPSLFPSACSTKSTFHAVPVHIWDXLMLFGATLXDQAFCCSRLMTYLRGISYKVTVGALVANEGWNASEDALTAVITAAYLTICHQRYLRTQAISKGMRRLEVEHAQKFITRLYSWLFEKSGRDYIPGRQLQFYAQCRRWLSAGFHLXPRXLVFDESVPCRCRTFLKKVAGKFCCFMRWLGQECTCFLEPAEGLVGDQGHDNEAYEGSEVDPAEPAHLDVSGTYAVHGHQLEALYRALNVPHDIAARASRLTATVELVASPDRLECRTVLGNKTFRTTVVDGAHLEANGPEEYVLSFDASRQSMGAGSHSLTYELTPAGLQVKISSNGLDCTATFPXGGAPSAAPGEVXAFCSALYRYNRFTQRHSLTGGLWLHPEGLLGIFPPFSPGHIWESANPFCGEGTLYTRTWSTSGFSSDFSPPEAAAPASAAAPGLPYPTPPVSDIWVLPPPSEESHVDAASVPSVPEPAGLTSPIVLTPPPPPPPVRKPATSPPPRTRRLLYTYPDGAKVYAGSLXESDCDWLVNASNPGHRPGGGLCHAFYQRFPEAFYSTEFIMREGLAAYTLTPRPIIHAVAPDYRVEQNPKRLEAAYRETCSRRGTAAYPLLGSGIYQVPVSLSFDAWERNHRPGDELYLTEPAAAWFEANKPAQPALTITEDTARTANLALEIDAATEVGRACAGCTISPGIVHYQFTAGVPGSGKSRSIQQGDVDVVVVPTRELRNSWRRRGFAAFTPHTAARVTIGRRVVIDEAPSLPPHLLLLHMQRASSVHLLGDPNQIPAIDFEHAGLVPAIRPELAPTSWWHVTHRCPADVCELIRGAYPKIQTTSRVLRSLFWNEPAIGQKLVFTQAAKAANPGAITVHEAQGATFTETTIIATADARGLIQSSRAHAIVALTRHTEKCVILDAPGLLREVGISDVIVNNFFLAGGEVGHHRPSVIPRGNPDQNLGTLQAFPPSCQISAYHQLAEELGHRPAPVAAVLPPCPELEQGLLYMPQELTVSDSVLVFELTDIVHCRMAAPSQRKAVLSTLVGRYGRRTKLYEAAHSDVRESLARFIPTIGPVRATTCELYELVEAMVEKGQDGSAVLELDLCNRDVSRITFFQKDCNKFTTGETIAHGKVGQGISAWSKTFCALFGPWFRAIEKEILALLPPNIFYGDAYEESVFAAAVSGAGSCMVFENDFSEFDSTQNNFSLGLECVVMEECGMPQWLIRLYHLVRSAWILQAPKESLKGFWKKHSGEPGTLLWNTVWNMAIIAHCXEFRDFRVAAFKGDDSVVLCSDYRQXRNAAALIAGCGLKLKVDYRPIGLYAGVVVAPGLGTLPDVVRFAGRLSEKNWGPGPERAEQLRLAVCDFLRGLTNVAQVCVDVVSRVYGVSPGLVHNLIGMLQTIADGKAHFTXNIKPVLDLTNSIIQRVE

SEQ ID NO: 167 - protein, 660 aa, Hepatitis E Virus
  Xaa = Unknown or Other at position 84
MRPRAVLLLLFVLLPMLPAPPAGQPSGRRRGRRSGGAGGGFWGDRVDSQPFALPYIHPTNPFAADVVSQPGAGTRPRQPPRPLXSAWRDQSQRPSAAPRRRSAPAGAAPLTAVSPAPDTAPVPDVDSRGAILRRQYNLSTSPLTSSVASGTNLVLYAAPLNPLLPLQDGTNTHIMATEASNYAQYRVVRATIRYRPLVPNAVGGYAISISFWPQTTTTPTSVDMNSITSXDVRILVQPGIASELVIPSERLHYRNQGWRSVETTGVAEEEATSGLVMLCIHGSPVNSYTNTPYTGALGLLDFALELEFRNLTPGNTNTRVSRYTSTARHRLRRGADGTAELTTTAATRFMKDLHFAGTNGVGEVGRGIALTLFNLADTLLGGLPTELISSAGGQLFYSRPVVSANGEPTVKLYTSVENAQQDKGITIPHDIDLGDSRVVIQDYDNQXEQDRPTPSPAPSRPFSVLRANDVLWLSLTAAEYDQTTYGSSTNPMYVSDTVTLVNVATGAQAVARSLDWSKVTLDGRPLTTIQQYSKTFYVLPLRGKLSFWEAGTTKAGYPYNYNTTASDQILIENAAGHRVAISTYTTSLGAGPTSISAVGVLAPHSALAVLEDTIDYPARAHTFDDFCPECRTLGLQGCAFQSTIAELQRLKMKVGKTRES

SEQ ID NO: 168 - protein, 122 aa, Hepatitis E Virus
  us2 orf3
MNNMSFASPMGSPCALGLFCCCSSCFCLCCPRHRPASRLAAVVGGAAAVPAVVSGVTGLILSPSPSPIFIQPTPSPPMSFHNPGLELALDSRPAPLXPLGVTSPSAPPLPPVVDLPQLGLRR

SEQ ID NO: 169 - protein, 33 aa, Hepatitis E Virus
  M 4-2
ANQPGHLAPLGEIRPSAPPLPPVADLPQPGLRR

SEQ ID NO: 170 - protein, 48 aa, Hepatitis E Virus
  M 3-2e
TFDYPGRAHTFDDFCPECRALGLQGCAFQSTVAELQRLKVKVGKTREL

SEQ ID NO: 171 - protein, 33 aa, Hepatitis E Virus
  B 4-2
ANPPDHSAPLGVTRPSAPPLPHVVDLPQLGPRR

SEQ ID NO: 172 - protein, 48 aa, Hepatitis E Virus
  B 3-2e
TLDYPARAHTFDDFCPECRPLGLQGCAFQSTVAELQRLKMKVGKTREL

SEQ ID NO: 173 - protein, 33 aa, Hepatitis E Virus
  OFR3 (u4.2)
DSRPAPSVPLGVTSPSAPPLPPVVDLPQLGLRR

SEQ ID NO: 174 - protein, 48 aa, Hepatitis E Virus
  ORF2 (u3.2e)
TVDYPARAHTFDDFCPECRTLGLQGCAFQSTIAELQRLKMKVGKTRES

SEQ ID NO: 175 - protein, 327 aa, Hepatitis E Virus
  US-1 SG3
GADGTAELTTTAATRFMKDLHFTGTNGVGEVGRGIALTLFNLADTLLGGLPTELISSAGGQLFYSRPVVSANGEPTVKLYTSVENAQQDKGITIPHDIDLGDSRVVIQDYDNQHEQDRPTPSPAPSRPFSVLRANDVLWLSLTAAEYXQTTYGSSTNPMYVSDTVTLVNVATGAQAVARSLDWSKVTLDGRPLTTIQQYSKKFYVLPLXGKLSFWEAGTTKAGYPYNYNTTASDQILIENAAGHRVAISTYTTSLGAGPTSXSAVGVLAPHSALAVLEDTVDYPARAHTFDDFCPECRTLGLQGCAFQSTIAELQRLKMKVGKTRES

SEQ ID NO: 176 - protein, 327 aa, Hepatitis E Virus
  US-2 SG3
GADGTAELTTTAATRFMKDLHFAGTNGVGEVGRGIALTLFNLADTLLGGLPTELISSAGGQLFYSRPVVSANGEPTVKLYTSVENAQQDKGITIPHDIDLGDSRVVIQDYDNQXEQDRPTPSPAPSRPFSVLRANDVLWLSLTAAEYDQTTYGSSTNPMYVSDTVTLVNVATGAQAVARSLDWSKVTLDGRPLTTIQQYSKTFYVLPLRGKLSFWEAGTTKAGYPYNYNTTASDQILIENAAGHRVAISTYTTSLGAGPTSISAVGVLAPHSALAVLEDTIDYPARAHTFDDFCPECRTLGLQGCAFQSTIAELQRLKMKVGKTRES

SEQ ID NO: 177 - DNA, 21 bp, Hepatitis E Virus
  HEVConsORF1-s2
ctgccytkgcgaatgctgtgg

SEQ ID NO: 178 - DNA, 24 bp, Hepatitis E Virus
  HEVConsORF1-a2
ggcagwrtaccarcgctgaacatc

SEQ ID NO: 179 - DNA, 294 bp, Hepatitis E Virus
  z12-orf1 (G.S.)
tggcattactactgccattgagcaagctgctctggctgcggccaattctgccttggcgaatgctgtggtggttcggccgtttttatctcgtttacagactgagattcttattaatttgatgcaaccccgacagttggtctttcgacctgaggtgttctggaaccatcccatccaacgtgttatacataatgaattggagcagtactgccgggcccgggccggtcgctgtctggaaattggagcccatccaaggtcaatcaatgataatcctaatgttctgcatcggtgtttcct

SEQ ID NO: 180 - DNA, 418 bp, Hepatitis E Virus
  z12-orf1.con
ctggcattactactgctattgagcaagctgctctgggtgcggccaattctgccttggcgaatgctgtggtggttcggccgtttttatctcgtttacagactgagattcttattaatttgatgcaaccccgacagttggtctttcgacctgaggtgttctggaaccatcccatccaacgtgttatacataatgaattggagcagtactgccgggcccgggccggtcgctgtctggaaattggagcccatccaaggtcaatcaatgataatcctaatgttctgcatcggtgctttttacgaccggtcgggagggacgttcagcgctggtactccgcccccacccgtggccccgcggccaactgccgccggtctgcgctgcgtggtctcccccctgtcgaccgcacttactgcctcgatgg

SEQ ID NO: 181 - DNA, 197 bp, Hepatitis E Virus
  z12-orf2.con
gacagaattaatttcgtcggctgggggtcaactgttctactcccgccctgtcgtctcagccaatggcgagccgactgtcaagttatacacatctgttgagaatgcacagcaggataaggggatagctattccacatgacatagatttgggcgactctcgtttggtaatccaggattatgataaccaacacgaacaag

SEQ ID NO: 182 - DNA, 25 bp, Hepatitis E Virus
  HEVConsORF2/3-s1
gtatcggkykgaatgaataacatgt

SEQ ID NO: 183 - DNA, 25 bp, Hepatitis E Virus
  HEVConsORF2/3-a1
aggggttggttggatgaatataggg

SEQ ID NO: 184 - DNA, 234 bp, Hepatitis E Virus
  z12.orf23.con
gtatcggkttgaatgaataacatgttttgtgcatcgcccatgggatcaccatgcgccctagggttgttctgttgttgttcctcgtgtttctgcctatgctgcccgcgccaccggccggccagycgactggccgccgtcgtgggcggcgcagcggcggtgccggcggtggtttctggggtgacagggttgattctcagcccttcgccctcccctatattcatccaaccaacccct

SEQ ID NO: 185 - DNA, 890 bp, Hepatitis E Virus
  z12-3p.race
gtcgtctcggccaatggcgagccgactgtcaagttatacacatctgttgagaatgcacagcaggataaggggatagctattccacatgacatagatttgggcgactctcgtttggtaatccaggattacgataatcagcacgagcaggaccggcccaccccttcgcccgccccgtctcgtcctttctcggtcctccgcgctaatgatgctttgtggctttctcttaccgctgctgagtatgaccagactacatatgggtcgtccaccaacccgatgtatgtctcagacactgttacatttgtcaatgtggccacaggggctcaggctgtcgcccgttctcttgattggtctaaagttaccctggacggccgccctcttactaccatccagcagtactctaagacattttatgttctcccacttcgcgggaagttatctttttgggaggctggcacaactaaagccggttacccttataattataacacaactgctagtgaccagattctgattgaaaacgcggctggccatcgtgtcgctatatctacttatactactagcctgggcgccggccctgtgtcagtttctgcggttggtgtgttagccccacactcgagccttgctattcttgaagacactgttgactatccggcccgtgctcacacttttgatgacttctgtccggaatgccgtgccctgggtctgcaggggtgtgcttttcaatctactatcgctgagctccagcgtcttaaaatgaaggtaggcaaaacccgggagttttaattaattcttcttgtgcccccttcacggttctcgctttatttctttcttctgcctcccgcgctccctggaaaaaaaaaaaaaaaaaaagtactagtcgacgcgtggcc

SEQ ID NO: 186 - DNA, 919 bp, Hepatitis E Virus
  z12-3p.con
gacagaattaatttcgtcggctgggggtcaactgttctactcccgccctgtcgtctcagccaatggcgagccgactgtcaagttatacacatctgttgagaatgcacagcaggataaggggatagctattccacatgacatagatttgggcgactctcgtttggtaatccaggattacgataatcagcacgagcaggaccggcccaccccttcgcccgccccgtctcgtcctttctcggtcctccgcgctaatgatgctttgtggctttctcttaccgctgctgagtatgaccagactacatatgggtcgtccaccaacccgatgtatgtctcagacactgttacatttgtcaatgtggccacaggggctcaggctgtcgcccgttctcttgattggtctaaagttaccctggacggccgccctcttactaccatccagcagtactctaagacattttatgttctcccacttcgcgggaagttatctttttgggaggctggcacaactaaagccggttacccttataattataacacaactgctagtgaccagattctgattgaaaacgcggctggccatcgtgtcgctatatctacttatactactagcctgggcgccggccctgtgtcagtttctgcggttggtgtgttagccccacactcgagccttgctattcttgaagacactgttgactatccggcccgtgctcacacttttgatgacttctgtccggaatgccgtgccctgggtctgcaggggtgtgcttttcaatctactatcgctgagctccagcgtcttaaaatgaaggtaggcaaaacccgggagttttaattaattcttcttgtgcccccttcacggttctcgctttatttctttcttctgcctcccgcgctccctggaaaaaaaaaaaaaaaaaaa

SEQ ID NO: 187 - protein, 138 aa, Hepatitis E Virus
  z12-orf1.pep
GITTAIEQAALGAANSALANAVVVRPFLSRLQTEILINLMQPRQLVFRPEVFWNHPIQRVIHNELEQYCRARAGRCLEIGAHPRSINDNPNVLHRCFLRPVGRDVQRWYSAPTRGPAANCRRSALRGLPPVDRTYCLD

SEQ ID NO: 188 - protein, 61 aa, Hepatitis E Virus
  z12-orf2-5′.pep
MRPRVVLLLFLVFLPMLPAPPAGQXTGRRRGRRSGGAGGGFWGDRVDSQPFALPYIHPTNP

SEQ ID NO: 189 - protein, 276 aa, Hepatitis E Virus
  z12-orf2-3′.pep
TELISSAGGQLFYSRPVVSANGEPTVKLYTSVENAQQDKGIAIPHDIDLGDSRLVIQDYDNQHEQDRPTPSPAPSRPFSVLRANDALWLSLTAAEYDQTTYGSSTNPMYVSDTVTFVNVATGAQAVARSLDWSKVTLDGRPLTTIQQYSKTFYVLPLRGKLSFWEAGTTKAGYPYNYNTTASDQILIENAAGHRVAISTYTTSLGAGPVSVSAVGVLAPHSSLAILEDTVDYPARAHTFDDFCPECRALGLQGCAFQSTIAELQRLKMKVGKTREF

SEQ ID NO: 190 - protein, 74 aa, Hepatitis E Virus
  z12-orf3.pep
MNNMFCASPMGSPCALGLFCCCSSCFCLCCPRHRPASRLAAVVGGAAAVPAVVSGVTGLILSPSPSPIFIQPTP

SEQ ID NO: 191 - DNA, 408 bp, Hepatitis E Virus
  pJOorf3-29.seq
gaattcatgaataacatgtcttttgcatcgcccatgggatcaccatgcgccctagggctgttctgttgttgctcttcgtgcttttgcctatgctgcccgcgccaccggccagccagccgtctggccgccgtcgtgggcggcgcagcggcggtgccggcggtggtttctggggtgacagggttgattctcagcccttcgccctcccctatattcatccaaccaaccccttcgccgccgatgtcgtttcacaacccggggctggaactcgccctcgacagccgccccgcccccttggctccgcttggcgtgaccagtcccagcgcccctccgctgccccccgtcgtcgatctgccccagcttggtctgcgccgcgactacaaggacgacgatgacaagtaataaggatcc

SEQ ID NO: 192 - DNA, 1026 bp, Hepatitis E Virus
  cksorf2m-2.seq
gaattcatgggtgctgatgggactgctgagcttactaccacagcagccacacgtttcatgaaggacctgcacttcgctggcacgaatggcgttggtgaggtgggtcgtggtatcgccctgacactgttcaatctcgctgatacgcttctcggcggtttaccgacagaattgatttcgtcggctgggggccaactgttttactcccgcccggttgtctcagccaatggcgagccaacagtaaagttatatacatctgttgagaatgcgcagcaagacaagggcatcaccattccacatgatatagacctgggtgactcccgtgtggttatccaggattatgataaccagcatgagcaagaccgacctactccgtcacctgccccctctcgccccttctcagttcttcgtgccaatgatgttttgtggctttccctcactgccgctgagtatgaccagactacgtatgggtcgtccaccaaccctatgtatgtctctgacacagttacgcttgttaatgtggctactggtgctcaggctgttgcccgctcccttgattggtctaaagttactctggacggccgcccccttactaccattcagcagtattctaagacattttatgttctcccgctccgcgggaagctgtccttttgggaggctggcacgactaaggccggctacccttacaattataatactaccgctagtgaccaaattttgattgagaatgcggccggccaccgtgtcgctatttccacctataccactagcttaggtgccggtcctacctcgatctctgcggtcggcgtactggctccacactctgcccttgccgttcttgaggatactattgattaccccgcccgtgcccatacttttgatgatttttgcccggagtgccgtaccctaggtttgcagggttgtgcattccagtctactattgctgagctccagcgtttaaaaatgaaggtaggtaaaacccgggagtctgactacaaggacgacgatgacaagtaataaggatcc

SEQ ID NO: 193 - DNA, 1389 bp, Hepatitis E Virus
  CKSORF32M-3.seq
gaattcatgaataacatgtcttttgcatcgcccatgggatcaccatgcgccctagggctgttctgttgttgctcttcgtgcttttgcctatgctgcccgcgccaccggccagccagccgtctggccgccgtcgtgggcggcgtagcggcggtgccggcggtggtttctggggtgacagggttgattctcagcccttcgccctcccctatattcatccaaccaaccccttcgccgccgatgtcgtttcacaacccggggctggaactcgccctcgacagccgccccgcccccttggctccgcttggcgtgaccagtcccagcgcccctccgctgccccccgtcgtcgatctgccccagcttggtctgcgccgcggtgctgatgggactgctgagcttactaccacagcagccacacgtttcatgaaggacctgcacttcgctggcacgaatggcgttggtgaggtgggtcgtggtatcgccctgacactgttcaatctcgctgatacgcttctcggcggtttaccgacagaattgatttcgtcggctgggggccaactgttttactcccgcccggttgtctcagccaatggcgagccaacagtaaagttatatacatctgttgagaatgcgcagcaagacaagggcatcaccattccacatgatatagacctgggtgactcccgtgtggttatccaggattatgataaccagcatgagcaagaccgacctactccgtcacctgccccctctcgccccttctcagttcttcgtgccaatgatgttttgtggctttccctcactgccgctgagtatgaccagactacgtatgggtcgtccaccaaccctatgtatgtctctgacacagttacgcttgttaatgtggctactggtgctcaggctgttgcccgctcccttgattggtctaaagttactctggacggccgcccccttactaccattcagcagtattctaagacattttatgttctcccgctccgcgggaagctgtccttttgggaggctggcacgactaaggccggctacccttacaattataatactaccgctagtgaccaaattttgattgagaatgcggccggccaccgtgtcgctatttccacctataccactagcttaggtgccggtcctacctcgatctctgcggtcggcgtactggctccacactctgcccttgccgttcttgaggatactattgattaccccgcccgtgcccatacttttgatgatttttgcccggagtgccgtaccctaggtttgcagggttgtgcattccagtctactattgctgagctccagcgtttaaaaatgaaggtaggtaaaacccgggagtctgactacaaggacgacgatgacaagtaataaggatcc

SEQ ID NO: 194 - DNA, 408 bp, Hepatitis E Virus
  plorf3-12.con
gaattcatgaataacatgtcttttgcatcgcccatgggatcaccatgcgccctagggctgttctgttgttgctcttcgtgcttttgcctatgctgcccgcgccaccggccggccagccgtctggccgccgtcgtgggcggcgcagcggcggtgccggcggtggtttctggggtgacagggttgattctcagcccttcgccctcccctatattcatccaaccaaccccttcgccgccgatgtcgtttcacaacccggggctggaactcgccctcgacagccgccccgcccccttggctccgcttggcgtgaccagtcccagcgcccctccgctgccccccgtcgtcgatctgccccagcttggtctgcgccgcgactacaaggacgacgatgacaagtaataaggatcc

SEQ ID NO: 195 - DNA, 1026 bp, Hepatitis E Virus; identical to SEQ ID NO: 192
  plorf2.2-6.seq

SEQ ID NO: 196 - DNA, 1389 bp, Hepatitis E Virus; identical to SEQ ID NO: 193
  PLORF32M-14-5.seq

SEQ ID NO: 197 - protein, 74 aa, Hepatitis E Virus
  z12-orf3-5′.pep
MNNMFCASPMGSPCALGLFCCCSSCFCLCCPRHRPAXRLAAVVGGAAAVPAVVSGVTGLILSPSPSPIFIQPTP

SEQ ID NO: 198 - DNA, 63 bp, Hepatitis E Virus
  Primer orf23p
tatatggatccttattacttgtcatcgtcgtccttgtagtcagactcccgggttttacctacc

SEQ ID NO: 199 - protein, 338 aa, Hepatitis E Virus
  cksorf2m-2.pep
EFMGADGTAELTTTAATRFMKDLHFAGTNGVGEVGRGIALTLFNLADTLLGGLPTELISSAGGQLFYSRPVVSANGEPTVKLYTSVENAQQDKGITIPHDIDLGDSRVVIQDYDNQHEQDRPTPSPAPSRPFSVLRANDVLWLSLTAAEYDQTTYGSSTNPMYVSDTVTLVNVATGAQAVARSLDWSKVTLDGRPLTTIQQYSKTFYVLPLRGKLSFWEAGTTKAGYPYNYNTTASDQILIENAAGHRVAISTYTTSLGAGPTSISAVGVLAPHSALAVLEDTIDYPARAHTFDDFCPECRTLGLQGCAFQSTIAELQRLKMKVGKTRESDYKDDDDK

SEQ ID NO: 200 - protein, 338 aa, Hepatitis E Virus; identical to SEQ ID NO: 199
  plorf2.2-6.pep

SEQ ID NO: 201 - DNA, 37 bp, Hepatitis E Virus
  Primer orf35p
tatatgaattcatgaataacatgtcttttgcatcgcc

SEQ ID NO: 202 - DNA, 68 bp, Hepatitis E Virus
  Primer orf33p
tatatggatccttattacttgtcatcgtcgtccttgtagtcgcggcgcagaccaagctggggcagatc

SEQ ID NO: 203 - protein, 132 aa, Hepatitis E Virus
  pJOorf3-29.pep
EFMNNMSFASPMGSPCALGLFCCCSSCFCLCCPRHRPASRLAAVVGGAAAVPAVVSGVTGLILSPSPSPIFIQPTPSPPMSFHNPGLELALDSRPAPLAPLGVTSPSAPPLPPVVDLPQLGLRRDYKDDDDK

SEQ ID NO: 204 - protein, 132 aa, Hepatitis E Virus; identical to SEQ ID NO: 203
  plorf3-12.pep

SEQ ID NO: 205 - DNA, 48 bp, Hepatitis E Virus
  Primer orf23
ctcagcagtcccatcagcaccgcggcgcagaccaagctggggcagatc

SEQ ID NO: 206 - protein, 459 aa, Hepatitis E Virus
  CKSORF32M-3.pep
EFMNNMSFASPMGSPCALGLFCCCSSCFCLCCPRHRPASRLAAVVGGVAAVPAVVSGVTGLILSPSPSPIFIQPTPSPPMSFHNPGLELALDSRPAPLAPLGVTSPSAPPLPPVVDLPQLGLRRGADGTAELTTTAATRFMKDLHFAGTNGVGEVGRGIALTLFNLADTLLGGLPTELISSAGGQLFYSRPVVSANGEPTVKLYTSVENAQQDKGITIPHDIDLGDSRVVIQDYDNQHEQDRPTPSPAPSRPFSVLRANDVLWLSLTAAEYDQTTYGSSTNPMYVSDTVTLVNVATGAQAVARSLDWSKVTLDGRPLTTIQQYSKTFYVLPLRGKLSFWEAGTTKAGYPYNYNTTASDQILIENAAGHRVAISTYTTSLGAGPTSISAVGVLAPHSALAVLEDTIDYPARAHTFDDFCPECRTLGLQGCAFQSTIAELQRLKMKVGKTRESDYKDDDDK

SEQ ID NO: 207 - protein, 459 aa, Hepatitis E Virus; identical to SEQ ID NO: 206
  PLORF32M-14-5.pep

SEQ ID NO: 208 - DNA, 36 bp, Hepatitis E Virus
  Primer orf2mid5p
tatatgaattcatgggtgctgatgggactgctgagc

SEQ ID NO: 209 - DNA, 418 bp, Hepatitis E Virus
  1440o1.seq
ctggcatyactactgcyattgagcaggctgctctggctgcggccaattccgccttggcgaatgctgtggtggttcggccgtttttatcccgtgttcaaactgatatccttattaacctgatgcaaccccgtcagcttgtgttccggcctgaagttctctggaaccatccgatccagcgagttatacataatgagctggaacaatactgtcgagcccgcgctggccgctgtcttgaggtgggcgctcacccaaggtctattaatgataaccccaatgttctgcaccggtgctttctccgcccggttgggagagacgtccagcgctggtattccgcccccactcgtggtccagcggctaactgccgccgttctgcgctacgcggtttgccccctgtcgaccgcacttactgtytygatgg

SEQ ID NO: 210 - protein, 138 aa, Hepatitis E Virus
  Xaa = Unknown or Other at position 2
GXTTXIEQAALAAANSALANAVVVRPFLSRVQTDILINLMQPRQLVFRPEVLWNHPIQRVIHNELEQYCRARAGRCLEVGAHPRSINDNPNVLHRCFLRPVGRDVQRWYSAPTRGPAANCRRSALRGLPPVDRTYCXD

SEQ ID NO: 211 - DNA, 197 bp, Hepatitis E Virus
  1440o2.seq
gacagaattratttcgtcggctggaggtcaactgttctactcccgcccggttgtctcagccaatggcgagccgactgttaagttatacacctctgtcgagaatgcacagcaggataagggcattgctataccacatgatatagacttaggggattcccgtgtggttatacaagattatgayaaccarcaygaacaag

SEQ ID NO: 212 - protein, 65 aa, Hepatitis E Virus
  Xaa = Unknown or Other at position 3
TEXISSAGGQLFYSRPVVSANGEPTVKLYTSVENAQQDKGIAIPHDIDLGDSRVVIQDYXNXXEQ

SEQ ID NO: 213 - DNA, 418 bp, Hepatitis E Virus
  2015-1.seq
ctggcatyactactgcyattgagcaggctgctctggctgcggctaactctgccttggcgaatgctgtggtggtccggccgttcctgtcccgcactcagactgatattcttattaatttgatgcaaccccggcaacttgtattccgccctgaggttttgtggaaccatccgatccagcgagtcatacataatgagctggagcagtattgccgtgctcgtgctggtcgctgcctggaggttggggctcatccaagatctatcaatgacaaccctaatgttctgcaccggtgtttcctccgtccggttgggcgagacgtacagcgttggtattctgcccctactcgcggcccggcggctaattgccgccgttccgcgttacgtggcctacctcctgtcgaccgcacttactgtytygatgg

SEQ ID NO: 214 - protein, 138 aa, Hepatitis E Virus
  Xaa = Unknown or Other at position 2
GXTTXIEQAALAAANSALANAVVVRPFLSRTQTDILINLMQPRQLVFRPEVLWNHPIQRVIHNELEQYCRARAGRCLEVGAHPRSINDNPNVLHRCFLRPVGRDVQRWYSAPTRGPAANCRRSALRGLPPVDRTYCXD

SEQ ID NO: 215 - DNA, 197 bp, Hepatitis E Virus
  2015o2.seq
gacagaattratttcgtcggctggaggccagctcttctactcccgcccagtcgtctcagccaatggcgagccgactgttaaattgtatacatccgtcgagaatgcgcagcaggacaagggcattgccataccacatgatatagatctaggagattcccgcgtggttatccaggattatgayaaccarcaygaacaag

SEQ ID NO: 216 - protein, 65 aa, Hepatitis E Virus; identical to SEQ ID NO: 212
  Xaa = Unknown or Other at position 3

SEQ ID NO: 217 - DNA, 251 bp, Hepatitis E Virus
  14404-2.seq
atattcatccaaccaacccctttgcctccgacgtcgtatcgcaatccggggctggagctcgccctcgacagccggcccgccccctcggctcctcttggcgtgaccagtcccagcgcccccccgctgtcccccgtcgtcgatctaccccaactggggctgcgccgctaactgctgtttcaccagcgcctgatacggccccagtccctgatgttgactctcgtggcgctatcttgcgccggcagtataaccta

SEQ ID NO: 218 - protein, 83 aa, Hepatitis E Virus
IHPTNPFASDVVSQSGAGARPRQPARPLGSSWRDQSQRPPAVPRRRSTPTGAAPLTAVSPAPDTAPVPDVDSRGAILRRQYNL

SEQ ID NO: 219 - protein, 55 aa, Hepatitis E Virus
  14404-2.seq orf3
IFIQPTPLPPTSYRNPGLELALDSRPAPSAPLGVTSPSAPPLSPVVDLPQLGLRR

SEQ ID NO: 220 - DNA, 251 bp, Hepatits E Virus
  20154-2.seq
atattcatccaaccaacccctttgccgccgacgtcgtatcacaacccggggctggagctcgccctcgacagccgccccgccccctcggctcctcttggcgtgatcagtcccagcgcccctccgctgccccccgtcgtcgatctaccccagctggggctgcgccgttaactgctgtttcccctgcgcccgatacggccccagtccccgacgttgattcccgtggtgccatcctgcgccggcagtataaccta

SEQ ID NO: 221 - protein, 83 aa, Hepatitis E Virus
IHPTNPFAADVVSQPGAGARPRQPPRPLGSSWRDQSQRPSAAPRRRSTPAGAAPLTAVSPAPDTAPVPDVDSRGAILRRQYNL

SEQ ID NO: 222 - protein, 55 aa, Hepatitis E Virus
  20154-2.seq orf3
IFIQPTPLPPTSYHNPGLELALDSRPAPSAPLGVISPSAPPLPPVVDLPQLGLRR

SEQ ID NO: 223 - protein, 48 aa, Hepatitis E Virus
  US-2 3-2e
TIDYPARAHTFDDFCPECRTLGLQGCAFQSTIAELQRLKMKVGKTRES

SEQ ID NO: 224 - protein, 33 aa, Hepatitis E Virus
  US-2 4-2
DSRPAPLVPLGVTSPSAPPLPPVVDLPQLGLRR

SEQ ID NO: 225 - DNA, 450 bp, Hepatitis E Virus
  5p.pile {hpesvp}
ggctcctggcatcactactgctattgagcaggctgctctagcagcggccaactctgccctggcgaatgctgtggtagttaggccttttctctctcaccagcagattgagatcctcattaacctaatgcaacctcgccagcttgttttccgccccgaggttttctggaatcatcccatccagcgtgtcatccataacgagctggagctttactgccgcgcccgctccggccgctgtcttgaaattggcgcccatccccgctcaataaatgataatcctaatgtggtccaccgctgcttcctccgccctgttgggcgtgatgttcagcgctggtatactgctcccactcgcgggccggctgctaattgccggcgttccgcgctgcgcgggcttcccgctgctgaccgcacttactgcctcgacgggttttctggctgtaactttcccgccga

SEQ ID NO: 226 - DNA, 450 bp, Hepatitis E Virus
  5p.pile {hpeuigh}
ggctcctggcatcactactgctattgagcaggctgctctagcagcggccaattctgcccttgcgaatgctgtggtagttaggccttttctctctcaccagcagattgagatccttattaacctaatgcaacctcgccagcttgttttccgccccgaggttttctggaaccaccccatccagcgtgtcatccataatgagctggagctttactgtcgcgcccgctccggccgctgccttgaaattggtgcccaccctcgctcaataaacgacaatcctaatgtggtccaccgctgcttcctccgccctgccgggcgtgatgttcagcgttggtatactgctcctacccgcgggccggctgctaattgccggggttccgcactgcgcgggctccccgctgctgaccgcacttactgcttcgacgggttttctggctgtaactttcccgccga

SEQ ID NO: 227 - DNA, 450 bp, Hepatitis E Virus
  5p.pile {hpea}
ggctcctggcatcactactgctattgagcaggctgctctagcagcggccaactctgcccttgcgaatgctgtggtagttaggccttttctctctcaccagcagattgagatccttattaacctaatgcaacctcgccagcttgttttccgccccgaggttttctggaaccatcccatccagcgtgttatccataatgagctggagctttactgtcgcgcccgctccggccgctgcctcgaaattggtgcccacccccgctcaataaatgacaatcctaatgtggtccaccgttgcttcctccgtcctgccgggcgtgatgttcagcgttggtatactgcccctacccgcgggccggctgctaattgccggcgttccgcgctgcgcgggctccccgctgctgaccgcacttactgcttcgacgggttttctggctgtaactttcccgccga

SEQ ID NO: 228 - DNA, 446 bp, Hepatitis E Virus
  5p.pile {840455p}
cctggcattactactgccattgagcaggctgctctggctgcggccaattctgccttggcgaatgctgtggtggttcggccgtttttatctcgcgtgcaaaccgagattcttattaatttgatgcaaccccggcagttggttttccgccctgaggtactttggaatcaccctatccagcgggttatacataatgaattagaacagtactgccgggctcgggctggtcgttgcttggaggttggagctcacccaagatccattaatgacaaccccaacgttctgcatcggtgtttccttagaccggttggccgagatgttcagcgctggtactctgcccccacccgcggccctgcggctaattgccgccgctccgcgttgcgtggtctcccccccgctgaccgcacttactgctttgatggattctcccgttgtgcttttgctgcaga

SEQ ID NO: 229 - DNA, 450 bp, Hepatitis E Virus
  5p.pile {hpenssp}
ggctcctggcatcactactgctattgagcaagcagctctagcagcggccaactccgcccttgcgaatgctgtggtggtccggcctttcctttcccatcagcaggttgagatccttataaatctcatgcaacctcggcagctggtgtttcgtcctgaggttttttggaatcacccgattcaacgtgttatacataatgagcttgagcagtattgccgtgctcgctcgggtcgctgccttgagattggagcccacccacgctccattaatgataatcctaatgtcctccatcgctgctttctccaccccgtcggccgggatgttcagcgctggtacacagccccgactaggggacctgcggcgaactgtcgccgctcggcacttcgtggtctgccaccagccgaccgcacttactgttttgatggctttgccggctgccgttttgccgccga

SEQ ID NO: 230 - DNA, 450 bp, Hepatitis E Virus
  5p Consensus
nnnncctggcatnactactgcnattgagcangcngctctngcngcggccaantcngccntngcgaatgctgtggtngtnnggccnttnntntcncnnnngcannnngagatnctnatnaanntnatgcaaccncgncagntngtnttncgnccngaggtnntntggaancanccnatncancgngtnatncataanganntngancnntantgncgngcncgnncnggncgntgnntngannttggngcncanccnngntcnatnaanganaanccnaangtnntncancgntgnttnctnnnnccngnnggncgngatgttcagcgntggtanncngcnccnacnngnggnccngcngcnaantgncgnngntcngcnntncgnggnctnccnncngcngaccgcacttactgnntnganggnttnncnngntgnnnntttncngcnga

SEQ ID NO: 231 - DNA, 300 bp, Hepatitis E Virus
  3p.pile {hpea} shown in Figure 9B
actgagtcagtgaagccagtgcttgacctgacaaattcaattctgtgtcgggtggaatgaataacatgtcttttgctgcgcccatgggttcgcgaccatgcgccctcggcctattttgctgttgctcctcatgtttctgcctatgctgcccgcgccaccgcccggtcagccgtctggccgccgtcgtgggcggcgcagcggcggttccggcggtggtttctggggtgaccgggttgattctcagcccttcgcaatcccctatattcatccaaccaaccccttcgcccccgatgtcaccgc

SEQ ID NO: 232 - DNA, 300 bp, Hepatitis E Virus
  3p.pile {hpeuigh} shown in Figure 9B
actgagtcggtgaagccagtgctcgacttgacaaattcaatcctgtgtcgggtggaatgaataacatgtcttttgctgcgcccatgggttggcgaccatgcgccctcggcctattttgctgttgctcctcatgtttctgcctatcgtgcccgcgccaccgcccggtcagccgtctggccgccgtcgtgggcggcgcagcggcggttccggcggtggtttctggggtgaccgggttgattctcagcccttcgcaatcccctatattcatccaaccaaccccttcgcccccgatgtcaccgc

SEQ ID NO: 233 - DNA, 300 bp, Hepatitis E Virus
  3p.pile {hpesvp} shown in Figure 9B
actgagtcagtaaaaccagtgctcgacttgacaaattcaatcttgtgtcgggtggaatgaataacatgtcttttgctgcgcccatgggttcgcgaccatgcgccctcggcctattttgttgctgctcctcatgtttttgcctatgctgcccgcgccaccgcccggtcagccgtctggccgccgtcgtgggcggcgcagcggcggttccggcggtggtttctggggtgaccgggttgattctcagcccttcgcaatcccctatattcatccaaccaaccccttcgcccccgatgtcaccgc

SEQ ID NO: 234 - DNA, 300 bp, Hepatitis E Virus
  3p.pile {hpenssp} shown in Figure 9B
acagagtctgttaagcctatacttgaccttacacactcaattatgcaccggtctgaatgaataacatgtggtttgctgcgcccatgggttcgccaccatgcgccctaggcctcttttgctgttgttcctcttgtttctgcctatgttgcccgcgccaccgaccggtcagccgtctggccgccgtcgtgggcggcgcagcggcggtaccggcggtggtttctggggtgaccgggttgattctcagcccttcgcaatcccctatattcatccaaccaacccctttgccccagacgttgccgc

SEQ ID NO: 235 - DNA, 297 bp, Hepatitis E Virus
  3p.pile {840453p} shown in Figure 9B
acagagactattaaacctgtacttgatctcacaaattccatcatacagcgggtggaatgaataacatgtcttttgcatcgcccatgggatcaccatgcgccctagggctgttctgttgttgttcctcatgtttctgcctatgctgcccgcgccaccggccggtcagccgtctggccgtcgccgtgggcggcgcagcggcggtgccggcggtggtttctggagtgacagggttgattctcagcccttcgccctcccctatattcatccaaccaaccccttcgccgccgatgtcgtttc

SEQ ID NO: 236 - DNA, 300 bp, Hepatitis E Virus
  3p Consensus shown in Figure 9B
acngagncnntnaanccnntnctnganntnacanantcnatnntnnnncggnnngaatgaataacatgtnntttgcnncgcccatgggntnnnnaccatgcgccctnggnctnttntgntgntgntcctcntgtttntgcctatnntgcccgcgccaccgnccggtcagccgtctggccgncgncgtgggcggcgcagcggcggtnccggcggtggtttctggngtgacngggttgattctcagcccttcgcnntcccctatattcatccaaccaaccccttngccncngangtnnnnnc

SEQ ID NO: 237 - DNA, 250 bp, Hepatitis E Virus
  3p.pile {hpea} shown in Figure 9C
agcgcttaccctgtttaaccttgctgacaccctgcttggcggtctaccgacagaattgatttcgtcggctggtggccagctgttctactctcgccccgtcgtctcagccaatggcgagccgactgttaagctgtatacatctgtggagaatgctcagcaggataagggtattgcaatcccgcatgacatcgacctcggggaatcccgtgtagttattcaggattatgacaaccaacatgagcaggaccga

SEQ ID NO: 238 - DNA, 250 bp, Hepatitis E Virus
  3p.pile {hpeuigh} shown in Figure 9C
agcgcttaccctgtttaaccttgctgacaccctgcttggcggtctaccgacagaattgatttcgtcggctggtggccagctgttctactctcgccccgtcgtctcagccaatggcgagccgactgttaagctgtatacatctgtagagaatgctcagcaggataagggtattgcaatcccgcatgacatcgacctcggggaatctcgagttgttattcaggattatgacaaccaacatgagcaggaccgg

SEQ ID NO: 239 - DNA, 250 bp, Hepatitis E Virus
  3p.pile {hpesvp} shown in Figure 9C
agccctcaccctgttcaaccttgctgacactctgcttggcggcctgccgacagaattgatttcgtcggctggtggccagctgttctactcccgtcccgttgtctcagccaatggcgagccgactgttaagttgtatacatctgtagagaatgctcagcaggataagggtattgcaatcccgcatgacattgacctcggagaatctcgtgtggttattcaggattatgataaccaacatgaacaagatcgg

SEQ ID NO: 240 - DNA, 250 bp, Hepatitis E Virus
  3p.pile {hpenssp} shown in Figure 9C
agctctaacattacttaaccttgctgacacgctcctcggcgggctcccgacagaattaatttcgtcggctggcgggcaactgttttattcccgcccggttgtctcagccaatggcgagccaaccgtgaagctctatacatcagtggagaatgctcagcaggataagggtgttgctatcccccacgatatcgatcttggtgattcgcgtgtggtcattcaggattatgacaaccagcatgagcaggatcgg

SEQ ID NO: 241 - DNA, 250 bp, Hepatitis E Virus
  3p.pile {840453p} shown in Figure 9C
tgccctgactctgtttaatcttgctgatacgcttcttggtggtttaccgacagaattgatttcgtcggctgggggtcaactgttttactcccgccctgttcagaattgatttcgtcggctgggggtcaactgttttactcccgccctgtttgcgcagcaagacaagggcatcaccattccacacgacatagatttaggtgactcccgtgtggttatccaggattatgataaccagcacgaacaagatcga

SEQ ID NO: 242 - DNA, 250 bp, Hepatitis E Virus
  3p Consensus shown in Figure 9C
ngcnctnacnntnntnaancttgctganacnctnctnggnggnntnccgacagaattnatttcgtcggctggnggncanctgttntantcncgnccngtngtctcngccaatggcgagccnacngtnaagntntanacatcngtngagaatgcncagcanganaagggnntnncnatnccncanganatnganntnggngantcncgngtngtnatncaggattatganaaccancangancangancgn

SEQ ID NO: 243 - DNA, 418 bp, Hepatitis E Virus
  Au1o1-w1abo1p1.pat
ctggcatyactactgcyattgagcaagctgctctggctgcggccaattctgccttggcgaatgctgtggtggttcggccgtttttatcccgtgtgcagactgagatccttattaacttgatgcaacctcggcagctggtgttccgacctgaggtgctttggaatcatcccattcagcgggttatccataatgagttagaacaatactgccgggcccgggccggccgttgcctagaggtgggggcccacccaaggtccattaacgataaccccaatgttttgcaccggtgttttctgcgaccggtcgggagggatgttcagcgctggtactctgcccccacccgcggccctgcggctaactgccgccgctccgctttgcgtggccttccccccgtcgaccgcacttactgtytygatgg

SEQ ID NO: 244 - protein, 138 aa, Hepatitis E Virus
  Xaa = Unknown or Other at position 2
GXTTXIEQAALAAANSALANAVVVRPFLSRVQTEILINLMQPRQLVFRPEVLWNHPIQRVIHNELEQYCRARAGRCLEVGAHPRSINDNPNVLHRCFLRPVGRDVQRWYSAPTRGPAANCRRSALRGLPPVDRTYCXD

SEQ ID NO: 245 - DNA, 197 bp, Hepatitis E Virus
  Au1o2-w1ao2.pat
gacagaattratttcgtcggctgggggacagttattctactcccgccctgtygtctcagccaatggcgagccgactgttaaattatatacatctgtagagaatgcgcagcaggacaaggggattgccatcccacatgatatagatctgggcgactctcgtgtggtgatccaggattatgayaaccarcaygaacaag

SEQ ID NO: 246 - protein, 65 aa, Hepatitis E Virus
  Xaa = Unknown or Other at position 3
TEXISSAGGQLFYSRPXVSANGEPTVKLYTSVENAQQDKGIAIPHDIDLGDSRVVIQDYXNXXEQ

SEQ ID NO: 247 - DNA, 418 bp, Hepatitis E Virus
  Ar1o1-f73o1p1.pat
ctggcatyactactgcyattgagcaagctgctctggctgcggccaactctgccttggcgaatgctgtggtggttcggccgtttttatcccgtgtgcagaccgagattcttattaacctaatgcaaccccggcagctggtttttcgtcctgaggtgctttggaaccatcctatccagcgggttattcataatgagttagaacagtactgtcgggctcgggctggtcgctgcctagaggtcggggcccacccaaggtccattaatgataaccctaatgttttgcaccggtgcttcctacgaccagtcgggagggatgttcaacgttggtattccgcccccacccgcggtcctgctgccaactgccgccgttccgctctgcgcggcctccctcccgtcgaccgcacttactgtytygatgg

SEQ ID NO: 248 - protein, 138 aa, Hepatitis E Virus; identical to SEQ ID NO: 244
  Xaa = Unknown or Other at position 2

SEQ ID NO: 249 - DNA, 145 bp, Hepatitis E Virus
  Ar1-f73o2p2.pat
gtygtctcrgccaatggcgagccgactgttaagctatatacatctgtagagaacgcgcagcaggataaagggatcgccattccacacgatatagatctgggcgattcccgtgtggtcattcaggattatgayaaccarcaygaac

SEQ ID NO: 250 - protein, 48 aa, Hepatitis E Virus
  Xaa = Unknown or Other at position 1
XVXANGEPTVKLYTSVENAQQDKGIAIPHDIDLGDSRVVIQDYXNXXE

SEQ ID NO: 251 - DNA, 418 bp, Hepatitis E Virus
  Ar2o1-f77o1p1.pat
ctggcatyactactgcyattgagcaagctgctctggctgcggctaactctgccttggcgaatgctgtggtggttcggccgtttctatcccgtgtgcagactgagatccttattaacttaatgcarccccggcarctggttttccgtcccgaggtgctttggaatcatcccattcaacgggttattcataatgaattagagcagtactgccggacccgggctggccgttgtttagaggtcggagcccatccaaggtccattaatgacaaccctaacgttcygcaccggtgcttcttacgaccagtcgggagggatgtccaacgatggtactcagcccccactcgcggccctgcggctaattgccgtcgttccgctttgcgtggtctccctcctgtcgaccgcacttactgtytygatgg

SEQ ID NO: 252 - protein, 138 aa, Hepatitis E Virus
  Xaa = Unknown or Other at position 2
GXTTXIEQAALAAANSALANAVVVRPFLSRVQTEILINLMXPRXLVFRPEVLWNHPIQRVIHNELEQYCRTRAGRCLEVGAHPRSINDNPNVXHRCFLRPVGRDVQRWYSAPTRGPAANCRRSALRGLPPVDRTYCXD

SEQ ID NO: 253 - DNA, 197 bp, Hepatitis E Virus
  Ar2o2-f7702.pat
gacagaattratttcgtcggctgggggtcagttgttttactcccgccctgtcgtctcagccaatggcgagccgactgttaagttgtatacatctgtggagaatgcgcagcaggataaaggaatcgccatcccacacgacatagatctgggcgattcccgtgtggttattcaggattatgayaaccarcaygaacaag

SEQ ID NO: 254 - protein, 65 aa, Hepatits E Virus; identical to SEQ ID NO: 212
  Xaa = Unknown or Other at position 3

SEQ ID NO: 255 - DNA, 23 bp, Hepatits E Virus
  HEVConsORF 1N-a1
ccrtcrarrcartaggtgcggtc

SEQ ID NO: 256 - DNA, 25 bp, Hepatits E Virus
  HEVConsORF 2N-a1
cytgytcrtgytggttrtcataatc

SEQ ID NO: 257 - DNA, 21 bp, Hepatits E Virus
  HEVConsORF 1N-s2
cygccytkgcgaatgctgtgg

SEQ ID NO: 258 - DNA, 25 bp, Hepatits E Virus
  HEVConsORF 2N-a2
gytcrtgytgrttrtcataatcctg

(19) United States
(12) Patent Application Publication
(10) Pub. No.: US 2003/0049601 A1
(43) Pub. Date: Mar. 13, 2003

(54) METHODS AND COMPOSITIONS FOR DETECTING HEPATITIS E VIRUS

(76) Inventors: GEORGE G. SCHLAUDER, SKOKIE, IL (US); JAMES C. ERKER, GRAYSLAKE, IL (US); SURESH M. DESAI, LIBERTYVILLE, IL (US); GEORGE J. DAWSON, LIBERTYVILLE, IL (US); ISA K. MUSHAHWAR, GRAYSLAKE, IL (US)

Correspondence Address: ABBOTT LABORATORIES, DEPT. 377 - AP6D-2, 100 ABBOTT PARK ROAD, ABBOTT PARK, IL, 60064-6050, US

(21) Appl. No.: 09/468,147

(22) Filed: Dec. 21, 1999

Related U.S. Application Data
(63) Continuation-in-part of application No. 09/173,141, filed on Oct. 15, 1998, now abandoned.
(60) Provisional application No. 60/061,199, filed on Oct. 15, 1997.

(51) Int. Cl. C12Q 1/70

(52) U.S. Cl. 435/5

(57) ABSTRACT

Disclosed herein are methods and compositions for detecting the presence in a sample of a US-type or a US-subtype hepatitis E virus, including naturally occurring variants thereof. In particular, the invention provides nucleic acid sequences corresponding to the genome of the US-type or US-subtype hepatitis E virus, amino acid sequences, including epitope sequences, encoded by the genomes of such viruses, and antibodies that bind specifically to such amino acid sequences. The invention further provides methods and compositions for immunizing individuals against infection by, or for treating individuals already infected with such a virus.

### RELATED APPLICATIONS

This application claims priority to U.S. Ser. No. 09/173,141, filed Oct. 15, 1998, now pending, which claims priority under 35 U.S.C. §119(e) to provisional application U.S. Ser. No. 60/061,199, filed Oct. 15, 1997, now abandoned, the disclosures of which are incorporated by reference herein.

### FIELD OF THE INVENTION

This invention relates generally to methods and compositions for detecting hepatitis E virus, and more particularly to methods and compositions for detecting in, or treating individuals infected with US-type and US-subtype strains of hepatitis E virus.

### BACKGROUND OF THE INVENTION

There are at least five major classes of hepatotropic viruses that cause inflammation of the liver (hepatitis). These viruses include hepatitis A virus (HAV), hepatitis B virus (HBV), hepatitis C virus (HCV), hepatitis D virus (HDV) and hepatitis E virus (HEV). Although only HBV, HCV and HDV cause chronic hepatitis, all five types cause acute disease either directly or as a result of superinfection/co-infection by, for example, HBV and HDV. HEV causes symptoms of hepatitis that are similar to those of other viral agents including abdominal pain, jaundice, malaise, anorexia, dark urine, fever, nausea and vomiting (see, for example, Reyes et al., “Molecular biology of non-A, non-B hepatitis agents: hepatitis C and hepatitis E viruses” in Advances in Virus Research (1991) 40: 57-102; Bradley, “Hepatitis non-A, non-B viruses become identified as hepatitis C and E viruses” in Progr. Med. Virol. (1990) 37: 101-135; Hollinger “Non-A, non-B hepatitis viruses” in Virology, Second Edition (1990), Second Edition, Raven Press, New York pp. 2239-2271; Gust et al., “Report of a workshop: waterborne non-A, non-B hepatitis” J. Infect. Dis. (1987) 156: 630-635; and Krawcyznski “Hepatitis E” Hepatology (1993) 17: 932-941). Unlike the other hepatoviruses, however, HEV generally has not been perceived as being a significant cause of hepatitis in the US.

Geographic regions where HEV is endemic include eastern and northern Africa, India, Pakistan, Burma and China (Reyes et al. (1991) supra). The case fatality rate of HEV infection is estimated to be between about 0.1% to about 1.0% in the general population, where HEV is endemic, and as high as about 20% among pregnant women in developing countries. Most fatalities result from fulminant hepatitis (Reyes et al. (1991) supra). The occasional reports of infection with HEV in the US, western Europe and Japan, usually are observed in travelers returning home from visits to areas where HEV in endemic. However, there is little information pertaining to the morbidity and/or mortality of infection with HEV in the US since HEV infections are not reported to a central agency. Extensive, systematic studies have not been performed to determine the importance of HEV in US. Further, if such studies were performed, the relative importance of HEV in US (and possibly Japan and Western Europe) may continue to be underestimated unless the proper reagents are developed to conduct such a study.

The basic features of HEV is that it is a non-enveloped virus, approximately 27-30 nm in diameter possessing a positive sense, single stranded RNA genome which comprises three discontinuous open-reading frames (ORFs), referred to in the art as open reading frame 1 (ORF 1), open reading frame 2 (ORF 2), and open reading frame 3 (ORF 3). Based on the overall morphology of the virus and the size and organization of the genome, the virus is tentatively classified as a member of the Caliciviridae. The first two isolates of HEV to be identified and sequenced were obtained from Burma and from Mexico. The overall nucleic acid identity across the genome of both isolates is 76% (Reyes et al. (1990) Science, 247: 1335-1339; Tam et al. (1991) Virology 185: 120-131; Huang et al. (1992) Virology 191:550-558). Many of the nucleotide differences were noted at the third codon position, such that the deduced similarities in amino acid sequences between the Burmese and Mexican strains of HEV were 83%, 93% and 87%, for open reading frames ORF 1, ORF 2, and ORF 3, respectively.

In the Burmese strain, there is a short non-translated region of about 27 nucleotides at the 5′ end of the genome which has not been identified in the Mexican strain. ORF 1 comprises approximately 5,100 nucleotides, which encode several conserved motifs including a putative methyltransferase domain, an RNA helicase domain, a putative RNA-dependent RNA polymerase (RDRP) domain, and a putative papain-like protease. A tripeptide sequence of Gly-Asp-Asp (GDD), found in all positive-sense RNA plant and animal viruses, is located within ORF 1 and usually signifies RDRP function. Conserved motifs suggestive of purine NTPases activity that is usually associated with cellular and viral helicases also are present in the ORF 1 sequence. There is no consistent immune response to gene products encoded in ORF 1.

The second open reading frame (ORF 2) occupies the carboxyl one-third of the viral genome. ORF 2 comprises approximately 2,000 nucleotides which encode a consensus signal peptide sequence at the amino terminus of ORF 2, and a putative capsid protein, translated in a antibodies that react with peptides or recombinant proteins derived from ORF 2.

The third open reading frame (ORF 3) partly overlaps both ORF 1 and ORF 2, and comprises 369 nucleotides translated in the +2 reading frame in relation to ORF 1. Although the function of the protein encoded by ORF 3 is unknown, the protein is antigenic, with most HEV infected individuals producing antibodies to this protein. Accordingly, peptides or recombinant proteins derived from ORF 2 and ORF 3 may serve as serologic markers useful in diagnosing exposure to HEV.

Recently, several additional HEV isolates have been identified and compared to the Burmese and Mexican strains of HEV. Most of the recent isolates are more closely related to the Burmese strain than to the Mexican strain of HEV. Except for a brief appearance in 1986-1987, there have been no additional isolates of the Mexican strain of HEV (Velasquez et al. (1992) JAMA, 263: 3281-3286).

One isolate, referred to as SAR-55, recently was isolated from an HEV-infected individual from Pakistan. The SAR-55 isolate is highly related to the Burmese strain with nucleotide and amino acid identities of 94% and 99%, respectively, across the entire genome. Several other recent isolates have been made from the Chinese province of Xuar, bordering on Pakistan. These Chinese isolates were more closely related to the Pakistani strain (approximately 98% nucleotide identity) than to the Burmese strain (approximately 93% nucleotide identity).

Prior to the sequencing of the viral genome and the availability of viral-encoded recombinant proteins and synthetic peptides, HEV infection was monitored by electron microscopy and immunofluoresence. Soon after the identification of the HEV genome, specific laboratory techniques for detecting HEV infection became available including (i) specific immunoassays, for example, western blot assays and ELISA's based on recombinant proteins and/or synthetic peptides, and (ii) polymerase chain reactions (PCR), for example, reverse transcriptase PCR (RT-PCR). RT-PCR has been used successfully to detect HEV RNA in samples of stool or serum in cases of acute hepatitis infections, and in epidemics of ET-NANBH. Furthermore, by using recombinant antigens derived from the Mexican and Burmese strains of HEV, specific IgG, IgM and, in some cases, IgA antibodies to HEV have been detected in specimens obtained from ET-NANBH outbreaks in Somalia, Burma, Borneo, Tashkent, Kenya, Pakistan and Mexico. Specific IgG, and sometimes IgM antibodies to HEV have been detected in cases of acute, sporadic hepatitis in geographic regions such as Egypt, India, Tajikistan and Uzbekistan as well as in acute hepatitis cases among patients in industrialized nations (for example, US, UK, Netherlands and Japan) who traveled to areas endemic for HEV.

To date, PCR and immunoassay-based tests based on the Burmese and Mexican isolates of HEV have established that various cases of “waterborne hepatitis” were caused by HEV. The antibody tests also were important in establishing HEV as a cause of acute, sporadic hepatitis in developing nations and among travelers to regions where HEV is endemic. However, it is unclear as to how many cases of acute HEV currently go undiagnosed due to the inability of current reagents to detect exposure to all strains of HEV. Accordingly, as new isolates of HEV are identified, it is desirable to develop new compositions and methods for detecting and/or treating hepatitis caused by the new HEV strains, which heretofore remain undetectable by the currently available test kits.

### SUMMARY OF THE INVENTION

The invention is based, in part, upon the discovery of a new family of human hepatitis E viruses. The newly discovered family of hepatitis E viruses fall within a class referred to hereinafter as a US-type hepatitis E virus. Furthermore, two members of the family were discovered in individuals living in the United States and exhibit considerable similarities when compared at the nucleotide and amino acid levels. The latter two members together belong to a subclass of the US-type hepatitis E virus, referred to hereinafter as US-subtype hepatitis E virus.

Accordingly, in one aspect, the invention provides a method for detecting the presence of a US-type or US-subtype hepatitis E virus in a test sample of interest. The method comprises the steps of (a) contacting the test sample with a binding partner that binds specifically to a marker (or target) for the virus, which if present in the sample binds to the binding partner to produce a marker-binding partner complex, and (b) detecting the presence or absence of the complex. The presence of the complex is indicative of the presence of the virus in the test sample.

In one embodiment, the marker is an anti-US-type or anti-US-subtype antibody, for example, an immunoglobulin G (IgG) or an immunoglobulin M (IgM) molecule, present in the sample of interest, and the binding partner is an isolated polypeptide chain defining an epitope that binds specifically to the marker. In such a case, it is contemplated that the test sample is a body fluid sample, for example, blood, serum or plasma, harvested from an individual under investigation. In a preferred embodiment, the polypeptide chain defining a US-type or US-subtype specific epitope is immobilized on a solid support. Thereafter, the immobilized polypeptide chain is combined with the sample under conditions that permit the marker antibody, for example, an anti-US-type or anti-US-subtype hepatitis E virus specific antibody, present in the sample to bind to the immobilized polypeptide. Thereafter, the presence or absence of bound antibody can be detected using, for example, a second antibody or an antigen binding fragment thereof, for example, an anti-human antibody or an antigen binding fragment thereof, labeled with a detectable moiety.

It is contemplated that many different US-type and US-subtype specific polypeptides may be useful as a binding partner in the practice of this embodiment of the invention. For example, in one preferred embodiment of the invention, it is contemplated that the binding partner may be at least a portion, for example, at least 5, preferably at least 8, more preferably at least 15 and even more preferably at least about 25 amino acid residues, of a polypeptide chain selected from the group consisting of SEQ ID NOS:91, 92 and 93, including naturally occurring variants thereof, and which represent a unique amino acid sequence when compared to the corresponding amino acid sequences of members of the Burmese and Mexican families. Similarly, it is contemplated that the binding partner may be a polypeptide chain comprising the amino acid sequence set forth in SEQ ID NOS:173, 174, or 175. In another preferred embodiment of the invention, it is contemplated that the binding partner may be at least a portion, for example, at least 5, preferably at least 8, more preferably at least 15 and even more preferably at least about 25 amino acid residues, of a polypeptide chain selected from the group consisting of SEQ ID NOS:166, 167 and 168, including naturally occurring variants thereof, and which represent a unique amino acid sequence when compared to the corresponding amino acid sequences of members of the Burmese and Mexican families. Similarly, it is contemplated that the binding partner may be a polypeptide chain comprising the amino acid sequence set forth in SEQ ID NOS:176, 223 or 224.

In another embodiment of the invention, the marker is a polypeptide chain unique for a member of the US-type or US-subtype families of HEV, and the binding partner preferably is an isolated antibody, for example, a polyclonal or monoclonal antibody, that binds to an epitope on the marker polypeptide chain. The binding partner may be either labeled with a detectable moiety or immobilized on a solid support. For example, it is contemplated that practice of this embodiment of the invention may be facilitated by immobilizing on a solid support, a first antibody that binds a first epitope on the marker polypeptide of interest. A test sample to be analyzed then is combined with the solid support under conditions that permit the immobilized antibody to bind the marker polypeptide. Thereafter, the presence or absence of bound marker polypeptide chain may be determined using, for example, a second antibody conjugated with a detectable moiety which binds to a second, different epitope on the marker polypeptide chain.

An antibody useful in the practice of this embodiment of the invention preferably is capable of binding specifically to a polypeptide chain selected from the group consisting of SEQ ID NOS:91, 92, and 93, including naturally occurring variants thereof, and has a higher binding affinity for such a polypeptide chain relative to the corresponding sequences of members of the Burmese and Mexican families. It is contemplated that an antibody useful in the practice of the invention preferably is capable of binding specifically to a polypeptide chain comprising the amino acid sequence set forth in SEQ ID NOS:173 or 175. This antibody being further characterized as, under similar conditions, preferably having a lower affinity for, and most preferably failing to bind the amino acid sequence set forth in SEQ. ID NOS:169 or 171 or to the regions in the Burmese and Mexican strains that correspond to SEQ ID NO: 175. Similarly, it is contemplated that an antibody useful in the practice of the invention preferably is capable of binding specifically to a polypeptide chain comprising the amino acid sequence set forth in SEQ ID NOS:174 or 176. This antibody being further characterized as, under similar conditions, preferably having a lower affinity for, and most preferably failing to bind the amino acid sequence set forth in SEQ. ID NOS:170 or 172 or to the regions in the Burmese and Mexican strains that correspond to SEQ ID NO:176.

Similarly, it is contemplated that an antibody useful in the practice of this embodiment of the invention preferably is capable of binding specifically to a polypeptide chain selected from the group consisting of SEQ ID NOS:166, 167, and 168, including naturally occurring variants thereof, and has a higher binding affinity for such a polypeptide chain relative to the corresponding sequences of members of the Burmese and Mexican families. It is contemplated that an antibody useful in the practice of the invention preferably is capable of binding specifically to a polypeptide chain comprising the amino acid sequence set forth in SEQ ID NO: 223. This antibody being further characterized as, under similar conditions, preferably having a lower affinity for, and most preferably failing to bind the amino acid sequences set forth in SEQ. ID NOS:170 or 172. Similarly, it is contemplated that an antibody useful in the practice of the invention preferably is capable of binding specifically to a polypeptide chain comprising the amino acid sequence set forth in SEQ ID NO:224. This antibody being further characterized as, under similar conditions, preferably having a lower affinity for, and most preferably failing to bind the amino acid sequence set forth in SEQ ID NOS:169 or 171.

In another embodiment of the invention, the marker is a nucleic acid sequence defining at least a portion of a genome of a US-type or US-subtype E virus, or a sequence complementary thereto. Similarly, it is contemplated that the binding partner is an isolated nucleic acid sequence, for example, a deoxyribonucleic acid (DNA), ribonucleic acid (RNA) or peptidyl nucleic acid (PNA) sequence, preferably comprising 8-100 nucleotides, more preferably comprising 10 to 75 nucleotides and mostly preferably comprising 15-50 nucleotides, which is capable of hybridizing specifically, for example, under specific hybridization conditions or under specific PCR annealing conditions, to the nucleotide sequence set forth in SEQ ID NOS:89 or 164.

Practice of this embodiment of the invention may be facilitated, for example, by isolating nucleic acids from the sample of interest. Thereafter, the resulting nucleic acids, may be fractionated by, for example, gel electrophoresis, transferred to, and immobilized onto a solid support, for example, nitrocellulose or nylon membrane, or alternatively may be immobilized directly onto the solid support via conventional dot blot or slot blot methodologies. The immobilized nucleic acid then may be probed with a preselected nucleic acid sequence labeled with a detectable moiety, that hybridizes specifically to the marker sequence. Alternatively, the presence of marker nucleic acid in a sample may be determined by standard amplification based methodologies, for example, polymerase chain reaction (PCR) wherein the production of a specific amplification product is indicative of the presence of marker nucleic acid in the sample.

In another aspect, the invention provides isolated US-type and US-subtype specific polypeptides sequences. These polypeptides include those described hereinabove in the section pertaining to US-type and US-subtype hepatitis E specific polypeptides chains useful as binding partners. In a preferred embodiment, the isolated polypeptide chain comprises an amino acid sequence set forth in SEQ ID NO:93, SEQ ID NO:168, SEQ ID NO:173, SEQ ID NO:174, SEQ ID NO:175, SEQ ID NO:176, SEQ ID NO:223 or SEQ ID NO:224. It is contemplated that these and other US-type and US-subtype specific polypeptide chains may be employed in an assay format for detecting the presence of anti-US-type of US-subtype hepatitis E specific antibodies in a sample. In addition, it is contemplated that these polypeptides may be used either alone or in combination with adjuvants for the production of antibodies in laboratory animals, or similarly, used in combination with pharmaceutically acceptable carriers as vaccines for either the prophylactic or therapeutic immunization of mammals.

In another aspect, the invention provides isolated anti-US-type or anti-US-subtype hepatitis E specific antibodies, which include those discussed hereinabove in the section pertaining to antibodies useful as binding partners. In a preferred embodiment, the isolated antibody is capable of binding specifically to a polypeptide chain selected from the group consisting of a polypeptide encoded by an ORF 1 sequence of a US-type or a US-subtype hepatitis E virus, a polypeptide encoded by an ORF 2 sequence of a US-type or a US-subtype hepatitis E virus, or a polypeptide encoded by an ORF 3 sequence of a US-type or a US-subtype hepatitis E virus. In particular, it is contemplated that useful antibodies are characterized in that they are capable of binding specifically to a polypeptide chain comprising the amino acid sequence set forth in SEQ ID NO:93, SEQ ID NO:168, SEQ ID NO: 173, SEQ ID NO:174, SEQ ID NO:175, SEQ ID NO:176, SEQ ID NO:223 or SEQ ID NO:224. It is contemplated that these antibodies and other antibodies may be used to advantage in immunoassays for detecting the presence in a sample of members of the US-type or US-subtype hepatitis E families. The antibody may be used either in a direct immunoassay wherein the antibody itself preferably is labeled with a detectable moiety or in an indirect immunoassay wherein the antibody itself provides a target for a second binding partner, e.g., a second antibody labeled with a detectable moiety. Furthermore, it is contemplated that these antibodies may be used in combination with, for example, a pharmaceutically acceptable carrier for use in the passive, therapeutic or prophylactic immunization of a mammal.

In another aspect, the invention provides isolated nucleic acid sequences such as those discussed in the previous section pertaining to the use of nucleic acids as a marker or a binding partner for detecting the presence of a US-type or US-subtype hepatitis E virus in a sample. In a preferred embodiment, the invention provides isolated nucleic acid sequences defining at least a portion of an ORF 1, ORF 2 or ORF 3 sequence of a US-type or US-subtype hepatitis E virus, or a sequence complementary thereto. It is contemplated that these and other nucleic acid sequences may be used, for example, as nucleotide probes and/or amplification primers for detecting the presence of a US-type or US-subtype hepatitis E virus in a sample of interest. In addition, it is contemplated the nucleic acid sequences or sequences complementary thereto may be combined with a pharmaceutically acceptable carrier for use in anti-sense therapy. Furthermore, it is contemplated the nucleic acid sequences may be integrated in vectors which may then be transformed or transfected into a host cell of interest. The host cells may then be combined with a pharmaceutically acceptable carrier and used as a vaccine, for example, a recombinant vaccine, for immunizing a mammal, either prophylactically or therapeutically, against a preselected US-type or US-subtype hepatitis E virus.

The foregoing and other objects, features and advantages of the present invention will be made more apparent from the following detailed description of preferred embodiments of the invention.

### BRIEF DESCRIPTION OF THE DRAWINGS

The objects and features of the invention may be better understood by reference to the drawings described below in which,

FIG. 1 is a schematic representation of a HEV genome showing the relative positions of the ORF 1, ORF 2, and ORF 3 regions.

FIG. 2 is a graph showing levels of serum aspartate aminotransferase (boxes) and serum total bilirubin (diamonds) in patient USP-1 from day 1 of a hospital admission through day 37 post admission.

FIG. 3 is a schematic representation of the HEV US-1 genome showing the relative positions of clones isolated during the course of this work.

FIG. 4 is a schematic representation of the HEV US-2 genome showing the relative positions of clones isolated during the course of this work.

FIG. 5 shows an unrooted phylogenetic tree depicting the relationship of nucleotide sequences from full length HEV US-1, HEV US-2, and 10 other HEV isolates. Branch lengths are proportional to the evolutionary distances between sequences. The scale representing nucleotide substitutions per position is shown. The internal node numbers indicate the bootstrap values (expressed as a percentage of all trees) obtained from 100 replicates. Isolates represented are Burmese, B1, B2; Chinese, C1, C2, C3, C4; Pakistan, P1; Indian, I1, I2; Mexican, M1; and United States, US-1, US-2.

FIG. 6 shows an unrooted phylogenetic tree depicting the relationship of nucleotide sequences from the ORF 2/3 regions (i.e., sequences corresponding to nucleotide residue numbers 5094-7114 of SEQ ID NO:89). Branch lengths are proportional to the evolutionary distances between sequences. The scale representing nucleotide substitutions per position is shown. The internal node numbers indicate the bootstrap values (expressed as a percentage of all trees) obtained from 100 replicates. Isolates represented are Burmese, B1, B2; Chinese, C1, C2, C3, C4; Pakistan, P1; Indian, I1, I2; Mexican, M1; Swine, S1; and United States, US-1, US-2.

FIG. 7 is a graph showing levels of alanine aminotransferase (boxes), serum aspartate transferase (circles), and gamma-glutamyltransferase (triangles) in a macaque before and after inoculation with sera harvested from patient USP-2. Also shown are times when HEV US-2 RNA were present in serum and fecal samples, as well as times when anti-HEV US-2 IgM and IgG were detectable.

FIG. 8 is a schematic representation of the It1 genome showing the relative positions of clones isolated during the course of this work.

FIGS. 9 shows aligments of Burmese (B1), Mexican (M1), Chinese (C1), Pakistan (P1) and US-1 showing the design of HEV consensus primers for ORF 1, ORF 2/3 and ORF 2. Preferred consensus primers are denoted by the highlighted boxes.

FIG. 10 shows an unrooted phylogenetic tree depicting the relationship of ORF 1 nucleotide sequences 371 nucleotides in length and corresponding to residues 26-396 of SEQ ID NO:89. The scale representing nucleotide substitutions per position is shown. The internal node numbers indicate the bootstrap values (expressed as a percentage of all trees) obtained from 1000 replicates. Isolates represented are Burmese, B1, B2; Chinese, C1, C2, C3, C4; Pakistan, P1; Indian, I1, I2; Mexican, M1; Italian, It1; Greek, G1, G2; and United States, US-1, US-2.

FIG. 11 shows an unrooted phylogenetic tree depicting the relationship of ORF 2 nucleotide sequences 148 nucleotides in length and corresponding to residues 6307-6454 of SEQ ID NO:89. The scale representing nucleotide substitutions per position is shown. The internal node numbers indicate the bootstrap values (expressed as a percentage of all trees) obtained from 1000 replicates. Isolates represented are Burmese, B1, B2; Chinese, C1, C2, C3, C4; Pakistan, P1; Indian, I1, I2; Mexican, M1; Italian, It1; Greek, G1, G2; Swine, S1; and United States, US-1 and US-2.

FIG. 12 shows a schematic representation of preferred HEV-US recombinant protein constructs.

In 12A, the ORF 2 and ORF 3 structural proteins of HEV are shown with the first and last amino acid positions designated. The presence of immunodominant epitopes are indicated by lines within the ORFs.

FIG. 12B shows an ORF 3 region that was cloned into an expression vector, with the first and last amino acid positions designated (SEQ ID NO:203 or SEQ ID NO:204).

FIG. 12C shows an ORF 2 region that was cloned into an expression vector, with the first and last amino acid positions designated (SEQ ID NO:199 or 200).

FIG. 12D shows an ORF 3/2 chimeric construct cloned into an expression vector with the first and last amino acid positions of each component of the chimeric construct designated (SEQ ID NO:206 or 207). The sequence omitted from the ORF 3/2 construct is indicated with a dashed line.

In FIGS. 12B-12D, the presence of a FLAG® peptide at the carboxyl terminus of each construct is indicated by a solid box.

FIG. 13 is a graph showing levels of alanine aminotransferase (square), IgG (circle) and IgM (star) in a macaque before and after inoculation with sera harvested from patient USP-2.

FIG. 14 shows an unrooted phylogenetic tree depicting the relationship of ORF 1 nucleotide sequences 371 nucleotides in length and corresponding to residues 26-396 of SEQ ID NO:89. The scale representing nucleotide substitutions per position is shown. The internal node numbers indicate the bootstrap values (expressed as a percentage of all trees) obtained from 1000 replicates. Isolates represented are Burmese, B1, B2; Chinese, C1, C2, C3, C4; Pakistan, P1; Indian, I1, I2; Mexican, M1; Italian, It1; Greek, G1, G2; Austrian, Au1; Argentine, Ar1, Ar2; and United States, US-1, US-2.

FIG. 15 shows an unrooted phylogenetic tree depicting the relationship of ORF 2 nucleotide sequences 148 nucleotides in length and corresponding to residues 6307-6454 of SEQ ID NO:89. The scale representing nucleotide substitutions per position is shown. The internal node numbers indicate the bootstrap values (expressed as a percentage of all trees) obtained from 1000 replicates. Isolates represented are Burmese, B1, B2; Chinese, C1, C2, C3, C4; Pakistan, P1; Indian, I1, I2; Mexican, M1; Italian, It1; Greek, G1, G2; Austrian, Au1; Argentine, Ar2; Swine, S1; and United States, US-1 and US-2.

FIG. 16 shows an unrooted phylogenetic tree depicting the relationship of ORF 2 nucleotide sequences 98 nucleotides in length and corresponding to residues 6354-6451 of SEQ ID NO:89. The scale representing nucleotide substitutions per position is shown. The internal node numbers indicate the bootstrap values (expressed as a percentage of all trees) obtained from 1000 replicates. Isolates represented are Burmese, B1, B2; Chinese, C1, C2, C3, C4; Pakistan, P1; Indian, I1, I2; Mexican, M1; Italian, It1; Greek, G1, G2; Austrian, Au1; Argentine, Ar1, Ar2; Swine, S1; and United States, US-1 and US-2.

### DETAILED DESCRIPTION OF THE INVENTION

As mentioned above, this invention is based, in part, upon the discovery of a new family of human hepatitis E viruses. The newly discovered family of hepatitis E viruses fall within a class referred to hereinafter as a US-type hepatitis E virus. Furthermore, as mentioned above, two members of the US-type family were identified in sera obtained from two individuals living in the United States of America. These two members together belong to a subclass of the US-type hepatitis E virus, referred to hereinafter as a US-subtype hepatitis E virus. The discovery of the US-type and US-subtype hepatitis E viruses enables the development of methods and compositions for detecting the presence of a US-type of US-subtype hepatitis E virus in individuals who heretofore have not been diagnosed as suffering from hepatitis based on commercially available hepatitis detection kits, as well as methods and compositions for immunizing an individual against such a virus.

In one aspect, the invention pertains to a method of detecting the presence of a US-type or US-subtype hepatitis E virus in a test sample. The method comprises the steps of (a) contacting the sample with a binding partner that binds specifically to a marker for such a virus, which if present in the sample binds to the binding partner to produce a marker-binding protein complex, and (b) detecting the presence or absence of the complex. The presence of the complex is indicative of the presence of the virus in the sample. Based on the discovery of the US-type and US-subtype hepatitis E virus disclosed herein, it will be apparent that a variety of assays, for example, protein- or nucleic acid-based assays, may be produced for detecting the presence of the virus in a sample. Protein-based assays may include, for example, conventional immunoassays, and nucleic acid-based assays may include, for example, conventional probe hybridization or nucleic acid sequence amplification assays, all of which are well known and thoroughly discussed in the art.

In another aspect, the invention provides reagents, for example, antibodies, epitope containing polypeptide chains, and nucleotide sequences that may be used to develop vaccines for immunizing, either prophylactically or therapeutically, an individual against a US-type or US-subtype hepatitis E virus.

I. Definitions

So that the invention may be more readily understood, certain terms as used herein are defined hereinbelow.

As used herein, the term “US-type” hepatitis E virus is understood to mean any human virus (i.e., capable of infecting a human) that is serologically distinct from hepatitis A virus (HAV), hepatitis B virus (HBV), hepatitis C virus (HCV), hepatitis D virus (HDV) and hepatitis G virus (HGV) and comprising a single stranded RNA genome defining at least one open reading frame and having a nucleotide sequence greater than 79.7% identity to the nucleotide sequence defined by residues 6307-6454 of SEQ ID NO:89.

As used herein, the term “US-subtype” hepatitis E is understood to mean any human virus (i.e., capable of infecting a human) that is serologically distinct from hepatitis A virus (HAV), hepatitis B virus (HBV), hepatitis C virus (HCV), hepatitis D virus (HDV) and hepatitis G virus (HGV) and comprising a single stranded RNA genome defining at least one open read frame and having a nucleotide sequence greater than 90.5% identity to the nucleotide sequence defined by residues 6307-6454 of SEQ ID NO:89.

As used herein, the term, “test sample” is understood to mean any sample, for example, a biological sample, which contains the marker (for example, an antibody, antigenic protein or peptide, or nucleotide sequence) to be tested. Preferred test samples include tissue or body fluid samples isolatable from an individual under investigation. Preferred body fluid samples include, for example, blood, serum, plasma, saliva, sputum, semen, urine, feces, bile, spinal fluid, breast exude, ascities, and peritoneal fluid. Another preferred test sample is a cell line and more preferably, a mammalian cell line. A most preferred cell line is a human fetal kidney cell line.

As used herein, the term “open reading frame” or “ORF” is understood to mean a region of a polynucleotide sequence capable of encoding one or more polypeptide chains. The region may represent an entire coding sequence, i.e., beginning with an initiation codon (e.g., ATG (AUG)) and ending at a termination codon (e.g., TAA (UAA), TAG (UAG), or TGA (UGA)), or a portion thereof.

As used herein, the term “polypeptide chain” is understood to mean any molecular chain of amino acids and does not refer to a specific length of the product. Thus, peptides, oligopeptides, and proteins are included within the definition of polypeptide chain.

As used herein, the term “epitope”, as used synonymously with “antigenic determinant”, is understood to mean at least a portion of an antigen capable of being specifically bound (i e., bound with an affinity greater than about 10⁵ M⁻¹, and more preferably with an affinity greater than about 10⁷ M⁻¹) by an antibody variable region. Conceivably, an epitope may comprise three amino acids in a spatial conformation unique to the epitope. Generally, an epitope comprises at least five amino acids, and more usually, at least eight to ten amino acids. Methods of examining spatial conformation are known in the art and include, for example, x-ray crystallography and two-dimensional nuclear magnetic resonance.

A polypeptide is “immunologically reactive” with an antibody when it binds to an antibody due to antibody recognition of a specific epitope defined by the polypeptide chain. Immunological reactivity may be determined by antibody binding, more particularly by the kinetics of antibody binding, and/or by a competitive binding study. If a preselected antibody is immunologically reactive with a first antigen but is not immunologically reactive or is less immunologically reactive with a second, different antigen, then the two antigens are considered to be serologically distinct. As used herein, the term “affinity” is understood to mean a measure of reversible interaction between two molecules (for example, between an antibody and an antigen). The higher the affinity, the stronger the interaction between the two molecules.

As used herein, the term “detectable moiety” is understood to mean any signal generating compound, for example, chromogen, a catalyst such as an enzyme, a luminescent compound such as dioxetane, acridinium, phenanthridinium and luminol, a radioactive element, and a visually detectable label. Examples of enzymes include alkaline phosphatase, horseradish peroxidase, beta-galactosidase, and the like. Although the selection of a particular detectable moiety is not critical, the detectable moiety will be capable of producing a signal either by itself or in conjunction with one or more additional substances.

As used herein, the term “solid support” is understood to mean any plastic, derivatized plastic, magnetic or non-magnetic metal, glass or silicon surface. Useful surfaces include, for example, the surface of a test tube, microtiter well, sheet, bead, microparticle, chip, sheep (or other suitable animal's) red blood cell, or duracyte. Suitable solid supports are not critical to the practice of the invention and can be selected by one skilled in the art. Suitable methods for immobilizing peptides on solid phases include ionic, hydrophobic, covalent interactions and the like. The solid support can be chosen for its intrinsic ability to attract and immobilize the capture reagent. Alternatively, the solid support can retain an additional receptor which has the ability to attract and immobilize the capture reagent.

It is contemplated that the solid support also may comprise any suitable porous material with sufficient porosity to allow access by detection antibodies and a suitable surface affinity to bind antigens. Microporous structures generally are preferred, but materials with gel structure in the hydrated state may be used as well. All of these materials may be used in suitable shapes, such as films, sheets, or plates, or they may be coated onto or bonded or laminated to appropriate inert carriers, such as paper, glass, plastic films, or fabrics.

Other embodiments which utilize various other solid supports also are contemplated and are within the scope of this invention. For example, ion capture procedures for immobilizing an immobilizable reaction complex with a negatively charged polymer, described in EP Publication No. 0 326 100 and EP Publication No. 0 406 473, can be employed according to the present invention to effect a fast solution-phase immunochemical reaction. An immobilizable immune complex is separated from the rest of the reaction mixture by ionic interactions between the negatively charged poly-anion/immune complex and the previously treated, positively charged porous matrix and detected by using various signal generating systems previously described, including those described in chemiluminescent signal measurements as described in EP Publication No. 0 273 115.

Also, the methods of the present invention can be adapted for use in systems which utilize microparticle technology including automated and semi-automated systems wherein the solid phase comprises a microparticle (magnetic or non-magnetic). Such systems include those described in U.S. Pat. Nos. 5,089,424 and 5,244,630, issued Feb. 18, 1992 and Sep. 14, 1993, respectively.

The use of scanning probe microscopy (SPM) for immunoassays also is a technology to which the monoclonal antibodies of the present invention are easily adaptable. In scanning probe microscopy, in particular in atomic force microscopy, the capture phase, for example, at least one of the monoclonal antibodies of the invention, is adhered to a solid phase and a scanning probe microscope is utilized to detect antigen/antibody complexes which may be present on the surface of the solid phase. The use of scanning tunneling microscopy eliminates the need for labels which normally must be utilized in many immunoassay systems to detect antigen/antibody complexes. The use of SPM to monitor specific binding reactions can occur in many ways. In one embodiment, one member of a specific binding partner (analyte specific substance which is the monoclonal antibody of the invention) is attached to a surface suitable for scanning. The attachment of the analyte specific substance may be by adsorption to a test piece which comprises a solid phase of a plastic or metal surface, following methods known to those of ordinary skill in the art. Or, covalent attachment of a specific binding partner (analyte specific substance) to a test piece which test piece comprises a solid phase of derivatized plastic, metal, silicon, or glass may be utilized. Covalent attachment methods are known to those skilled in the art and include a variety of means to irreversibly link specific binding partners to the test piece. If the test piece is silicon or glass, the surface must be activated prior to attaching the specific binding partner. Also, polyelectrolyte interactions may be used to immobilize a specific binding partner on a surface of a test piece by using techniques and chemistries described in EP Publication No. 0 322 100 and EP Publication No. 0 406 473. The preferred method of attachment is by covalent attachment. Following attachment of a specific binding member, the surface may be further treated with materials such as serum, proteins, or other blocking agents to minimize non-specific binding. The surface also may be scanned either at the site of manufacture or point of use to verify its suitability for assay purposes. The scanning process is not anticipated to alter the specific binding properties of the test piece.

As used herein, the terms “nucleotide sequence” or “nucleic acid sequence” is understood to mean any polymeric form of nucleotides of any length, either ribonucleotides or deoxyribonucleotides. The term refers to the primary structure of the molecule. Thus, the term includes double- and single-stranded DNA, as well as double- and single-stranded RNA. It also includes modifications, for example, by methylation and/or by capping, and unmodified forms of the polynucleotide.

As used herein, the term “primer” is understood to mean a specific oligonucleotide sequence complementary to a target nucleotide sequence which is capable of hybridizing to the target nucleotide sequence and serving as an initiation point for nucleotide polymerization catalyzed by DNA polymerase, RNA polymerase or reverse transcriptase.

When referring to a nucleic acid fragment, such a fragment is considered to “specifically hybridize” or to “specifically bind” to an HEV US-type or US-subtype polynucleotide or variants thereof, if, within the linear range of detection, the hybridization results in a stronger signal relative to the signal that would result from hybridization to an equal amount of a polynucleotide from other than an HEV US-type, US-subtype or variant thereof. A signal which is “stronger” than another is one which is measurable over the other by the particular method of detection.

Also, when referring to a nucleic acid fragment, such a fragment is considered to hybridize under specific hybridization conditions if it specifically hybridizes under (i) typical hybridization and wash conditions, such as those described, for example, in Maniatis, (1st Edition, pages 387-389, 1982) where preferred hybridization conditions are those of lesser stringency and more preferred, higher stringency; or (ii) standard PCR conditions (Saiki, R. K. et al.) or “touch-down” PCR conditions (Roux, K. H., (1994), Biotechiques, 16:812-814).

As used herein, the term “probe” is understood to mean any nucleotide or nucleotide analog (e.g., PNA) containing a sequence which can be used to identify specific DNA or RNA present in samples bearing the complementary sequence.

As used herein, the term “PNA” is used to mean peptide nucleic acid analog which may be utilized in a procedure such as an assay described herein to determine the presence of a target. “MA” denotes a “morpholino analog” which may be utilized in a procedure such as an assay described herein to determine the presence of a target. See, for example, U.S. Pat. No. 5,378,841, which is incorporated herein by reference. PNAs typically are neutrally charged moieties which can be directed against RNA targets or DNA. PNA probes used in assays in place of, for example, the DNA probes of the present invention, offer advantages not achievable when DNA probes are used. These advantages include manufacturability, large scale labeling, reproducibility, stability, insensitivity to changes in ionic strength and resistance to enzymatic degradation which is present in methods utilizing DNA or RNA. These PNAs can be labeled with such signal generating compounds as fluorescein, radionucleotides, chemiluminescent compounds, and the like. PNAs or other nucleic acid analogs such as MAs thus can be used in assay methods in place of DNA or RNA. Although assays are described herein utilizing DNA probes, it is within the scope of the routine that PNAs or MAs can be substituted for RNA or DNA with appropriate changes if and as needed in assay reagents.

When referring to a nucleic acid fragment, such a fragment is considered to “specifically hybridize” or to “specifically bind” to an HEV US-type or US-subtype polynucleotide or variants thereof, if, within the linear range of detection, the hybridization results in a stronger signal relative to the signal that would result from hybridization to an equal amount of a polynucleotide from other than an HEV US-type, US-subtype or variant thereof. A signal which is “stronger” than another is one which is measurable over the other by the particular method of detection.

Also, when referring to a nucleic acid fragment, such a fragment is considered to hybridize under specific hybridization conditions if it specifically hybridizes under (i) typical hybridization and wash conditions, such as those described, for example, in Maniatis, (1st Edition, pages 387-389, 1982) where preferred hybridization conditions are those of lesser stringency and more preferred, higher stringency; or (ii) standard PCR conditions (Saiki, R. K. et al) or “touch-down” PCR conditions (Roux, K. H., (1994), Biotechiques, 16:812-814).

II. Detection Methods and Reagents

It is contemplated that the detection methods of the invention may employ a variety of protein-based or nucleic acid-based assays which are described in detail below.

It is contemplated that a reagent for the detection of virus or markers thereof may be either an anti-US-type and/or US-subtype hepatitis E virus antibody, a US-type and/or US-subtype specific polypeptide, or a nucleic acid defining at least a portion of the genome of a US-type and/or US-subtype hepatitis E virus or a nucleic acid sequence complementary thereto.

II (i) Protein-based Assays

A. Marker Antibodies: It is contemplated that if the viral marker is an anti-US-type or anti-US-subtype specific antibody, for example, an IgG or an IgM, molecule circulating in the blood stream of an individual of interest, the binding partner preferably is a polypeptide defining an epitope that binds specifically to the marker.

In a preferred protocol for detecting the presence of anti-US-type or anti-US-subtype hepatitis E virus antibodies in a test sample, the protocol preferably comprises the following steps which include: (a) providing an antigen comprising an immunologically reactive US-type or US-subtype specific polypeptide chain comprising at least 5, more preferably at least 8, even more preferably at least 15, and most preferably at least 25 contiguous amino acid residues and bindable by the antibody; (b) incubating the antigen with the test sample under conditions that permit formation of an antibody-antigen complex; and (c) detecting the presence of the complex.

It is contemplated that many, different US-type or US-subtype specific polypeptides may be useful as a binding partner for the detection of anti-US-type or anti-US-subtype antibodies. For example, it is contemplated that the polypeptide chain may be an amino acid sequence defined by SEQ ID NOS:91, 92 or 93 or an immunologically reactive fragment thereof containing, preferably at least 5, more preferably at least 8, even more preferably at least 15, and most preferably at least about 25 contiguous amino acid residues, of the polypeptide chain set forth in SEQ ID NOS:91, 92, or 93, and which represent a unique amino acid sequence when compared to the corresponding amino acid sequences of members of the Burmese and Mexican families. The Burmese family i.e., “Burmese-like” strains, as used herein, presently comprises strains referred to herein as B1, B2, I1, I2, C1, C2, C3, C4 and P1 and the Mexican family presently comprises strain M1.

It is contemplated that the binding partner may be a polypeptide selected from the group consisting of polypeptides defined by SEQ ID NOS:91, 92, and 93, including naturally occurring variants thereof. As used herein the term “naturally occurring variants thereof” with respect to the polypeptide defined by SEQ ID NO:91 is understood to mean any amino acid sequence that is at least 84%, preferably at least 86%, more preferably at least 89% and even more preferably at least 95% identical to residues 1 through 1698 of SEQ ID NO:91. As used herein the term “naturally occurring variants thereof” with respect to the polypeptide defined by SEQ ID NO:92 is understood to mean any amino acid sequence that is at least 93%, preferably at least 95%, and even more preferably at least 98% identical to residues 1 through 660 of SEQ ID NO:92. As used herein the term “naturally occurring variants thereof” with respect to the polypeptide defined by SEQ ID NO:93 is understood to mean any amino acid sequence that is at least 85.4%, preferably at least 87.4%, more preferably at least 90.4% and even more preferably at least 95% identical to residues 1 through 122 of SEQ ID NO:93.

Furthermore, it is contemplated that the binding partner may be a polypeptide encoded by a portion of an ORF 1 sequence. Proteins encoded by the ORF 1 sequence include, for example, a methyltransferase protein, a protease, a Y domain protein, an X domain protein, a helicase protein, a hypervariable region protein, and an RNA-dependent RNA polymerase protein. Accordingly, it is contemplated that a useful methyltransferase protein preferably has at least 92.3%, more preferably has at least 94.3%, and most preferably has at least 97.3% identity to residues 1-231 of SEQ ID NO:91. Also, it is contemplated that a useful protease protein preferably has at least 70.3%, more preferably has at least 72.3%, and most preferably has at least 75.3% identity to residues 424-697 of SEQ ID NO:91. Also, it is contemplated that a useful Y domain protein preferably has at least 94.6%, more preferably has at least 96.6% and most preferably has at least 99.6% identity to residues 207-424 of SEQ ID NO:91. Also it is contemplated that a useful X domain protein preferably has at least 83.4%, more preferably has at least 85.4% and most preferably has at least 88.4% identity to residues 789-947 of SEQ ID NO:91. Also, it is contemplated that a useful helicase protein has at least 92%, more preferably has at least 94% and most preferably at least 93% identity to residues 965-1197 of SEQ ID NO:91. Also, it is contemplated that a useful hypervariable region protein has at least 28.7%, more preferably has at least 30.7%, and most preferably has at least 33.7% identity to the residues 698-788 of SEQ ID NO:91. Also, it is contemplated that a useful RNA-dependent RNA polymerase has at least 88.8%, more preferably has at least 90.8%, and most preferably has at least about 93.8% identity to residues 1212-1698 of SEQ ID NO:91.

Furthermore, it is contemplated that the binding partner may be a polypeptide chain having an amino acid sequence defined by SEQ ID NOS:166, 167 or 168, or an immunologically reactive fragment thereof containing 5, preferably at least 8, more preferably at least 15 and most preferably at least 25 contiguous amino acid residues of the polypeptide chain set forth in SEQ ID NOS:166, 167 or 168, and which represent a unique amino acid sequence when compared to the corresponding amino acid sequences of members of the Burmese and Mexican families. Similarly, it is contemplated that the binding partner may be a polypeptide selected from the group consisting of SEQ ID NOS:166, 167 and 168, including naturally occurring variants thereof. As used herein, the term “naturally occurring variants thereof” with respect to the polypeptide defined by SEQ ID NO:166 is understood to mean any amino acid sequence that is at least 83.9%, preferably at least 85.9%, more preferably at least 88.9%, and most preferably at least 95% identical to residues 1 through 1708 of SEQ ID NO: 166. As used herein, the term “naturally occurring variants thereof” with respect to the polypeptide defined by SEQ ID NO:167 is understood to mean any amino acid sequence that is at least 93%, preferably at least 95%, and most preferably at least 98% identical to residues 1 through 660 of SEQ ID NO:167. As used herein, the term “naturally occurring variants thereof” with respect to the polypeptide defined by SEQ ID NO:168 is understood to mean any amino acid sequence that is at least 85.4%, preferably at least 87.4%, more preferably at least 90.4%, and even more preferably at least 95% identical to residues 1 through 122 of SEQ ID NO:168.

Furthermore, it is contemplated that the binding partner may be a polypeptide encoded by a portion of the HEV US-2 ORF 1, including, for example, a methyltransferase protein, a protease, a Y domain protein, an X domain protein, a helicase protein, a hypervariable region protein and an RNA-dependent RNA polymerase protein, or a variant thereof. Accordingly, it is contemplated that a useful methyltransferase protein preferably has at least 92.7%, more preferably has at least 94.7%, and most preferably has at least 97.7% identity to residues 1-240 of SEQ ID NO:166. Also, it is contemplated that a useful protease protein preferably has at least 69.6%, more preferably has at least 71.6%, and most preferably has at least 74.6% identity to residues 433-706 of SEQ ID NO:166. Also, it is contemplated that a useful Y domain protein preferably has at least 94.6%, more preferably has at least 96.6%, and most preferably has at least 99.6% identity to residues 216-433 of SEQ ID NO:166. Also it is contemplated that a useful X domain protein preferably has at least 82.8%, more preferably has at least 84.8%, and most preferably has at least 87.8% identity to residues 799-957 of SEQ ID NO:166. Also, it is contemplated that a useful helicase protein has at least 92.8%, more preferably has at least 94.8%, and most preferably has at least 97.8% identity to residues 975-1207 of SEQ ID NO:166. Also, it is contemplated that a useful hypervariable region protein has at least 27%, more preferably has at least 29%, and most preferably has at least 31% identity to the residues 707-798 of SEQ ID NO:166. Also, it is contemplated that a useful RNA-dependent RNA polymerase has at least 88.7%, more preferably has at least 90.7%, and most preferably has at least 93.7% identity to residues 1222-1708 of SEQ ID NO:166.

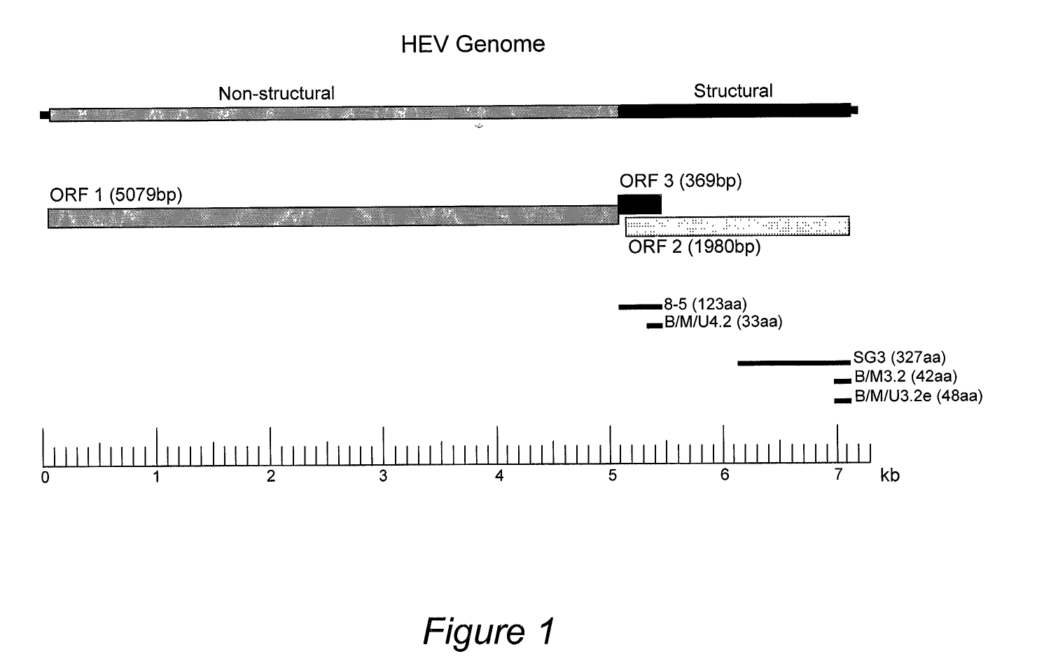

With regard to the identification of US-type or US-subtype specific epitopes, it is contemplated that one skilled in the art in possession of nucleic acid sequences defining and/or amino acid sequences encoded by at least a portion of the genome of a US-type or US-subtype hepatitis E virus can map potential epitope sites using conventional technologies well known and thoroughly discussed in the art. In addition to the use of commercially available software packages which identify potential epitope sites in a given sequence, it is possible to identify potential epitopes by comparison of amino acid sequences encoded by such a genome with sequences encoded by the genomes of other strains of HEV whose antigenic sites have already been elucidated. See, for example, U.S. Pat. Nos. 5,686,239, 5,741,490 and 5,770,689. Epitopes currently identified are shown in FIG. 1, and include epitopes referred to in the art as 8-5 (SEQ ID NOS:93 AND 168), 4-2 (position 90-122 of SEQ ID NOS:93 and 168), SG3 (SEQ ID NOS:175 AND 176), 3-2 (position 613-654 of SEQ ID NOS:92 and 167) and 3-2e (position 613-660 of SEQ ID NOS:92 and 167). A method for calculating antigenic index is described by Jameson and Wolf (CABIOS, 4(1), 181-186 [1988]).

For example, two epitopes of interest are discussed in detail below and are referred to as 3-2e and 4-2 which are encoded by portions of ORF 2 and ORF 3 of the hepatitis E genome, respectively. These epitopes were identified in the Burmese strains of HEV (referred to below as B 3-2e (SEQ ID NO:172) and B 4-2 (SEQ IS NO:171)), and in the Mexican strain of HEV (referred to below as M 3-2e (SEQ ID NO:170) and M 4-2 (SEQ ID NO:169)). Similar epitopes were identified in HEV US-1 based on amino acid sequence comparisons, and are referred to below as U3-2e (SEQ ID NO:174) and U4-2 (SEQ ID NO:173). Similar epitopes were identified in HEV US-2, also based on amino acid sequence comparisons, and are referred to below as US-2 3-2e (SEQ ID NO:223) and US-2 4-2 (SEQ ID NO:224).

In addition, potential epitopes may be identified using screening procedures well known and thoroughly documented in the art. For example, based on the nucleic acid sequences defining either the entire or portions of the HEV US-1 or the HEV US-2 genome, it is possible to generate an expression library, which, after expression can be screened to identify epitopes.

For example, nucleic acid fragments representative of the HEV US-1 or the HEV US-2 genome can be cloned into the lambda-gt11 expression vector to produce a lambda-gt11 library, for example, a cDNA library. The library then is screened for encoded epitopes that can bind specifically with sera derived from individuals identified as being infected with HEV US-1 or HEV US-2. See, for example, Glover (1985) in “DNA Cloning Techniques, A Practical Approach”, IRL Press, pp. 49-78. Typically, about 10⁶-10⁷ phage are screened, from which positive phage are identified, purified, and then tested for specificity of binding to sera from different individuals previously infected with HEV US-1 or HEV US-2. Phage which bind selectively to antibodies present in sera or plasma from the individual are selected for additional characterization. Once identified, an amino acid sequence of interest may be produced in large scale either by use of conventional recombinant DNA methodologies or by conventional peptide synthesis methodologies, well known and thoroughly documented in the art.

b. Marker Polypeptides:

It is contemplated that if the marker is a US-type or US-subtype virus or a specific polypeptide thereof, the binding partner useful in the practice of the invention preferably is an antibody, for example, a polyclonal or monoclonal antibody, that binds to an epitope on the virus or marker polypeptide. The binding partner may be either labeled with a detectable moiety or immobilized on a solid support. In particular, the antibodies useful in the practice of this embodiment preferably are capable of binding specifically to a US-type or US-subtype specific polypeptide chain preferably at least 5, more preferably at least 8, even more preferably at least 15, and most preferably at least 25 contiguous amino acid residues in length which is unique with respect to the corresponding amino acid sequence found in members of the Burmese and Mexican families.

An antibody useful in the practice of this embodiment of the invention preferably is capable of binding specifically to a polypeptide chain selected from the group consisting of SEQ ID NOS:91, 92, and 93, including naturally occurring variants thereof, and has a higher binding affinity for such a polypeptide chain relative to the corresponding sequences of members of the Burmese and Mexican families. It is contemplated that an antibody useful in the practice of the invention preferably is capable of binding specifically to a polypeptide chain comprising the amino acid sequence set forth in SEQ ID NO:173 or 175. This antibody being further characterized as, under similar conditions, preferably having a lower affinity for, and most preferably failing to bind the amino acid sequence set forth in SEQ. ID NOS:169 or 171 or regions in the Burmese and Mexican strains that correspond to SEQ ID NO:175. Similarly, it is contemplated that an antibody useful in the practice of the invention preferably is capable of binding specifically to a polypeptide chain comprising the amino acid sequence set forth in SEQ ID NOS:174 or 176. This antibody being further characterized as, under similar conditions, preferably having a lower affinity for, and most preferably failing to bind the amino acid sequence set forth in SEQ ID NOS:170 or 172 or regions in the Burmese and Mexican strains that correspond to SEQ ID NO:176.

Similarly, it is contemplated that an antibody useful in the practice of this embodiment of the invention preferably is capable of binding specifically to a polypeptide chain selected from the group consisting of SEQ ID NOS:166, 177, and 168, including naturally occurring variants thereof, and has a higher binding affinity for such a polypeptide chain relative to the corresponding sequences of members of the Burmese and Mexican families. It is contemplated that an antibody useful in the practice of the invention preferably is capable of binding specifically to a polypeptide chain comprising the amino acid sequence set forth in SEQ ID NO:223. This antibody being further characterized as, under similar conditions, preferably having a lower affinity for, and most preferably failing to bind the amino acid sequences set forth in SEQ. ID NOS:170 or 172. Similarly, it is contemplated that an antibody useful in the practice of the invention preferably is capable of binding specifically to a polypeptide chain comprising the amino acid sequence set forth in SEQ ID NO:224. This antibody being further characterized as, under similar conditions, preferably having a lower affinity for, and most preferably failing to bind the amino acid sequence set forth in SEQ ID NOS:169 or 171.

The antibodies or antigen binding fragments thereof as described herein can be provided individually to detect US-type or US-subtype specific antigens. Combinations of the antibodies (and antigen binding fragments thereof) provided herein also may be used together as components in a mixture or “cocktail” of at least two antibodies, both having different binding specificities to separate US-type or US-subtype specific antigens.

c. Antibody Production:

It is contemplated that one skilled in the art, in possession of the nucleic acid sequences defining, or amino acid sequences encoded by at least a portion of the ORF 1, ORF 2 and/or ORF 3 sequences of a US-type or a US-subtype hepatitis E virus may be able to produce specific antibodies using techniques well known and thoroughly documented in the art. See, for example, Practical Immunology, Butt, N. R., ed., Marcel Dekker, NY, 1984. Briefly, an isolated target protein is used to raise antibodies in a xenogenic host, such as a mouse, pig, goat or other suitable mammal. Preferred antibodies are antibodies that bind specifically to an epitope on the target protein, preferably having a binding affinity greater than 10⁵M⁻¹, and most preferably having a binding affinity greater than 10⁷M⁻¹ for that epitope. Typically, the target protein is combined with a suitable adjuvant capable of enhancing antibody production in the host, and injected into the host, for example, by intraperitoneal administration. Any adjuvant suitable for stimulating the host's immune response may be used to advantage. A commonly used adjuvant is Freund's complete adjuvant (an emulsion comprising killed and dried microbial cells, e.g., from Calbiochem Corp., San Diego, Calif. or Gibco, Grand Island, N.Y.). Where multiple antigen injections are desired, the subsequent injections comprise the antigen in combination with an incomplete adjuvant (e.g., cell-free emulsion).

Polyclonal antibodies may be isolated from the antibody-producing host by extracting serum containing antibodies to the protein of interest. Monoclonal antibodies may be produced by isolating host cells that produce the desired antibody, fusing these cells with myeloma cells using standard procedures known in the immunology art (See for example, Kohler and Milstein, Nature (1975) 256:495), and screening for hybrid cells (hybridomas) that react specifically with the target protein and have the desired binding affinity.

In addition, it is contemplated that when small peptides are used their immunogenicity may be enhanced by coupling to solid supports. For example, an epitope or antigenic region or fragment of a polypeptide generally is relatively small, and may comprise about 8 to 10 amino acids or less in length. Fragments of as few as 3 amino acids may characterize an antigenic region. These polypeptides may be linked to a suitable carrier molecule when the polypeptide of interest provided folds to provide the correct epitope but yet is too small to be antigenic.

Preferred linking reagents and methodologies for their use are well known in the art and may include, without limitation, N-succinimidyl-3-(2-pyrdylthio)propionate (SPDP) and succinimidyl 4-(N-maleimidomethyl)cyclohexane-l-carboxylate (SMCC). Furthermore, polypeptides lacking sulfhydryl groups can be modified by adding a cysteine residue. These reagents create a disulfide linkage between themselves and peptide cysteine residues on one protein and an amide linkage through the epsilonamino on a lysine, or other free amino group in the other. A variety of such disulfide/amide-forming agents are known. Other bifunctional coupling agents form a thioester rather than a disulfide linkage. Many of these thioether-forming agents are commercially available and are known to those of ordinary skill in the art. The carboxyl groups can be activated by combining them with succinimide or 1-hydroxyl-2-nitro-4-sulfonic acid, sodium salt. Any carrier which does not itself induce the production of antibodies harmful to the host can be used. Suitable carriers include proteins, polysaccharides such as latex functionalized sepharose, agarose, cellulose, cellulose beads, polymeric amino acids such as polyglutamic acid, polylysine, and no acid copolymers and inactive virus particles, among others. Examples of protein substrates include serum albumins, keyhole limpet hemocyanin, immunoglobulin molecules, thyroglobulin, ovalbumin, tetanus toxoid, and yet other proteins known to those skilled in the art.

In addition, it is contemplated that biosynthetically produced antibody binding domains wherein the amino acid sequence of the binding domain is manipulated to enhance binding affinity to a preferred epitope also may be useful in the practice of the invention. A detailed description of their preparation can be found, for example, in Practical Immunology, Butt, W. R., ed., Marcel Dekker, New York, 1984. Optionally, a monovalent antibody fragment such as an Fab or an Fab′ fragment may be utilized. Additionally, genetically engineered biosynthetic antibody binding sites may be utilized which comprise either 1) non-covalently associated or disulfide bonded synthetic V_{H} and V_{L} dimers, 2) covalently linked V_{H}-V_{L} single chain binding sites, 3) individual V_{H} or V_{L} domains, or 4) single chain antibody binding sites, as disclosed, for example, in U.S. Pat. Nos. 5,091,513 and 5,132,405.

It is contemplated that intact antibodies (for example, monoclonal or polyclonal antibodies), antibody fragments or biosynthetic antibody binding sites that bind a US-type or US-subtype hepatitis E virus specific epitope, will be useful in diagnostic and prognostic applications, and also, will be useful in passive immunotherapy.

d. Assay Formats:

It is contemplated that both polypeptides which react immunologically with serum containing anti-US-type or anti-US-subtype hepatitis E virus specific antibodies, or antibodies raised against US-type or US-subtype hepatitis E specific epitopes will be useful in immunoassays to detect the presence of such a virus in a test sample of interest. Furthermore, it is contemplated that the presence of US-type or US-subtype hepatitis E virus in a sample may be detected using any of a wide range of immunoassay techniques, for example, direct assays, sandwich assays, and/or competition assays, currently known and thoroughly documented in the art. A variety of preferred assay formats are described in more detail below.

In one preferred format, the assay employs a sandwich format. Sandwich immunoassays typically are highly specific and very sensitive, provided that labels with good limits of detection are used. A detailed review of immunological assay design, theory and protocols can be found in numerous texts in the art, including Practical Immunology, Butt, W. R., ed., Marcell Dekker, New York, 1984.

In one type of sandwich format, a polypeptide (binding partner) which has been immobilized onto a solid support and is immunologically reactive with an anti-US-type or anti-US-subtype hepatitis E virus antibody (marker), is contacted with a test sample from an individual suspected of having been infected with the US-type or US-subtype hepatitis E virus, to form a mixture. The mixture then is incubated for a time and under conditions sufficient to form polypeptide/antibody complexes. Then, an indicator reagent comprising a monoclonal or a polyclonal antibody or a fragment thereof, which specifically binds to the test sample antibody, and labeled with a detectable moiety, is contacted with the antigen/antibody complexes to form a second mixture. The second mixture then is incubated for a time and under conditions sufficient to form antigen/antibody/antibody complexes. The presence of anti-US-type or anti-US-subtype hepatitis E antibody, if any, in the test sample is determined by detecting the presence of detectable moiety immobilized to the solid support. The amount of antibody present in the test sample is proportional to the signal generated. The use of biotin and antibiotin, biotin and avidin, biotin and streptavidin, and the like, may be used to enhance the generated signal in the assay systems described herein.

In an alternative format of the above-described assay, the immunologically reactive polypeptide may be immobilized “indirectly” to the solid support, i.e. through a monoclonal or polyclonal antibody or fragment thereof which specifically binds that polypeptide. Alternatively, in another format, the assay components may be used in the reverse configuration, such that an antibody or antigen binding fragment thereof, which specifically binds the test sample antibody, i.e., marker antibody (for example, IgG or IgM) and immobilized on the solid support is contacted with the test sample, for a time and under conditions sufficient to permit formation of antibody/antibody complexes. Then, an indicator reagent, for example, a US-type or US-subtype hepatitis E polypeptide immunologically reactive with captured test sample antibody and labeled with a detectable moiety, is incubated with the antibody/antibody complexes to form a second mixture for a time and under conditions sufficient to permit formation of antibody/antibody/antigen complexes. As above, the presence of antibody in the test sample, if any, that is captured by the capture antibody or antigen binding fragment thereof immobilized on the solid support is determined by detecting the measurable signal generated by the detectable moiety.

It is contemplated that the aforementioned sandwich assays also may be used to test for the presence of a US-type or US-subtype hepatitis E virus, or immunologically reactive polypeptides thereof in a test sample by routine modification of the above-described assay configurations. It is contemplated that such modifications would be well known to one skilled in the art.

In addition to the aforementioned sandwich assays, it is contemplated that competitive assays may also be employed in the practice of the invention. In this format, one or a combination of at least two antibodies, preferably monoclonal antibodies, which specifically bind to a US-type or US-subtype hepatitis E specific polypeptide chain can be employed as a competitive probe for the detection of antibodies to the US-type or the US-subtype specific protein. For example, a first HEV US-1 specific polypeptide chain such as one of the polypeptides disclosed herein, acting as a binding partner for the marker, is immobilized on a solid support. A test sample suspected of containing antibody to HEV US-1 antigen then is incubated with the solid support together with an indicator reagent comprising, for example, an isolated anti-US-type or anti-US-subtype antibody that binds the immobilized HEV US-1 specific polypeptide chain and labeled with a detectable moiety, for a time and under conditions sufficient to form antigen/antibody complexes immobilized to the solid support. If the marker antibody is present in the test sample, then the marker antibody competes with the labeled indicator reagent for binding the immobilized polypeptide. As the amount of marker antibody present in the test sample increases, the amount of labeled indicator reagent that binds the immobilized polypeptide decreases. A reduction in the amount of indicator reagent bound to the solid phase can be quantitated. A measurable reduction in signal compared to the signal generated from a confirmed negative non-A, non-B, non-C, non-D, non-E hepatitis test sample also is indicative of the presence of anti-HEV US-1 antibody in the test sample. It is contemplated that similar protocols may be used to identify the presence in a test sample of other hepatitis E viruses falling within the US-type or US-subtype classes.

In yet another detection method, the antibodies of the present invention may be employed to detect the presence of US-type or US-subtype hepatitis E specific antigens in fixed tissue sections, as well as fixed cells by immunohistochemical analysis. Cytochemical analysis wherein these antibodies are labeled directly with a detectable moiety (e.g., fluorescein, colloidal gold, horseradish peroxidase, alkaline phosphatase, etc.) or are labeled indirectly, for example, by means of a secondary antibody labeled with a detectable moiety also may be used in the practice of the invention.

In another assay format, the presence of antibody and/or antigen can be detected by means of a simultaneous assay, for example, as described in EP Publication No. 0 473 065. For example, a test sample is contacted simultaneously with (i) a capture reagent of a first analyte, wherein the capture reagent comprises a first binding member specific for a first analyte immobilized on a solid support and (ii) a capture reagent for a second analyte, wherein the capture reagent comprises a first binding member for a second analyte immobilized on a second different solid support, to produce a mixture. The mixture then is incubated for a time and under conditions sufficient to form capture reagent/first analyte and capture reagent/second analyte complexes. The complexes so-formed then are contacted with a first indicator reagent comprising a member of a binding pair specific for the first analyte labeled with a detectable moiety and a second indicator reagent comprising a member of a binding pair specific for the second analyte labeled with a detectable moiety, to produce a second mixture. The second mixture then is incubated for a time and under conditions sufficient to produce both capture reagent/first analyte/first indicator reagent and capture reagent/second analyte/second indicator reagent complexes. The presence of one or more analytes is determined by detecting a signal generated by the complexes formed on either or both solid phases as an indication of the presence of one or more analytes in the test sample.

Other assay systems may employ an antibody which specifically binds US-type or US-subtype hepatitis E viral particles or sub-viral particles encapsulating the viral genome (or fragments thereof) by virtue of a contact between the specific antibody and the viral protein (peptide, etc.). The captured particles then can be analyzed by methods such as LCR or PCR to determine whether the viral genome is present in the test sample. The advantage of utilizing such an antigen capture amplification method is that it can separate the viral genome from other molecules in the test specimen by use of a specific antibody. Such a method has been described in EP 0 672 176, published Sep. 20, 1995.

In general, immunoassay design considerations include preparation of antibodies (e.g., monoclonal or polyclonal antibodies or antigen binding fragments thereof) having sufficiently high binding specificity for the target protein to form a complex that can be distinguished reliably from products of nonspecific interactions. Typically, the higher the antibody binding specificity, the lower the concentration of target that can be detected.

Both the polypeptide and antibody reagents of the invention may be used to develop assays as described herein to detect either the presence of an antigen from or an antibody that binds to a US-type or US-subtype hepatitis E virus. In addition to their use in immunoassays, it is contemplated that the aforementioned polypeptides may be used either alone or in combination with adjuvants for use in the production of antibodies in laboratory animals, or similarly, used in combination with pharmaceutically acceptable carriers as vaccines for either the prophylactic or therapeutic immunization of individuals. Also, it is contemplated that, in addition to their use in immunoassays, the antibodies of the invention may be used in combination with, for example, a pharmaceutically acceptable carrier for use in passive, therapeutic or prophylactic immunization of an individual. These latter uses are described in more detail in section (III) below. The antibodies of the invention can also be used for the generation of chimeric antibodies for therapeutic use, or other similar applications.

Kits suitable for immunodiagnosis and containing the appropriate reagents may be constructed by packaging the appropriate materials, including, for example, a polypeptide defining a specific epitope of interest or antibodies that bind such epitopes in suitable containers. In addition, the kit optionally may include additional reagents, for example, suitable detection systems and buffers.

In addition, these antibodies, preferably monoclonal, can be bound to matrices similar to CNBr-activated Sepharose and used for the affinity purification of US-type or US-subtype hepatitis E specific proteins from cell cultures, or biological tissues such as blood and liver such as to purify recombinant and native viral antigens and proteins.

II. (ii) Nucleic Acid-based Assays

It is contemplated that if the marker is a US-type or US-subtype specific nucleotide sequence, the binding partner preferably also is a nucleotide sequence or an analog thereof that hybridizes specifically to the marker sequence or to regions adjacent thereto. Based on the unique polynucleotide sequences disclosed herein, it is contemplated that a binding partner may be a nucleotide sequence complementary to a US-type or US-subtype specific nucleotide sequence, for example, a nucleotide sequence or analog thereof complementary to at least a portion of an ORF 1 sequence, an ORF 2 sequence, or an ORF 3 sequence of a US-type or US-subtype hepatitis E virus, which is unique when compared to the corresponding nucleotide sequences of the Burmese and Mexican families. Furthermore, it is contemplated that noncoding portions of the genome of US-type and US-subtype hepatitis E viruses which are unique relative to the genomes of the Burmese and Mexican families of hepatitis E also may provide useful markers in the practice of the invention. Such nucleotide sequences (either primers or probes) are of a length which allow detection of US-type or US-subtype specific sequences by hybridization and/or amplification and may be prepared using routine, standard methods, including automated oligonucleotide synthesis methodologies, well known and thoroughly discussed in the art. A complement of any unique portion of the HEV US-1 genome will be satisfactory. Complete complementarity is desirable for use as probes, although it may be unnecessary as the length of the fragment is increased.

Similarly, it is contemplated that the binding partner may be a polynucleotide sequence, for example, a DNA, RNA or PNA sequence, preferably comprising 8-100 nucleotides more preferably comprising 10-75 nucleotides and most preferably comprising 15-50 nucleotides, which is capable of hybridizing specifically to the target sequence. It is understood that the target sequence may be a nucleotide sequence defining at least a portion of a genome of a US-type or US-subtype hepatitis E virus, or a sequence complementary thereto. It is known in the art that the particular stringency conditions selected for a hybridization reaction depend largely upon the degree of complementarity of the binding partner nucleic acid sequence with the target sequence, the composition of the binding sequence and the length of the binding sequence. The parameters for determining stringency conditions are well known to those of ordinary skill in the art or are deemed to be readily ascertained from standard textbooks (see for example, Maniatis et al., Molecular Cloning: A Laboratory Manual, (Cold Spring Harbor Press, N.Y., 1989)).

The sequences provided herein may be used to produce probes which can be used in assays for the detection of nucleic acids in test samples. The probes may be designed from conserved nucleotide regions of the polynucleotides of interest or from non-conserved nucleotide regions of the polynucleotide of interest. The design of such probes for optimization in assays is within the skill of the routineer. Generally, nucleic acid probes are developed from non-conserved or unique regions when maximum specificity is desired, and nucleic acid probes are developed from conserved regions when assaying for nucleotide regions that are closely related to, for example, different members of a multigene family or in related species like mouse and man.

One preferred protocol provides a method of detecting the presence or absence of a US-type or US-subtype hepatitis E virus in a test sample. The method comprises the steps of (a) providing a probe comprising a polynucleotide sequence containing at least 15 contiguous nucleotides from a US-type or US-subtype isolate, wherein the sequence is not present in other members of the hepatitis E Burmese and Mexican families; (b) contacting the test sample and the probe under conditions that permit formation of a polynucleotide duplex between the probe and its complement, in the absence of substantial polynucleotide duplex formation between the probe and non US-type and non US-subtype hepatitis polynucleotide sequences present in the test sample; and (c) detecting the presence of any polynucleotide duplexes containing the probe.

Preferred nucleotide sequences may comprise nucleotide residue numbers 1 through 5097 of SEQ ID NO:89, or a naturally occurring sequence variant thereof. With regard to this sequence, the term “a naturally occurring sequence variant” includes any nucleic acid sequence that is at least 73.3%, preferably at least 75.3%, more preferably at least 78.3%, and most preferably at least 95% identical to residues 1 through 5097 of SEQ ID NO:89. Other preferred marker or binding partner sequences may comprise nucleotide residue numbers 5132 through 7114 of SEQ ID NO:89, or a naturally occurring sequence variant thereof. With regard to this sequence, the term “naturally occurring sequence variant” includes any nucleic acid sequence that is at least 87.4%, preferably at least 89.4%, more preferably at least 92.4%, and most preferably at least 95% identical to residues 5132 through 7114 of SEQ ID NO:89. Other preferred marker or binding partner sequences may comprise nucleotide residue numbers 5094 through 5462 of SEQ ID NO:89, or a naturally occurring sequence variant thereof. With regard to this sequence, the term “naturally occurring sequence variant” includes any nucleic acid sequence that is at least 88.3% identical, preferably at least 90.3% identical, more preferably at least 93.3% identical, and most preferably at least 95% identical to residues 5094 through 5462 of SEQ ID NO:89.

Furthermore, it is contemplated that useful nucleotide sequences may include, for example, portions of the ORF 1 sequence encoding, for example, a protein selected from the group consisting of the methyltransferase protein, the protease protein, the Y domain protein, the X domain protein, the helicase protein, the hypervariable region protein and the RNA-dependent RNA polymerase protein, or a variant thereof. Accordingly, it is contemplated that a useful methyltransferase encoding region of ORF 1 preferably has at least 78%, more preferably has at least 80%, and most preferably has at least 83% identity to residues 1-693 of SEQ ID NO:89. Also, it is contemplated that a useful protease encoding region of ORF 1 preferably has at least 66.1%, more preferably has at least 68.1%, and most preferably has at least 71.1% identity to residues 1270-2091 of SEQ ID NO:89. Also, it is contemplated that a useful Y domain encoding region of ORF 1 has at least 80%, more preferably has at least 82%, and most preferably has at least 85% identity to residues 619-1272 of SEQ ID NO:89. Also, it is contemplated that a useful X domain encoding region of ORF 1 has at least 73.5%, more preferably has at least 75.5%, and most preferably has at least 78.5% identity to residues 2365-2841 of SEQ ID NO:89. Also, it is contemplated that a useful helicase encoding region of ORF 1 has at least 77.5%, and most preferably has at least 79.5%, and most preferably has at least 81.5% identity to residues 2893-3591 of SEQ ID NO:89. Also, it is contemplated that a useful hypervariable region encoding region of ORF 1 has at least 51.2%, more preferably has at least 53.2%, and most preferably has at least 56.2% identity to residues 2092-2364 of SEQ ID NO:89. Also, it is contemplated that a useful RNA-dependent RNA polymerase encoding region of ORF 1 has at least 76.3%, more preferably has at least 78.3%, and most preferably has at least 81.3% identity to residues 3634-5094 of SEQ ID NO:89.

Preferred nucleotide sequences may comprise nucleotide residue numbers 36 through 5162 of SEQ ID NO:164, or a naturally occurring sequence variant thereof. With regard to this sequence, the term “a naturally occurring sequence variant” includes any nucleic acid sequence that is at least 73.6%, preferably at least 75.6%, more preferably at least 78.6% and more preferably at least 95% identical to residues 36 through 5162 of SEQ ID NO:164. Other preferred marker or binding partner sequences may comprise nucleotide residue numbers 5197 through 7179 of SEQ ID NO:164, or a naturally occurring sequence variant thereof. With regard to this sequence, the term “naturally occurring sequence variant” includes any nucleic acid sequence that is at least 80.7%, preferably at least 82.7%, more preferably at least 85.7% and most preferably at least95% identical to residues 5197 through 7179 of SEQ ID NO:164. Other preferred marker or binding partner sequences may comprise nucleotide residue numbers 5159 through 5527 of SEQ ID NO:164, or a naturally occurring sequence variant thereof. With regard to this sequence, the term “naturally occurring sequence variant” includes any nucleic acid sequence that is at least 87.9% identical, preferably at least 89.9% identical, more preferably at least 92.9% identical and even more preferably at least 95% identical to residues 5159 through 5527 of SEQ ID NO:164.

Furthermore, it is contemplated that useful HEV US-2 nucleotide sequences may include, for example, portions of the ORF 1 sequence encoding, for example, at least a portion of a protein selected from the group consisting of the methyltransferase protein, the protease protein, the Y domain protein, the X domain protein, the helicase protein, the hypervariable region protein and the RNA-dependent RNA polymerase protein, or a variant thereof. Accordingly, it is contemplated that a useful methyltransferase encoding region of ORF 1 preferably has at least 79.5%, more preferably has at least 81.5%, and most preferably has at least 84.5% identity to residues 36-755 of SEQ ID NO:164. Also, it is contemplated that a useful protease encoding region of ORF 1 preferably has at least 66.1%, more preferably has at least 68.1%, and most preferably has at least 71.1% identity to residues 1332-2153 of SEQ ID NO:164. Also, it is contemplated that a useful Y domain encoding region of ORF 1 has at least 80.7%, more preferably has at least 82.7%, and most preferably has at least 85.7% identity to residues 680-1334 of SEQ ID NO:164. Also, it is contemplated that a useful X domain encoding region of ORF 1 has at least 73.7%, more preferably has at least 75.7%, and most preferably has at least 78.7% identity to residues 2430-2906 of SEQ ID NO: 164. Also, it is contemplated that a useful helicase encoding region of ORF 1 has at least 76.4%, and most preferably has at least 78.4%, and most preferably has at least 81.4% identity to residues 2958-3656 of SEQ ID NO:164. Also, it is contemplated that a useful hypervariable region encoding region of ORF 1 has at least 50.4%, more preferably has at least 52.8%, and most preferably has at least 55.8% identity to residues 2154-2429 of SEQ ID NO:164. Also, it is contemplated that a useful RNA-dependent RNA polymerase encoding region of ORF 1 has at least 76.8%, more preferably has at least 78.8%, and most preferably has at least 81.8% identity to residues 3699-5159 of SEQ ID NO:164.

Other useful nucleotide sequences comprise the nucleotide sequences that encode the amino acid sequences selected from the group consisting of SEQ ID NOS:93, 168, 173, 174, 175, 176, 223, and 224 and nucleotide sequences complementary thereto.

It is contemplated that the nucleic acid sequences provided herein may be used to determine the presence of US-type or US-subtype hepatitis E virus in a test sample by conventional nucleic acid based assays, for example, by polymerase chain reaction (PCR) and/or by blot hybridization studies (described in detail below). In addition to their use in nucleic acid based assays, it is contemplated the aforementioned nucleic acid sequences may be integrated in vectors which may then be transformed or transfected into a host cell of interest, for example, vaccinia or mycobacteria. The resulting host cells may then be combined with a pharmaceutically acceptable carrier and used, for example, as a recombinant vaccine for immunizing a mammal, either prophylactically or therapeutically, against a preselected US-type or US-subtype hepatitis E virus.

The polymerase chain reaction (PCR) is a technique for amplifying a desired nucleic acid sequence (target) contained in a nucleic acid or mixture thereof. In PCR, a pair of primers typically are employed in excess to hybridize at the outside ends of complementary strands of the target nucleic acid. The primers are each extended by a polymerase, for example, a thermostable polymerase, using the target nucleic acid as a template. The extension products become target sequences themselves, following dissociation from the original target strand. New primers then are hybridized and extended by a polymerase, and the cycle is repeated to geometrically increase the number of target sequence molecules. PCR is disclosed in U.S. Pat. Nos. 4,683,195 and 4,683,202.

The Ligase Chain Reaction (LCR) is an alternate method for nucleic acid amplification. In LCR, probe pairs are used which include two primary (first and second) and two secondary (third and fourth) probes, all of which are employed in molar excess of the target nucleic acid sequence. The first probe hybridizes to a first segment of the target strand and the second probe hybridizes to a second segment of the target strand, the first and second segments being contiguous so that the primary probes abut one another in 5′ phosphate-3′hydroxyl relationship, and so that a ligase can covalently fuse or ligate the two probes into a fused product. In addition, a third (secondary) probe can hybridize to a portion of the first probe and a fourth (secondary) probe can hybridize to a portion of the second probe in a similar abutting fashion. Once the ligated strand of primary probes is separated from the target strand, it will hybridize with the third and fourth probes which can be ligated to form a complementary, secondary ligated product. The ligated products are functionally equivalent to either the target or its complement. By repeated cycles of hybridization and ligation, amplification of the target sequence is achieved. This technique is described more completely in EP-A-320 308 to K. Backman published Jun. 16, 1989 and EP-A-439 182 to K. Backman et al, published Jul. 31, 1991.

For amplification of mRNAs, it is within the scope of the present invention to reverse transcribe mRNA into cDNA followed by polymerase chain reaction (RT-PCR); or, to use a single enzyme for both steps as described in U.S. Pat. No. 5,322,770; or to reverse transcribe mRNA into cDNA followed by asymmetric gap ligase chain reaction (RT-AGLCR) as described by R. L. Marshall, et al., PCR Methods and Applications 4: 80-84 (1994).

Other known amplification methods which can be utilized herein include but are not limited to the so-called “NASBA” or “3 SR” technique described in Proc. Natl. Acad. Sci. USA 87: 1874-1878 (1990) and also described in Nature 350 (No. 6313): 91-92 (1991); Q-beta amplification as described in published EP 4544610; strand displacement amplification (as described in G. T. Walker et al., Clin. Chem. 42: 9-13 [1996]) and EP 684315; and target mediated amplification, as described by PCT Publication WO 9322461.

In one embodiment, the present invention generally comprises the steps of contacting a test sample suspected of containing a target polynucleotide sequence with amplification reaction reagents comprising an amplification primer, and a detection probe that can hybridize with an internal region of the amplicon sequences. Probes and primers employed according to the method herein provided are labeled with capture and detection labels wherein probes are labeled with one type of label and primers are labeled with the other type of label. Additionally, the primers and probes are selected such that the probe sequence has a lower melt temperature than the primer sequences. The amplification reagents, detection reagents and test sample are placed under amplification conditions whereby, in the presence of target sequence, copies of the target sequence (an amplicon) are produced. The double stranded amplicon then is thermally denatured to produce single stranded amplicon members. Upon formation of the single stranded amplicon members, the mixture is cooled to allow the formation of complexes between the probes and single stranded amplicon members.

After the probe/single stranded amplicon member hybrids are formed, they are detected. Standard heterogeneous assay formats are suitable for detecting the hybrids using the detection labels and capture labels present on the primers and probes. The hybrids can be bound to a solid phase reagent by virtue of the capture label and detected by virtue of the detection label. In cases where the detection label is directly detectable, the presence of the hybrids on the solid phase can be detected by causing the label to produce a detectable signal, if necessary, and detecting the signal. In cases where the label is not directly detectable, the captured hybrids can be contacted with a conjugate, which generally comprises a binding member attached to a directly detectable label. The conjugate becomes bound to the complexes and the conjugates presence on the complexes can be detected with the directly detectable label. Thus, the presence of the hybrids on the solid phase reagent can be determined. Those skilled in the art will recognize that wash steps may be employed to wash away unhybridized amplicon or probe as well as unbound conjugate.

Test samples for detecting target sequences can be prepared using methodologies well known in the art such as by obtaining a sample and, if necessary, disrupting any cells contained therein to release target nucleic acids. In the case where PCR is employed in this method, the ends of the target sequences are usually known. In cases where LCR or a modification thereof is employed in the preferred method, the entire target sequence is usually known. Typically, the target sequence is a nucleic acid sequence such as, for example, RNA or DNA.

While the length of the primers and probes can vary, the probe sequences are selected such that they have a lower melt temperature than the primer sequences. Hence, the primer sequences are generally longer than the probe sequences. Typically, the primer sequences are in the range of between 20 and 50 nucleotides long, more typically in the range of between 20 and 30 nucleotides long. Preferred primer sequences typically are greater than 20 nucleotides long. The typical probe is in the range of between 10 and 25 nucleotides long more typically in the range of between 15 and 20 nucleotides long. Preferred probe sequences typically are greater than 15 nucleotides long.

Alternatively, a probe may be involved in the amplifying a target sequence, via a process known as “nested PCR”. In nested PCR, the probe has characteristics which are similar to those of the first and second primers normally used for amplification (such as length, melting temperature etc.) and as such, may itself serve as a primer in an amplification reaction. Generally in nested PCR, a first pair of primers (P₁ and P₂) are employed to form primary extension products. One of the primary primers (for example, P₁) may optionally be a capture primer (i.e. linked to a member of a first reactive pair), whereas the other primary primer (P₂) is not. A secondary extension product is then formed using a probe (P_{1′}) and a probe (P_{2′}) which may also have a capture type label (such as a member of a second reactive pair) or a detection label at its 5′ end. The probes are complementary to and hybridize at a site on the template near or adjacent the site where the 3′ termini of P₁ and P₂ would hybridize if still in solution. Alternatively, a secondary extension product can be formed using the P₁ primer with the probe (P_{2′}) or the P₂ primer with the probe (P_{1′}) sometimes referred to as “hemi-nested PCR”. Thus, a labeled primer/probe set generates a secondary product which is shorter than the primary extension product. Furthermore, the secondary product may be detected either on the basis of its size or via its labeled ends (by detection methodologies well known to those of ordinary skill in the art). In this process, probe and primers are generally employed in equivalent concentrations.

Various methods for synthesizing primers and probes are well known in the art. Similarly, methods for attaching labels to primers or probes are also well known in the art. For example, it is a matter of routine experimentation to synthesize desired nucleic acid primers or probes using conventional nucleotide phosphoramidite chemistry and instruments available from Applied Biosystems, Inc., (Foster City, Calif.), Dupont (Wilmington, Del.), or Milligen (Bedford Mass.). Many methods have been described for labeling oligonucleotides such as the primers or probes of the present invention. Enzo Biochemical (New York, N.Y.) and Clontech (Palo Alto, Calif.) both have described and commercialized probe labeling techniques. For example, a primary amine can be attached to a 3′ oligo terminus using 3′-Amine-ON CPG™ (Clontech, Palo Alto, Calif.). Similarly, a primary amine can be attached to a 5′ oligo terminus using Aminomodifier II™ (Clontech). The amines can be reacted to various haptens using conventional activation and linking chemistries. In addition, WO 92/10506, published Jun. 25, 1992 and U.S. Pat. No. 5,290,925, issued Mar. 1, 1994, teach methods for labeling probes at their 5′ and 3′ termini, respectively. In addition, WO 92/11388 published Jul. 9, 1992 teaches methods for labeling probes at their ends. According to one known method for labeling an oligonucleotide, a label-phosphoramidite reagent is prepared and used to add the label to the oligonucleotide during its synthesis. See, for example, N. T. Thuong et al., Tet. Letters 29(46): 5905-5908 (1988); or J. S. Cohen et al., published U.S. patent application Ser. No. 07/246,688 (NTIS ORDER No. PAT-APPL-7-246,688) (1989). Preferably, probes are labeled at their 3′ and 5′ ends.

Capture labels are carried by the primers or probes and can be a specific binding member which forms a binding pair with the solid phase reagent's specific binding member. It will be understood, of course that the primer or probe itself may serve as the capture label. For example, in the case where a solid phase reagent's binding member is a nucleic acid sequence, it may be selected such that it binds a complementary portion of the primer or probe to thereby immobilize the primer or probe to the solid phase. In cases where the probe itself serves as the binding member, those skilled in the art will recognize that the probe will contain a sequence or “tail” that is not complementary to the single stranded amplicon members. In the case where the primer itself serves as the capture label, at least a portion of the primer will be free to hybridize with a nucleic acid on a solid phase because the probe is selected such that it is not fully complementary to the primer sequence.

Generally, probe/single stranded amplicon member complexes can be detected using techniques commonly employed to perform heterogeneous immunoassays. Preferably, in this embodiment, detection is performed according to the protocols used by the commercially available Abbott LCx® instrumentation (Abbott Laboratories, Abbott Park, Ill.).

Other useful procedures known in the art include solution hybridization, and dot and slot blot hybridization protocols. The amount of the target nucleic acid present in a sample optionally may be quantitated by measuring the radioactivity of hybridized fragments, using standard procedures known in the art.

III. Vaccines

It is contemplated that vaccines may be prepared from one or more immunogenic polypeptides based on US-type and/or US-subtype specific protein sequences or antibodies that bind to such protein sequences. In addition, it is contemplated that vaccines also may comprise dead, live but attenuated US-type or US-subtype hepatitis E virus, or a live, recombinant vaccine comprising a heterologous host cell, for example, a vaccinia virus, expressing a US-type or US-subtype hepatitis E virus specific antigen.

With regard to the polypeptide based vaccines, the polypeptide must define at least one epitope. It is contemplated, however, that the vaccine may comprise a plurality of different epitopes which are defined by one or more polypeptide chains. Furthermore, it is contemplated that nonstructural proteins as well as structural proteins may provide protection against viral pathogenicity, even if they do not cause the production of neutralizing antibodies. Considering the above, multivalent vaccines against the US-type or US-subtype virus may comprise one or more structural proteins, and/or one or more nonstructural proteins. These immunogenic epitopes can be used in combinations, i.e., as a mixture of recombinant proteins, synthetic peptides and/or polypeptides isolated from the virion; which may be co-administered at the same or administered at different time.

Methodologies for the preparation of protein or peptide based vaccines which contain at least one immunogenic peptide as an active ingredient are well known in the art. Typically, such vaccines are prepared as injectables, either as liquid solutions or suspensions. The preparation may be emulsified or the protein may be encapsulated in liposomes. The active immunogenic ingredients may be mixed with pharmacologically acceptable excipients which are compatible with the active ingredient. Suitable excipients include, without limitation, water, saline, dextrose, glycerol, ethanol or a combination thereof. The vaccine also may contain small amounts of auxiliary substances such as wetting or emulsifying reagents, pH buffering agents, and/or adjuvants which enhance the effectiveness of the vaccine. For example, such adjuvants can include aluminum hydroxide, N-acetyl-muramyl-L-threonyl-D-isoglutamine (thr-DMP), N-acetyl-nomuramyl-L-alanyl-D-isoglutamine (CGP 11687, also referred to as nor-MDP), N-acetyl-muramyul-L-alanyl-D-isoglutaminyl-L-alanine-2-(1′2′-dipalmitoyl sn-glycero-3-hydroxphosphoryloxy)-ethylamine (CGP 19835A, also referred to as MTP-PE), and RIBI (MPL+TDM+CWS) in a 2% squalene/Tween-80® emulsion. The effectiveness of an adjuvant may be determined by measuring the amount of antibodies directed against an immunogenic polypeptide containing a US-type or US-subtype specific antigenic sequence resulting from administration of this polypeptide in vaccines which also comprise various adjuvants under investigation.

The vaccines usually are administered by intravenous or intramuscular injection. Additional formulations which are suitable for other modes of administration include suppositories and, in some cases, oral formulations. For suppositories, traditional binders and carriers may include but are not limited to polyalkylene glycols or triglycerides. Such suppositories may be formed from-mixtures containing the active ingredient in the range of from about 0.5% to about 10%, preferably, from about 1% to about 2% (w/w). Oral formulation may include excipients including, for example, mannitol, lactose, starch, magnesium stearate, sodium saccharine, cellulose, magnesium carbonate and the like. These compositions may take the form of solutions, suspensions, tablets, pills, capsules, sustained release formulations or powders and contain about 10% to about 95% of active ingredient, preferably about 25% to about 70% (w/w).

The polypeptide chains used in the vaccine may be formulated into the vaccine as neutral or salt forms. Pharmaceutically acceptable salts include, for example, acid addition salts formed by the addition of inorganic acids such as hydrochloric or phosphoric acids, or such organic acids such as acetic, oxalic, tartaric, maleic, or other acids known to those skilled in the art. Salts formed with the free carboxyl groups also may be derived from inorganic bases such as sodium, potassium, ammonium, calcium or ferric hydroxides and the like, and organic bases such as isopropylamine, trimethylamine, 2-ethylamino ethanol, histidine procaine, or other bases known to those skilled in the art.

Vaccines typically are administered in a way compatible with the dosage formulation, and in such amounts that will be effective prophylactically and/or therapeutically. The quantity to be administered generally ranges from about 5 μg to about 250 μg of antigen per dose, however the actual dose will depend upon the health and size of the subject, the capacity of the subject's immune system to synthesize antibodies, and the degree of protection sought. The vaccine may be given in a single or multiple dose schedule. A multiple dose is one in which a primary course of vaccination may be with one to ten separate doses, followed by other doses given at subsequent time intervals required to maintain and/or to reinforce the immune response, for example, at one to four months for a second dose, and if required by the individual, a subsequent dose(s) several months later. In addition, the dosage regimen may be determined, at least in part, by the need of the individual, and may be dependent upon the practitioner's judgment.

With regard to dead or otherwise inactivated US-type or US-subtype hepatitis E virus containing vaccines, inactivation may be facilitated using conventional methodologies well known and thoroughly documented in the art. Preferred inactivation methods include, for example, exposure to one or more of (i) organic solvents, (ii) detergents, (iii) formalin, and (iv) ionizing radiation. It is contemplated that some of the proteins in attenuated vaccines may cross-react with other known viruses, and thus shared epitopes may exist between a US-type or US-subtype hepatitis E virus and other members of the HEV family (for example, members of the Burmese or Mexican families) and thus give rise to protective antibodies against one or more of the disorders caused by these pathogenic agents. Preferred formulations and modes of administration are thoroughly documented in the art and so are not discussed in detail herein. The various factors to be considered may include one or more features discussed hereinabove for the peptide based vaccines.

With regard to the live, but attenuated vaccines, it may be possible to produce attenuated virus using any of the attenuation methods known and used in the art. Briefly, attenuation may be accomplished by passage of the virus at low temperatures or by introducing missense mutations or deletions into the viral genome. Preferred formulations and modes of administration are thoroughly documented in the art and so are not discussed in detail herein. The various factors to be considered may include one or more features discussed hereinabove for the peptide based vaccines.

With regard to live, recombinant vaccines (vector vaccines), these may be developed by incorporating into the genome of a living but harmless virus or bacterium, a gene or nucleic acid sequence encoding a US-type or US-subtype hepatitis E specific polypeptide chain defining an antigenic determinant. The resulting vector organism may then be administered to the intended host. Typically, for such a vaccine to be successful, the vector organism must be viable, and either naturally non-virulent or have an attenuated phenotype. Preferred host organisms include, vaccinia virus, adenovirus, adeno-associated virus, salmonella and mycobacteria. Live strains of vaccinia virus and mycobacteria have been administered safely to humans in the forms of the smallpox and tuberculosis (BCG) vaccines, respectively. In addition, they have been shown to express foreign proteins and exhibit little or no conversion into virulent phenotypes. Vector vaccines are capable of carrying a plurality of foreign genes or nucleic acid sequences thereby permitting simultaneous vaccination against a variety of preselected antigenic determinants. Preferred formulations and modes of administration are thoroughly documented in the art and so are not discussed in detail herein.

IV. Identification of Molecules With Anti-US-type or Anti-US-subtype Hepatitis E Virus Activity.

In view of the discovery of specific HEV US-type sequences, it is contemplated that one skilled in the art may be able to identify molecules which either inactivate or reduce the activity of HEV US-type specific proteins, e.g., the helicase, methyltransferase, or protease proteins encoded by the ORF 1 portions of the HEV genome. An exemplary protocol for identifying molecules that inhibit the HCV protease is described in U.S. Pat. No. 5,597,691, the disclosure of which is incorporated herein by reference. Although, the method pertains to the identification of HCV protease inhibitors, it is contemplated that the same or similar protocols maybe used to identify HEV protease inhibitors, or any other protein encoded by a HEV US-type sequence.

Briefly, a method for identifying HEV protease inhibitors is as follows. Typically, a substrate is employed which mimics the proteases natural substrate, but which provides a quantifiable signal when cleaved. The signal preferably is detectable by calorimetric or fluorometric means; however, other methods such as HPLC or silica gel chromatography, nuclear magnetic resonance, and the like may also be useful. After optimum substrate and protease concentrations have been determined, candidate protease inhibitors are added one at a time to the reaction mixture at a range of concentrations. The assay conditions preferably resemble the conditions under which the protease is to be inhibited in vivo, i.e., under physiologic pH, temperature, ionic strength, etc. Suitable inhibitors exhibit strong protease inhibition at concentrations which do not raise toxic side effects in the subject. Inhibitors which compete for binding to the protease active site may require concentrations equal to or greater than the substrate concentration, while inhibitors capable of binding irreversibly to the protease active site may be added in concentrations on the order of the enzyme concentration.

It is contemplated that the inhibitors may be organic compounds, which, for example, mimic the cleavage site recognized by the HEV protease, or alternatively, may be proteins, for example, antibodies or antibody fragments capable of binding specifically to and inactivating or reducing the activity of the HEV protease. Once identified, the protease inhibitors may be administered by a variety of methods, such as intravenously, orally, intramuscularly, intraperitoneally, bronchially, intranasally, and so forth. The preferred route of administration will depend upon the nature of inhibitor. Inhibitors prepared as organic compounds may be administered orally (which is generally preferred) if well absorbed. Protein-based inhibitors (such as most antibodies or antibody derivatives) generally are administered by parenteral routes.

### EXAMPLES

Practice of the invention will be more fully understood from the following examples, which are presented herein for illustrative purposes only, and should not be construed as limiting the invention in any way. All citations to the literature, both supra and infra, including Patents, Patent applications and scientific publications are incorporated by reference herein, in their entirety.

### Example 1

### Case Study

HEV strain US-1 was identified in the serum of a patient (USP-1) suffering from acute hepatitis. The patient was a 62 year old, white male who was hospitalized in Rochester, Minn. after a three-week history of fever, abdominal pain, jaundice, and pruritis. Onset of signs and symptoms began two weeks after returning home following a ten day trip to San Jose, Calif.

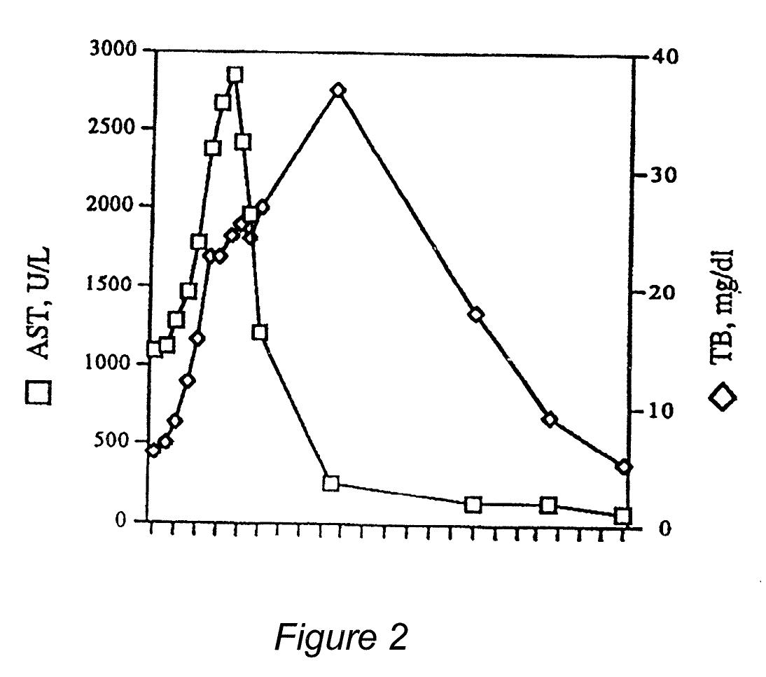

His past medical history included a nephrectomy for autosomal dominant polycystic kidney disease accompanied by mild renal insufficiency, and a laparoscopic cholecystectomy for symptomatic cholelithiasis. The patient had osteoanthritis and was hypertensive. Lisinopnil therapy had been initiated three months prior to admission. Physical examination revealed an ill appearing icteric white male with an enlarged tender liver, and no asterixis. Serum aspartate aminotransferase (AST), alanine aminotransferase (ALT), and bilirubin levels were markedly elevated at the time of hospital admission and peaked 8 days and 16 days after hospitalization, respectively (FIG. 2). Lisinopril was discontinued on admission. Serologies for hepatitis A (IgM and IgG anti-HAV), hepatitis B (HBsAg, IgM and IgG anti-HBc), hepatitis C (anti-HCV), and HCV RNA were negative. Ceruloplasmin, iron, transferrin, anti-nuclear and anti-smooth muscle antibodies, toxin and drug screen were all normal. Careful questioning of the patient revealed no history of ethanol use. Abdominal ultrasound and computed tomography scan, and endoscopic retrograde cholangiopancreatogram were also normal. A liver biopsy showed a severe, acute lobular hepatitis with striking pyknotic and ballooning degeneration of hepatocytes consistent with autoimmune, drug, or viral hepatitis.

The patient made a complete clinical recovery within 2 months, with normalization of AST, ALT, and bilirubin noted about 5 months after hospital admission. No risk factors for acquiring HEV were identified. He had not traveled outside the US for over 10 years. In the 6 weeks prior to illness onset, the only meals he reported eating that were not prepared at home were at a Mexican restaurant and a large fast food restaurant chain. He had no exposure to untreated drinking water, did not report eating raw shellfish, and had no known exposure to farm animals. None of the food handlers at the Mexican restaurant or the fast food restaurant reported foreign travel since less than 5 months from admission date and none reported signs and/or symptoms of hepatitis. No other cases of non-ABC hepatitis were reported in the county health department where the patient stayed in California, and where the patient lived in Minnesota during the period of admission. No family members had signs and/or symptoms of hepatitis either during the patient's trip to California or in the subsequent 10 weeks. Serum obtained from 6 family members in California, and from his spouse who lived with him in Minnesota over the period of interest were negative for anti-HEV by EIA.

### Example 2

### Identification of Unique Isolate of HEV US-1

The presence of HEV was determined by RT-PCR using HEV primer sequences. described (Schlauder et al. (1995) J. Virological Methods 46: 81-89). Ethanol precipitated nucleic acids were resuspended in 3 μL of diethyl pyrocarbonate (DEPC) treated water. cDNA synthesis and PCR were performed using the GeneAmp RNA PCR kit from Perkin-Elmer (Norwalk, Conn.) in accordance with the manufacturer's instructions. RNA (1 μL) was used as a template for each 10 μL cDNA reaction. cDNA synthesis was primed with specific primers added to a final concentration of 4 μM. The subsequent amplification of cDNA was primed with oligonucleotides added to a final concentration of 0.8 to 1.0 μM. PCR was performed for 40 cycles (94° C., 20 sec; 55° C., 30 sec; 72° C., 30 sec; followed by an extension cycle of 72° C. for 3 min). The initial PCR reaction (2 μL) then was used as a template for a second round of amplification using a nested set of PCR primers. PCR was performed using the GeneAmp PCR kit from Perkin-Elmer in accordance with the manufacturer's instructions. Briefly, primers were added to a final concentration of 1 μM. The initial set of experiments used three sets of primers. Two from the 5′-end of ORF 1 based on sequences from the Burmese and Mexican strains. One set from the 3′-end of ORF 1 based on the Mexican strain sequence. The three sets of primers used were as follows:

| | | |
|---|---|---|
| Primer | Sequence | SEQ ID NO: |

Primer Set 1

| | | |
|---|---|---|
| 5′-ORF 1-Mexican primer C375M | CTGAACATCCCGGCCGAC | SEQ ID NO:1 |
| PCR primer A1-350M | AGAAAGCAGCGATGGAGGA | SEQ ID NO:2 |
| PCR primer S1-34M | GCCCACCAGTTCATTAAGGCT | SEQ ID NO:3 |
| nested PCR primer A2-320M | TCATTAATGGAGCGTGGGTG | SEQ ID NO:4 |
| nested PCR primer S2-55M | CCTGGCATCACTACTGCTAT | SEQ ID NO:5 |

Primer Set 2

| | | |
|---|---|---|
| 5′-ORF 1-Burmese cDNA primer C375 | CTGAACATCACGCCCAAC | SEQ ID NO:6 |
| PCR primer A1-350 | AGGAAGCAGCGGTGGACCA | SEQ ID NO:7 |
| PCR primer S1-34 | GCCCATCAGTTTATTAAGGC | SEQ ID NO:8 |
| nested PCR primer A2-320 | TCATTTATTGAGCGGGGATG | SEQ ID NO:9 |
| nested PCR primer S2-55 | CCTGGCATCACTACTGCTAT | SEQ ID NO:10 |

Primer Set 3

| | | |
|---|---|---|
| 3′-ORF 1-Mexican cDNA primer M1PR6 | CCATGTTCCACACCGTATTCCAGAG | SEQ ID NO:11 |
| PCR primer 54294M | GTGTTCTACGGGGATGCTTATGACG | SEQ ID NO:12 |
| nested PCR primer M1PF6 | GACTCAGTATTCTCTGCTGCCGTGG | SEQ ID NO:13 |
| nested PCR primer A4556 | GGCTCACCAGAATGCTTCTTCCAGA | SEQ ID NO:14 |

The 5′-ORF 1-Burmese primers are described in Schlauder et al. (1993) Lancet 341: 378. Primers M1PR6 and M1PF6 are described in McCaustland et al. (1991) J. Virological Methods 35: 331-342. The PCR products were separated by agarose gel electrophoresis and visualized by UV irradiation after ethidium bromide staining. The resulting PCR products were hybridized to a radiolabelled probe after Southern blot transfer to a nitrocellulose filter.

Radiolabelled probes were generated from PCR products purified with the QIAEX gel extraction purification kit by Qiagen (Chatsworth, Calif.). Radiolabel was incorporated using the Stratgene® (La Jolla, Calif.) Prime-It II kit according to the manufacturer's instructions. Filters were prehybridized in Rapid-hyb buffer from Amersham (Arlington Heights, Ill.) for 3-5 hours, and then hybridized in Fast-Pair Hybridization Solution with 100-200 cpm/cm2 at 42° C. for 15-25 hours. Filters then were washed as described in Schlauder et al. (1992) J. Virol. Methods 37: 189-200. Phosphorimages of the probed filters were obtained with a Molecular Dynamics Phosphorimager 425E (Sunnyvale, Calif.).

Ethidium bromide stained bands were detected with the primers from the 5′-end of ORF 1. However, only the primers based on the Mexican strain resulted in a nested product of the expected size of 266 base pairs. Hybridization to a probe derived from a Burmese-like strain (identity >90%) infected patient resulted in a very weak hybridization signal to the patient USP-1 derived products relative to the signal from the Burmese positive control. These results gave the first indication that this isolate was not closely related to the Burmese isolate. No probe was available from the Mexican strain.

To confirm these results, RNA was extracted from additional serum aliquots of patient USP-1. RT-PCR was performed using the 5′-ORF 1-Mexican primers, SEQ ID NOS:1-5, as described above. Following agarose gel electrophoresis and staining with ethidium bromide, a 342 bp product was visualized in each sample. The PCR products were extracted from the agarose gel using the QIAEXII Agarose Gel Extraction Kit by Qiagen (Chatsworth, Calif.) and cloned into pT7 Blue T-vector plasmid by Novagen (Madison, Wis.). The cloned products were sequenced using the SEQUENASE VERSION 2.0 sequencing kit (USB, Cleveland, Ohio) in accordance with the manufacturers instructions.

The nucleotide sequences obtained from the product of the latter two samples were identical and are shown in SEQ ID NO:15. These results indicate that only the cDNA primer and primer S1 from both the Burmese and Mexican strains resulted in an ethidium bromide stainable product from the patient USP-1 samples. Only the Mexican strain based nested primers, S2 and A2 generated an ethidium bromide stainable product of the expected size.

In order to determine the degree of relatedness between the HEV US-1 isolate and other known isolates of HEV, alignments of the nucleotide and amino acid sequences were performed using the program GAP of the Wisconsin Sequence Analysis Package (Version 9), available from the Genetics Computer Group, Inc., 575 Science Drive, Madison, Wis., 53711. The program employs the algorithm of Needleman and Wunsch (J. Mol. Biol. (1970) 48:443-453) to calculate the degree of similarity and identity, which are expressed as percentages between the two sequences being aligned. The gap creation and gap extension penalties were 50 and 3.0, respectively, for nucleic acid sequence alignments, and 12 and 4, respectively, for amino acid sequence comparisons.

The complete nucleotide and amino acid sequences of the two ‘prototype’ HEV isolates from Burma and Mexico, as well as other sequences used for analyses were obtained from GenBank, with their respective accession numbers are indicated in Table 1 below. Each of the these sequences are incorporated herein by reference.

TABLE 1

| Isolate | Genbank Accession Number |
|---|---|
| Mexican (M1) | M74506 |
| Burmese (B1) | M73218 |
| Pakistan (P1) | M80581 |
| Chinese (C4) | D11093 |

A 303 base pair sequence of HEV US-1 (homologous to residues 1-303 of SEQ ID NO:89) was compared against the homologous regions identified in the Mexican, Burmese, Pakistani, and Chinese strains. The resulting percent identities are summarized in Table 2 below.

TABLE 2
Identity over 303 nucleic acids from the 5'-end ORF 1 product

| | US-1 | Mexican | Burmese | Pakistan |
|---|---|---|---|---|
| Mexican | 77.2 | | | |
| Burmese | 74.9 | 83.2 | | |
| Pakistan | 75.9 | 83.2 | 95.7 | |
| Chinese | 75.9 | 83.5 | 95.7 | 97.4 |

The results in Table 2 indicate that the fragment from the 5′-end of ORF 1 from the USP-1 isolate showed a nucleic acid identity from about 74.9 to about 77.2% relative to other known isolates of HEV. This was less than the identity between the prototype Mexican and Burmese isolates (83.2%). These results indicate that the product likely was derived from a unique isolate of HEV not previously identified.

### Example 3

### Genome Extension and Sequencing of HEV US-1

The clone obtained and sequenced as described in Example 2 (SEQ ID NO:15) hereinabove was derived from a unique HEV genome, HEV US-1. To obtain sequences from additional regions of the HEV US-1 genome, several reverse transcriptase-polymerase chain reaction (RT-PCR) walking experiments were performed.

Total nucleic acids were extracted by the procedure described in Example 2 (for SEQ ID NO:19 only) or by one of the following procedures. Aliquots (25 μL) of patient USP-1 serum were extracted using the Total Nucleic Acid Extraction procedure in accordance with the manufacturers instructions (United States Biochemical) in the presence of 10 mg yeast tRNA as carrier. Nucleic acids were precipitated and resuspended in 3.75 μL RNase/DNase free water. Alternatively, total RNA was isolated from 100 μL of serum using the ToTALLY RNA isolation kit as recommended by the manufacturer (Ambion, Inc.). The resulting RNAs were treated with DNase and column purified with reagents from S.N.A.P. Total RNA isolation kit (Invitrogen, San Diego, Calif.). Thereafter, RNA was precipitated with 0.1 volumes of 3M sodium acetate, 2 μL pellet paint (Novagen) as carrier, and 2 volumes ethanol. RNA pellets were dissolved in 50 μL DEPC treated water.

RT-PCR was performed using the-GeneAmp RNA PCR kit in accordance with the manufacturers instructions (Perkin-Elmer). Random hexamers were used to prime cDNA synthesis in a total volume of 25 μL except for the isolation of SEQ ID NO:19 which utilized cDNA specifically primed with primer PA2-5560 (SEQ ID NO:16), as described in Example 2 above. US 1 -gap was generated with specifically primed cDNA generated using RNA extracted from 12.5 μL serum equivalents, primer US1 gap-a0.5 (SEQ ID NO:46), and Superscript II (3′ RACE Kit: GIBCO BRL). PCR was performed with the cDNA encompassing one-fifth of the total reaction volume (2 μL for 10 μL reaction or 5 μL for 25 μL reaction, etc.). Standard PCR was performed in the presence of 2 mM MgCl₂ and 0.5 to 1.0 μM of each primer. Modified reactions contained lx PCR Buffer and 20% Q Solution (Qiagen) in accordance with the manufacturer's instructions for the isolation of SEQ ID NOS:33 and 41. Reactions used two HEV consensus primers (Table 3), one HEV consensus primer and one HEV-US-1 specific primer (Table 4), two HEV US-1 specific primers (Table 5), one HEV US-1 specific primer and one HEV US-2 (see Example 5) specific primer (Table 6), or two HEV US-2 specific primers (Table 7). Reactions were subjected to thermal cycling as follows:

SEQ ID NOS:19, 24, 27, 30, 33, 41, 44, 60, 64, 68, 73, 78, and 83 were obtained by touchdown PCR. Amplification involved 43 cycles of 94° C. for 30 seconds, 55° C. for 30 seconds (−0.3° C./cycle), and 72° C. for 1 minute. This was followed by 10 cycles of 94° C. for 30 seconds, 40° C. for 30 seconds, and 72° C. for 1 minute. For SEQ ID NOS:38, 49, 52, and 55, cycling involved 35 rounds of 94° C. for 30 seconds, 55° C. for 30 seconds, and 72° C. for 1 minute. All amplifications were preceded by 1-2 minutes at 94° C. and followed by 72° C. for 5 to 10 minutes. The reactions were held at 4° C. prior to agarose gel analysis.

The isolation of SEQ ID NO:19 required a second round of touch down amplification to isolate the desired product. Here, 1 μL of first round was placed into a second round 25 μL reaction. The second round amplification utilized hemi-nested primers as indicated in Table 3 by reactions 1.1.1 and 1.1.2. The isolation of SEQ ID NO:24 required a second round of nested touch down amplification as described above and indicated in Table 4 as reactions 2.1.1 and 2.1.2. The isolation of SEQ ID NOS:38 and 49 required a second round of nested PCR (Table 5) utilizing 1 μL of first round into a 25 μL reaction as described above. The isolation of SEQ ID NOS:60, 64, 68, and 73 required nested PCR in which 1 μl of the first round was amplified in a 25 μL second round reaction (Table 6). Products SEQ ID NOS:78 and 83 were generated from two rounds of amplification (Table 7).

Agarose gel electrophoresis was performed on a fraction or all of the PCR reaction in a 0.8% to 2% agarose TAE gel in the presence of 0.2 mg/mL ethidium bromide. Products were visualized by UV irradiation and products of the desired molecular weight were excised, purified using GeneClean in accordance with the manufacturers' instructions (BIO 101, Inc.), and cloned into pT7-Blue T-Vector plasmid (Novagen) II or pGEM-T Easy Vector (Promega) in accordance with the manufacturers' instructions. Cloned products were sequenced as described in Example 2 or on a ABI Model 373 DNA Sequencer using ABI Sequencing Ready Reaction Kit as specified by the manufacturer. Results of these experiments are presented hereinbelow in Tables 3, 4, 5, 6, and 7.

TABLE 3

| Reaction | Primer 1 | Primer 2 | Approx. Prod. Size/SEQ ID |
|---|---|---|---|
| 1.1.1 | SEQ ID NO:17 | SEQ ID NO:16 | none |
| 1.1.2 | SEQ ID NO:18 | SEQ ID NO:16 | 251 bp/SEQ ID NO:19 |
| 1.2 | SEQ ID NO:28 | SEQ ID NO:29 | 168 bp/SEQ ID NO:30 |

TABLE 4

| | | | Approx. Product Size/ |
|---|---|---|---|
| Reaction | Primer 1 | Primer 2 | SEQ ID NO |
| 2.1.1 | SEQ ID NO:20 | SEQ ID NO:22 | none |
| 2.1.2 | SEQ ID NO:21 | SEQ ID NO:23 | 899 bp/SEQ ID NO:24 |
| 2.2 | SEQ ID NO:25 | SEQ ID NO:26 | 846 bp/SEQ ID NO:27 |
| 2.3 | SEQ ID NO:31 | SEQ ID NO:32 | 424 bp/SEQ ID NO:33 |
| 2.4 | SEQ ID NO:39 | SEQ ID NO:40 | 460 bp/SEQ ID NO:41 |
| 2.5 | SEQ ID NO:42 | SEQ ID NO:43 | 235 bp/SEQ ID NO:44 |

TABLE 5

| | | | Approx. Product |
|---|---|---|---|
| Reaction | Primer Set PCR 1 | Primer Set PCR 2 | Size/SEQ ID NO: |
| 3.1 | SEQ ID NO:34/SEQ ID NO:35 | SEQ ID NO:36/SEQ ID NO:37 | 1186 bp/SEQ ID NO:38 |
| 3.2 | SEQ ID NO:45/SEQ ID NO:46 | SEQ ID NO:47/SEQ ID NO:48 | 545 bp/SEQ ID NO:49 |
| 3.3 | SEQ ID NO:50/SEQ ID NO:51 | | 344 bp/SEQ ID NO:52 |
| 3.4 | SEQ ID NO:53/SEQ ID NO:54 | | 194 bp/SEQ ID NO:55 |

TABLE 6

| | | | Approx. Product |
|---|---|---|---|
| Reaction | Primer Set PCR 1 | Primer Set PCR 2 | Size/SEQ ID NO: |
| 4.1 | SEQ ID NO:56/SEQ ID NO:57 | SEQ ID NO:58/SEQ ID NO:59 | 464 bp/SEQ ID NO:60 |
| 4.2 | SEQ ID NO:61/SEQ ID NO:62 | SEQ ID NO:63/SEQ ID NO:62 | 433 bp/SEQ ID NO:64 |
| 4.3 | SEQ ID NO:65/SEQ ID NO:66 | SEQ ID NO:65/SEQ ID NO:67 | 382 bp/SEQ ID NO:68 |
| 4.4 | SEQ ID NO:69/SEQ ID NO:70 | SEQ ID NO:71/SEQ ID NO:72 | 451 bp/SEQ ID NO:73 |

TABLE 7

| | | | Approx. Product |
|---|---|---|---|
| Reaction | Primer Set PCR 1 | Primer Set PCR 2 | Size/SEQ ID NO: |
| 5.1 | SEQ ID NO:74/SEQ ID NO:75 | SEQ ID NO:76/SEQ ID NO:77 | 334 bp/SEQ ID NO:78 |
| 5.2 | SEQ ID NO:79/SEQ ID NO:80 | SEQ ID NO:81/SEQ ID NO:82 | 413 bp/SEQ ID NO:83 |

To obtain the sequence at the 3′ end of the genome, amplification utilized the 3′ RACE System of GIBCO BRL in accordance with the manufacturer's instructions. It was assumed that, as an HEV strain, the 3′ end of the HEV-US-1 genome would contain a poly-adenosine tail similar to the Mexican, Burmese, and Pakistani strains. RNA extracted as described above from the equivalent of 50 μL of serum was reverse transcribed utilizing the oligo dT adapter primer 5′-GGCCACGCGTCGACTAGTACTTTTTTTTTTTTTTTTT-3′ of (SEQ ID NO:84) supplied by the manufacturer. First round PCR utilized the AUAP primer supplied 5′-GGCCACGCGTCGACTAGTAC-3′ (SEQ ID NO:85) and a HEV US-specific primer (Table 8) at 0.2 mM final concentration with PCR Buffer, MgCl₂, and cDNA concentrations as recommended. Amplification involved 35 cycles of 94° C. for 30 seconds, 55° C. for 30 seconds, and 72° C. for 1 minute. Amplification was preceded by a 1 minute incubation at 94° C. and followed by a 72° C., 10 minute extension. A second round of amplification used 1 μL of first round in a 50 μL reaction. PCR buffer was 1× final concentration with 2 mM MgCl₂, and 0.5 mM of each of the primers. Primers were hemi-nested with the AUAP primer and a HEV-US-1 specific primer (Table 8). Amplification conditions were the same as first round. The products were analyzed by agarose gel electrophoresis, cloned, and sequenced as above.

TABLE 8

| | | | Approx. Product |
|---|---|---|---|
| Reaction | Primer Set PCR 1 | Primer Set PCR 2 | Size/SEQ ID NO: |
| 8.1 | SEQ ID NO:86/SEQ ID NO:85 | SEQ ID NO:87/SEQ ID NO:85 | 960 bp/SEQ ID NO:88 |

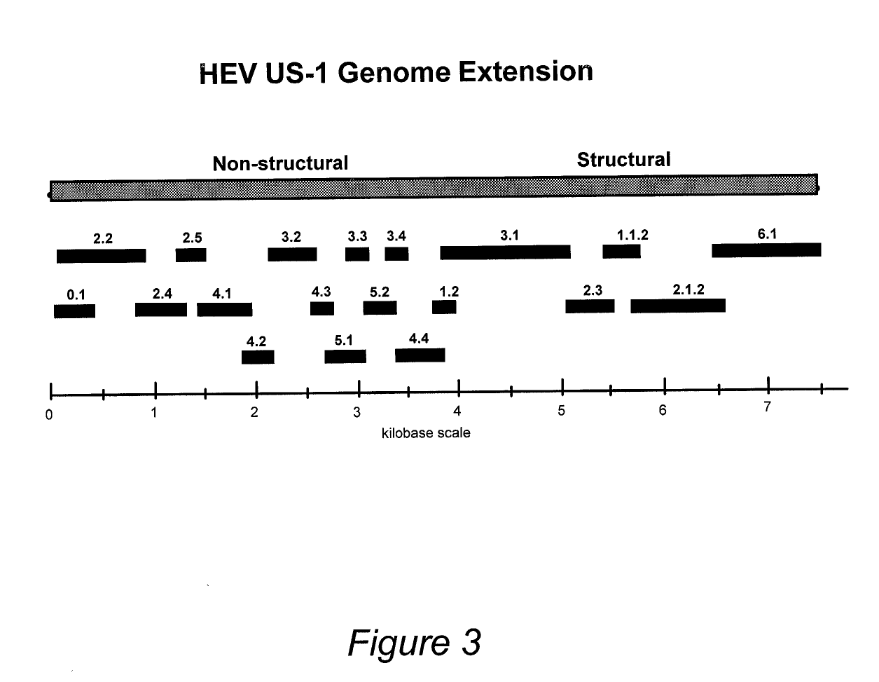

The sequences obtained from the products described in Tables 3, 4, 5, 6, 7, and 8 hereinabove, and the initial PCR product near the 5′ end of the genome, SEQ ID NO: 15, were assembled into contigs using the programs of the GCG package (Genetics Computer Group, Madison, Wis., version 9) and a consensus sequence determined. A schematic of the assembled contig is presented in FIG. 3, The HEV US-1 genome is 7202 bp in length, all of which has been sequenced (SEQ ID NO:89). This sequence was translated into three open reading frames, two of which are shown in SEQ ID NO:90 (the third ORF is positioned at nucleotide positions 5094-5462 but cannot be shown in SEQ ID NO:90 due to overlap with the other two ORFs). The resulting translations (ORF 1, ORF 2, and ORF 3) are set forth in SEQ ID NO:91, SEQ ID NO:92, and SEQ ID NO:93, respectively.

### Example 4

### Identification of Unique Isolate of HEV US-2

A patient from the US suffering from acute hepatitis, who tested for IgG class antibodies in the HEV EIA test, also tested positive by means of a US-1 strain-specific ELISA. This patient (USP-2) diagnosed with acute hepatitis, was a 62 year old male who was admitted to the hospital with jaundice and fatigue. Initial laboratory studies indicated an ALT of 1270 U/L (normal 0-40 U/L). Since there was a recent outbreak of hepatitis A virus (HAV) in the area, it was suspected that this individual was infected with HAV. However, the anti-HAV IgM test, HAVAB-M EIA (Abbott Laboratories) was negative as were tests for serologic markers for hepatitis B virus and hepatitis C virus. This patient's history included a visit to Cancun, Mexico, several weeks prior to the onset of his illness.

The sample from the patient then was analyzed for the presence of HEV specific sequences via PCR amplification using HEV US-1 specific PCR primers. RNA was extracted using Ultraspec as described in Example 2. Random primed cDNA synthesis was performed as described in Example 3 and PCR was performed using standard conditions as described in Example 2 with HEV US-1 specific primers SEQ ID NO:94 and SEQ ID NO:96. Nested PCR was performed with primers SEQ ID NO:95 and SEQ ID NO:97. Sequencing of the PCR product was performed as described in Example 3. The sequence of the resulting PCR product is set forth in SEQ ID NO:98. GAP analysis as described in Example 2 showed that the nucleotide sequence, SEQ ID NO:98 was 95% identical to the corresponding or homologous homologous region from HEV US-1.

### Example 5

### Genome Extension and Sequencing of HEV US-2

The clone obtained and sequenced in Example 4 (SEQ ID NO:98) was derived from a HEV isolate most closely related to HEV US-1. To obtain additional regions of the HEV US-2 genome, several RT-PCR walking experiments were performed as described in Example 3.

RNA was extracted using the Total Nucleic Acid Extraction procedure (United States Biochemical). Reverse transcription was random primed using the GeneAmp RNA PCR kit (Perkin-Elmer). Standard PCR was performed in the presence of 2 mM MgCl₂ and 0.5 to 1.0 μM of each primer. Modified reactions contained lx PCR Buffer and 20% Q Solution (Qiagen) for the isolation of SEQ ID NOS:129, 141 and 146. Reactions used two HEV US-1 specific primers (Table 9), one HEV US-1 specific primer and one HEV consensus primer (Table 10), one HEV US-2 specific primer and one HEV consensus primer (Table 11), two HEV US-2 specific primers (Table 12), or two Burmese, Mexican, and US derived Consensus primers (described hereinbelow, Table 13).

The products shown in SEQ ID NOS:101, 102, 105, 108, 110, 113, 117, 120, 124, 149 and 151 were obtained by touchdown PCR. Amplification involved 43 cycles of 94° C. for 30 seconds, 55° C. for 30 seconds (−0.3° C./cycle), and 72° C. for 1 minute. This was followed by 10 cycles of 94° C. for 30 seconds, 40° C. for 30 seconds, and 72° C. for 1 minute. Cycling involving 35 cycles of 94° C. for 30 seconds, 55° C. for 30 seconds, and 72° C. for 1 minute was used to amplify SEQ ID NOS:129, 132, 136, 141 and 146. All amplifications were preceded by 1-2 minutes at 94° C. and followed by 72° C. for 5-10 minutes. The reactions were held at 4° C. prior to agarose gel analysis. Isolation of many products required a second round of nested or hemi-nested PCR as shown in Tables 9-13. In these reactions 1 μL of the PCR1 product was added to 25-50 μL of the PCR2 reaction mixture and the resulting mixture cycled as in PCR1.

Reactions were analyzed and products cloned and sequenced as described in Example 3 above. The results of these experiments are presented below in Tables 9-13.

TABLE 9

| | | | Approx. Product |
|---|---|---|---|
| Reaction | Primer set PCR 1 | Primer set PCR 2 | Size/SEQ ID NO: |
| 7.1 | SEQ ID NO:99/SEQ ID NO:100 | | 331 bp/SEQ ID NO:101 |
| 7.2 | SEQ ID NO:34/SEQ ID NO:35 | SEQ ID NO:36/SEQ ID NO:37 | 1186 bp/SEQ ID NO:102 |
| 7.3 | SEQ ID NO:103/SEQ ID NO:104 | | 130 bp/SEQ ID NO:105 |
| 7.4 | SEQ ID NO:106/SEQ ID NO:107 | SEQ ID NO:39/SEQ ID NO:107 | 564 bp/SEQ ID NO:108 |
| 7.5 | SEQ ID NO:86/SEQ ID NO:109 | SEQ ID NO:87/SEQ ID NO:109 | 678 bp/SEQ ID NO:110 |

TABLE 10

| | | | Approx. Product |
|---|---|---|---|
| Reaction | Primer set PCR 1 | Primer set PCR 2 | Size/SEQ ID NO: |
| 8.1 | SEQ ID NO:111/SEQ ID NO:112 | | 580 bp/SEQ ID NO:113 |
| 8.2 | SEQ ID NO:114/SEQ ID NO:116 | SEQ ID NO:116/SEQ ID NO:115 | 734 bp/SEQ ID NO:117 |

TABLE 11

| | | | Approx. Product Size/ |
|---|---|---|---|
| Reaction | Primer set PCR1 | Primer set PCR2 | SEQ ID NO: |
| 9.1 | SEQ ID NO:118/SEQ ID NO:119 | | 483 bp/SEQ ID NO:120 |
| 9.2 | SEQ ID NO:121/SEQ ID NO:122 | SEQ ID NO:121/SEQ ID NO:123 | 431 bp/SEQ ID NO:124 |
| 9.3 | SEQ ID NO:125/SEQ ID NO:126 | SEQ ID NO:127/SEQ ID NO:128 | 1020 bp/SEQ ID NO:129 |

TABLE 12

| | | | Approx. Product |
|---|---|---|---|
| Reaction | Primer set PCR1 | Primer set PCR2 | Size/SEQ ID NO.: |
| 10.1 | SEQ ID NO:130/SEQ ID NO:131 | | 407 bp/SEQ ID NO:132 |
| 10.2 | SEQ ID NO:133/SEQ ID NO:134 | SEQ ID NO:135/SEQ ID NO:134 | 547 bp/SEQ ID NO:136 |
| 10.3 | SEQ ID NO:137/SEQ ID NO:138 | SEQ ID NO:139/SEQ ID NO:140 | 903 bp/SEQ ID NO:141 |
| 10.4 | SEQ ID NO:142/SEQ ID NO:143 | SEQ ID NO:144/SEQ ID NO:145 | 503 bp/SEQ ID NO:146 |

TABLE 13

| | | Approx. Product Size/ |
|---|---|---|
| Reaction | Primer set | SEQ ID NO.: |
| 11.1 | SEQ ID NO:147/SEQ ID NO:148 | 418 bp/SEQ ID NO:149 |
| 11.2 | SEQ ID NO:150/SEQ ID NO:126 | 197 bp/SEQ ID NO:151 |

To obtain the sequence at the 3′ end of the genome, amplification utilized the 3′ RACE System of GIBCO BRL in accordance with the manufacturer's instructions as described Example 3. cDNA was generated using SEQ ID NO:84. PCR1 utilized primers SEQ ID NO:150 and SEQ ID NO:85. PCR2 primers were SEQ ID NO:152 and SEQ ID NO:85 (reaction 12.1). The resulting product was 901 bp (SEQ ID NO:153).

The isolation of new sequences located at the 5′-terminus of the HEV US-2 viral genome was achieved by inverse PCR (M. Zeiner and U. Gehring, Biotechniques 17: 1051-1053, 1994). Due to limited availability of sera from USP-1 and USP-2, fecal material from a HEV US-2 infected macaque (described in Example 9 below) was chosen as the source material. A product of 462 nucleotides was amplified from macaque fecal material from within the hypervariable/proline rich hinge region using RNA extracted, reverse transcribed, and PCR amplified as described in Example 3 using primers SEQ ID NOS:154, 155, 156 and 157. This product (SEQ ID NO:158) was 100% identical to HEV US-2 sequences. Therefore, it is contemplated that, any sequences identified at the 5′ end of the HEV genome from macaque feces should accurately represent the 5′ end of the HEV US-2 genome. Total nucleic acids were extracted from 200 μL of a 10% fecal suspension as described above. Reverse transcription reactions, which utilized HEV US specific primers (SEQ ID NO:159), were performed using a kit obtained from BMB (as described in M. Zeiner and U. Gehring, Biotechniques, supra), except that nucleic acids were denatured at 70° C. for 5 min and then placed on ice prior to initiation of the RT reaction. Generation of double-stranded, circular cDNAs was performed as described in M. Zeiner and U. Gehring, Biotechniques, supra. The resulting circular cDNA molecules served as template for subsequent PCR reactions. The primers used in the first PCR reaction (PCR1) are shown in SEQ ID NOS:160 and 161. The nested primers used in the second PCR reaction (PCR 2) were as shown in SEQ ID NOS:162 and 163.

Products from PCR2 (reaction 13.1) were cloned into pGEM-EasyT Vector (Promega) and sequenced using an Applied Biosystems 373 Automated sequencer. One product of 221 nucleotides was identified as having the appropriate primers and HEV US-2 sequences, identifying 63 nucleotides upstream of known HEV US-2 sequences. Additional clones were identified with the appropriate primers and portions of this new sequence. Primer extension experiments performed on RNA from 100 μL of USP-2 serum or 100 μL of a 10% fecal suspension using the sequences shown in SEQ ID NOS:163 and 161 as primers were unsuccessful in confirming the length of this sequence. Pair-wise comparisons of the 63 nucleotides to 5′ NTR sequences of Burmese-like isolates revealed identities greater than 94% suggesting that this is the true sequence of HEV US-2.

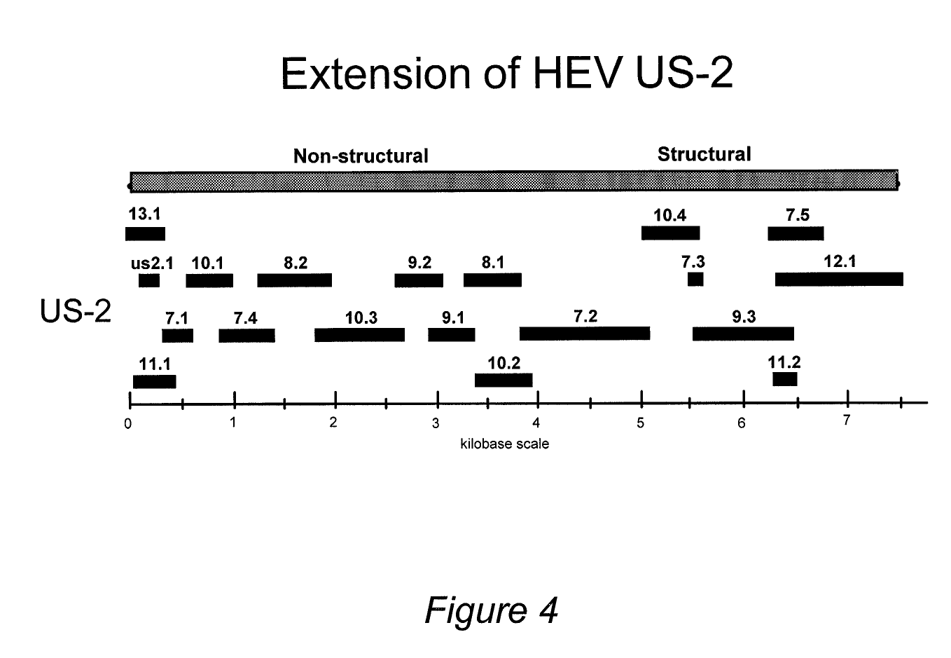

The sequences obtained from the products described in this Example and those described in Example 4 were assembled into contigs using programs in the GCG package (Genetics Computer Group, Madison, Wis., version 9) and a consensus sequence determined. A schematic of the assembled contigs is presented in FIG. 4. The genome of the HEV US-2 strain is 7277 bp in length, all of which has been sequenced and is set forth in SEQ ID NO:164. This sequence was translated into three open reading frames as indicated in SEQ ID NO:165, with the translation products of the ORF 1 and ORF 2 sequences only being shown (the third ORF is positioned at nucleotide positions 5159-5527 but cannot be shown within SEQ ID NO:165 due to overlap with the other two ORFs). The resulting translations of the ORF 1, ORF 2, and ORF 3 sequences are shown in SEQ ID NOS:166, 167 and 168, respectively.

### Example 6

### Sequence Comparisons

Information about the degree of relatedness of viruses typically can be obtained by performing comparisons such as alignments of nucleotide and deduced amino acid sequences. Alignments of the sequences of the US isolates of HEV (e.g., HEV US-1 and HEV US-2) with corresponding sequences of other isolates of HEV provide a quantitative assessment of the degree of similarity and identity between the sequences. In general, the calculation of the similarity between two amino acid sequences is based upon the degree of likeness exhibited between the side chains of an amino acid pair in an alignment. The degree of likeness is based upon the physical-chemical characteristics of the amino acid side chains, i.e. size, shape, charge, hydrogen-bonding capacity, and chemical reactivity. Thus similar amino acids possess side chains that have similar physical-chemical characteristics. The calculation of identity between two aligned amino acid or nucleotide sequences is, in general, an arithmetic calculation that counts the number of identical pairs of amino acids or nucleotides in an alignment and divides this number by the length of the sequence(s) in the alignment. The calculation of similarity between two aligned nucleotide sequences sometimes uses different values for transitions and transversions between paired (i.e. matched) nucleotides at various positions in the alignment. However, the magnitude of the similarity and identity scores between pairs of nucleotide sequences, are usually very close, i.e. within one to two percent.

The degree of similarity and identity was determined using the program GAP of the Wisconsin Sequence Analysis Package (Version 9). The gap creation and gap extension penalties were 50 and 3.0, respectively, for nucleic acid sequence alignments, and 12 and 4, respectively, for amino acid sequence comparisons.

As indicated previously, a partial identity exists between the initial 5′-end ORF 1 clone and other isolates of HEV, which supports the proposition that the HEV infection associated with patient USP-1 is due to a unique isolate of HEV. In order to more extensively determine the degree of relatedness between this isolate and other known isolates of HEV, alignments of the extended nucleotide and deduced amino acid sequences were performed.

Pair-wise nucleotide and amino acid comparisons of HEV US-1, HEV US-2, and 10 other full length HEV genomes (obtained from a publicly-available database, see Table 14) were performed, as described above, to determine the relationship of the US isolates to each other and to the known variants of HEV.

TABLE 14

| Isolate | Genbank Accession Number |
|---|---|
| Mexican (M1) | M74560 |
| Burmese (B1) | M73218 |
| Burmese (B2) | D10330 |
| Pakistan (P1) | M80581 |
| Chinese (C1) | D11092 |
| Chinese (C2) | L25547 |
| Chinese (C3) | M94177 |
| Chinese (C4) | D11093 |
| Indian (I1) | X98292 |
| Indian (I2) | X99441 |

Nucleotide identity across the entire genomes of US-1, US-2, B1, B2, I2, C1, C2, C3, P1, C4 and I1 strains is presented in Table 15. The nucleotide identities of ORF 1, ORF 2, and ORF 3 are shown in Tables 16, 17 and 18, respectively. Tables 17 and 18 also contain comparisons against a recently isolated swine (S1) sequence, available under GenBank accession number AF011921.

TABLE 15
Nucleotide Identity Across Genome

| | US-1 | US-2 | B1 | B2 | I2 | C1 | C2 | C3 | P1 | C4 | I1 |
|---|---|---|---|---|---|---|---|---|---|---|---|
| US-2 | 92.0 | | | | | | | | | | |
| B1 | 73.9 | 74.0 | | | | | | | | | |
| B2 | 73.8 | 74.0 | 98.5 | | | | | | | | |
| I2 | 73.5 | 73.8 | 96.1 | 95.4 | | | | | | | |
| C1 | 74.2 | 74.3 | 93.9 | 93.4 | 92.3 | | | | | | |
| C2 | 74.2 | 74.3 | 93.5 | 93.0 | 92.0 | 98.7 | | | | | |
| C3 | 74.1 | 74.3 | 93.7 | 93.0 | 92.0 | 98.2 | 98.7 | | | | |
| P1 | 74.1 | 74.1 | 93.6 | 92.8 | 92.0 | 98.2 | 98.8 | 98.3 | | | |
| C4 | 73.7 | 73.9 | 94.5 | 94.1 | 92.7 | 97.1 | 97.2 | 96.8 | 96.7 | | |
| I1 | 74.4 | 74.4 | 93.5 | 93.0 | 92.2 | 93.8 | 94.0 | 93.8 | 93.9 | 93.5 | |
| M1 | 73.7 | 74.5 | 75.9 | 75.7 | 75.0 | 75.9 | 75.9 | 75.9 | 76.1 | 75.7 | 75.7 |

TABLE 16
Nucleotide Identity Across ORF 1

| | US-1 | US-2 | B1 | B2 | I2 | C1 | C2 | C3 | P1 | C4 | I1 |
|---|---|---|---|---|---|---|---|---|---|---|---|
| US-1 | | | | | | | | | | | |
| US-2 | 92.0 | | | | | | | | | | |
| B1 | 71.7 | 71.6 | | | | | | | | | |
| B2 | 71.7 | 71.8 | 98.6 | | | | | | | | |
| I2 | 71.2 | 71.5 | 95.7 | 95.1 | | | | | | | |
| C1 | 72.1 | 72.1 | 93.5 | 93.1 | 91.8 | | | | | | |
| C2 | 72.2 | 72.3 | 93.1 | 92.7 | 91.5 | 98.6 | | | | | |
| C3 | 71.9 | 72.2 | 93.3 | 92.8 | 91.4 | 98.1 | 98.7 | | | | |
| P1 | 72.2 | 72.1 | 93.1 | 92.6 | 91.4 | 98.2 | 99.0 | 98.4 | | | |
| C4 | 71.5 | 71.7 | 94.6 | 94.4 | 92.3 | 96.7 | 98.8 | 96.3 | 96.4 | | |
| I1 | 72.3 | 72.3 | 93.2 | 92.8 | 91.5 | 93.6 | 94.0 | 93.7 | 93.9 | 93.3 | |
| M1 | 72.0 | 72.6 | 73.6 | 73.5 | 72.5 | 73.7 | 73.8 | 73.8 | 73.9 | 73.4 | 73.5 |

TABLE 17
Nucleotide Identity Across ORF 2

| | US-1 | US-2 | B1 | B2 | I2 | C1 | C2 | C3 | P1 | C4 | I1 | M1 |
|---|---|---|---|---|---|---|---|---|---|---|---|---|
| US-1 | | | | | | | | | | | | |
| US-2 | 92.2 | | | | | | | | | | | |
| B1 | 79.2 | 79.6 | | | | | | | | | | |
| B2 | 86.4 | 79.4 | 98.5 | | | | | | | | | |
| I2 | 79.0 | 79.5 | 99.2 | 98.4 | | | | | | | | |
| C1 | 79.3 | 79.5 | 94.4 | 98.4 | 98.4 | | | | | | | |
| C2 | 79.2 | 79.4 | 94.3 | 97.8 | 97.8 | 98.9 | | | | | | |
| C3 | 79.3 | 79.4 | 94.4 | 97.8 | 97.8 | 98.9 | 98.4 | | | | | |
| P1 | 79.0 | 79.3 | 93.8 | 98.1 | 98.7 | 99.7 | 99.2 | 99.2 | | | | |
| C4 | 78.8 | 79.3 | 94.0 | 97.8 | 97.8 | 98.9 | 98.4 | 98.4 | 97.4 | | | |
| I1 | 79.4 | 79.7 | 94.1 | 97.6 | 97.3 | 97.9 | 97.0 | 94.0 | 93.7 | 93.9 | | |
| M1 | 78.0 | 79.3 | 81.1 | 90.1 | 98.5 | 90.6 | 90.1 | 81.0 | 81.4 | 90.3 | 90.3 | |
| S1 | 92.0 | 98.9 | 79.8 | 84.6 | 85.4 | 85.4 | 85.1 | 80.2 | 80.1 | 84.8 | 85.1 | 84.6 |

TABLE 18
Nucleotide Identity Across ORF 3

| | US-1 | US-2 | B1 | B2 | I2 | C1 | C2 | C3 | P1 | C4 | I1 | M1 |
|---|---|---|---|---|---|---|---|---|---|---|---|---|
| US-1 | | | | | | | | | | | | |
| US-2 | 96.2 | | | | | | | | | | | |
| B1 | 87.0 | 86.6 | | | | | | | | | | |
| B2 | 86.4 | 86.3 | 99.2 | | | | | | | | | |
| I2 | 86.4 | 86.9 | 97.8 | 99.2 | | | | | | | | |
| C1 | 87.3 | 86.3 | 99.2 | 98.4 | 98.4 | | | | | | | |
| C2 | 86.4 | 86.1 | 98.1 | 97.3 | 97.8 | 98.9 | | | | | | |
| C3 | 86.7 | 85.6 | 98.1 | 97.3 | 97.8 | 98.9 | 98.4 | | | | | |
| P1 | 87.0 | 86.6 | 98.9 | 98.1 | 98.7 | 99.7 | 99.2 | 99.2 | | | | |
| C4 | 86.2 | 85.8 | 98.1 | 97.6 | 97.8 | 98.9 | 98.4 | 98.4 | 99.2 | | | |
| I1 | 86.4 | 86.6 | 97.8 | 97.6 | 97.6 | 97.9 | 97.0 | 97.0 | 97.8 | 97.8 | | |
| M1 | 84.6 | 85.2 | 87.8 | 90.1 | 89.5 | 90.6 | 90.1 | 90.1 | 90.9 | 90.3 | 90.3 | |
| S1 | 94.9 | 96.7 | 85.1 | 84.6 | 85.4 | 85.4 | 85.1 | 84.8 | 85.6 | 84.8 | 85.1 | 84.6 |

In addition, the ORF 1 nucleotide sequences encoding the methyltransferase proteins were compared between each of the US-1, US-2, M1 and P1 isolates. The methyltransferase encoding region of the HEV US-1 genome is represented by residues 1-693 of SEQ ID NO:89, whereas the methyltransferase encoding region of the HEV US-2 genome is represented by residues 36-755 of SEQ ID NO:164. The comparison results are set forth in Table 19.

TABLE 19
Methyltransferase Region

| | % IDENTITY | | | |
|---|---|---|---|---|
| | US-1 | US-2 | M1 | P1 |
| US-1 | — | 93.4 | 77.0 | 75.2 |
| US-2 | — | — | 78.5 | 76.0 |
| M1 | — | — | — | 78.8 |

The ORF 1 nucleotide sequences encoding the Y domain proteins were compared between each of the US-1, US-2, M1 and P1 isolates. The Y domain protein encoding region of the HEV US-1 genome is represented by residues 619-1272 of SEQ ID NO:89, whereas the Y domain protein encoding region of the HEV US-2 genome is represented by residues 680-1334 of SEQ ID NO:164. The comparison results are set forth in Table 20.

TABLE 20
Y Domain

| | % IDENTITY | | | |
|---|---|---|---|---|
| | US-1 | US-2 | M1 | P1 |
| US-1 | — | 94.0 | 79.0 | 77.2 |
| US-2 | — | — | 79.7 | 76.8 |
| M1 | — | — | — | 78.3 |

The ORF 1 nucleotide sequences encoding the protease proteins were compared between each of the US-1, US-2, M1 and P1 isolates. The protease protein encoding region of the HEV US-1 genome is represented by residues 1270-2091 of SEQ ID NO:89, whereas the protease protein encoding region of the HEV US-2 genome is represented by residues 1332-2153 of SEQ ID NO:164. The comparison results are set forth in Table 21.

TABLE 21
Protease Region

| | % IDENTITY | | | |
|---|---|---|---|---|
| | US-1 | US-2 | M1 | P1 |
| US-1 | — | 91.8 | 65.1 | 64.0 |
| US-2 | — | — | 65.1 | 63.1 |
| M1 | — | — | — | 68.1 |

The ORF 1 nucleotide sequences encoding the hypervariable region were compared between each of the US-1, US-2, M1 and P1 isolates. The hypervariable region encoding region of the HEV US-1 genome is represented by residues 2092-2364 of SEQ IS NO:89, whereas the hypervariable region encoding region of the HEV US-2 genome is represented by residues 2194-2429 of SEQ ID NO: 164. The comparison results are set forth in Table 22.

TABLE 22
Hypervariable Region

| | % IDENTITY | | | |
|---|---|---|---|---|
| | US-1 | US-2 | M1 | P1 |
| US-1 | — | 83.9 | 40.3 | 50.2 |
| US-2 | — | — | 45.8 | 49.8 |
| M1 | — | — | — | 40.4 |

The ORF 1 nucleotide sequences encoding the X domain proteins were compared between each of the US-1, US-2, M1 and P1 isolates. The X domain protein encoding region of the HEV US-1 genomes represented by residues 2365-2841 of SEQ ID NO:89, whereas the X domain probe encoding region of the HEV US-2 genome is represented by residues 2430-2906 of SEQ ID NO:164. The comparison results are set forth in Table 23.

TABLE 23
X Domain

| | % IDENTITY | | | |
|---|---|---|---|---|
| | US-1 | US-2 | M1 | P1 |
| US-1 | — | 91.6 | 72.5 | 71.3 |
| US-2 | — | — | 72.7 | 70.9 |
| M1 | — | — | — | 72.9 |

The ORF 1 nucleotide sequences encoding the helicase proteins were compared between each of the US-1, US-2, M1 and P1 isolates. The helicase encoding region of the HEV US-1 genomes represented by residues 2893-3591 of SEQ ID NO:89, whereas the helicase encoding region of the HEV US-2 genome is represented by residues 2958-3656 of SEQ ID NO:164. The comparison results are set forth in Table 24.

TABLE 24
Helicase Region

| | % IDENTITY | | | |
|---|---|---|---|---|
| | US-1 | US-2 | M1 | P1 |
| US-1 | — | 92.8 | 76.5 | 75.2 |
| US-2 | — | — | 75.4 | 74.1 |
| M1 | — | — | — | 76.2 |

The ORF 1 nucleotide sequences encoding the RNA-dependent RNA polymerase proteins were compared between each of the US-1, US-2, M1 and P1 isolates. The polymerase encoding region of the HEV US-1 genome is represented by residues 3634-5094 of SEQ ID NO:89, whereas the polymerase encoding region of the HEV US-2 genome is represented by residues 3699-5159 of SEQ ID NO:164. The comparison results are set forth in Table 25.

TABLE 25
RNA-dependent RNA Polymerase Region

% IDENTITY

| | US-1 | US-2 | M1 | P1 |
|---|---|---|---|---|
| US-1 | — | 93.1 | 72.9 | 75.3 |
| US-2 | — | — | 73.6 | 75.8 |
| M1 | — | — | — | 77.1 |

In addition, the amino acid identities/similarities of the proteins encoded by the ORF 1, ORF 2, and ORF 3 sequences of US-1, US-2, B1, B2, I2, C1, C2, C3, P1, C4 and I1 strains are shown in Tables 26, 27 and 28 respectively. In addition, Tables 27 and 28 also contain comparisons against the swine sequence (S1). In Tables 26, 27 and 28, the similarities are presented in the upper right hand halves of the tables and the identities are presented in the lower left hand halves of the tables.

TABLE 26
Amino Acid Similarity/Identity Across ORF 1

| | % SIMILARITY | | | | | | | | | | | |
|---|---|---|---|---|---|---|---|---|---|---|---|---|
| | US-1 | US-2 | B1 | B2 | 12 | C1 | C2 | C3 | P1 | C4 | I1 | M1 |
| % | | | | | | | | | | | | |
| IDENTITY | | | | | | | | | | | | |
| US-1 | | 97.8 | 86.0 | 85.7 | 84.4 | 85.9 | 86.2 | 84.9 | 86.4 | 85.7 | 86.3 | 85.4 |
| US-2 | 97.5 | | 86.2 | 85.8 | 84.5 | 85.8 | 86.0 | 85.0 | 86.3 | 85.7 | 86.3 | 85.5 |
| B1 | 82.4 | 82.6 | | 98.7 | 96.8 | 98.4 | 98.5 | 97.1 | 98.5 | 98.1 | 98.2 | 87.0 |
| B2 | 82.3 | 82.3 | 98.6 | | 96.2 | 97.8 | 97.9 | 96.3 | 97.8 | 97.6 | 97.6 | 86.6 |
| I2 | 80.7 | 80.7 | 96.3 | 95.7 | | 96.3 | 96.4 | 95.0 | 96.3 | 95.9 | 95.9 | 85.2 |
| C1 | 82.5 | 82.3 | 98.2 | 97.5 | 95.7 | | 99.5 | 97.9 | 99.4 | 99.0 | 98.2 | 86.9 |
| C2 | 82.8 | 82.6 | 98.4 | 97.8 | 95.9 | 99.4 | | 98.2 | 99.6 | 99.2 | 98.4 | 87.0 |
| C3 | 81.6 | 81.6 | 96.9 | 96.1 | 94.4 | 97.7 | 98.1 | | 98.1 | 97.6 | 97.0 | 85.9 |
| P1 | 83.0 | 82.9 | 98.4 | 97.7 | 95.9 | 99.2 | 99.6 | 98.0 | | 99.0 | 98.4 | 87.1 |
| C4 | 82.5 | 82.3 | 98.0 | 97.6 | 95.4 | 98.8 | 99.1 | 97.4 | 98.9 | | 97.8 | 86.5 |
| I1 | 82.9 | 82.9 | 98.1 | 97.5 | 95.5 | 98.1 | 98.4 | 96.9 | 98.4 | 97.8 | | 87.3 |
| M1 | 82.0 | 82.0 | 83.8 | 83.4 | 81.8 | 83.7 | 83.9 | 82.8 | 84.0 | 83.4 | 84.2 | |

TABLE 27
Amino Acid Similarity/Identity Across ORF 2

| | % SIMILARITY | | | | | | | | | | | | |
|---|---|---|---|---|---|---|---|---|---|---|---|---|---|
| | US-1 | US-2 | B1 | B2 | I2 | C1 | C2 | C3 | P1 | C4 | I1 | M1 | S1 |
| % | | | | | | | | | | | | | |
| IDENTITY | | | | | | | | | | | | | |
| US-1 | | 98.3 | 93.3 | 93.0 | 93.0 | 93.5 | 93.2 | 92.9 | 93.2 | 92.4 | 92.6 | 91.5 | 97.1 |
| US-2 | 98.0 | | 93.3 | 93.0 | 93.3 | 93.3 | 93.3 | 93.0 | 93.3 | 92.6 | 92.7 | 91.7 | 99.1 |
| B1 | 91.8 | 91.8 | | 98.9 | 99.1 | 99.8 | 99.2 | 99.2 | 99.5 | 98.8 | 98.9 | 94.8 | 93.0 |
| B2 | 91.5 | 91.5 | 98.9 | | 98.3 | 99.1 | 98.5 | 98.5 | 98.8 | 98.2 | 98.2 | 94.1 | 92.7 |
| I2 | 91.5 | 91.8 | 99.1 | 98.3 | | 99.2 | 98.9 | 98.6 | 99.2 | 98.5 | 98.6 | 94.5 | 91.5 |
| C1 | 92.0 | 92.0 | 99.7 | 98.9 | 99.1 | | 99.4 | 99.1 | 99.7 | 98.9 | 99.1 | 95.0 | 93.2 |
| C2 | 91.7 | 92.0 | 99.1 | 98.3 | 98.8 | 99.4 | | 98.8 | 99.4 | 98.6 | 98.8 | 94.7 | 93.0 |
| C3 | 91.4 | 91.7 | 99.1 | 98.3 | 98.5 | 99.1 | 98.8 | | 99.1 | 98.3 | 98.5 | 94.4 | 92.7 |
| P1 | 91.7 | 92.0 | 99.4 | 98.6 | 99.1 | 99.7 | 99.4 | 99.1 | | 98.9 | 99.1 | 95.0 | 93.0 |
| C4 | 90.9 | 91.2 | 98.6 | 98.0 | 98.4 | 98.9 | 98.6 | 98.3 | 98.9 | | 98.3 | 94.2 | 92.3 |
| I1 | 91.1 | 91.4 | 98.5 | 97.7 | 98.2 | 98.8 | 98.5 | 98.2 | 98.8 | 98.0 | | 94.7 | 92.4 |
| M1 | 90.1 | 90.6 | 93.2 | 92.4 | 92.9 | 93.3 | 93.0 | 92.9 | 93.3 | 92.6 | 93.0 | | 91.2 |
| S1 | 97.7 | 98.9 | 91.7 | 91.4 | 91.9 | 91.8 | 91.7 | 91.4 | 91.7 | 90.9 | 91.1 | 90.2 | |

TABLE 28
Amino Acid Similarity/Identity Across ORF 3

| | % SIMILARITY | | | | | | | | | | | | |
|---|---|---|---|---|---|---|---|---|---|---|---|---|---|
| | US-1 | US-2 | B1 | B2 | I2 | C1 | C2 | C3 | P1 | C4 | I1 | M1 | S1 |
| % | | | | | | | | | | | | | |
| IDENTITY | | | | | | | | | | | | | |
| US-1 | | 96.7 | 85.2 | 84.4 | 85.2 | 85.2 | 83.6 | 85.2 | 85.2 | 83.6 | 85.2 | 79.5 | 93.5 |
| US-2 | 96.7 | | 85.2 | 84.4 | 85.2 | 85.2 | 83.6 | 83.6 | 85.2 | 83.6 | 85.2 | 81.1 | 96.7 |
| B1 | 84.4 | 84.4 | | 98.4 | 100.0 | 100.0 | 98.4 | 98.4 | 100.0 | 98.4 | 98.4 | 87.0 | 83.7 |
| B2 | 83.6 | 83.6 | 98.4 | | 98.4 | 98.4 | 96.7 | 96.7 | 98.4 | 96.7 | 96.7 | 87.0 | 82.9 |
| I2 | 84.4 | 84.4 | 100.0 | 98.4 | | 100.0 | 98.4 | 98.4 | 100.0 | 98.4 | 98.4 | 87.0 | 83.7 |
| C1 | 84.4 | 84.4 | 100.0 | 98.4 | 100.0 | | 98.4 | 98.4 | 100.0 | 98.4 | 98.4 | 87.0 | 83.7 |
| C2 | 82.8 | 82.8 | 98.4 | 96.7 | 98.4 | 98.4 | | 96.7 | 98.4 | 97.6 | 96.7 | 85.4 | 82.1 |
| C3 | 84.4 | 82.8 | 98.4 | 96.7 | 98.4 | 98.4 | 96.7 | | 98.4 | 96.7 | 96.7 | 85.4 | 82.1 |
| P1 | 84.4 | 84.4 | 100.0 | 98.4 | 100.0 | 100.0 | 98.4 | 98.4 | | 98.4 | 98.4 | 87.0 | 83.7 |
| C4 | 82.8 | 82.8 | 98.4 | 96.7 | 98.4 | 98.4 | 97.6 | 96.7 | 98.4 | | 96.7 | 85.4 | 82.1 |
| I1 | 84.4 | 84.4 | 98.4 | 96.7 | 98.4 | 98.4 | 96.7 | 96.7 | 98.4 | 96.7 | | 88.6 | 83.7 |
| M1 | 78.7 | 80.3 | 87.0 | 87.0 | 87.0 | 87.0 | 85.4 | 85.4 | 87.0 | 85.4 | 88.6 | | 79.7 |
| S1 | 93.5 | 96.7 | 82.9 | 82.1 | 82.9 | 82.9 | 81.3 | 81.3 | 82.9 | 81.3 | 82.9 | 78.9 | |

In addition, the ORF 1 amino acid sequences defining the methyltransferase proteins were compared between each of the US-1, US-2, M1 and P1 isolates. The methyltransferase protein encoded by the HEV US-1 genome is represented by residues 1-231 of SEQ ID NO:91, whereas the methyltransferase protein encoded by the HEV US-2 genome is represented by residues 1-240 of SEQ ID NO: 166. The comparison results are set forth in Table 29.

TABLE 29
Methyltransferase Region

% IDENTITY

| | US-1 | US-2 | M1 | P1 |
|---|---|---|---|---|
| % | | | | |
| SIMILARITY | | | | |
| US-1 | — | 98.7 | 91.3 | 88.7 |
| US-2 | 98.7 | — | 91.7 | 89.1 |
| M1 | 91.8 | 92.0 | — | 92.9 |
| P1 | 90.0 | 90.4 | 91.2 | — |

The ORF 1 amino acid sequences defining the protease proteins were compared between each of the US-1, US-2, M1 and P1 isolates. The protease protein encoded by the HEV US-1 genome is represented by residues 424-697 of SEQ ID NO:91, whereas the protease protein encoded by the HEV US-2 genome is represented by residues 433-706 of SEQ ID NO:166. The comparison results are set forth in Table 30.

TABLE 30
Protease Region

% IDENTITY

| | US-1 | US-2 | M1 | P1 |
|---|---|---|---|---|
| % | | | | |
| SIMILARITY | | | | |
| US-1 | — | 98.5 | 67.5 | 69.3 |
| US-2 | 97.8 | — | 67.1 | 68.6 |
| M1 | 73.3 | 73.3 | — | 76.6 |
| P1 | 74.4 | 74.0 | 72.2 | — |

The ORF 1 amino acid sequences defining Y domain proteins were compared between each of the US-1, US-2, M1 and P1 isolates. The Y domain protein encoded by the HEV US-1 genome is represented by residues 207-424 of SEQ ID NO:91, whereas the Y domain protein encoded by the HEV US-2 genome is represented by residues 216-433 of SEQ ID NO:166. The comparison results are set forth in Table 31.

TABLE 31
Y Domain

% IDENTITY

| | US-1 | US-2 | M1 | P1 |
|---|---|---|---|---|
| % | | | | |
| SIMILARITY | | | | |
| US-1 | — | 98.2 | 92.7 | 93.6 |
| US-2 | 98.2 | — | 92.7 | 93.6 |
| M1 | 94.0 | 94.0 | — | 93.1 |
| P1 | 94.5 | 94.5 | 91.7 | — |

The ORF 1 amino acid sequences defining the X domain proteins were compared between each of the US-1, US-2, M1 and P1 isolates. The X domain encoded by the HEV US-1 genome is represented by residues 789-947 of SEQ ID NO:91, whereas the X domain protein encoded by the HEV US-2 genome is represented by residues 799-957 of SEQ ID NO: 166. The comparison results are set forth in Table 32.

TABLE 32
X Domain

% IDENTITY

| | US-1 | US-2 | M1 | P1 |
|---|---|---|---|---|
| % | | | | |
| SIMILARITY | | | | |
| US-1 | — | 97.5 | 82.4 | 80.5 |
| US-2 | 97.5 | — | 81.8 | 79.9 |
| M1 | 88.0 | 87.4 | — | 86.1 |
| P1 | 84.3 | 83.6 | 83.0 | — |

The ORF 1 amino acid sequences defining helicase proteins were compared between each of the US-1, US-2, M1 and P1 isolates. The helicase encoded by the HEV US-1, US-2, M1 and P1 isolates. The helicase encoded by the HEV US-1 genome is represented by residues 965-1197 of SEQ ID NO:91, whereas the helicase encoded by the HEV US-2 genome is represented by residues 975-1207 of SEQ ID NO:166. The comparison results are set forth in Table 33.

TABLE 33
Helicase Region

% IDENTITY

| | US-1 | US-2 | M1 | P1 |
|---|---|---|---|---|
| % | | | | |
| SIMILARITY | | | | |
| US-1 | — | 99.1 | 89.7 | 91.0 |
| US-2 | 99.1 | — | 90.6 | 91.8 |
| M1 | 93.1 | 94.0 | — | 95.2 |
| P1 | 94.0 | 94.8 | 91.0 | — |

The ORF 1 amino acid sequence defining the hypervariable regions were compared between each end of the US-1, US-2, M1 and P1 isolates. The hypervariable region encoded by the HEV US-1 genome is represented by residues 698-788 of SEQ ID NO:91, whereas the hypervariable region encoded by the HEV US-2 genome is represented by residues 707-798 of SEQ ID NO:166. The comparison results are set forth in Table 34.

TABLE 34
Hypervariable Region

% IDENTITY

| | US-1 | US-2 | M1 | P1 |
|---|---|---|---|---|
| % | | | | |
| SIMILARITY | | | | |
| US-1 | — | 82.4 | 25.0 | 27.7 |
| US-2 | 79.1 | — | 25.0 | 21.0 |
| M1 | 25.0 | 25.0 | — | 20.8 |
| P1 | 31.9 | 21.0 | 18.0 | — |

The ORF 1 amino acid sequence defining the RNA-dependent RNA polymerase proteins were compared between each of the US-1, US-2, M1 and P1 isolates. The polymerase encoded by the HEV US-1 genome is represented by residues 1212-1698 of SEQ ID NO:91, whereas the polymerase encoded by the HEV US-2 genome is represented by residues 1222-1708 of SEQ ID NO:166. The comparison results are set forth in Table 35.

TABLE 35
RNA-dependent RNA Polymerase Domain

% IDENTITY

| | US-1 | US-2 | M1 | P1 |
|---|---|---|---|---|
| % | | | | |
| SIMILARITY | | | | |
| US-1 | — | 99.0 | 86.0 | 87.8 |
| US-2 | 99.0 | — | 86.2 | 87.7 |
| M1 | 89.7 | 89.9 | — | 92.6 |
| P1 | 91.6 | 91.6 | 89.5 | — |

In addition to the foregoing, several additional HEV isolates belonging to the HEV US-type family were identified during the course of this work (see, Example 13 below). The additional isolates are denoted as It1 (Italian strain), G1 (first Greek strain) and G2 (second Greek strain). Additional sequence comparisons were performed and include the It1, G1 and G2 sequences, the results of which are presented below in Tables 36 and 37. Table 36 shows the nucleotide and deduced amino acid identities between isolates of HEV over a 371 base (123 amino acids) ORF 1 fragment. The ORF 1 fragment corresponds to residues 26-396 of SEQ ID NO:89. Table 37 shows the nucleotide and deduced amino acid identities between isolates of HEV over a 148 base (49 amino acid) ORF 2 fragment. The ORF 2 fragment corresponds to residues 6307-6454 of SEQ ID NO:89. In both Tables 36 and 37, the isolates represented are Burmese (B1, B2), Chinese (C1, C2, C3, C4), Indian (I1, I2), Pakistan (P1), Mexican (M1), Swine (S1), United States (US-1, US-2), Greek (G1, G2) and Italian (It1).

Pairwise comparisons of the full length nucleotide sequences were preferred using the nucleotide sequences of the respective genomes of HEV US-1 and HEV US-2 together with the other genomes of the other HEV isolates identified in Table 14. The results of the comparison are shown in Table 15. At the nucleotide level, HEV US-1 and HEV US-2 were most closely related to each other, with 92.0% identity across the entire genome. The full length Burmese-like isolates demonstrated similar identities ranging from 92.0 to 98.8%. The US isolates were 73.5 to 74.5% identical to the Burmese-like and Mexican isolates. This is similar to the identity seen between any one Burmese-like isolate and the Mexican isolate, 75.0 to 76.1% nucleotide identity. These data indicate that the US isolates are members of a new strain variant of HEV, distinct from the Burmese and Mexican strains.

Similar degrees of identity are found when smaller portions of each genome are analyzed, such as the individual ORFs. These values are presented in Tables 16, 17 and 18 for ORF 1, ORF 2, and ORF 3, respectively. Across each region, the Burmese and Pakistani isolates demonstrate the highest degree of identity ranging from 93.1 to 98.9% identity. The Mexican isolate is distinct, with identities of 73.6 to 90.1% to the Burmese-like isolates. HEV US-1 nucleotide sequence analysis reveals a significant degree of divergence with ORF 1 sequences being less than 72% identical to the Burmese-like and Mexican isolates. Similarly, ORF 2 and ORF 3 sequences were less than 79.1% and 86.9% identical to the Burmese-like and Mexican isolates, respectively.

The variability seen at the nucleotide level is reflected in the amino acid similarity and identity of the translated open reading frames. ORF 1 is the most divergent product, potentially due to the presence of a hypervariable region. The US isolates possess 97.5% amino acid identity across this region (Table 26). This is similar to the 94.4 to 99.6% identity seen between Burmese-like ORF 1 proteins. The US ORF 1 products are 80.7 to 83.0% identical to Burmese-like and Mexican proteins (Table 26). These values are similar to those observed between any one Burmese-like isolates and the Mexican isolate, ranging from 81.8 to 84.2% identity. Amino acid similarity values are generally up to 3.5% higher than the identity value, reflecting a large number of conservative amino acid substitutions. The ORF 2 product is the most conserved, potentially due to its role as the viral capsid protein. The US ORF 2 products are 98.0% identical to each other, while being 90.1 to 92% identical to Burmese and Mexican ORF 2 proteins (Table 27). Again, these ranges mirror those observed between Burmese isolates (97.7 to 99.7% identity). Identity between Burmese and Mexican isolates is slightly greater than that between the US variant and other variants, being 92.4 to 93.3%. Amino acid similarity across ORF 2 adds approximately 1.5% to the identity value. The ORF 3 product of HEV US-1 and HEV US-2 shared 96.7% amino acid identity. The Burmese isolates showed 96.7 to 100% amino acid identity. ORF 3 amino acid identities of the US isolates to the Burmese and Mexican isolates were 78.7 to 84.4%, slightly less than that observed between Burmese and Mexican isolates, 85.4 to 88.6% identity (Table 28). Amino acid similarity across ORF 3 was generally the same as the identity values, however, some comparisons demonstrated similarity values less than 1.0% greater than the identity value. These amino acid similarity and identity values indicate that the analysis of short amino acid sequences produce similar results to full length and partial nucleotide analyses, indicating that the US isolates are closely related and genetically distinct from previously characterized isolates of HEV.

Tables 27 and 28 also include pairwise amino acid sequence comparisons with a HEV-like isolate recently identified in swine (Meng et al. (1997) Proc. Natl. Acad. Sci. USA 94: 9860-9865. Only 2021 bp across the ORF 2/3 region have been characterized (GenBank Accession Number: AF011921). The US swine sequence is 92% identical to the corresponding region of HEV US-1 at the nucleotide level. It is noted that HEV US-1 is very similar at the amino acid level to the recently identified swine virus. For example, the HEV US-1 and swine strains exhibit 97.1% and 93.5% identity over the respective ORF 2 and ORF 3 sequences (Tables 27 and 28, respectively).

Partial sequences of 210 nucleotides from two HEV isolates from China referred to as G9 and G20 (Genbank Accession numbers X87306 and X87307, respectively) recently have been described in the literature by (Huang et al. (1995) J. Med Virology 47: 303-308). These fragments represent nucleotide sequences homologous to residue numbers 4533 to 4742 of SEQ ID NO:89. Their encoded amino acid sequences (69 amino acid residues in-length) are homologous to residue numbers 1512-1580 of SEQ ID NO:91. The results from the pairwise comparisons of the nucleotide sequences and the predicted amino acid sequences of these sequences are shown in Tables 38 and 39. Results indicate that the G9 and G20 isolates are 89% identical to one another at the nucleotide level across this region. The closely related Burmese and Pakistan isolates are 92.9% identical over this range. The US-1 isolate exhibits a 77.1 and 81.0 across this region suggesting that the US-1 isolate also is unique from these isolates. Although the G9 and G20 sequences are most closely related at the nucleotide level, the deduced amino acid translation of G20 is most similar/identical to the US sequence from the US-1 isolate (Table 38). This is most likely due to the short length of amino acids utilized in the analysis.

TABLE 38
Identity across 210 nucleotides of ORF 1

| | Pak | Mex | US-1 | G20 | G9 |
|---|---|---|---|---|---|
| Bur | 92.9 | 74.8 | 75.7 | 78.1 | 76.7 |
| Pak | | 75.2 | 76.7 | 78.1 | 76.7 |
| Mex | | | 77.1 | 75.2 | 71.9 |
| US-1 | | | | 81.0 | 77.1 |
| G20 | | | | | 89.0 |

TABLE 39
Similarity/identity across 69 amino acids of ORF 1

| | Pak | Mex | US-1 | G20 | G9 |
|---|---|---|---|---|---|
| Bur | 98.6/98.6 | 92.8/88.4 | 92.8/85.5 | 92.8/88.4 | 82.6/79.7 |
| Pak | | 94.2/89.9 | 91.3/84.1 | 91.3/87.0 | 84.1/81.2 |
| Mex | | | 89.9/87.0 | 89.9/87.0 | 81.2/78.3 |
| US-1 | | | | 100/95.7 | 88.4/88.1 |
| G20 | | | | | 88.4/87.0 |

### Example 7

### Phylogenetic Analyses

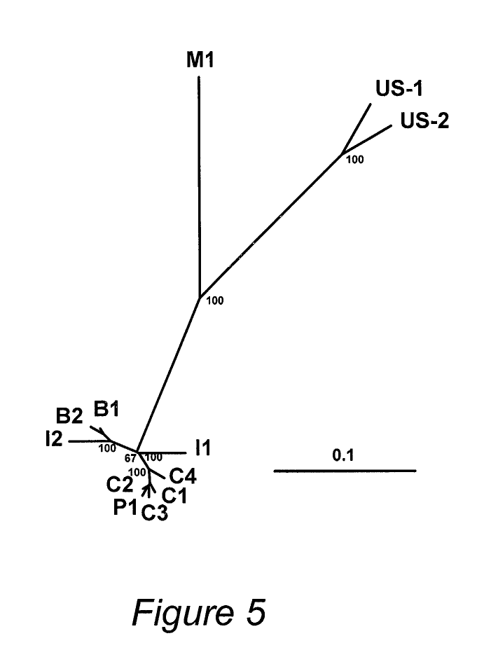

Alignments of nucleotide and amino acid sequences were performed in order to determine the phylogenetic relationships between the novel US-type isolates and other isolates of HEV. The alignments were made using the program PILEUP of the Wisconsin Sequence Analysis Package, version 9 (Genetics Computer Group, Madison, Wis.). Evolutionary distances between sequences were determined using the DNADIST program (Kimura 2-parameter method) with a transition-transversion ratio of 2.0 and PROTDIST (Dayhoff PAM matrix) program of the PHYLIP package, version 3.5c (Felsenstein 1993, Department of Genetics, University of Washington, Seattle). The computed distances were used for the construction of phylogenetic trees using the program FITCH (Fitch-Margoliash method). The robustness of the trees was determined by bootstrap resampling of the multiple-sequence alignments (100 sets or 1,000 sets) with the programs SEQBOOT, DNADIST, the neighbor-joining method of the program NEIGHBOR, and CONSENSE (PHYLIP package). Bootstrap values of less than 70% are regarded as not providing evidence for a phylogenetic grouping (Muerhoff et al., (1997) Journal of Virology, 71: 6501-6508). The final trees were produced using RETREE (PHYLIP) with the midpoint rooting option and the graphical output was created with TREEVIEW (Page, (1996) Computer Applied Biosciences 12: 357-358), the results of which are presented in FIGS. 5, 6, 10, and 11.

Phylogenetic Analysis With Complete Genomes.

To more extensively determine the degree of relatedness between HEV US-1, HEV US-2, and other known isolates of HEV, nucleotide alignments were performed. The full length HEV US-1 and HEV US-2 genomes were aligned with 10 other isolates of HEV from which complete genomes are available (Table 14).

Examination of the phylogenetic distances based upon alignments of the HEV-US isolates and other isolates of HEV demonstrate that there is considerable evolutionary distance between those from the US and those from other geographical areas as determined using the DNADIST program (Kimura 2-parameter method) with a transition-transversion ratio of 2.0 (Table 40). The distances calculated also show the close relationship between the isolates originating from Asia. Within this Burmese-like group the maximum distance calculated from the full length alignment is 0.0850 nucleotide substitutions per base. The minimum distance between a member of this group and a US isolate is 0.3322 substitutions. The Mexican strain shows similar distances to the Burmese-like group of 0.3055 to 0.3132 substitutions and 0.3322 to 0.3462 substitutions to the US isolate. The genetic distance between HEV US-1 and HEV US-2 of 0.0812 substitutions is similar to that seen between Burmese-like isolates. The relative evolutionary distances between the viral sequences analyzed are readily apparent upon inspection of the unrooted phylogenetic tree presented in FIG. 5, where the branch lengths are proportional to the evolutionary distances. In the phylogenetic tree, the Burmese-like isolates, the Mexican isolate and the US isolates each represent a major branch. In addition, the branching of the prototype viruses are supported with bootstrap values of 100%. Analysis of smaller segments of the genome (e.g. ORF 1, ORF 2, or ORF 3) were individually analyzed resulting in trees analogous to those obtained with the full length sequence and shown in FIG. 5. These analyses demonstrate that the HEV US isolates represent a distinct strain or variant of HEV and that HEV US-1 and HEV US-2 are as similar to each other as are the most divergent Burmese-like isolates.

TABLE 40
Phylogenetic distances over the full length sequence

| | B1 | B2 | C1 | C2 | C3 | C4 | I1 | I2 | P1 | M1 | US-1 |
|---|---|---|---|---|---|---|---|---|---|---|---|
| B1 | | | | | | | | | | | |
| B2 | 0.0149 | | | | | | | | | | |
| C1 | 0.0643 | 0.0697 | | | | | | | | | |
| C2 | 0.0680 | 0.0733 | 0.0136 | | | | | | | | |
| C3 | 0.0663 | 0.0734 | 0.0178 | 0.0132 | | | | | | | |
| C4 | 0.0574 | 0.0611 | 0.0304 | 0.0290 | 0.0329 | | | | | | |
| I1 | 0.0677 | 0.0728 | 0.0645 | 0.0625 | 0.0647 | 0.0681 | | | | | |
| I2 | 0.0403 | 0.0477 | 0.0820 | 0.0849 | 0.0846 | 0.0776 | 0.0832 | | | | |
| P1 | 0.0693 | 0.0751 | 0.0178 | 0.0120 | 0.0172 | 0.0335 | 0.0633 | 0.0850 | | | |
| M1 | 0.3096 | 0.3120 | 0.3086 | 0.3089 | 0.3091 | 0.3132 | 0.3120 | 0.3259 | 0.3055 | | |
| US-1 | 0.3406 | 0.3418 | 0.3360 | 0.3345 | 0.3367 | 0.3445 | 0.3322 | 0.3464 | 0.3363 | 0.3462 | |
| US-2 | 0.3413 | 0.3408 | 0.3370 | 0.3361 | 0.3374 | 0.3445 | 0.3333 | 0.3461 | 0.3377 | 0.3367 | 0.0812 |

Comparison to ORF 2/ORF 3 from Swine HEV.

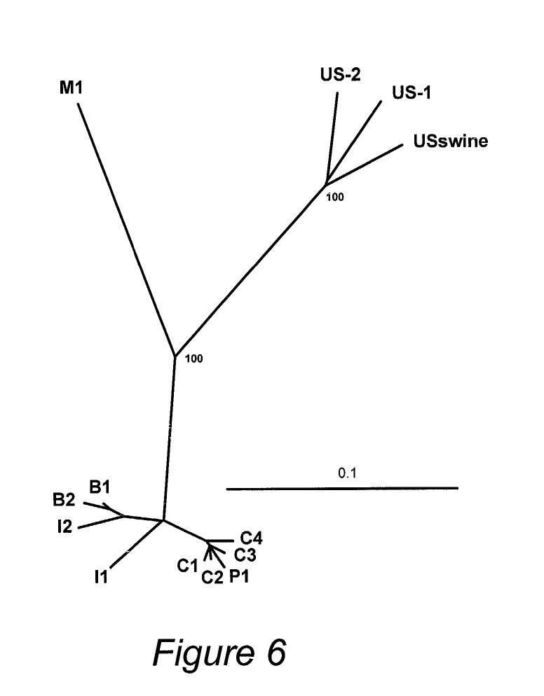

In order to determine the relationship between a recently described swine-HEV and the human HEV US-1 and HEV US-2 isolates, comparisons of the nucleotide sequences across the complete ORF 2 and ORF 3 were performed using analogous regions from the 10 full length sequences utilized above (Table 14). Phylogenetic analysis produces genetic distances of 0.0799 to 0.0810 nucleotide substitutions per position between the US and swine HEV isolates (Table 41). These values are similar to those observed between the most distant Burmese-like isolates. The US and swine isolates group closely on an unrooted phylogenetic tree when the ORF 2/3 nucleotide sequences are analyzed (See, FIG. 6). These isolates form a phylogenetic group distinct from the Mexican isolate and the Burmese-like isolates. These grouping are supported by bootstrap values of 100%.

TABLE 41
Phylogenetic distances between USswine and human HEV isolates

| | US-2 | USswine | Burmese | Mexican |
|---|---|---|---|---|
| US-1 | 0.0799 | 0.0810 | 0.2441-0.2495 | 0.2671 |
| US-2 | | 0.0795 | 0.2409-0.2479 | 0.2486 |
| USswine | | | 0.2348-0.2485 | 0.2615 |
| Burmese | | | 0.0119-0.0716 | 0.2183-0.2248 |

### Example 8

### HEV Serologic Studies

A. Background

Early studies indicate that epitopes useful for diagnosis of HEV infections are located near the carboxyl terminus of ORF 2 and ORF 3 of both the Burmese and Mexican strains of HEV. The two antigens from the Mexican strain, referred to hereinafter as M 3-2 and M 4-2, comprise 42 and 32 amino acids near the carboxyl terminus of ORF 2 and ORF 3, respectively (Yarbough et al. (1991) Journal of Virology, 65: 5790-5797). The two antigens from the Burmese strain of HEV, referred to hereinafter as B 3-2 and B 4-2 proteins, comprise 42 and 33 amino acids near the carboxyl terminus of ORF 2 and ORF 3, respectively (Yarbough et al. (1991) supra). Diagnostic tests designed to detect IgG, IgA and IgM class antibodies to HEV have been developed based on these antigenic regions. Additional HEV recombinant proteins have been generated that encompass full-length ORF 3 (Dawson et al. (1992) Journal of Virology Methods, 38: 175-186) or additional amino acid sequences from the ORF 2 protein (Dawson et al. (1993) supra), to potentially enhance the detection of antibodies to HEV. Comparative studies indicate that the original recombinant proteins and synthetic peptides (B4-2, B3-2, M3-2, M4-2) were as effective as the larger recombinant proteins in detecting antibodies to HEV in known cases of acute HEV infection. A licensed test to detect antibodies to HEV is manufactured by Abbott Laboratories and consists of the full length Burmese strain ORF 3 protein and the carboxyl 327 amino acids of the Burmese strain ORF 2 protein.

After initial serological studies demonstrating the utility of B 3-2, B 4-2, M 3-2 and M 4-2, it was established that six additional amino acids reside at the carboxyl terminus of ORF 2 of both the Burmese and Mexican strains of HEV which do not form part of the M 3-2 and B 3-2 antigenic peptides. Since the carboxyl ends of ORF 2 and ORF 3 have been shown to be of value for the Burmese and Mexican strains of HEV, synthetic peptides corresponding to the these regions of the genome were generated for the US-1 strain of HEV. The synthetic peptides corresponding to the 48 amino acids at the carboxyl end of the ORF 2 were generated for the Burmese and Mexican strains of HEV (SEQ ID NOS:172 and 170, respectively), and are referred to as B 3-2e and M 3-2e (where “e” designates extended amino acid sequence). In addition, synthetic peptides representing the 33 amino acids at the carboxyl end of the HEV US-1 ORF 3 were generated for the Burmese and Mexican strains of HEV (SEQ ID NOS:171 and 169, respectively), and are referred to as B4-2 and M4-2. The synthetic peptide based on the epitope from within ORF 2 for the HEV US-1 strain (SEQ ID NO:174) is referred to as the US 3-2e. The synthetic peptide based on the epitope at the carboxyl end of the HEV US-1 ORF 3 (SEQ ID NO:173) is referred to as US 4-2. Each of these peptides derived from the Mexican, Burmese and US strains of HEV were synthesized, coated on a solid phase and utilized in ELISA tests to determine the relative usefulness of these synthetic peptides.

As noted in Table 42, the amino acid identity between HEV US-1 and the Burmese, Mexican, and Pakistani strains of HEV range from about 87.5% to about 91.7% for the amino acids comprising the 3-2e epitopes within ORF 2, and from about 63.6 to about 72.7% for the amino acids comprising the 4-2 epitopes within ORF 3. Without wishing to be bound by theory, given the degree of variability in the regions encoding for epitopes, it is likely that there may be strain specific antibody responses to theses viruses.

TABLE 42
(Similarity/Identify)

| | 3-2e Peptide | | | 4-2 Peptide | | |
|---|---|---|---|---|---|---|
| | Pak | Mex | US-1 | Pak | Mex | US-1 |
| Bur | 100/ | 91.7/91.7 | 93.7/91.7 | 100/100 | 72.7/72.7 | 72.7/72.7 |
| | 97.9 | | | | | |
| Pak | | 91.7/91.7 | 93.7/91.7 | | 72.7/72.7 | 72.7/72.7 |
| Mex | | | 89.6/87.5 | | | 63.6/63.6 |

B. Use of ELISA's in Diagnosing Acute HEV Infection

It has been reported that most cases of acute HEV infection in man are accompanied by IgM class antibodies which bind to one or more HEV recombinant proteins or synthetic peptides. If a person does not have IgM class antibodies to HEV, the basis for diagnosis of acute HEV infection cannot be made on serology alone but may require, RT-PCR and/or other tests to verify HEV as the etiologic agent.

C. Generation of Synthetic Peptides

Peptides were prepared on a Rainin Symphony Multiple Peptide Synthesizer using standard FMOC solid phase peptide synthesis on a 0.025 μmole scale with (HBTU) coupling chemistry by in situ activation provided by N-methyl-morpholine, with 45 minute coupling times at each residue, and double coupling at predetermined residues. Standard cleavage of the resin provided the unprotected peptide, followed by ether precipitation and washing. The peptides synthesized are shown in Table 43.

TABLE 43

| Peptide | Sequence | SEQ ID NO: |
|---|---|---|
| B 3-2e | TLDYPARAHTFDDFCPECRPLGLQGCAFQSTVAELQRLKMKVGKTREL | SEQ ID NO:172 |
| B 4-2 | ANPPDHSAPLGVTRPSAPPLPHVVDLPQLGPRR | SEQ ID NO:171 |
| M 3-2e | TFDYPGRAHTFDDFCPECRALGLQGCAFQSTVAELQRLKVKVGKTREL | SEQ ID NO:170 |
| M 4-2 | ANQPGHLAPLGEIRPSAPPLPPVADLPQPGLRR | SEQ ID NO:169 |
| US 3-2e | TVDYPARAHTFDDFCPECRTLGVQGCAFQSTIAEVQRLKMKVGKTREV | SEQ ID NO:174 |
| US 4-2 | DSRPAPSVPLGVTSPSAPPLPPVVDLPQLGLRC | SEQ ID NO:173 |

D. Analysis of Synthesized Peptides

The synthesized peptides were analyzed for their amino acid composition as follows. The crude peptides from the small scale syntheses (0.025 μmole) were analyzed for their quality by C18 reverse phase high pressure liquid chromatography using an acetonitrile/water gradient with 0.1% (v/v) 2 trifluoracetic acid (TFA) in each solvent. From the analytical chromatogram, the major peak from each synthesis was collected and the effluent analyzed by mass spectrometry (electrospray and/or laser desorption mass spectrometry. Purification of the peptides (small and/or large scale) was achieved using C18 reverse phase HPLC with an acetonitrile/water gradient with 0.1% TFA in each solvent. The major peak was collected, and lyophilized until use.

E. ELISA Test

The utility of the HEV US-1 epitopes was determined by coating 1/4 inch polystyrene beads with each peptide. Specifically, the peptides were solubilized in water or water plus glacial acetic acid and diluted to contain 10 μg/mL in phosphate buffer (pH 7.4). A total of 60 polystyrene beads were added to a scintillation vial along with 14 mL of peptide solution (10 μg/mL) and incubated at 56° C. for two hours phosphate buffered saline (PBS). After incubation, the liquid was aspirated and replaced with a buffer containing 0.1% Triton-X100®. The beads were exposed to this solution for 60 minutes, the fluid aspirated and the beads washed twice with PBS buffer. The beads then were incubated with 5% bovine serum albumin solution for 60 minutes at 40° C. After incubation, the fluid was aspirated and the beads rinsed with PBS. The resulting beads were soaked in PBS containing 5% sucrose for 30 minutes. The fluids then were aspirated and the beads air-dried.

In one study, one-quarter inch polystyrene beads were coated with various concentrations of the synthetic peptide (approximately 50 beads per lot) and evaluated in an ELISA test (described below) using serum from an anti-HEV seronegative human as a negative control and convalescent sera from an HEV-infected person as a positive control. The bead coating conditions providing the highest ratio of positive control signal to negative control signal were selected for scaling up the bead coating process. Two 1,000 bead lots were produced for both HEV US-1 ORF 2 and ORF 3 epitopes and then used as follows.

A sample of sera or plasma was diluted in specimen diluent and mixed with antigen-coated solid phase under conditions that permit an antibody in the sample to bind to the immobilized antigen. After washing, the resulting beads were mixed with horseradish peroxidase (HRPO)-labeled anti-human antibodies that bind to either tamarin or human antibodies bound to the solid phase. Specimens which produced signals above a cutoff value were considered reactive.

More specifically, the preferred ELISA format requires contacting the antigen-coated solid phase with serum pre-diluted with specimen diluent (buffered solution containing animal sera and non-ionic detergents). Specifically, 10 μL of serum was diluted in 150 μL of specimen diluent and vortexed. Then 10 μl of this pre-diluted specimen was added to each well of an ELISA plate, followed by the addition of 200 μL of specimen diluent and an antigen coated polystyrene beads. The ELISA plate then was incubated in a Dynamic Incubator (Abbott Laboratories) with constant agitation at room temperature for 1 hour. After the incubation, the fluids were aspirated, and the wells washed three times in distilled water (5 mL per wash). Next, 200 μL of HRPO-labeled goat anti-human immunoglobulin diluted in a conjugate diluent (buffered solution containing animal sera and non-ionic detergents) was added to each well and the ELISA plate incubated for 1 hour, as indicated above. The wells then were washed three times in distilled water, the beads containing antigen and bound immunoglobulins removed from each well, and then placed in a test tube with 300 μL of a solution of 0.1M citrate buffer (pH 5.5), 0.3% o-phenylenediamine-2 HCl and 0.02% hydrogen peroxide. After 30 minutes at room temperature, the reaction was terminated by the addition of 1 N sulphuric acid. The resulting absorbance at 492 nm was the recorded. The intensity of the color produced was directly proportional to the amount of antibody present in the test sample. For each group of specimens, a preliminary cutoff value was set to separate specimens which presumably contained antibodies to the HEV epitope from those specimens which did not.

Panel 1: Testing of Pre-screened Panels

In order to demonstrate the utility of epitopes derived from the HEV US-1 strain, a panel of specimens was tested by an ELISA based on the HEV US-1 amino acid sequences (Table 44 These samples had been pre-screened for antibodies to HEV, using a combination of existing peptides and a licensed anti-HEV (Abbott Laboratories) as described above and in published reports (Dawson et al. (1993) supra; Paul et al. (1993) supra).

The first 10 members of the panel consisted of specimens obtained from US volunteer blood donors whose sera was negative for antibodies to HEV following analysis using a combination of peptides and recombinant proteins derived from Burmese and Mexican strains of HEV. All the specimens were non-reactive with ELISA's derived from HEV US-1. Five additional specimens were obtained from individuals suffering from acute hepatitis, and who were diagnosed with acute HEV infection because their sera was reactive for both IgG and IgM class antibodies to HEV recombinant antigens and synthetic peptides based on the Burmese and Mexican strains of HEV. Three of the five samples were from Egypt, one from India and one from Norway (a traveler). HEV RNA was detected by RT-PCR in all five of these individuals. These five members were tested for antibodies to the HEV US-1 isolate and both IgG and IgM class antibodies were detected in each of the cases (Table 44). Thus, these data support the use of synthetic peptides from the US-1 strain of HEV as having utility in diagnosing exposure to HEV and for diagnosing acute HEV infections.

TABLE 44

| Test | | | US Isolate | | | |
|---|---|---|---|---|---|---|
| Specimens | Licensed anti HEV | | IgG | | IgM | |
| Tested | IgG | IgM | 4-2 | 3-2e | 4-2 | 3-2e |
| Neg. Control | 0.061 | 0.084 | 0.031 | 0.041 | 0.071 | 0.109 |
| Pos. Control | 0.567 | 1.051 | 1.606 | 1.619 | 1.376 | 1.798 |
| US | | | | | | |
| Volunteer | | | | | | |
| Donors | | | | | | |
| TG 827 | − | − | − | − | − | − |
| EG 549 | − | − | − | − | − | − |
| EC 760 | − | − | − | − | − | − |
| RF 762 | − | − | − | − | − | − |
| RF 762 | − | − | − | − | − | − |
| RG 730 | − | − | − | − | − | − |
| NH 770 | − | − | − | − | − | − |
| AS 705 | − | − | − | − | − | − |
| BW 494 | − | − | − | − | − | − |
| CD 648 | − | − | − | − | − | − |
| Egypt | | | | | | |
| 7 | + | + | + | + | + | + |
| 9 | + | + | + | + | + | + |
| 12 | + | + | + | − | + | + |
| India | + | + | + | + | + | + |
| 543 | | | | | | |
| Norway | + | + | + | + | + | + |
| M1 | | | | | | |

Panel 2: Detection of Antibodies to HEV in Biological Source of HEV US-1 Isolate

Serial bleeds were obtained form the patient described in Example 1, whose serum served as the biological source for the HEV US-1 strain. Based on serological data obtained for the Burmese and Mexican strains of HEV, this patient would have been misdiagnosed as HEV negative because of the lack of detectable IgM class antibodies to HEV. However, both IgM class (Table 45) and IgG class (Table 46) antibodies to the HEV US-1 strain were detected on all four bleed dates (Tables 45 and 46. Had this patient's sera been analyzed for the presence of IgG and IgM class antibodies to the HEV US 3-2e and US 4-2 peptides, a positive diagnosis of acute HEV infection would have been made. This diagnosis is further supported by the observation that the individual had acute hepatitis and most importantly, had detectable HEV US-1 strain RNA in serum samples. These data indicate that synthetic peptides derived form the HEV US-1 strain may be useful in more accurately diagnosing acute infection due to HEV.

TABLE 45

| | IgM: | | | IgM: | | |
|---|---|---|---|---|---|---|
| | ORF 3 synthetic peptide 4-2 | | | ORF 2 synthetic peptide 3-2e | | |
| Specimens | ISOLATES | | | ISOLATES | | |
| Tested | Burmese | Mexican | US-1 | Burmese | Mexican | US-1 |
| Negative | 0.059 | 0.081 | 0.031 | 0.142 | 0.065 | 0.109 |
| Control | | | | | | |
| Positive | 0.854 | 0.985 | 1.363 | 1.309 | 0.579 | 1.798 |
| Control | | | | | | |
| USP-1 | | | | | | |
| 8 days | − | − | + | − | − | + |
| post | | | | | | |
| admission | | | | | | |
| 9 days | − | − | + | − | − | + |
| post | | | | | | |
| admission | | | | | | |
| 10 days | − | − | + | − | − | + |
| post | | | | | | |
| admission | | | | | | |
| 37 days | − | − | + | − | − | + |
| post | | | | | | |
| admission | | | | | | |

TABLE 46

| | IgG: ORF 3 synthetic peptide 4-2 | | | IgG: ORF 2 synthetic peptide 3-2e | | |
|---|---|---|---|---|---|---|
| Specimens | ISOLATES | | | ISOLATES | | |
| Tested | Burmese | Mexican | US-1 | Burmese | Mexican | US-1 |
| Negative Control | 0.039 | 0.055 | 0.031 | 0.034 | 0.057 | 0.041 |
| Positive Control | 1.296 | 0.666 | 0.941 | 1.322 | 0.893 | 1.041 |
| USP-1 | − | − | + | − | − | + |
| 8 days post admission | − | − | + | − | − | + |
| 9 days post admission | − | − | + | − | − | + |
| 10 days post admission | − | − | + | − | − | + |
| 37 days post admission | − | − | + | − | − | + |

Panel 3—Other Cases of Potential Acute HEV Infection

A panel of sera from 50 patients diagnosed with acute hepatitis who were negative for IgM class antibodies to the Burmese and Mexican strains was assembled. Ten of 50 sera samples were positive for antibodies to the US strain of HEV (Tables 47 and 48). RT-PCR was performed on these samples, but none of the 10 were positive for HEV RNA. Thus, as demonstrated in this example, when patient sera is analyzed for the presence of antibodies to HEV US-1, occult viral hepatitis may be diagnosed as acute HEV infection.

TABLE 47

| | IgG: ORF 3 synthetic peptide 4-2 | | | IgG: ORF 2 synthetic peptide 3-2e | | |
|---|---|---|---|---|---|---|
| Specimens | ISOLATES | | | ISOLATES | | |
| Tested | Burmese | Mexican | US-1 | Burmese | Mexican | US-1 |
| Negative Control | 0.059 | 0.081 | 0.031 | 0.142 | 0.065 | 0.109 |
| Positive Control | 0.854 | 0.985 | 1.363 | 1.309 | 0.579 | 1.798 |
| US | − | − | − | − | − | + |
| Acute non A-E | − | − | − | − | − | + |
| SH 755 | − | − | − | − | − | + |
| DT 314 | − | − | − | − | − | + |
| EH 673 | − | − | − | − | − | + |
| SG560 | − | − | − | − | − | + |
| SR681 | − | − | − | − | − | − |
| N11C10 | − | − | + | − | − | + |
| 35 | − | − | + | − | − | + |
| 52 | − | − | − | − | − | + |
| 161 | − | − | − | − | − | + |
| 175 | | | | | | |

TABLE 48

| | IgG: ORF 3 synthetic peptide 4-2 | | | IgG: ORF 2 synthetic peptide 3-2e | | |
|---|---|---|---|---|---|---|
| Specimens | ISOLATES | | | ISOLATES | | |
| Tested | Burmese | Mexican | US-1 | Burmese | Mexican | US-1 |
| Negative Control | 0.039 | 0.055 | 0.031 | 0.034 | 0.057 | 0.041 |
| Positive Control | 1.296 | 0.666 | 0.941 | 1.322 | 0.893 | 1.041 |
| US | − | − | − | − | − | − |
| Acute non A-E | − | − | − | − | − | − |
| SH 755 | − | − | − | − | − | − |
| DT 314 | − | − | − | − | − | − |
| EH 673 | − | − | − | − | − | − |
| SG560 | − | − | − | − | − | − |
| SR681 | − | − | − | − | − | + |
| N11C10 | − | − | − | − | − | − |
| 35 | − | − | − | − | − | + |
| 52 | − | − | − | − | − | − |
| 161 | − | − | − | − | − | − |
| 175 | | | | | | |

### Example 9

### Animal Transmission Studies

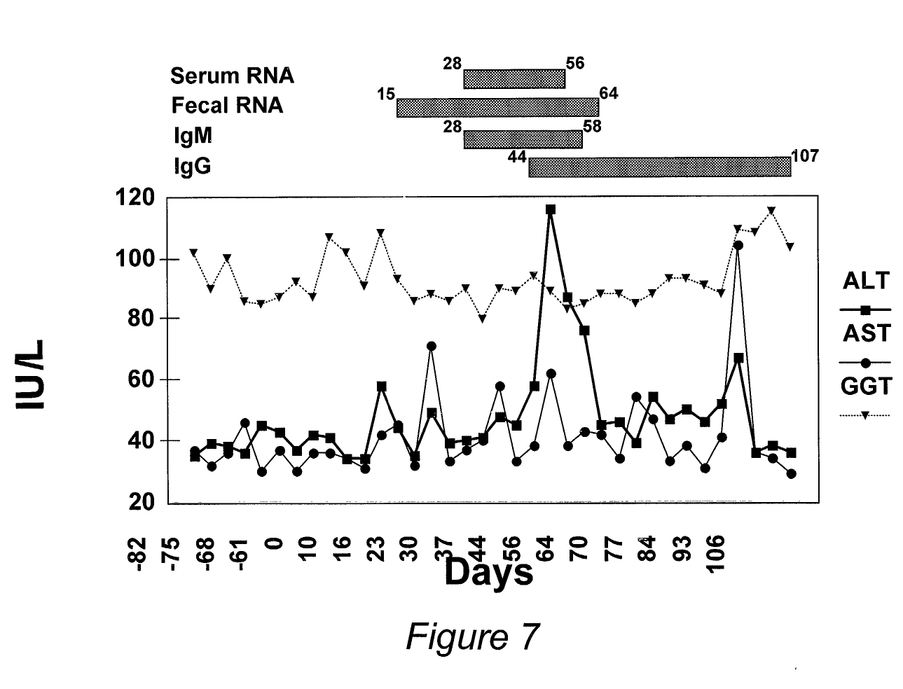

Cynomolgus macaques (Macaca fascicularis) were obtained through the Southwest Foundation for Biomedical Research (SFBR) in San Antonio, Tex. The animals were maintained and monitored in accordance with guidelines established by SFBR to ensure humane care and the ethical use of primates. Sera were obtained twice weekly for at least four weeks prior to inoculation in order to establish the baseline levels for serum ALT. Cut-off (CO) values were determined based on the mean of the baseline plus 3.75 times the standard deviation. Two macaques were inoculated intravenously with 0.4-0.625 mL of HEV positive USP-1 serum and one macaque was inoculated with 2.0 mL of HEV positive USP-2 serum. Serum and fecal samples were collected twice weekly for up to 16 weeks post-inoculation (P1). Sera were tested for changes in ALT and values greater than the CO were considered positive and suggestive of liver damage. Sera samples were tested for antibodies to HEV as described hereinabove in Example 8 (Table 49, FIG. 7). Sera and fecal samples were tested for HEV RNA by RT-PCR. 25-100 μL of macaque sera was extracted using the QIAamp Viral RNA Kit (Qiagen). 10% fecal suspension were extracted as described in Example 1. RT PCR was performed as described below in Example 12 (FIG. 7).

Although intravenous inoculation of 0.4-0.625 mL of USP-1 sera into two cynomolgus macaques failed to produce infection (data not shown), inoculation of 2.0 mL of sera from patient US-2 resulted in viremia and elevations of liver enzyme levels in the serum (FIG. 7). HEV RNA was first detected in fecal material on day 15 PI and remained positive through 64 days PI. Serum specimens collected between days 28-56 PI were HEV RNA positive. Elevated ALT values were noted on days 15, 44-58, 72 and 93 PI, with the peak ALT value (116 IU/L) on day 51 PI.

Six ELSIAs based on the Burmese, Mexican and US sequences for the 4-2 and 302e peptides were utilized to assess antibody response. Measurable response was found only to the US 3-2e peptide assay (Table 49) with no noted crossreactivity to the Burmese or Mexican peptides. IgM class antibody directed against HEV was detectable between 28 and 58 days PI. This was followed by a strong anti-HEV-IgG response at day 44 PI.

TABLE 49

| Date | DPI | ALT | AST | GGT | IgG S/N |
|---|---|---|---|---|---|
| 06/04/97 | −82 | 35 | 37 | 102 | 1.4 |
| 06/06/97 | −80 | 39 | 32 | 90 | |
| 06/11/97 | −75 | 38 | 36 | 100 | |
| 06/13/97 | −73 | 36 | 46 | 86 | |
| 06/18/97 | −68 | 45 | 30 | 85 | |
| 06/20/97 | −66 | 43 | 37 | 87 | |
| 06/25/97 | −61 | 37 | 30 | 92 | |
| 06/27/97 | −59 | 42 | 36 | 87 | |
| 08/25/97 | 0 | 41 | 36 | 107 | 1 |
| 08/27/97 | 2 | | | | |
| 09/02/97 | 8 | 34 | 34 | 102 | |
| 09/04/97 | 10 | 34 | 31 | 91 | |
| 09/09/97 | 15 | 58 | 42 | 108 | 0.8 |
| 09/10/97 | 16 | 44 | 45 | 93 | |
| 09/15/97 | 21 | 35 | 32 | 86 | |
| 09/17/97 | 23 | 49 | 71 | 88 | |
| 09/22/97 | 28 | 39 | 33 | 86 | 1.2 |
| 09/24/97 | 30 | 40 | 37 | 90 | |
| 09/29/97 | 35 | 41 | 40 | 80 | |
| 10/01/97 | 37 | 48 | 58 | 90 | 1.1 |
| 10/03/97 | 39 | | | | |
| 10/06/97 | 42 | 45 | 33 | 89 | |
| 10/08/97 | 44 | 58 | 38 | 94 | 6.2 |
| 10/15/97 | 51 | 116 | 62 | 89 | 11.9 |
| 10/20/97 | 56 | 87 | 38 | 83 | 33.6 |
| 10/22/97 | 58 | 76 | 43 | 85 | 29.9 |
| 10/28/97 | 64 | 45 | 42 | 88 | 17.2 |
| 10/29/97 | 65 | 46 | 34 | 88 | |
| 11/03/97 | 70 | 39 | 54 | 85 | |
| 11/05/97 | 72 | 54 | 47 | 88 | 13.3 |
| 11/10/97 | 77 | 47 | 33 | 93 | |
| 11/12/97 | 79 | 50 | 38 | 93 | 12.4 |
| 11/17/97 | 84 | 46 | 31 | 91 | 10.4 |
| 11/19/97 | 86 | 52 | 41 | 88 | |
| 11/26/97 | 93 | 67 | 104 | 109 | 7.2 |
| 12/03/97 | 100 | 36 | 36 | 108 | |
| 12/09/97 | 106 | 38 | 34 | 115 | |
| 12/10/97 | 107 | 36 | 29 | 103 | 2.1 |

### Example 10

### Recombinant Protein ELISAs

A. Recombinant Constructs

E. coli derived recombinant proteins encoded by HEV-US sequence from the ORF 2 and ORF 3 regions of the HEV-US genome were expressed as fusion proteins with CMP-KDO synthetase (CKS), designated as pJOorf3-29 (SEQ ID NO:191); cksorf2m-2 (SEQ ID NO:192); and CKSORF32M-3 (SEQ ID NO:193), or as non-fusion proteins, designated as plorf3-12 (SEQ ID NO:194); plorf2-2.6 (SEQ ID NO:195); and PLORF-32M-14-5 (SEQ ID NO:196). The cloning vector pJO201, as described in U.S. Pat. No. 5,124,255, was used in the construction of the recombinant fusion proteins. This vector was digested with the restriction endonucleases Eco RI and Bam HI to allow cloning of HEV-US sequences in frame with CKS. The lambda pL expression vector pKRR826 was utilized in the construction of recombinant non-fusion proteins. This vector was digested with the restriction endonucleases Eco RI and Bam HI to allow for cloning of HEV-US sequences immediately down stream of the ribosome binding site. Since the vector system contains strong lambda promoter, induction of heterologous protein synthesis is accomplished by shift in the temperature from 30° C. to 42° C. which inactivates the temperature sensitive repressor protein. The constructs were cloned and transformed into E. coli K12 strain HS36 cells for the expression of these HEV proteins. HEV-US sequences were amplified from nucleic acids extracted from HEV US-2 human serum or macaque 13906 fecal material and reverse transcribed as described above in Example 5. The ORF 2 sequence, encompassing the carboxyl half of ORF 2 (i. e., encoding amino acid residue numbers 334-660 of SEQ ID NO:167), was generated using a sense primer, SEQ ID NO:208, which contained an Eco RI restriction site as well as an ATG start codon and an antisense primer, SEQ ID NO:198, which contained a unique peptide sequence termed FLAG (Eastman Kodak), two consecutive TAA termination codons, and a Bam HI restriction site. A 50 μl PCR reaction was set up using LA TAQ (Takara) reagents as recommended by the manufacturer. Cycling conditions involved 40 cycles of 94° C. for 20 seconds, 55° C. for 30 seconds, 72° C. for 2 minute. Amplifications were preceded by 1 minute at 94° C. and followed by 10 minutes at 72° C. Products were digested with Eco RI and Bam HI and ligated into the desired vector. The nucleotide sequence of the CKS fusion clone, between the restriction sites, is set forth in SEQ ID NO:192, the translation of which is set forth in SEQ ID NO:199. The nucleotide sequence of the non-fusion clone, between restriction sites, is set forth in SEQ ID NO:195, the translation of which is set forth in SEQ ID NO:200. The ORF 3 sequences, encompassing the entire ORF 3 (amino acids 1-122), was generated using a sense primer, SEQ ID NO:201, which contained an Eco RI restriction site as well as an ATG start codon and an antisense primer, SEQ ID NO:202, which contained a unique peptide sequence termed FLAG, two consecutive TAA termination codons, and a Bam HI restriction site. A 50 μL PCR reaction was set up using Qiagen reagents as described in Example 5. Cycling conditions comprised 35 cycles of 94° C. for 30 seconds, 55° C. for 30 seconds, 72° C. for 1 minute. Amplifications were preceded by incubation for 1 minute at 94° C., followed by 10 minutes at 72° C. The resulting products were digested with Eco RI and Bam HI and ligated into the desired vector. The nucleotide sequence of the CKS fusion clone, between the restriction sites, is set forth in SEQ ID NO:191, the translation of which is set forth in SEQ ID NO:203. The nucleotide sequence of the clone representing the non-fusion construct, between the restriction sites, is set forth in SEQ ID NO:195, the translation of which is set forth in SEQ ID NO:204.

Additionally, a chimeric construct encompassing the full length ORF 3 (amino acids 1-123) and the carboxyl half of ORF 2 (amino acids 334-660) was generated. Approximately 100 ng of the plasmids containing SEQ ID NO:191 and SEQ ID NO:192 were utilized as template in 100 μL PCR reactions. PCR buffers and enzymes were from the LA TAQ kit (Takara), and used in accordance with the manufacturer's instructions. ORF 3 was amplified with primers set forth in SEQ ID NOS:201 and 205. The antisense primer of SEQ ID NO:205 eliminates the FLAG sequences and stop codons from the carboxyl end of SEQ ID NO:191 and contains the sequence identical to SEQ ID NO:192 which will eliminate the ATG start codon. ORF 2 was amplified with primers of SEQ ID NOS:208 and 198. Cycling conditions were as described above using LA TAQ. The resulting products were fractionated on a 1.2% agarose gel and excised. DNA was isolated from the gel slices using GeneClean II as described by the manufacturer (Bio101). Products were eluted off the glass beads into 15 μL H₂O. Approximately equal molar ratios of each product (10 μL of ORF 3 product and 1 μL of ORF 2 product) were mixed in a 25 μL end fill reaction using lx PCR buffer, 0.5 μl dNTPs, and 0.25 μL LA TAQ (Takara). This reaction was cycled as follows: 94° C. for 1 minute, 10 cycles of 94° C. for 20 seconds, 55° C. for 30 seconds, and 72° C. for 1.5 minutes, followed by 72° C. for 10 minutes. 5 μL of this reaction was placed into a 100 μL amplification reaction utilizing LA TAQ kit (Takara) and primers of SEQ ID NOS:201 and 198. Cycling conditions were 94° C. for 1 minute followed by 35 cycles of 90° C. for 20 seconds, 55° C. for 30 seconds, and 72° C. for 1.5 minutes. This was followed by 10 minutes at 72° C. and a 4° C. soak. Products of the appropriate size were digested with restriction enzymes Eco RI and Bam HI. This product was ligated into pJO201 and clones with the appropriate sequence identified (SEQ ID NO:193, the translation of which is set forth in SEQ ID NO:206). The resulting product was ligated into pKRR826 and clones with the appropriate sequence (SEQ ID NO:196, the translation of which is set forth in SEQ ID NO:207) identified.

B. Protein Expression and Purification

The CKS constructs were expressed in two 500 mL cultures (4 hour induction), as described in U.S. Pat. No. 5,312,737. PL constructs were expressed as described above. Frozen cell pellets of the induced E. coli cultures were used as the starting material for the purification of protein. Cells were lysed in buffer containing lysozyme, DNase and proteinase inhibitors. Soluble protein was separated from insoluble (inclusion body) protein by centrifugation at 11,000× g. The solubility of the recombinant protein was estimated via sodium dodecyl sulfate (SDS) polyacrylamide gel electrophoresis (PAGE) and Western blotting using a FLAG® M2 antibody. Soluble recombinant protein was purified by affinity chromatography using FLAG® M2 antibody affinity gel after exchange into suitable buffer (Surowy et al. (1997) Journal of General Virology, 78:1851-1859). If necessary, additional purification was performed via Sephacryl® S-200 gel filtration chromatography, in which the sample and chromatography buffers contained 10 mM β-mercaptoethanol. Purified protein was quantitated by measurement of absorbance at 280 nm. An assumed extinction coefficient of 1 was used to convert absorbance to mg of protein. Protein purity was determined by scanning densitometry (Molecular Dynamics) of protein fractioned by SDS PAGE, using standards of pre-determined purity.

C. ELISA

In order to determine potential utility of the recombinant HEV US constructs, solid phase ELISA's were developed and evaluated. All recombinant HEV US proteins were coated onto solid phase as described below. Briefly, 1/4″ polystyrene beads were coated with varying amounts of (PJOORF3-29) which ranged in concentration from 0.5 to 10 μg/mL diluted in 100 mM sodium phosphate buffer, pH 7.6. Sixty beads per concentration condition were coated in approximately 14 mL of buffer and rotated end-over-end at 40° C. for 2 hours. The coating solution was aspirated and the remainder of the coating procedure was performed as described above in Example 8, section E, paragraph 1.

An ELISA was developed using the pJOorf3-29 coated beads. Briefly, sera or plasma was diluted 1:16 in Specimen Diluent (SpD) as described above. A 10 μL aliquot of this pre-dilution then was added into the well of a reaction tray, followed by the addition of 200 μL of SpD. One coated bead was added per well and incubated for 1 hour at 37° C. in dynamic mode using a Dynamic Incubator (Abbott Laboratories). After incubation, the fluid was aspirated and each bead washed 3 times with deionized water (5 mL per wash). The beads then were incubated with 200 μL HRPO-labeled goat anti-human IgG or IgM conjugate, diluted in conjugate diluent (described above) and incubated for 30 minutes at 37° C. The conjugate then was aspirated and the beads washed as above. Color development and absorbance readings were performed as described in Example 8, section E.

To validate the immunoreactivity of this construct, serial bleed specimens from Macaque #13903 experimentally infected with HEV US-2 (described in Example 9) were tested for IgM and IgG antibody to pJOorf3-29. As shown in FIG. 1, IgM antibody was detected at day 51 post-infection (PI) and continued to be elevated through day 72 and corresponded to the peak elevations in ALT values. IgG antibody to pJOorf3-29 was first detected on day 56 PI and remained positive through day 107 (Table 50).

A second construct, plorf3-12, representing HEV US ORF 3 but lacking the CKS fusion partner was also evaluated in an ELISA format identical to that described above. IgG antibody to plorf3-12 was evaluated on several serial bleeds from the same experimentally infected macaque. IgG antibody to plorf3-12was detected on day 58 PI and remained positive through day 107 (Table 50).

TABLE 50

pJOorf3-29

| | |
|---|---|
| Mean | plorf3-12 |

| Sample | OD | S/N | Mean OD | S/N |
|---|---|---|---|---|
| SpD | | | 0.01 | |
| “pre-bleed” | 0.02 | | 0.01 | |
| Post-inoculation bleeds - Days Post- | | | | |
| inoculation (DPI) | | | | |
| DPI | | | | |
| 44 | 0.02 | 0.96 | 0.02 | 1.07 |
| 51 | 0.05 | 2.35 | 0.03 | 2.25 |
| 56 | 0.24 | 10.35 | 0.05 | 3.43 |
| 58 | 0.44 | 19 | 0.16 | 11.57 |
| 63 | 1.14 | 49.57 | 0.32 | 22.82 |
| 65 | | NT | 0.53 | 37.54 |
| 70 | | NT | 1.19 | 85.04 |
| 72 | 2.22 | 96.52 | 0.92 | 65.71 |
| 98 | 0.89 | 38.87 | 0.39 | 27.86 |
| 107 | 0.49 | 21.43 | 0.27 | 19.36 |

NT: not tested

Due to the high percent homology between Swine HEV and the US-2 isolate, the pJOorf3-29 ELISA also was used to measure the prevalence of both immunoreactive IgG and IgM in sera isolated from U.S. swine herds (Table 51). The assay was performed as described above with the exception of substituting HRPO-conjugated labeled anti-swine immunoglobulin (either IgG or IgM) for the anti-human conjugate.

TABLE 51
Prevalence of Antibody to HEV orf3 in U.S. Swine
(pJOorf3-29)

| | | | | No. IgM | |
|---|---|---|---|---|---|
| | IgG | No. IgG | IgM Only | Only | Total |
| Swine | Reactive | Confirmed by | Reactive | Confirmed | Exposure |
| Source | No./Total | Blocking or Blot | No./Total | by Blot | Confirmed |
| State | (%) | (%) | (%) | (%) | Only |
| New Jersey | 9/14 | 9 | 0/14 | | 64% |
| | (64) | (100) | | | |
| Texas | 25/50 | 20 | 0/50 | | 40% |
| | (50) | (80) | | | |
| Iowa | 7/64 | 1 | 0/64 | | 2% |
| | (11) | (14) | | | |
| Oregon | 7/36 | 5 | 1/36 | 1/1 | 14% |
| | (19) | (71) | (3) | (100) | |
| Total | 48/164 | 35 | 1/164 | 1/1 | 36/164 |
| | (29) | (73) | (0.6) | (100) | (22%) |

NOTE: A total of 4 pigs (all Texas herd) had IgM in addition to IgG.

In order to confirm reactive specimens, a blocking assay was developed. Briefly, a 10 μL aliquot of the 1:16 specimen pre-dilution was added to duplicate wells of a reaction tray; one well to be used for the standard assay and one well to be used for the blocking assay. The ELISA for the standard assay was performed as described above with the exception that there was a 30 minute room temperature pre-incubation step prior to addition of the pJOorf3-29 antigen coated bead. For the blocking assay, pJOorf3-29 was added to the SpD (blocking reagent) at a 10-fold molar excess to that on the solid phase. 200 μL of blocking reagent was added per reaction and a 30 minutes room temperature pre-incubation was performed prior to addition of the pJOorf3-29 antigen coated bead. The rest of the assay was performed as described above for the swine assay, except that the HRPO-conjugated anti-swine conjugate (IgG) was used in place of the anti-human conjugate.

The % blocking was determined using the equation:

[(A_{492 nm} standard assay−A_{492 nm} blocking assay)/A_{492 nm} standard assay]×100

Specimens that showed blocking rates of 50% or greater were considered to be reactive for IgG antibody to HEV pJOorf3-29. Representative IgG positive and IgG negative swine samples and their blocking results are shown in Table 52.

TABLE 52
Blocking Assay With pJOorf3-29 and PL-12 at 10-fold molar excess

| | | | | Blocking Assay w/ pJOorf3-29 at | | | |
|---|---|---|---|---|---|---|---|
| Standard Assay | | | | 10-fold molar excess | | | |
| | | | MEAN | | MEAN | % | BLOCKING |
| | SAMPLE | OD | OD | OD | OD | BLOCKING | RESULTS |
| | | 0.02 | | 0.02 | | | |
| | NC | 0.02 | 0.02 | 0.03 | 0.02 | | |
| | | 1.09 | | 0.56 | | | |
| | PC | 1.01 | 1.05 | 0.48 | 0.52 | 50.4% | + |

Oregon Swine Panel Positives

| | | | | | |
|---|---|---|---|---|---|
| 1 | NJ5 | 0.65 | 0.15 | 76.5% | + |
| 2 | NJ12 | 1.78 | 0.46 | 74.0% | + |
| 3 | NJ21 | 0.48 | 0.16 | 66.7% | + |
| 4 | NJ23 | 0.52 | 0.09 | 81.9% | + |
| 5 | T5 | 2 | 0.81 | 59.5% | + |
| 6 | T9 | 0.52 | 0.18 | 64.3% | + |
| 7 | T32 | 2 | 0.9 | 54.9% | + |
| 8 | T33 | 0.3 | 0.13 | 57.8% | + |
| 9 | T48 | 0.53 | 0.14 | 73.7% | + |
| 10 | T49 | 0.33 | 0.09 | 73.3% | + |

Oregon Swine Panel Negatives

| | | | | | |
|---|---|---|---|---|---|
| 11 | T43 | 0.08 | 0.07 | 13.3% | − |
| 12 | T46 | 0.12 | 0.08 | 29.1% | − |
| 13 | I-23 | 0.12 | 0.08 | 32.2% | − |
| 14 | I-24 | 0.07 | 0.06 | 13.2% | − |
| 15 | I-27 | 0.1 | 0.08 | 12.6% | − |
| 16 | I-28 | 0.15 | 0.12 | 20.4% | − |
| 17 | I-33 | 0.15 | 0.12 | 19.9% | − |
| 18 | I-39 | 0.23 | 0.14 | 37.4% | − |
| 19 | I-61 | 0.19 | 0.14 | 25.9% | − |
| 20 | O-4 | 0.15 | 0.12 | 22.7% | − |

In addition to the blocking assay, western blots were run on a subset of swine specimens. Briefly, 50 μg of HEV pJOorf3-29 and 50 μg of “CKS only” proteins were fractionated by SDS-PAGE and the fractionated proteins transferred to nitrocellulose. 3 mm strips of the nitrocellulose were cut and incubated overnight at room temperature on an orbital rotator with primary antibody at a 1:100 dilution in protein based buffer containing 10% E. coli lysate. On the following day, strips were washed three times with 0.3% Tween/TBS (TBST), followed by the addition of HRPO-conjugated anti-swine IgG conjugate diluted to 0.5 μg/mL in TBST. Strips were incubated with rotation for 4 hours at room temperature. Blots then were washed three times in TBST, followed by 2 washes in TBS. Blots were developed using 4-chloro-1-naphthol as a substrate. The reaction was stopped by the addition of water and band intensities recorded. Specimens were determined to have specific reactivity to HEV if they showed a band at the correct molecular weight for pJOorf3-29 (approx. 40 kD) and had no reactivity in the region where “CKS only” bands (approx. 29 kD). Results for 20 swine sera run on the pJOorf3-29 western blot are shown in Table 53. No swine sera showed non-specific reactivity with the “CKS-only” band.

TABLE 53

BAND INTENSITY

| Swine ID Number | pJOorf3-29 | CKS only |
|---|---|---|
| NJ4 | + | — |
| NJ7 | + | — |
| NJ14 | +++ | — |
| NJ18 | + | — |
| NJ25 | ++++ | — |
| T6 | ++++ | — |
| T10 | ++++ | — |
| T14 | — | — |
| T15 | + | — |
| T18 | ++ | — |
| T28 | +++ | — |
| T29 | — | — |
| T30 | + | — |
| T34 | — | — |
| T36 | ++++ | — |
| T37 | — | — |
| T43 | — | — |
| T44 | ++++ | — |
| T45 | ++++ | — |
| T46 | — | — |

These data suggest that HEV US recombinant proteins are useful in diagnosing exposure to HEV.

### Example 11

### Consensus Primers

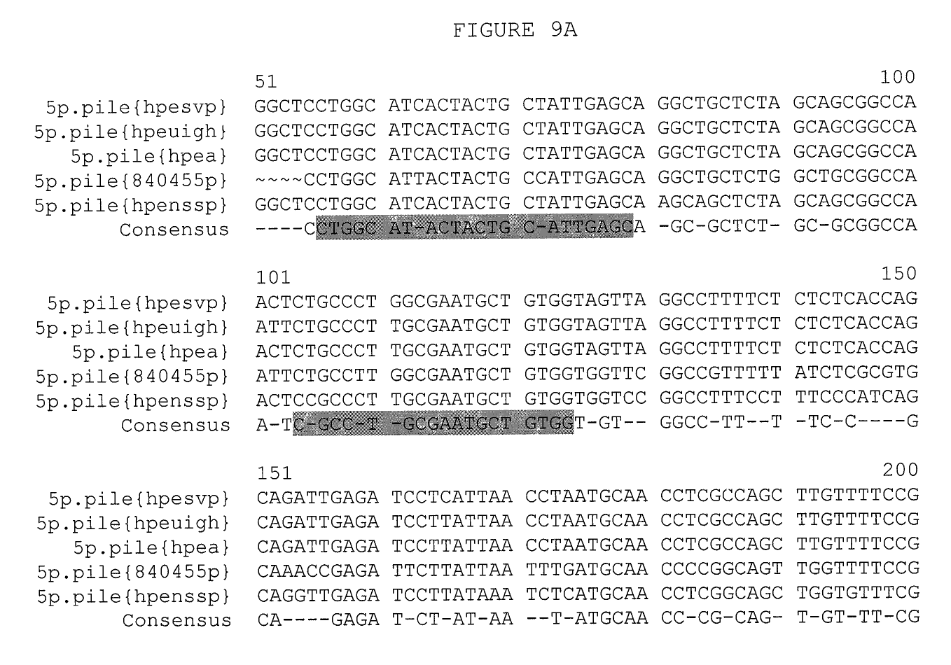

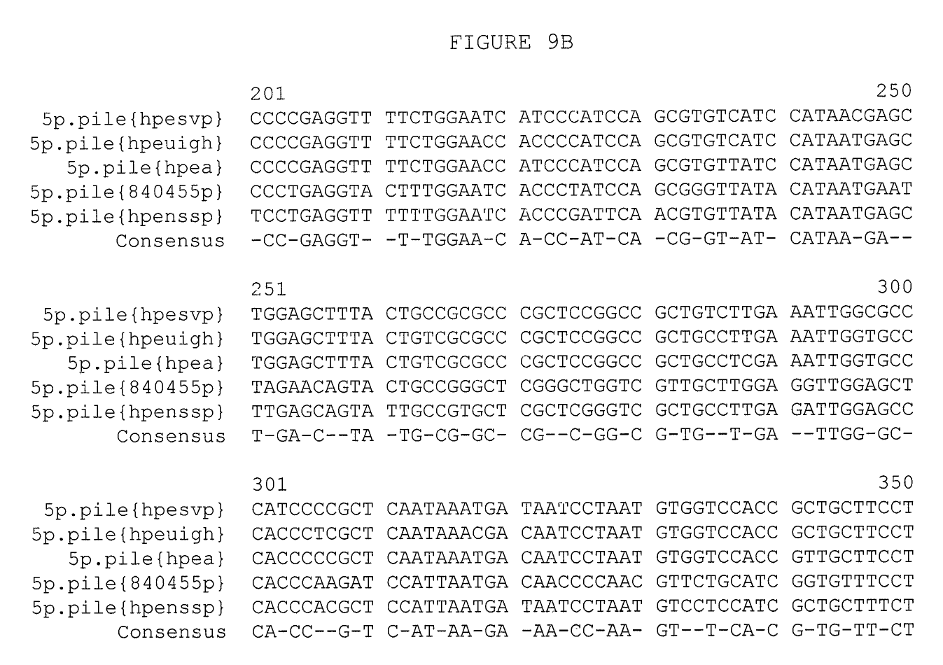

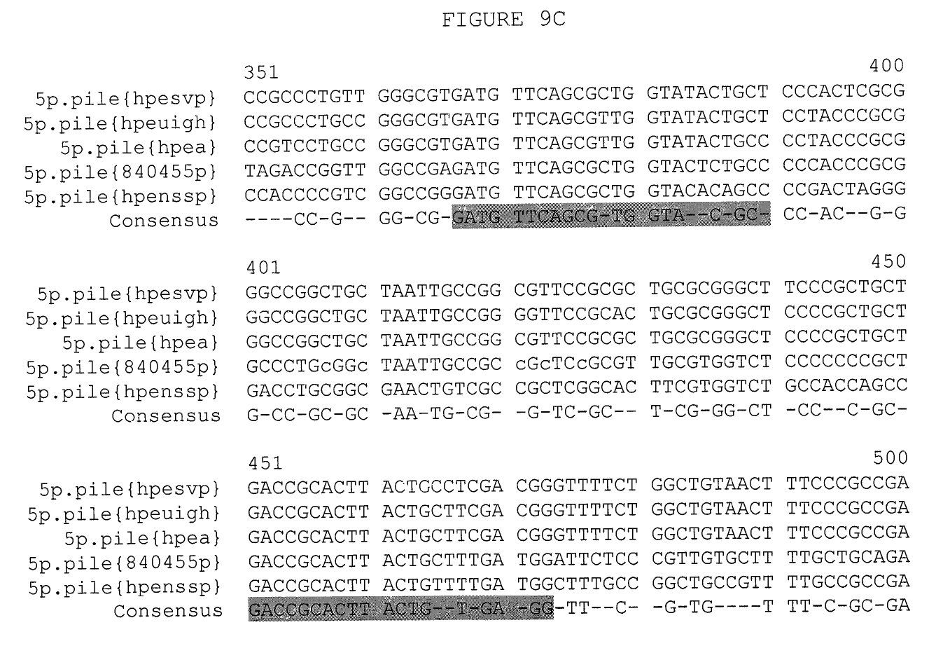

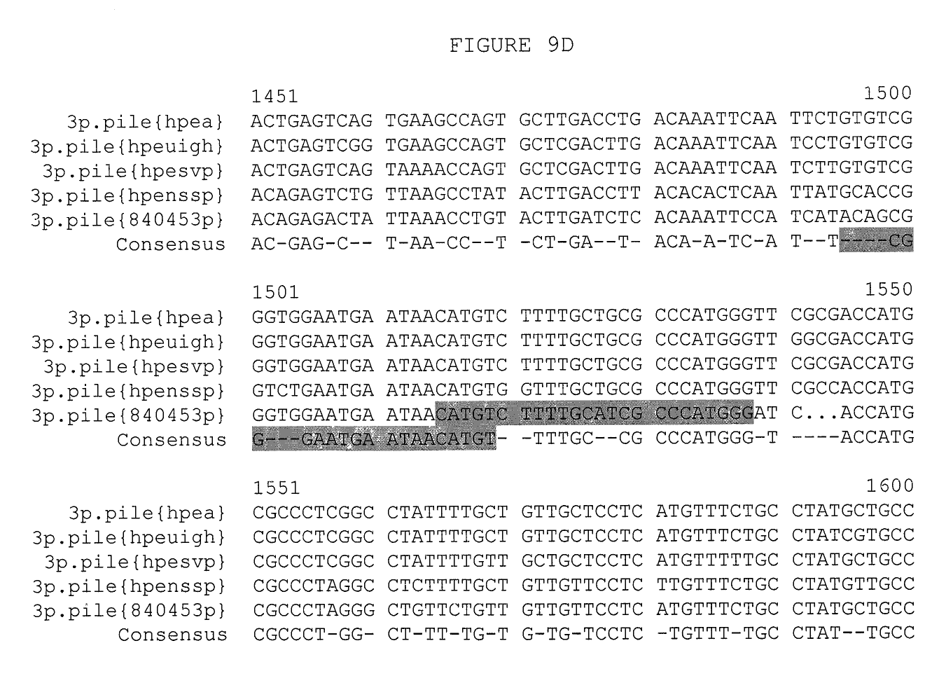

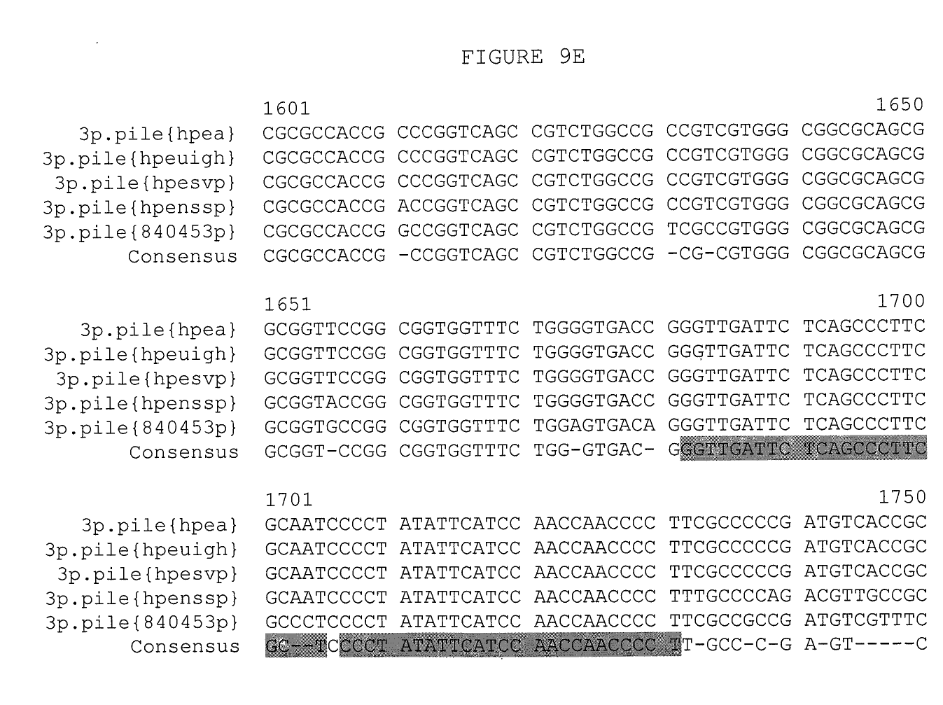

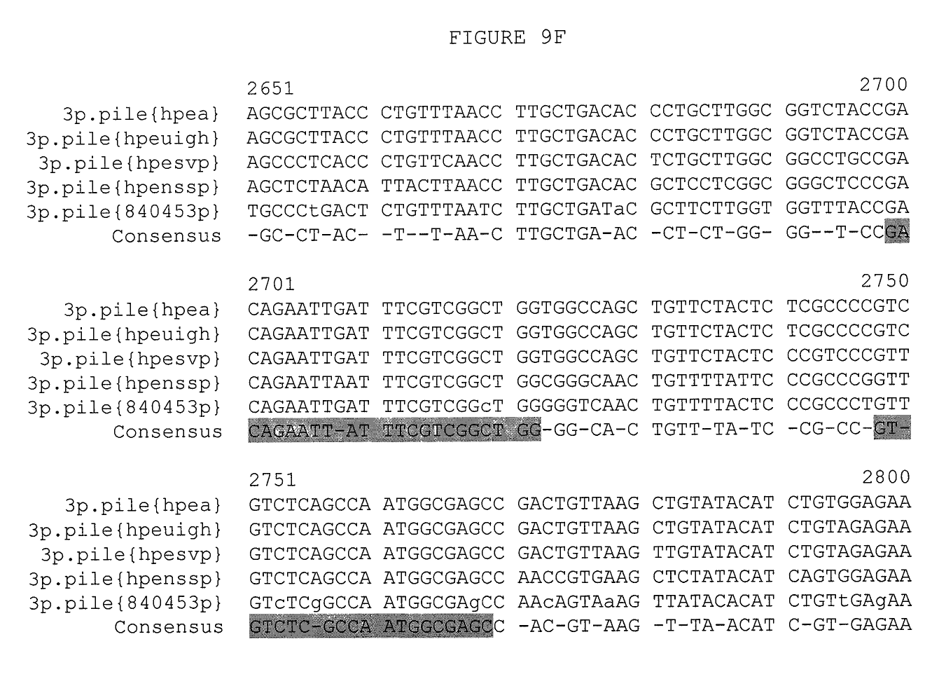

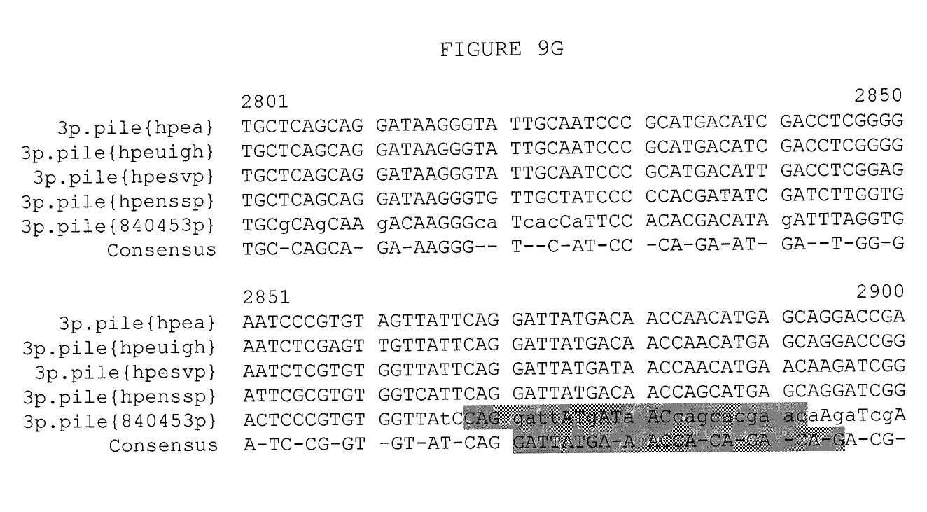

Consensus oligonucleotide primers for HEV ORF 1 ORF 2 and ORF 3 were designed based on conserved regions between the full length sequences of isolates from Asia, Mexico, and the US (FIG. 9). The ORF 1 primers are positioned within the methyltransferase region at nucleotides 56-79 and 473-451 of the Burmese isolate (GenBank accession number M73218), and amplify a product 418 nucleotides in length. The ORF 1 primers include:

HEVConsORF 1-s1; CTGGCATYACTACTGCYATTGAGC (SEQ ID NO:147); and

HEVConsORF 1-a1; CCATCRARRCAGTAAGTGCGGTC (SEQ ID NO:148).

The ORF 2 primers, at positions 6298-6321 and 6494-6470 of the Burmese isolate, produce a product 197 nucleotides in length. The ORF 2 primers include:

| | |
|---|---|
| HEVConsORF 2-s1; | |
| GACAGAATTRATTTCGTCGGCTGG; and | (SEQ ID NO:150) |
| HEVConsORF 2-a1; | |
| CTTGTTCRTGYTGGTTRTCATAATC. | (SEQ ID NO:126) |

For a second round of amplification, internal primers can be used to produce products 287 and 145 nucleotides in length for ORF 1 and ORF 2, respectively. The ORF 1 primers include:

| | |
|---|---|
| HEVConsORF 1-s2; | |
| CTGCCYTKGCGAATGCTGTGG; and | (SEQ ID NO:177) |
| HEVConsORF 1-a2; | |
| GGCAGWRTACCARCGCTGAACATC. | (SEQ ID NO:178) |

The ORF 2 primers include:

| | |
|---|---|
| HEVConsORF 2-s2; | |
| GTYGTCTCRGCCAATGGCGAGC; and | (SEQ ID NO:152) |
| HEVConsORF 2-a2; | |
| GTTCRTGYTGGTTRTCATAATCCTG. | (SEQ ID NO:128) |

PCR reactions contained 2 mM MgCl₂ and 0.5 μM of each oligonucleotide primer as per the manufacturer's instructions (Perkin-Elmer) and amplified using Touch-down PCR as described in Example 5. Amplified products were separated on a 1.5% agarose gel and analyzed for the presence of PCR products of the appropriate size. The primers were used to detect the presence of virus in serum and feces containing HEV US-2 as described above in Example 8 and FIG. 7. In addition, these primers were found to be reactive with a number of different variants of HEV that included Burmese-like strains 6A, 7A, 9A and 12 A as well as two distinct isolates from Greece (see Example 13 below) as well as a unique isolate from Italy and the two isolates from the US (see Example 13 below). In addition, these primers have been used to identify an isolate from a patient with a clinical diagnosis of acute sporadic hepatitis from the Liaoning province of China (S15). The results are presented in Table 54 below.

TABLE 54

| | | | | ORF 2 - |
|---|---|---|---|---|
| Sample | ORF 1 -PCR1 | ORF 1 -PCR 2 | ORF 2 - PCR1 | PCR2 |
| 6A | neg | pos | pos | Pos |
| 7A | neg | pos | neg | Pos |
| 9A | neg | neg | neg | Pos |
| 12A | pos | pos | neg | Neg |
| G1 | pos | pos | pos | Pos |
| G2 | pos | pos | pos | Pos |
| It1 | pos | pos | pos | Pos |
| S15 | nd | pos | nd | Pos |
| US-2 | pos | pos | pos | Pos |

### Example 12

### Detection of HEV RNA in Primary Human Fetal Kidney Cells

Frozen cell pellets containing 10×10⁶ cells were thawed and resuspended in 1.0 mL Dulbecco's phosphate buffered saline. RNA was extracted from 20 μL (2×10⁵ cells) of the cell pellet using the Ultraspec Isolation System as described in Example 1. cDNA synthesis was performed on the above extracted nucleic acid (RNA) and primed with random hexamers. PCR then was performed on the above cDNA using degenerate primers from the ORF-1 and ORF-2 regions of the viral genome at a final concentration of 0.5 μM as described in Example 11.

To monitor the performance of the above assay, a positive control utilizing primary human kidney cells and HEV US-2 positive serum was included in the experimental design. Two positive control sets were prepared by spiking 2×10⁵ HEV negative primary human kidney cells with 2.5 μL and 25 μL of a documented HEV US-2 positive serum specimen. The positive control serum also was tested without the addition of the human kidney cells.

Nineteen primary human kidney cell pellet lots were tested using the above assay method utilizing the 2 degenerate primer sets from ORF 1 and ORF 2. The results are summarized in Table 55 below. None of the cell pellet lots tested gave positive results as seen in the positive controls.

TABLE 55

| CELL LINES | PCR RESULTS |
|---|---|
| 1757 | — |
| 1851 | — |
| 1690 | — |
| 1853 | — |
| 1906 | — |
| 1935 | — |
| 1838 | — |
| 1955 | — |
| 1893 | — |
| 1895 | — |
| 1699 | — |
| 1877 | — |
| 1942 | — |
| 1844 | — |
| 1840 | — |
| 1875 | — |
| 1921 | — |
| 1946 | — |
| 1846 | — |
| cells + 25 μL serum | + |
| cells + 2.5 μL serum | + |
| 25 μL serum | + |

### Example 13

### Identification and Extension of Additional US-type Isolates

A. Identification of Isolate from Italy, Referred to as It1

RNA was extracted from 25 to 50 μL of serum using the QIAamp Viral RNA kit (Qiagen) as described by the manufacturer except that 25 to 50 μL of serum was diluted to 100 μL with PBS and the final elution was performed with 100 μL of RNase-free water. RT reactions were random primed. PCR utilized the HEV US-1 primer as described hereinabove in Example 5. A 294 bp product was generated after amplification with primers SEQ ID NO:94 and SEQ ID NO:96. The product was cloned and sequenced as described in Example 3 and is shown in SEQ ID NO:179.

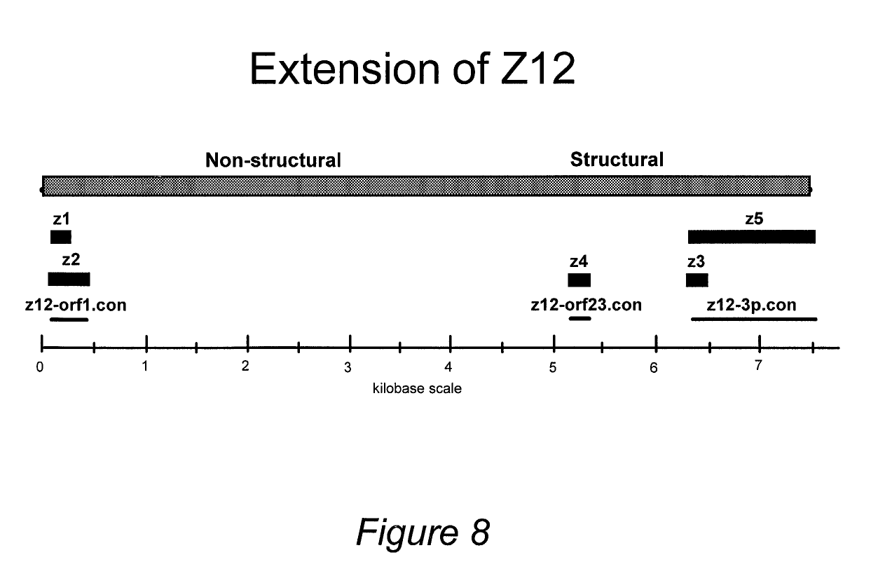

Extension of the It1 isolate genome was performed as follows. RNA was extracted from 25 to 50 μL of serum as described hereinabove in Example 5. RT reactions were random primed. PCR utilized the HEV CONSENSUS primers described above in Example 11 using touchdown PCR, as described hereinabove in Example 3. Primers shown in SEQ ID NOS:147 and 148 were used to generate a product having the sequence set forth in SEQ ID NO:180 (reaction z2, 418 bp). Primers as shown in SEQ ID NOS:150 and 126 were used to generate a product having the sequence set forth in SEQ ID NO:181 (reaction z3, 197 bp). In the presence of 1× PCR Buffer and 20% Q Solution (Qiagen), primers as shown in SEQ ID NOS:182 and 183 were used to generate a product having a sequence set forth in SEQ ID NO: 184 (reaction z4, 234 bp). The 3′ end of the genome was isolated by 3′ RACE as described above in Example 3 using primers shown in SEQ ID NOS:150 and 85 in PCR1, and primers shown in SEQ ID NOS:152 and 85 in PCR2, to produce a product having the sequence shown in SEQ ID NO:185 (reaction z5, 890 bp). Products were cloned and sequenced as described in Example 3 and consensus sequences generated. These regions are shown in FIG. 8 and are set forth in SEQ ID NOS:180, 184 and 186. The amino acid translations of these regions are represented by the amino acid sequences set forth in SEQ ID NOS:187, 188; 189; 190; and 197.

B. Identification of Two Isolates from Greece Referred to as G1 and G2

Two patients with acute hepatitis who had no history of travel to endemic areas had been analyzed with primers based on the Burmese isolate (Psichogiou M. A., et al., (1995) “Hepatitis E virus (HEV) infection in a cohort of patients with acute non-A, non-B hepatitis,” Journal of Hepatology, 23, 668-673). Only patient G2 was found to be PCR positive. RNA was isolated as described hereinabove in Example 12 and PCR performed with the consensus primers described above in Example 11. The ORF 1 and ORF 2 primer sets generated products of the expected size from both patients. The products were cloned and sequenced as described above in Example 3. The products generated using the ORF 1 and ORF 2 consensus primers from patient GI are shown in SEQ ID NOS:209 and 211, respectively. The products generated using the ORF 1 and ORF 2 consensus primers from patient G2 are shown in SEQ ID NOS:213 and 215, respectively. The identification of GI as being PCR positive demonstrates the utility of the consensus primers over Burmese base strain specific primers.

Additional sequence from G1 and G2 was also obtained using primers SEQ ID NO:16, SEQ ID No:17, and SEQ ID NO:18 as for the generation of SEQ ID NO:19 as described above in Example 3 except that random primed cDNA was used for PCR and amplification involved 10 cycles of 94° C. for 20 seconds, 60° C. for 30 seconds, and 72° C. for 1 minute, followed by 10 cycles of 94° C. for 20 seconds, 55° C. for 30 seconds, and 72° C. for 1 minute followed by 30 cycles of 94° C. for 20 seconds, 50° C. for 30 seconds (−0.3° C./cycle), and 72° C. for 1 minute. This was followed by an extension cycle of 72° C. for 7 minutes. The product generated from patient GI is shown in SEQ ID NO:217. The product generated from patient G2 is shown in SEQ ID NO:220.

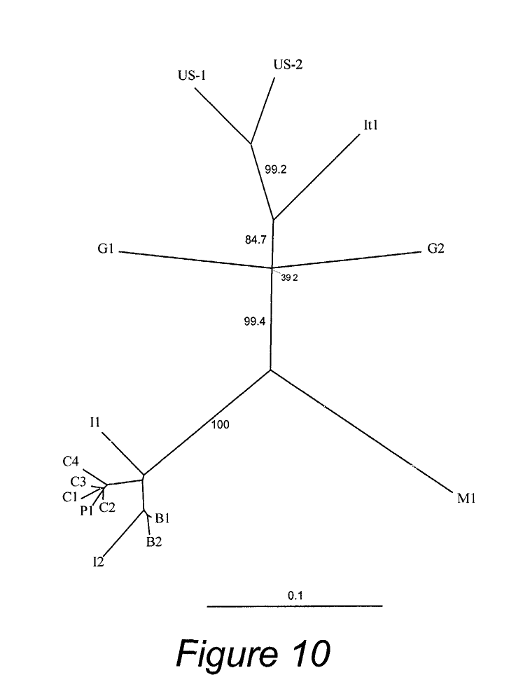

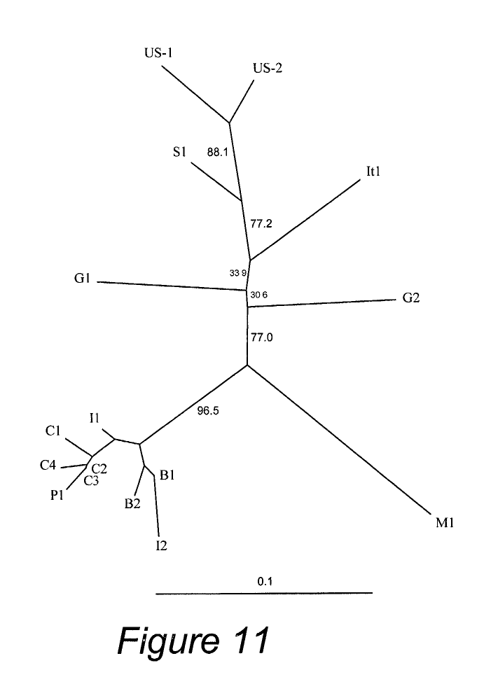

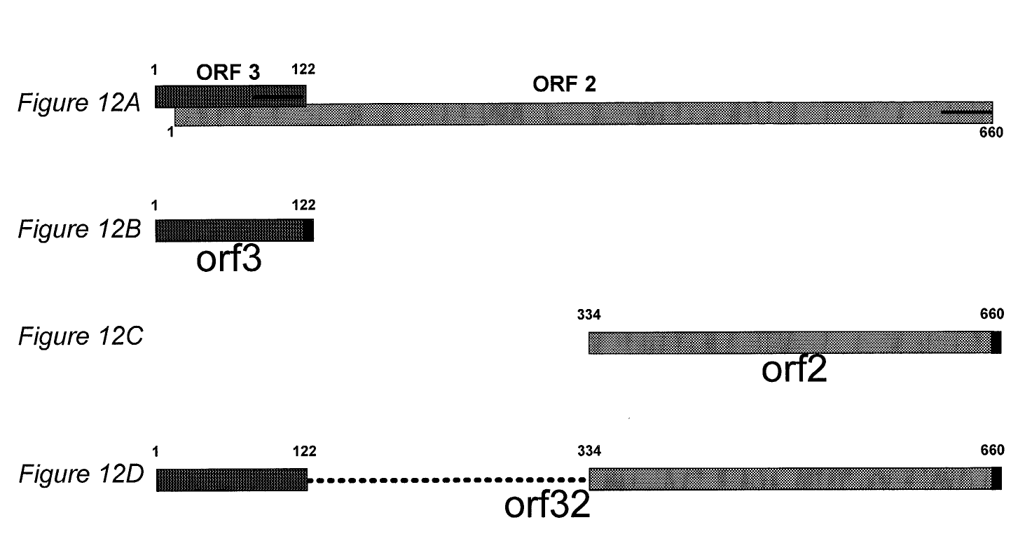

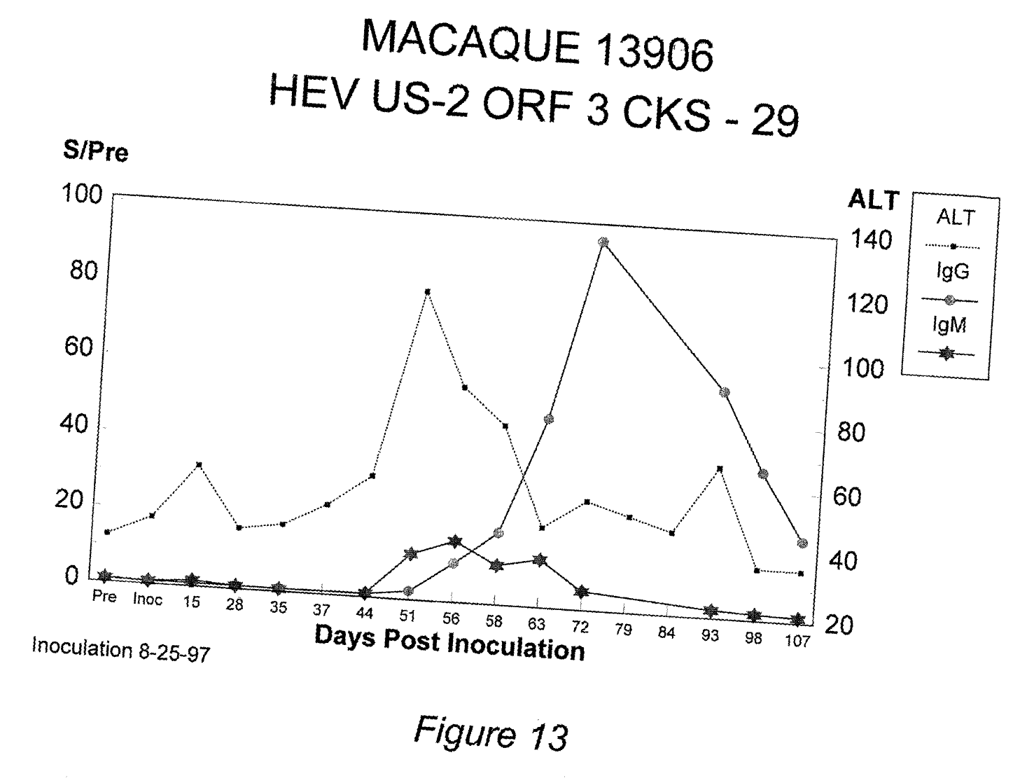

Alignments of the nucleotide sequences of the US, Chinese, Greek, Italian, Mexican and Burmese-like isolates, were performed to determine the relationship of these isolates to each other. The divergence of the Italian isolate is supported by the comparisons of the product from the ORF 1 region of the genome which has a percent nucleic acid identity of 77.6%, 78.4%, and 84.6% with the prototype isolates from Burma (B1), Mexico (M1) and the US (US-1), respectively (Table 36). The divergence of the Italian isolate also is supported by the comparisons of the product from the ORF 2 region of the genome which had a percent nucleic acid identity of 83.3%, 79.7%, and 87.8% with the prototype isolates from Burma, Mexico and the US, respectively (Table 37). The nucleotide identities between the prototype isolates from Burma, Mexico and the US, range between 75.5% to 82.4% over these two regions. Over these same regions, the isolates that comprise the Burmese-like group have much higher identities of 91.2% or greater. Comparisons of the ORF 1 and ORF 2 amplified sequences indicate that the isolates from the two patients from Greece are quite distinct from each other, exhibiting 84.4% and 87.2% nucleotide sequence identity over these regions of ORF 1 and ORF 2, respectively. At the nucleotide level, the percent identities between the Greek, Italian and US isolates range from 81.9% to 86.8% for the ORF 1 product (Table 36) and 82.4% to 87.8% for the ORF 2 product (Table 37). These values are lower than the lowest percent nucleotide identities between any Burmese-like isolates, which are greater than 91.2% for both ORF 1 and ORF 2. Comparisons of the amino acid identities derived from the ORF 1 fragment between the US, Italian or Greek isolates and the Burmese or Mexican isolates range from 87.8% to 93.5% (Table 36). These values are equal to or less than the differences between the Burmese and Mexican isolates (93.5% to 95.1%) (Table 36), indicating that the isolates from non-endemic regions are distinct from the isolates originating from endemic regions. The relative evolutionary distances between the viral sequences analyzed are readily apparent upon inspection of the unrooted phylogenetic trees generated from the pairwise distances, where the branch lengths are proportional to the relative genetic relationships between the isolates. The phylogenetic trees based on alignments of either ORF 1 (FIG. 10) or ORF 2 (FIG. 11) sequences are quite similar in overall topology. The Burmese-like isolates and the Mexican isolate represent major branches at one end of the tree. The human US isolates form a distinct group distal to the Mexican and Burmese isolates The swine HEV-like sequence from ORF 2 is closely related to the US human isolates. The three European isolates form three additional distinct branches with the Italian isolate being most closely related to the US isolates.

### Example 14

### Identification Additional US-type Isolates from Austria and Argentina

RNA was isolated from serum from three patients with acute hepatitis who had no history of travel to areas considered endemic for HEV as described hereinabove in Example 12 and PCR performed with the consensus primers described above in Example 11. One patient was from Austria, Au1, (Worm, et al., (1998) “Sporadic hepatitis E in Austria,” New England Journal of Medicine, 339, 1554-1555) while the other two patients were from Argentina. The ORF 1 and ORF 2 primer sets generated products of the expected size from all patients. The products were cloned and sequenced as described above in Example 3. The products generated using the ORF 1 and ORF 2 consensus primers from patient Au1 are shown in SEQ ID NOS:243 and 245, respectively. The products generated using the ORF 1 and ORF 2 consensus primers from patient Ar1 are shown in SEQ ID NOS:247 and 249, respectively. The products generated using the ORF 1 and ORF 2 consensus primers from patient Ar2 are shown in SEQ ID NOS:251 and 253, respectively. PCR products were obtained after both the first round of ORF 1 PCR with the a1 and s1 primers as well as the second round of nested ORF 1 PCR with the a2 and s2 primers for Au1, Ar1 and Ar2. PCR products were obtained after both the first round of ORF2 PCR with the a1 and s1 primers as well as the second round of nested ORF2 PCR with the a2 and s2 primers for Au1 and Ar2. Product from Ar1 was detected only after the second round of nested ORF2 PCR with the a2 and s2 primers.

Alignments of the nucleotide sequences of the US, Chinese, Greek, Italian, Austrian, Argentine, Mexican and Burmese-like isolates, were performed to determine the relationship of these isolates to each other as described in Example 6. The divergence of the Austrian isolate, Au1, is supported by the comparisons of the product from the ORF 1 region of the genome which has a percent nucleic acid identity of 77.1%, 78.2%, and 87.9% with prototype isolates from Burma (B1), Mexico (M1) and the US (US-1), respectively (Table 56). The divergence of the Austrian isolate also is supported by the comparisons of the product from the ORF 2 region of the genome which had a percent nucleic acid identity of 85.1%, 79.1%, and 83.1% with the prototype isolates from Burma (B1), Mexico (M1) and the US (US-1), respectively (Table 57). The divergence of the Argentine isolate, Ar2, is supported by the comparisons of the product from the ORF 1 region of the genome which has a percent nucleic acid identity of 76.0%, 76.0%, and 84.9% with the prototype isolates from Burma (B1), Mexico (M1) and the US (US-1), respectively (Table 56). The divergence of the Ar2 isolate also is supported by the comparisons of the product from the ORF 2 region of the genome which had a percent nucleic acid identity of 85.8%, 82.4%, and 85.8% with the prototype isolates from Burma (B1), Mexico (M1) and the US (US-1), respectively (Table 57). The divergence of the Argentine isolate, Ar1, is supported by the comparisons of the product from the ORF 1 region of the genome which has a percent nucleic acid identity of 76.6%, 77.6%, and 85.7% with the prototype isolates from Burma (B1), Mexico (M1) and the US (US-1), respectively (Table 56). The nucleotide identities between the prototype isolates from Burma (B1), Mexico (M1) and the US (US-1), range between 75.5% to 82.4% over these two regions. Over these same regions, the isolates that comprise the Burmese-like group have much higher identities of 91.2% or greater. Although only a nested ORF2 PCR product was obtained from the Argentine isolate, Ar1, the divergence of the Ar2 isolate also is supported by the comparisons of this smaller product from the ORF 2 region of the genome which had a percent nucleic acid identity of 80.6% with the prototype isolates from Burma (B1), Mexico (M1) and the US (US-1) (Table 57). At the nucleotide level, the percent identities between the Austrian, Argentine, Greek, Italian and US isolates (excluding the identity between US-1 and US-2) range from 80.6% to 89.8% for the ORF 1 product (Table 56). At the nucleotide level, the percent identities between the Austrian, Argentine, Greek, Italian and US isolates (excluding the identity between US-1 and US-2 and Ar-1 and Ar-2) range from 80.6% to 89.2% for the ORF 2 product (Table 57). These values are lower than the lowest percent nucleotide identities between any Burmese-like isolates, which are 91.2% or greater for ORF 1 and ORF 2.

TABLE 56
Nucleotide and deduced amino acid identity between
isolates of HEV over 371 base (123 amino acid) ORF 1 fragment
Nucleotide Identity

| | | | | | | | | | | | | | | | | | | |
|---|---|---|---|---|---|---|---|---|---|---|---|---|---|---|---|---|---|---|
| Ar1 | 88.4 | 89.8 | 84.4 | 81.9 | 85.4 | 85.7 | 85.2 | 82.5 | 76.6 | 76.6 | 79.0 | 78.2 | 79.2 | 77.1 | 78.4 | 76.8 | 78.7 | 77.6 |
| 98.4 | Ar2 | 87.9 | 80.6 | 81.1 | 84.4 | 84.9 | 85.4 | 83.3 | 76.0 | 76.6 | 74.9 | 75.7 | 75.7 | 74.1 | 77.4 | 76.8 | 75.7 | 76.0 |
| 100 | 98.4 | Au1 | 85.2 | 81.1 | 86.0 | 87.9 | 87.1 | 84.6 | 77.1 | 77.6 | 76.8 | 76.6 | 77.6 | 76.6 | 77.4 | 78.2 | 77.1 | 78.2 |
| 99.1 | 97.6 | 99.1 | G1 | 84.4 | 84.1 | 81.9 | 82.5 | 83.0 | 78.4 | 77.9 | 77.6 | 78.2 | 77.9 | 76.6 | 77.6 | 78.2 | 77.4 | 76.6 |
| 98.4 | 96.7 | 98.4 | 99.2 | G2 | 81.7 | 83.8 | 83.0 | 81.9 | 78.2 | 77.6 | 78.2 | 77.9 | 78.4 | 77.1 | 77.9 | 77.6 | 78.7 | 76.8 |
| 97.6 | 95.9 | 97.6 | 96.7 | 96.7 | It1 | 84.6 | 86.8 | 84.9 | 77.6 | 77.6 | 77.1 | 77.4 | 77.4 | 76.3 | 77.6 | 77.4 | 77.6 | 78.4 |
| 99.2 | 97.6 | 99.2 | 98.4 | 97.6 | 96.7 | US-1 | 91.9 | 90.8 | 75.5 | 74.9 | 75.2 | 75.2 | 75.7 | 75.2 | 76.6 | 75.5 | 76.0 | 76.6 |
| 100 | 98.4 | 100 | 99.2 | 98.4 | 97.6 | 99.2 | US-2 | 89.9 | 75.2 | 75.4 | 75.2 | 75.4 | 76.0 | 74.9 | 75.7 | 75.7 | 76.3 | 77.6 |
| 97.6 | 95.9 | 97.6 | 96.7 | 95.9 | 95.1 | 96.7 | 97.6 | S1 | 76.6 | 76.6 | 74.7 | 74.9 | 74.4 | 73.6 | 75.2 | 74.9 | 75.2 | 75.7 |
| 91.1 | 90.2 | 91.1 | 90.2 | 90.2 | 92.7 | 91.9 | 91.1 | 88.6 | B1 | 98.7 | 94.6 | 94.6 | 94.9 | 94.3 | 94.3 | 96.0 | 94.6 | 79.0 |
| 91.1 | 90.2 | 91.1 | 90.2 | 90.2 | 92.7 | 90.2 | 91.1 | 89.6 | 98.4 | B2 | 93.8 | 93.8 | 94.1 | 93.5 | 93.5 | 95.1 | 93.8 | 78.4 |
| 89.4 | 88.6 | 89.4 | 88.6 | 88.6 | 91.1 | 90.2 | 89.4 | 87.0 | 98.4 | 96.7 | C1 | 97.8 | 98.1 | 96.8 | 92.7 | 91.6 | 97.3 | 79.8 |
| 90.2 | 89.4 | 90.2 | 89.4 | 89.4 | 91.9 | 91.1 | 90.2 | 87.8 | 99.2 | 97.6 | 99.2 | C2 | 98.7 | 97.6 | 93.8 | 91.6 | 98.4 | 79.5 |
| 90.2 | 89.4 | 90.2 | 89.4 | 89.4 | 91.9 | 91.1 | 90.2 | 87.8 | 99.2 | 97.6 | 99.2 | 100 | C3 | 97.3 | 93.5 | 91.9 | 98.1 | 79.5 |
| 89.4 | 88.6 | 89.4 | 88.6 | 88.6 | 91.9 | 90.2 | 89.4 | 87.0 | 98.4 | 96.7 | 99.2 | 99.2 | 99.2 | C4 | 93.0 | 91.9 | 97.0 | 78.7 |
| 90.2 | 89.4 | 90.2 | 89.4 | 89.4 | 91.9 | 91.1 | 90.2 | 87.8 | 99.2 | 97.6 | 97.6 | 98.4 | 98.4 | 97.6 | I1 | 91.4 | 93.3 | 79.2 |
| 88.6 | 87.8 | 88.6 | 87.8 | 87.8 | 90.2 | 89.4 | 88.6 | 86.2 | 97.6 | 95.9 | 95.9 | 96.7 | 96.7 | 95.9 | 96.7 | I2 | 91.6 | 78.4 |
| 90.2 | 89.4 | 90.2 | 89.4 | 89.4 | 91.9 | 91.1 | 90.2 | 87.8 | 99.2 | 97.6 | 99.2 | 100 | 100 | 99.2 | 98.4 | 96.7 | P1 | 78.7 |
| 92.7 | 91.1 | 92.7 | 91.9 | 91.9 | 94.3 | 93.5 | 92.7 | 90.2 | 95.9 | 95.1 | 94.3 | 95.1 | 95.1 | 94.3 | 95.1 | 93.5 | 95.1 | M1 |

Amino Acid Identity

TABLE 57
Nucleotide and deduced amino acid identity between isolates
of HEV over 148 base (49 amino acid)* ORF 2 fragment
Nucleotide Identity

| | | | | | | | | | | | | | | | | | | |
|---|---|---|---|---|---|---|---|---|---|---|---|---|---|---|---|---|---|---|
| Ar1 | 91.8 | 87.8 | 81.6 | 82.7 | 83.7 | 80.6 | 82.7 | 87.8 | 80.6 | 80.6 | 80.6 | 80.6 | 80.6 | 80.0 | 82.7 | 79.6 | 80.6 | 80.6 |
| 100 | Ar2 | 88.5 | 83.8 | 86.5 | 87.2 | 85.8 | 85.1 | 90.5 | 85.8 | 85.1 | 83.8 | 85.1 | 85.1 | 84.5 | 85.1 | 85.1 | 86.5 | 82.4 |
| 100 | 100 | Au1 | 83.1 | 88.5 | 89.2 | 83.1 | 85.8 | 87.8 | 85.1 | 83.8 | 83.1 | 83.8 | 83.8 | 83.1 | 84.5 | 83.1 | 82.4 | 79.1 |
| 100 | 100 | 100 | G1 | 87.2 | 87.8 | 84.5 | 85.1 | 85.1 | 84.5 | 82.4 | 82.4 | 83.1 | 83.1 | 82.4 | 83.1 | 82.4 | 83.8 | 81.1 |
| 100 | 100 | 100 | 100 | G2 | 83.1 | 82.4 | 85.1 | 87.8 | 85.1 | 84.5 | 82.4 | 83.8 | 83.8 | 83.8 | 83.8 | 83.1 | 84.5 | 79.1 |
| 100 | 100 | 100 | 100 | 100 | It1 | 87.8 | 85.8 | 85.8 | 83.8 | 83.1 | 82.4 | 83.1 | 83.1 | 82.4 | 83.8 | 81.8 | 82.4 | 79.7 |
| 96.9 | 98.0 | 98.0 | 98.0 | 98.0 | 98.0 | US-1 | 93.9 | 90.5 | 79.0 | 78.4 | 76.4 | 77.0 | 77.0 | 76.4 | 77.0 | 78.4 | 77.7 | 79.7 |
| 96.9 | 98.0 | 98.0 | 98.0 | 98.0 | 98.0 | 100 | US-2 | 91.2 | 82.4 | 80.4 | 79.7 | 80.4 | 80.4 | 79.3 | 80.4 | 81.8 | 81.1 | 81.8 |
| 96.9 | 98.0 | 98.0 | 98.0 | 98.0 | 98.0 | 100 | 100 | S1 | 83.8 | 84.5 | 82.4 | 83.1 | 83.1 | 82.4 | 83.1 | 83.1 | 83.8 | 83.8 |
| 96.9 | 98.0 | 98.0 | 98.0 | 98.0 | 98.0 | 95.9 | 95.9 | 95.9 | B1 | 98 | 94.6 | 95.3 | 95.3 | 94.6 | 96.6 | 97.3 | 93.9 | 82.4 |
| 96.9 | 95.9 | 95.9 | 95.9 | 95.9 | 95.9 | 93.9 | 93.9 | 93.9 | 98.0 | B2 | 93.9 | 94.6 | 94.6 | 93.9 | 95.9 | 95.3 | 93.2 | 80.4 |
| 96.9 | 98.0 | 98.0 | 98.0 | 98.0 | 98.0 | 95.9 | 95.9 | 95.9 | 100 | 98.0 | C1 | 98.0 | 98.0 | 96.6 | 96.6 | 91.9 | 96.6 | 81.8 |
| 96.9 | 98.0 | 98.0 | 98.0 | 98.0 | 98.0 | 95.9 | 95.9 | 95.9 | 100 | 98.0 | 100 | C2 | 100 | 98.6 | 97.3 | 92.6 | 98.6 | 82.4 |
| 96.9 | 98.0 | 98.0 | 98.0 | 98.0 | 98.0 | 95.9 | 95.9 | 95.9 | 100 | 98.0 | 100 | 100 | C3 | 98.6 | 97.3 | 92.6 | 98.6 | 82.4 |
| 96.9 | 98.0 | 98.0 | 98.0 | 98.0 | 98.0 | 95.9 | 95.9 | 95.9 | 100 | 98.0 | 100 | 100 | 100 | C4 | 96.6 | 91.9 | 97.3 | 81.8 |
| 96.9 | 98.0 | 98.0 | 98.0 | 98.0 | 98.0 | 95.9 | 95.9 | 95.9 | 100 | 98.0 | 100 | 100 | 100 | 100 | I1 | 93.9 | 95.9 | 83.8 |
| 93.8 | 95.9 | 95.9 | 95.9 | 95.9 | 95.9 | 93.9 | 93.9 | 93.9 | 98.0 | 95.9 | 98.0 | 98 | 98.0 | 98.0 | 98.0 | I2 | 91.2 | 83.8 |
| 96.9 | 98.0 | 98.0 | 98.0 | 98.0 | 98.0 | 95.9 | 95.9 | 95.9 | 100 | 98.0 | 100 | 100 | 100 | 100 | 100 | 98.0 | P1 | 83.1 |
| 96.9 | 98.0 | 98.0 | 98.0 | 98.0 | 98.0 | 95.9 | 95.9 | 95.9 | 95.9 | 93.9 | 95.9 | 95.9 | 95.9 | 95.9 | 95.9 | 93.9 | 95.9 | M1 |

Amino Acid Identity
* Over 98 base (32 amino acid) fragment for Ar1

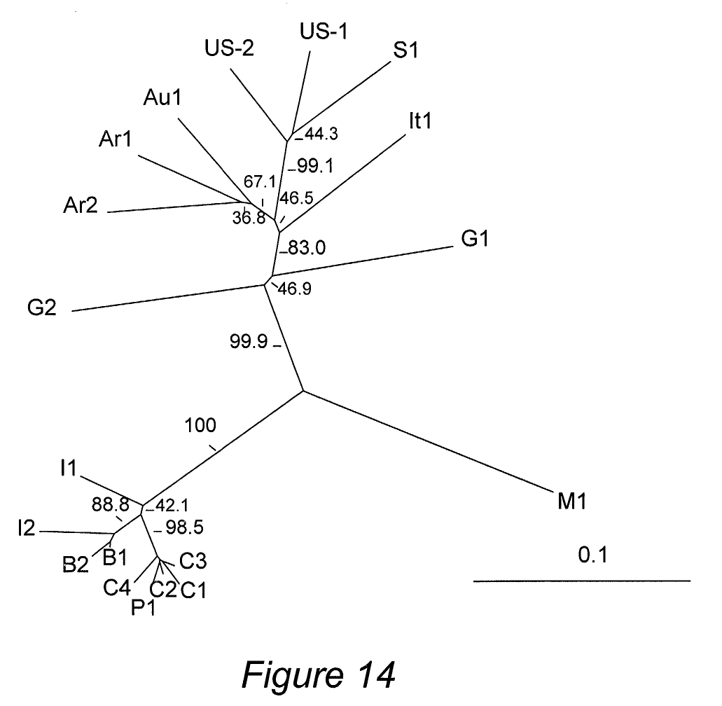

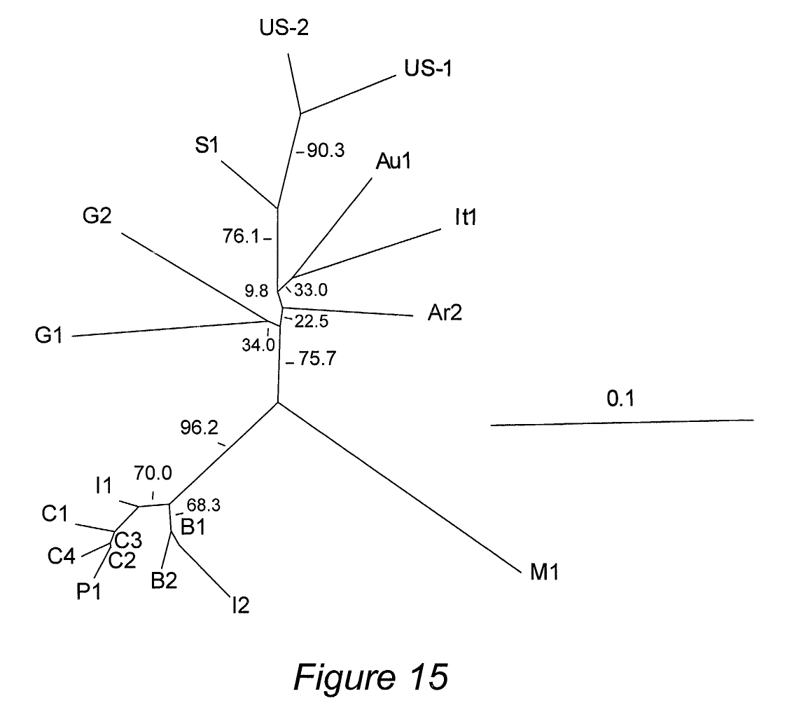

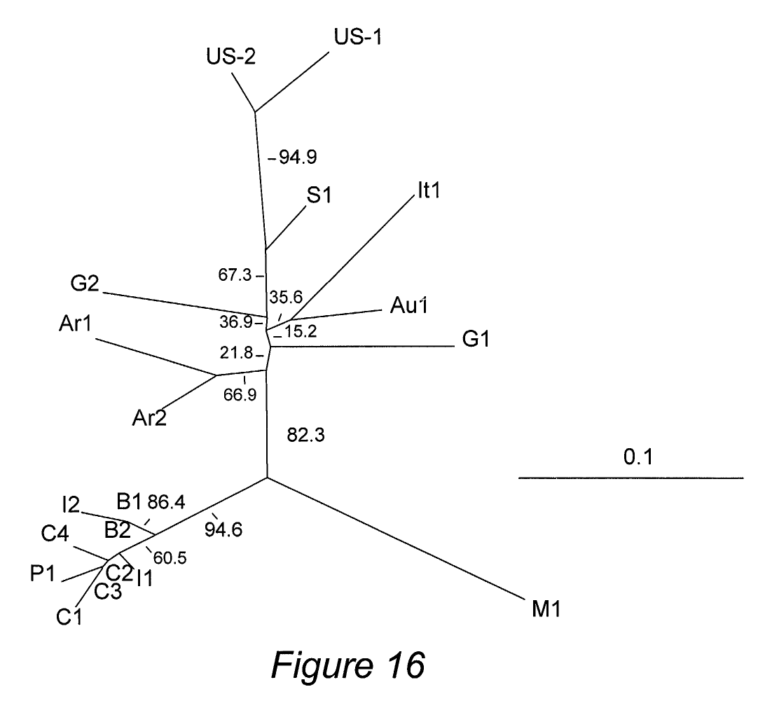

Comparisons of the ORF 1 and ORF 2 amplified sequences indicate that the isolates from the two patients from Argentina are quite distinct from each other, exhibiting 88.4% and 91.8% nucleotide sequence identity over these regions of ORF 1 and ORF 2, respectively. The value for ORF 1 is lower than the lowest percent nucleotide identities between any Burmese-like isolates, which is 91.4%. for ORF 1. However for ORF2, the nucleotide identity of 91.8% between the two isolates from Argentina is in the range observed for identities between the Burmese-like isolates and ORF 2, which may be due to the shorter length of the fragment. Phylogenetic analyses were performed as described in Example 7. The relative evolutionary distances between the viral sequences analyzed are readily apparent upon inspection of the unrooted phylogenetic trees generated from the pairwise distances, where the branch lengths are proportional to the relative genetic relationships between the isolates. The phylogenetic trees based on alignments of either 371 nucleotides from ORF 1 (FIG. 14), 148 nucleotides from ORF 2 (FIG. 15) which excludes Ar1, or 98 nucleotides from ORF 2 (FIG. 16), which includes Ar1, are quite similar in overall topology. The Burmese-like isolates and the Mexican isolate represent major branches at one end of the tree. The human US isolates form a distinct group distal to the Mexican and Burmese isolates. The swine HEV-like sequence is closely related to the US human isolates. The four European isolates and two Argentine isolates also form branches distal to the Mexican and Burmese isolates. The major branch between the US-type isolates, represented by the US, Greek, Italian, Austrian and Argentine isolates, and the Burmese-like and Mexican isolates is supported by a bootstrap value of 75.7% and greater in all trees.

### Example 15

### New Degenerate Primers

Degenerate primers derived from consensus oligonucleotide primers for HEV ORF 1 and ORF 2 were designed based on conserved regions between the full length sequences of isolates from Asia, Mexico, US as described in Example 11, as well as isolates from Greece and Italy. The ORF 1 primer is positioned within the methyltransferase region at nucleotides and 473-451 of the Burmese isolate (GenBank accession number M73218), and amplifies a product 417 nucleotides in length when used in combination with HEVConsORF 1-s1, SEQ ID NO:147; as described in Example11. The new ORF 1 primer combination includes:

| | |
|---|---|
| HEVConsORF 1-s1; | |
| CTGGCATYACTACTGCYATTGAGC; and | (SEQ ID NO:147) |
| HEVConsORF 1N-a1; | |
| CCRTCRARRCARTAGGTGCGGTC. | (SEQ ID NO:255) |

The new ORF 2 primer, at positions 6494-6470 of the Burmese isolate, produces a product 197 nucleotides in length when used in combination with HEVConsORF 2-s1; (SEQ ID NO:150); as described in Example11. The ORF 2 primers include:

| | |
|---|---|
| HEVConsORF 2-s1; | |
| GACAGAATTRATTTCGTCGGCTGG; and | (SEQ ID NO:150) |
| HEVConsORF 2N-a1; | |
| CYTGYTCRTGYTGGTTRTCATAATC. | (SEQ ID NO:256) |

For a second round of amplification, internal primers can be used to produce products 287 and 145 nucleotides in length for ORF 1 and ORF 2, respectively, as described in Example 11. The new combination of ORF 1 primers include:

| | |
|---|---|
| HEVConsORF 1N-s2; | |
| CYGCCYTKGCGAATGCTGTGG; and | (SEQ ID NO:257) |
| HEVConsORF 1-a2; | |
| GGCAGWRTACCARCGCTGAACATC. | (SEQ ID NO:178) |

The ORF 2 primers include:

| | |
|---|---|
| HEVConsORF 2-s2; | |
| GTYGTCTCRGCCAATGGCGAGC; and | (SEQ ID NO:152) |
| HEVConsORF 2N-a2; | |
| GYTCRTGYTGRTTRTCATAATCCTG. | (SEQ ID NO:258) |

PCR reactions contained 2 mM MgCl₂ and 0.5 μM of each oligonucleotide primer as per the manufacturer's instructions (Perkin-Elmer) and amplified using Touch-down PCR as described in Example 5. Amplified products were separated on a 1.5% agarose gel, stained with ethidium bromide, and analyzed for the presence of PCR products of the appropriate size. The primers were used to detect the presence of virus in serum containing HEV as described above and showed a marked increase in sensitivity over previous primers sets used in Example 11. These new primer combinations were found to be more sensitive with a number of different variants of HEV that included two new isolates from Argentina, Ar1 and Ar2, and a new isolate from Austria, Au1 (see example 14 above), as well as isolates from Greece, G1, and Egypt, Eg46. The results are presented in Table 58 below in which NT represents samples not tested, “−” represents no product band detectable by ethidium bromide staining, “+/−”represents a weak product band detectable by ethidium bromide staining, and “2+”, “3+” and “4+” represent increasing amounts of product as detected by ethidium bromide staining.

TABLE 58

| | ORF1 | | | | ORF2 | | | |
|---|---|---|---|---|---|---|---|---|
| | PCR1 | | PCR2 | | PCR1 | | PCR2 | |
| SAMPLE | Old Set | New Set | Old Set | New Set | Old Set | New Set | Old Set | New Set |
| Ar 1 | − | 2+ | 2+ | 4+ | 2+ | 4+ | 3+ | 4+ |
| Ar 2 | − | 2+ | 3+ | 4+ | +/− | +/− | − | 3+ |
| Au 1 | − | 2+ | 3+ | 4+ | − | 3+ | 3+ | 4+ |
| Eg46 | NT | NT | NT | NT | − | 3+ | 3+ | 4+ |
| G1 | − | − | 2+ | − | 3+ | 3+ | 3+ | 4+ |

Equivalents

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1. A method of detecting the presence of a US-type or US-subtype hepatitis E virus (HEV) or a naturally occurring variant thereof in a test sample, the method comprising the steps of:
(a) contacting the sample with a binding partner that binds specifically to a marker for said virus, which if present in the sample binds to the binding partner to produce a markers binding partner complex, and
(b) detecting the presence of said complex, the presence of said complex being indicative of the presence of said virus in the sample.

2. The method of claim 1, wherein said marker is an antibody capable of binding said Virus.

3. The method of claim 2, wherein said antibody is an immunoglobulin G or an immunoglobulin M.

4. The method of claim 2, wherein said binding partner is an isolated polypeptide chain.

5. The method of claim 4, wherein said polypeptide chain is immobilized on a solid support.

6. The method of claim 4, wherein said binding partner is a polypeptide chain selected from the group consisting of SEQ ID NOS:91, 92, and 93, including naturally occurring variants thereof.

7. The method of claim 4, wherein said binding partner is a polypeptide chain comprising the amino acid sequence set forth in SEQ ID NO:173 or SEQ ID NO:175.

8. The method of claim 4, where said binding partner is a polypeptide chain comprising the amino acid sequence set forth in SEQ ID NO:174 or SEQ ID NO:176.

9. The method of claim 4, wherein said binding partner is a polypeptide chain selected from the group consisting of SEQ ID NOS:166, 167 and 168, including naturally occurring variants thereof.

10. The method of claim 4, wherein said binding partner is a polypeptide comprising the amino acid sequence set forth in SEQ ID NO:223.

11. The method of claim 4, wherein said binding partner is a polypeptide comprising the amino acid sequence set forth in SEQ ID NO:224.

12. The method of claim 1, wherein said binding partner is an isolated antibody capable of binding specifically to a polypeptide chain selected from the group consisting of SEQ ID NOS:91, 92, 93, 166, 167, and 168, including naturally occurring variants thereof.

13. The method of claim 12, wherein said antibody is a monoclonal antibody.

14. The method of claim 1, wherein said marker is a polypeptide chain.

15. The method of claim 14, wherein said polypeptide chain is selected from the group consisting of SEQ ID NOS:91, 92, and 93, including naturally occurring variants thereof.

16. The method of claim 14, wherein said polypeptide chain comprises the amino acid sequence set forth in SEQ ID NO:173 or SEQ ID NO:175.

17. The method of claim 14, wherein said polypeptide chain comprises the amino acid sequence set forth in SEQ ID NO:174 or SEQ ID NO:176.

18. The method of claim 14, wherein said polypeptide chain is selected from the group consisting of SEQ ID NOS:166, 167, and 168, including naturally occurring variants thereof.

19. The method of claim 14, wherein said polypeptide chain comprises the amino acid sequence set forth in SEQ ID NO:223.

20. The method of claim 14, wherein said polypeptide chain comprises the amino acid sequence set forth in SEQ ID NO:224.

21. The method of claim 1, wherein said marker is a nucleic acid sequence defining at least a portion of a genome of said virus, or a complementary strand thereof.

22. The method of claim 1 wherein said binding partner is an isolated nucleic acid sequence that is capable of hybridizing under specific hybridization conditions to the nucleic acid sequences set forth in SEQ ID NOS:89 and 164.

23. The method of claim 1 wherein said binding partner is selected from the group consisting of SEQ ID NOS:126, 128, 147, 148, 150, 152, 177, 178, 255, 256, 257, and 258.

24. The method of claim 1 wherein said binding partner is an isolated polypeptide chain.

25. The method of claim 1 wherein said test sample is a mammalian cell line.

26. The method of claim 41 wherein said mammalian cell line is a human fetal kidney cell line.

27. A method of detecting the presence of a hepatitis E virus (HEV) in a test sample, the method comprising the steps of:
(a) contacting the sample with a binding partner selected from the group consisting of SEQ ID NOS: 126, 128, 147, 148, 150, 152, 177, 178, 255, 256, 257, and 258 that binds specifically to a marker for said virus, which if present in the sample binds to the binding partner to produce a marker-binding partner complex, and
(b) detecting the presence of said complex, the presence of said complex being indicative of the presence of said virus in the sample.

28. An isolated polypeptide chain comprising the amino acid sequence set forth in SEQ ID NO:173, SEQ ID NO:174, SEQ ID NO:175, SEQ ID NO:176, SEQ ID NO:223 and SEQ ID NO:224.

29. An isolated antibody capable of binding specifically to a polypeptide chain selected from the group consisting of a polypeptide encoded by an ORF 1 sequence of a US-type or a US-subtype HEV, a polypeptide encoded by an ORF 2 sequence of a US-type or a US-subtype HEV, and a polypeptide encoded by an ORF 3 sequence of a US-type or a US-subtype HEV.

30. An isolated antibody capable of binding specifically to a polypeptide chain comprising the amino acid sequence set forth in SEQ ID NO:173, SEQ ID NO:175 or SEQ ID NO:224.

31. An isolated antibody capable of binding specifically to a polypeptide chain comprising the amino acid sequence set forth in SEQ ID NO:174, SEQ ID NO:176 or SEQ ID NO:223.

32. The isolated antibody of claim 30, wherein said antibody, under similar conditions, has a lower affinity for a polypeptide chain comprising the amino acid sequence set forth in SEQ ID NO:169 or 171.

33. The isolated antibody of claim 31, wherein said antibody, under similar conditions, has a lower affinity for a polypeptide chain comprising the amino acid sequence set forth SEQ ID NO:170 or 172.

34. The isolated antibody of claim 29 further comprising a detectable moiety.

35. An isolated nucleic acid sequence defining at least a portion of an ORF 1, ORF 2 or ORF 3 sequence of a US-type or US-subtype hepatitis E virus, or a sequence complementary thereto.

36. An isolated nucleic acid sequence capable of hybridizing under specific hybridization conditions to the nucleotide sequence set forth in SEQ ID NOS:89 and 164.

37. A vector comprising the isolated nucleic acid sequence of claim 35.

38. A host cell containing the vector of claim 37.

39. A method of immunizing a mammal against a US-type or US-subtype HEV, the method comprising administering to the mammal the polypeptide of claim 28 in an amount sufficient to stimulate the production of an antibody capable of binding specifically to the US-type or US-subtype hepatitis E virus.

40. A method of immunizing a mammal against a US-type or US-subtype HEV 1, the method comprising administering to said mammal the antibody of claim 29 in an amount sufficient to immunize said mammal against the US-type or US-subtype hepatitis E virus.

41. A method of immunizing a mammal against a US-type or US-subtype HEV 1, the method comprising administering to said mammal the antibody of claim 30 in an amount sufficient to immunize said mammal against the US-type or US-subtype hepatitis E virus.

42. A method of immunizing a mammal against a US-type or US-subtype HEV 1, the method comprising administering to said mammal the antibody of claim 31 in an amount sufficient to immunize said mammal against the US-type or US-subtype hepatitis E virus.

43. A method of immunizing a mammal against a US-type or US-subtype HEV, the method comprising administering to said mammal the host cell of claim 38 in an amount sufficient to immunize said mammal against the US-type or US-subtype hepatitis E virus.
